(19) United States
(12) Patent Application Publication
(10) Pub. No.: US 2012/0034613 A1
(43) Pub. Date: Feb. 9, 2012

(54) Apparatus and Method for Testing Relationships Between Gene Expression and Physical Appearance of Skin

(75) Inventors: Remona Gopaul, Spanish Fork, UT (US); Dale G. Kern, Hyde Park, UT (US); Janet F. Lephart, Orem, UT (US)

(73) Assignee: NSE Products, Inc., Provo, UT (US)

(21) Appl. No.: 13/196,288

(22) Filed: Aug. 2, 2011

Related U.S. Application Data
(60) Provisional application No. 61/370,190, filed on Aug. 3, 2010.

(51) Int. Cl. C12Q 1/68; C12M 1/34

(52) U.S. Cl. 435/6.12; 435/6.13; 435/287.2

(57) ABSTRACT

The disclosure is directed to apparatus and methods for testing relationships between gene expression and physical appearance of skin and methods of assessing the efficacy of skin anti-aging agents.

### CROSS-REFERENCE TO RELATED APPLICATIONS

This application claims priority to U.S. Provisional Application No. 61/370,190 filed Aug. 3, 2010, entitled “Apparatus and Method for Testing Relationships Between Gene Expression and Physical Appearance of Skin,” the entire content of which is hereby incorporated herein by reference in its entirety.

### FIELD

The present disclosure relates to apparatus and methods for testing to identify and select genes associated with certain physical attributes of skin and methods of assessing the efficacy of a skin anti-aging agent.

### BACKGROUND

The sequencing of the human genome and the continual development of analytical methods to quickly and inexpensively measure whole-genome gene expression changes has created an overload of information and a need for methods to organize, focus and reduce the data resulting from gene level analyses of biological processes.

Analysis of the biological process of aging may be performed at the genomic level. Skin is the largest organ of the human body. The skin aging process is influenced by many different extrinsic (e.g., environmental) or intrinsic (including genetic and biochemical pathway) factors.

However, examining all the changes at the human genome or genomic product level can be overwhelming, particularly when strategies of anti-aging are examined. In skin, there are thousands of changes in gene expression with chronological aging and photo-aging (Robinson et al., Genomic-driven insights into changes in aging skin, J. Drugs Dermatol., 2009, 8(7 Suppl):s8-11). Not only does genome-wide testing produce massive data sets, the literature reporting on genetic research is also constantly growing, making it difficult to bring together all desired knowledge and test results.

Focusing research resources on a smaller group of genes and/or associated proteins, and the biochemical pathways associated with the groups of genes and/or proteins may be used to reduce confusion and distraction from the many unknown or irrelevant factors and variables. In addition, there is a cost to including genes in an analysis. If fewer genes are analyzed due to a better focus, the costs of research are reduced.

On the other hand, for an organ as complex as skin and a problem such as aging that has multiple dimensions, too narrow a focus also may be problematic, by excluding genetic pathways that play a role in skin aging. An approach including an overall strategy to slow down physical manifestations of the aging process in skin by examining at the gene level several mechanisms of aging simultaneously may be used, instead of in-depth analysis of each individual gene.

The present disclosure has been developed against this backdrop.

### SUMMARY

In a first aspect, the present disclosure is directed to a method of testing to identify genes associated with one or more physical attributes of skin aging. The methods comprise exposing a first sample of human skin cells or tissue to an agent, determining a first set of expression levels of a plurality of genes in the first sample of human skin, comparing the first set of expression levels to a second set of expression levels, the second set of expression levels corresponding to expression levels of human skin tissue not exposed to the agent, to identify a first subset of genes having a fold change difference in expression level between the exposed and unexposed samples that meets a first, selected biological relevance level, selecting from the first subset of genes a second subset of genes, each gene being associated with a biochemical pathway associated with physical appearance of skin aging, selecting from the second subset of genes, at least one skin attribute subset of genes, each gene in the skin attribute subset being associated with a biochemical pathway relating to the skin attribute that is shown in the comparing step to have been regulated in a more youthful direction for that biochemical pathway and skin attribute, exposing a second sample of human skin tissue to the agent, determining the levels of expression for the at least one skin attribute subset of genes in the second sample of human skin tissue using a method for determining expression levels that is different than that used for the first sample of human skin tissue, and selecting a third subset of genes from the at least one skin attribute subset of genes whose expression levels in the second sample of human skin tissue meet a second, selected biological relevance level and whose direction of regulation conforms to the more youthful direction used in selecting the at least one skin attribute subset of genes.

In some embodiments the biochemical pathway associated with the physical appearance of skin aging comprises at least one of skin structural protein synthesis, skin structural degradation and maintenance, extracellular matrix assembly, cellular differentiation, skin barrier component synthesis, skin barrier integrity, water regulation, or regulation of melanin production and control.

In some embodiments, the skin attribute for the at least one skin attribute subset of genes is skin structure, skin pigmentation, skin hydration or cell turnover.

In another embodiment, the first, selected biological relevance level is about a two fold difference between the exposed and unexposed samples.

In some embodiments, the human skin tissue comprises skin cells comprising at least one of keratinocytes, fibroblasts, adipocytes, melanocytes or combinations thereof.

In another embodiment, the first set of expression levels of a plurality of gene comprises expression levels for essentially the full human genome.

In other embodiments, the method of determining expression levels that is different than that used for the first sample of human tissue is a method using an RNA quantification metric.

In some embodiments, in the step of selecting from the second subset of genes, at least one skin attribute subset of genes, each gene in the skin attribute subset being associated with a biochemical pathway relating to the skin attribute that is shown in the comparing step to have been regulated in a more youthful direction for that biochemical pathway and skin attribute comprising performing this step for a plurality of skin attribute subsets of genes, and the step selecting a third subset of genes from the at least one skin attribute subset of genes whose expression levels in the second sample of human skin tissue meet a second, selected biological relevance level and whose direction of regulation conforms to the more youthful direction used in selecting the at least one skin attribute subset of genes comprising performing this step for a plurality of skin attribute subsets of genes.

In another embodiment, the plurality of skin attribute subsets of genes are two or more skin attribute subset of genes selected from the group consisting of skin structure, skin pigmentation, skin hydration and cell turnover.

In other embodiments, the method further comprises determining the levels of expression for additional genes associated with a biochemical pathway associated with skin aging in the second sample of human skin tissue using a method for determining expression levels that is different than that used for the first sample of human tissue, and selecting for the third subset of genes those genes from the additional genes associated with a biochemical pathway associated with skin aging whose expression levels in the second sample of human skin tissue meet a second, selected biological relevance level and whose direction of regulation conforms to the more youthful direction of regulation of the associated biochemical pathway.

In a second aspect, a computer based system of testing to identify genes associated with one or more physical attributes of skin aging comprises a first instrument for exposing a first sample of human skin tissue to an agent and determining a first set of expression levels of a plurality of genes in the first sample of human skin, a computer module for comparing the first set of expression levels to a second set of expression levels, the second set of expression levels corresponding to expression levels of human skin tissue not exposed to the agent to identify a first subset of genes having a fold change difference in expression level between the exposed and unexposed samples that meet a first, selected biological relevance level, a computer module for accessing a stored data set identifying genes, each gene being associated with a biochemical pathway associated with physical appearance of skin aging and for selecting from the first subset a second subset comprising those genes also in the second subset, a computer module for selecting from the second subset of genes, at least one skin attribute subset of genes, each gene in the skin attribute subset being associated with a biochemical pathway relating to the skin attribute that is shown in the comparing step to have been regulated in a more youthful direction for that biochemical pathway and skin attribute, a second instrument for exposing a second sample of human skin tissue to the agent and for determining the levels of expression for the at least one skin attribute subset of genes in the second sample of human skin tissue using a method for determining expression levels that is different than that used for the first sample of human tissue, a computer module for selecting a third subset of genes from the at least one skin attribute subset of genes whose expression levels in the second sample of human skin tissue meet a second, selected biological relevance level and whose direction of regulation conforms to the more youthful direction used in selecting the at least one skin attribute subset of genes.

In some embodiments in the system, the skin attribute for the at least one skin attribute subset of genes is skin structure, skin pigmentation, skin hydration or cell turnover. In another embodiment in the system, the first, selected biological relevance level is about a two fold difference between the exposed and unexposed samples.

In other embodiments in the system, the human skin tissue comprises skin cells comprising at least one of keratinocytes, fibroblasts, adipocytes, melanocytes or combinations thereof.

In some embodiments in the system the first set of expression levels of a plurality of genes comprises expression levels for essentially the full human genome.

In another embodiment in the system, the second instrument for determining expression levels that is different than that used for the first sample of human tissue is an instrument using an RNA quantification metric.

In a third aspect, methods of assessing the efficacy of a skin anti-aging agent are disclosed. The methods comprise exposing a first sample of human skin tissue to an agent, determining a first set of expression levels of a plurality of genes in the first sample of human skin, comparing the first set of expression levels to a second set of expression levels, the second set of expression levels corresponding to expression levels of human skin tissue not exposed to the agent, to identify a first subset of genes having a fold change difference in expression level between the exposed and unexposed samples that meets a first, selected biological relevance level, selecting from the first subset of genes a second subset of genes, each gene being associated with a biochemical pathway associated with physical appearance of skin aging, selecting from the second subset of genes, at least one skin attribute subset of genes, each gene in the skin attribute subset being associated with a biochemical pathway relating to the skin attribute that is shown in the comparing step to have been regulated in a more youthful direction for that biochemical pathway and skin attribute, exposing a second sample of human skin tissue to the agent, determining the levels of expression for the at least one skin attribute subset of genes in the second sample of human skin tissue using a method for determining expression levels that is different than that used for the first sample of human skin tissue, selecting a third subset of genes from the at least one skin attribute subset of genes whose expression levels in the second sample of human skin tissue meet a second, selected biological relevance level and whose direction of regulation conforms to the more youthful direction used in selecting the at least one skin attribute subset of genes, and comparing the third subset of genes to a previously determined third subset of genes for a second agent, thereby showing the efficacy of the skin anti-aging agent.

In some embodiments of the method, the skin attribute for the at least one skin attribute subset of genes is skin structure, skin pigmentation, skin hydration or cell turnover.

In another embodiment, the method for determining expression levels that is different than that used for the first sample of human tissue is a method using an RNA quantification metric.

### BRIEF DESCRIPTION OF THE DRAWINGS

Those skilled in the art will understand that the drawings, described herein, are for illustration purposes only. The drawings are not intended to limit the scope of the present disclosure.

FIGS. 1A-1K are a flowchart schematically showing, in a simplified example, steps in a process for choosing genes for inclusion in a functional youth gene assembly.

FIG. 2 depicts percent of change over time for different sub-categories of wrinkles, including crow's feet, under eye, and cheek areas.

FIG. 3 depicts percent change over time for a variety of facial attributes.

FIG. 4 depicts percent change over time for corneometer measurements of skin hydration.

FIG. 5 depicts percent change over time for cutometer measurements of skin extensibility.

FIG. 6 depicts percent change over time for ultrasound measurements of skin density.

FIG. 7 shows a schematic diagram for a system, including gene expression level testing devices and data-processing components for carrying out the method disclosed, and data sets developed and used as the method proceeds.

FIG. 8 shows the relationships of various data sets and subsets developed leading to a confirmed skin attribute subset defining a functional youth gene assembly.

### DETAILED DESCRIPTION

### Definitions

The term “functional youth gene assembly” refers to groups of genes encompassing one or more biochemical pathways or mechanisms of aging, addressable for functional restoration or stabilization of a more youthful state in the skin.

The term “skin attributes” refers to characteristics or qualities of human skin.

The term “biochemical pathway associated with skin” refers to a sequence of reactions and interactions among genes/proteins leading to a specific biochemical end product relevant to at least one skin biological processes.

The term “biochemical pathway associated with physical appearance of skin aging” refers to a biochemical pathway that leads to biochemical end products that cause a less youthful state in the skin.

The term “skin anti-aging agent” refers to a substance that causes a biological or chemical change in the skin to reflect a more youthful state in the skin.

Reference is now made in detail to certain embodiments of systems and methods. The disclosed embodiments are not intended to be limiting of the claims. To the contrary, the claims are intended to cover all alternatives, modifications, and equivalents.

The present disclosure provides a system and method in which the genes expressed in human derived skin cells in a human equivalent skin tissue model are tested for linkage to the physical appearance of human facial skin aging. Human derived skin cells include, for example, fibroblasts, keratinocytes, adipocytes and melanocytes.

Gene expression profiling using high-throughput methodologies such as DNA microarrays has proven to be a powerful approach in exploring the complex processes of aging, which involve many genetic pathways. This technique allows researchers to scan essentially the entire genome for age-related changes in gene expression, or changes in gene expression as a result of anti-aging strategies. Such analyses have demonstrated an association between aging and widespread gene expression. (Solene M, Fortunel N O, Pageon H, Asselineau D, Aging alters functionally human dermal papillary fibroblasts but not reticular fibroblasts: A new view of skin morphogenesis and aging. PLoS ONE 3(12):e4066, 2008).

If one first examines genes associated with specific mechanisms of aging, then by grouping genes from several mechanisms (associated with attributes of aging or preserving youthfulness) one can compile a functional youth gene profile expression or a functional youth gene assembly that is associated with a specific tissue, such as the skin, and specific attributes of it.

### Agents

An agent generally refers to a substance that causes a change in tissue observed. An agent is chosen based on the ability (or expected ability) of the agent to affect signs of aging, presumably by reason of an effect on expression levels of genes or gene products, including epigenetic effects.

Salicin is an agent that has shown effects on multiple signs of skin aging. See Applicant's co-pending patent application Ser. No. 12/058,201, publication number US 2009/0246152 A1, hereby incorporated by reference in its entirety.

In some embodiments, the skin anti-aging agent chosen for the experimental testing is salicin. Salicin (C₁₃H₁₈O₇) or 2-(Hydroxymethyl)phenyl β-D-glucospyranoside) is an alcoholic beta-glycoside that contains D-glucose. Salicin is obtained from several species of the white willow bark tree. Salicin is commercially available as a white, crystalline, water soluble powder from, for example, Sigma-Aldrich (St. Louis, Mo.).

Other agents may be chosen based on the ability of the substance to bring about a biological or chemical effect in tissue to reflect a more youthful state of the tissue. Choice of an agent is dependent upon the objective one is trying to obtain. An agent could be chosen for its apparent wide-spectrum anti-aging benefits, including effect on skin hydration, skin structure, skin pigmentation and skin cell turnover, or an agent could be chosen specifically for a single objective, e.g., hydration of the skin, associated with a more youthful state of epidermal tissue. For example, ingredients such as retinoids, niacinamide, N-acetyl glucosamine have a wide spectrum of anti-aging benefits.

### DNA Microarray Technology

### Data Processing Overview

In various aspects, the present disclosure relates to the analysis of skin tissue samples by microarray-based technology and transforming the microarray output data into useful subsets of data identifying particular genes of interest.

In some embodiments, the methods of the present disclosure comprise analyzing at least one test sample of a skin anti-aging agent on human or human-derived skin tissue, by using microarray-based technology to obtain information relating to changes in expression levels, if any.

A reference sample is a sample that lacks the presence of a skin anti-aging agent. Test and reference samples may be obtained from a biological source comprising human or human-derived skin cells or human or human-equivalent tissue, by any suitable method of nucleic acid isolation and/or extraction. In various aspects, the test sample and the reference sample are extracted RNA.

Array hybridization experiments allow the analysis of thousands of genes in one experiment. Microarrays are solid supports made of either nylon or silicon which house thousands of transcripts at fixed locations. The DNA is printed, spotted or synthesized on the support. This method is based on hybridization probing which uses fluorescently labeled nucleic acids as probes to identify complementary sequences. Single stranded DNA is made up of 4 different nucleotides, adenine (A), thymine (T), guanine (G), and cytosine (C). Adenine pairs with thymine and guanine pairs with cytosine. Hybridization occurs when a group of nucleotides finds their complementary partners. Microarray experiments measure the level of hybridization of each DNA on the support via fluorescently labeled tags.

There are three different types of probes that are commonly used in hybridization experiments: genomic DNA probes, cDNA probes and oligonucleotide probes. This provides the different terms namely “DNA array”, “cDNA array” or “oligonucleotide array” depending on what type of probe is used. (Kumar, A., Goel, G., Fehrenbach, E., Puniya, K. A., Singh, K. Microarrays: The Technology, Analysis and Application, Engineering in Life Sciences 5(3), 215-222 (2005); Jiang, N. et al. Methods for evaluating gene expression from Affymetrix microarray datasets, BMC Bioinformatics 9, 284 (2008); Auer, H., Newsom, D. L., Kornacker, K. Expression Profiling Using Affymetrix GeneChip Microarrays, Methods Mol Biol. 509, 35-46 (2009)).

In conducting a DNA microarray experiment, total RNA is extracted from the samples to be tested. The purified RNA is then analyzed for quality and quantity (>1 micrograms). Reverse transcriptase is then used to transcribe the mRNA into cDNA. The nucleotides used to synthesize the cDNA are labeled with either a green or red dye, one color for reference conditions or the other color for experimental conditions. The test samples and the reference samples may be differentially labeled with any detectable substance or moieties. The detectable substances or moieties may be selected such that they generate signals that can be readily measured and such that the intensity of the signals is proportional to the amount of labeled nucleic acids present in the sample. The detectable substances or moieties may also be selected such that they generate localized signals, thereby allowing resolution of the signals from each spot on an array.

Methods for labeling nucleic acids are well-known in the art. For exemplary reviews of labeling protocols, label detection techniques and recent developments in the field, (see e.g., Kricka, Ann, Clin. Biochem. (2002), 39: 114-129; van Gijlswijk et al., Expert Rev. Mol. Diagn. (2001), 1:81-91; and Joos et al., J. Biotechnol. (1994), 35: 135-153). Standard nucleic acid labeling methods include: incorporation of radioactive agents, direct attachment of fluorescent dyes or of enzymes, chemical modification of nucleic acids to make them detectable immunochemically or by other affinity reactions, and enzyme-mediated labeling methods including, without limitation, random priming, nick translation, PCR and tailing with terminal transferase. Other suitable labeling methods include psoralen-biotin, photoreactive azido derivatives, and DNA alkylating agents. In various embodiments, test sample and reference sample nucleic acids are labeled by Universal Linkage System, which is based on the reaction of monoreactive cisplatin derivatives with the N7 position of guanine moieties in DNA (see, e.g., Heetebrij et al., Cytogenet. Cell. Genet. (1999), 87: 47-52).

Any of a wide variety of detectable substances or moieties can be used to label test and/or reference samples. Suitable detectable substances or moieties include, but are not limited to: various ligands; radioneuclides such as, for example, ³²P, ³⁵S, ³H, ¹⁴C, ¹²⁵I, ¹³¹I, and others; fluorescent dyes; chemiluminescent agents such as, for example, acridinium esters, stabilized dioxetanes, and others; microparticles such as, for example, quantum dots, nanocrystals, phosphors and others; enzymes such as, for example, those used in an ELISA, horseradish peroxidase, beta-galactosidase, luciferase, alkaline phosphatase and others; colorimetric labels such as, for example, dyes, colloidal gold and others; magnetic labels such as, for example, Dynabeads™ particles; and biotin, dioxigenin or other haptens and proteins for which antisera or monoclonal antibodies are available.

The microarray or chip used for testing is then incubated overnight with both reference and experimental cDNAs. Certain cDNA will hybridize with the complementary strands from its gene that is covalently bound to a grid spot on the chip. The chips are then washed to remove any unbound cDNAs. Two computerized images are then produced by scanning first to detect the grid spots containing cDNAs labeled with green dye, and second to detect the spots containing the red-labeled cDNAs. The computer also produces a combination of the two images showing a yellow spot for grids spots containing both red and green labeled cDNAs. These yellow spots represent transcripts that are expressed under both sets of conditions.

In addition to producing images, microarray experiments yields quantitative data for each spot on the chip, resulting in large datasets where bioinformatics tools are needed for complete analysis. Parametric t-test with a Benjamini and Hochberg false discovery rate correction is the most common statistical parameter used for microarrays to identify genes with a statistically significant p value equal to or less than 0.05 and with a fold change of 2.0 and greater (or other suitable expression level criterion, expressed as a fold change threshold or otherwise). Genes are then either grouped by biological function or relation to a particular disease, depending on the objectives of the study.

These array-based methods of genetic analyses for skin cell samples allow the research analyst to develop data for essentially the entire human genome as an initial step, but economics and the need to limit focus make it desirable to analyze this data with a goal of limiting the number of genes addressed in later analysis steps. Techniques to permit focusing of resources on particular conditions, mechanisms and interventions can save time and cost.

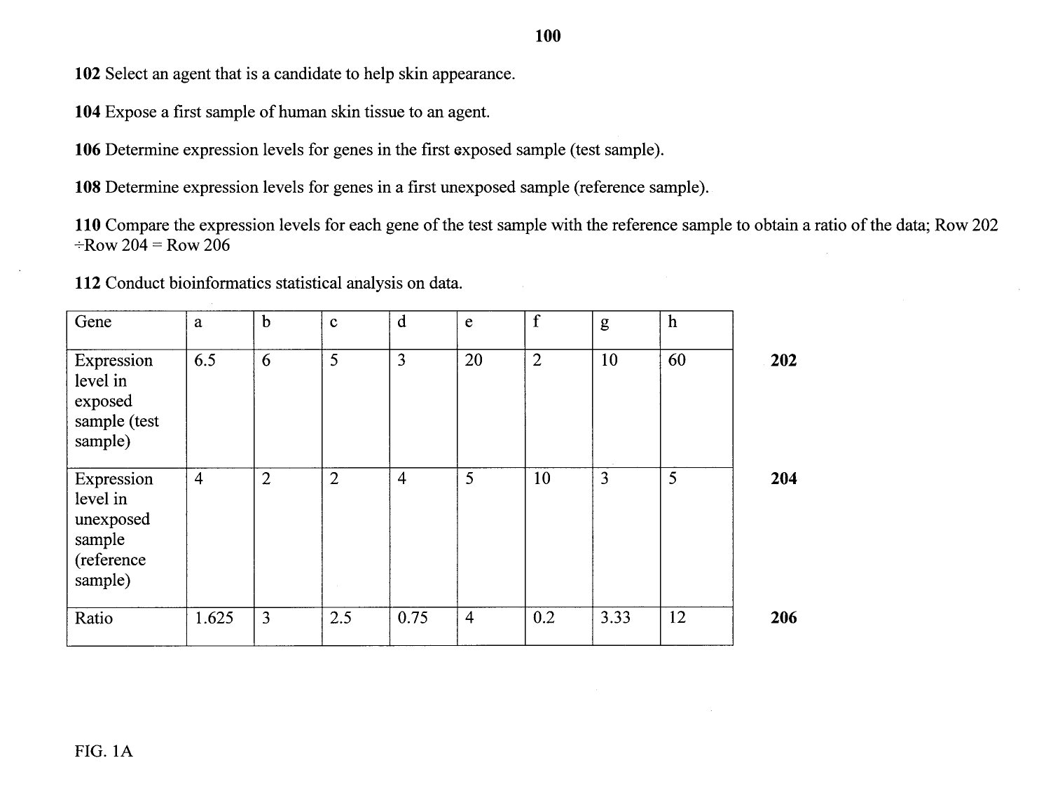

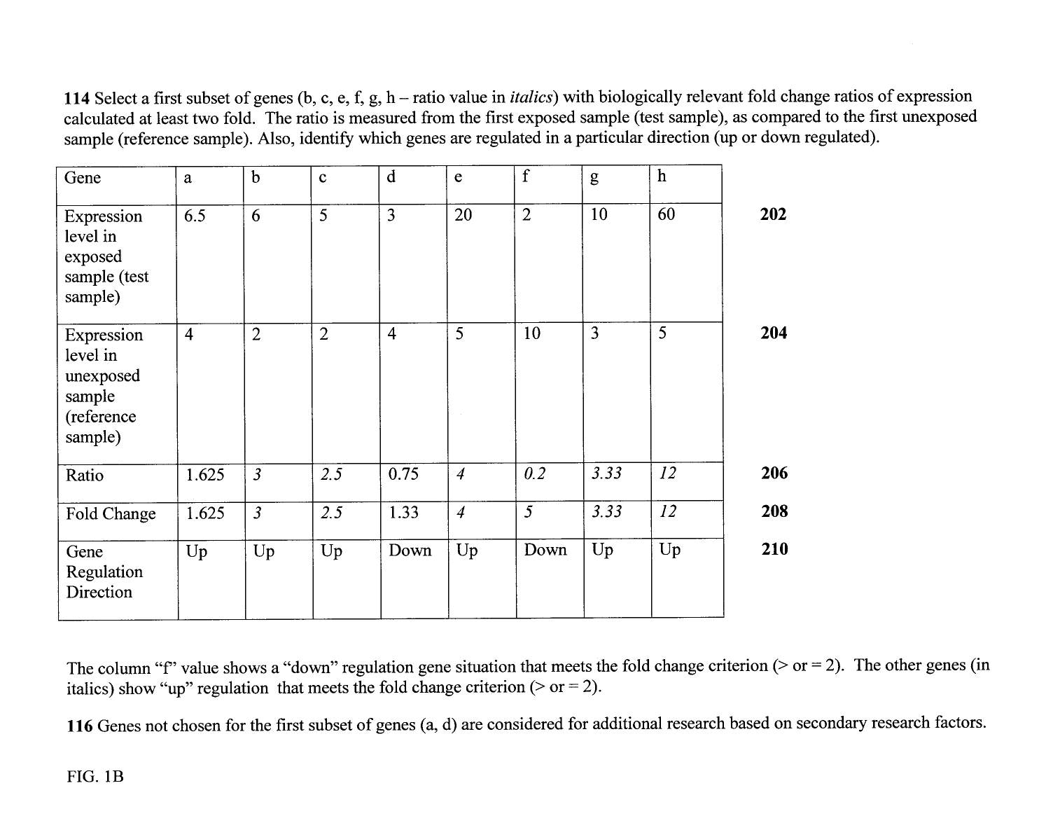

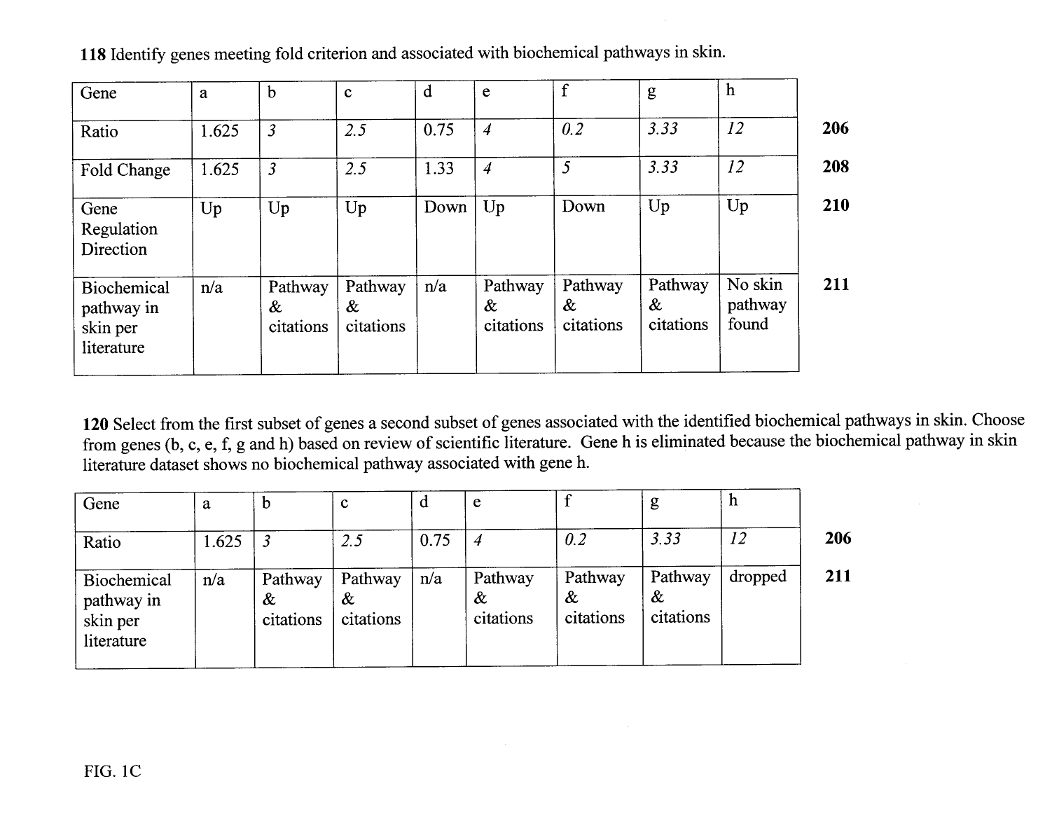

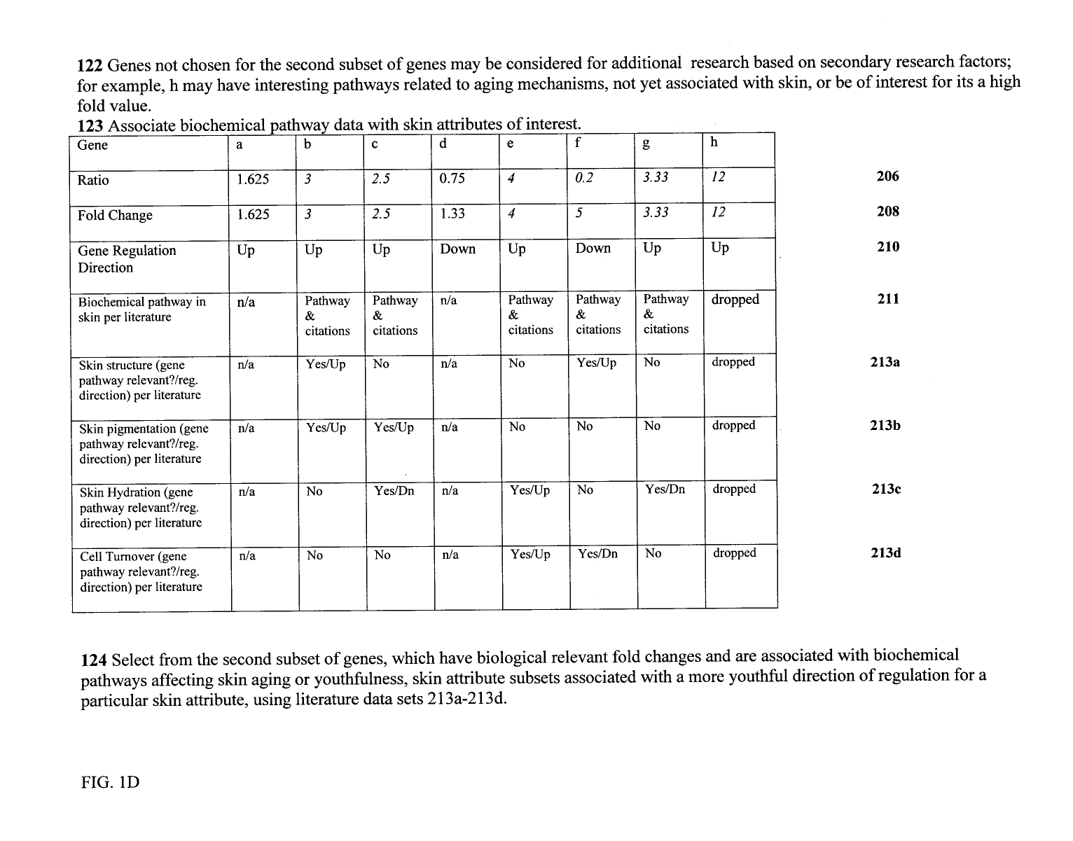

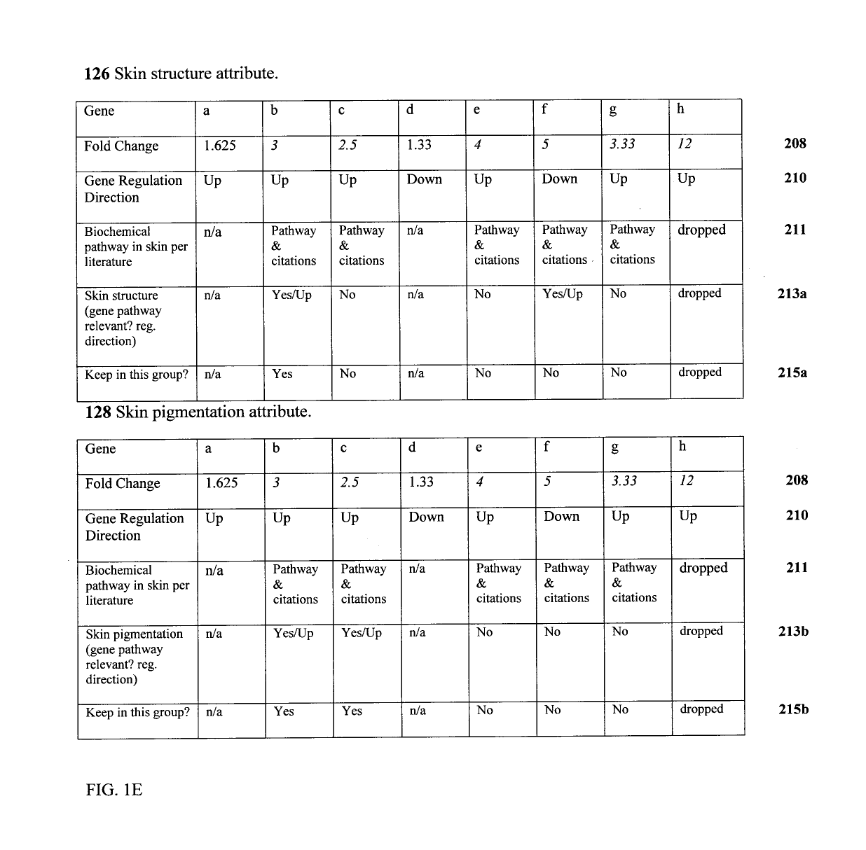

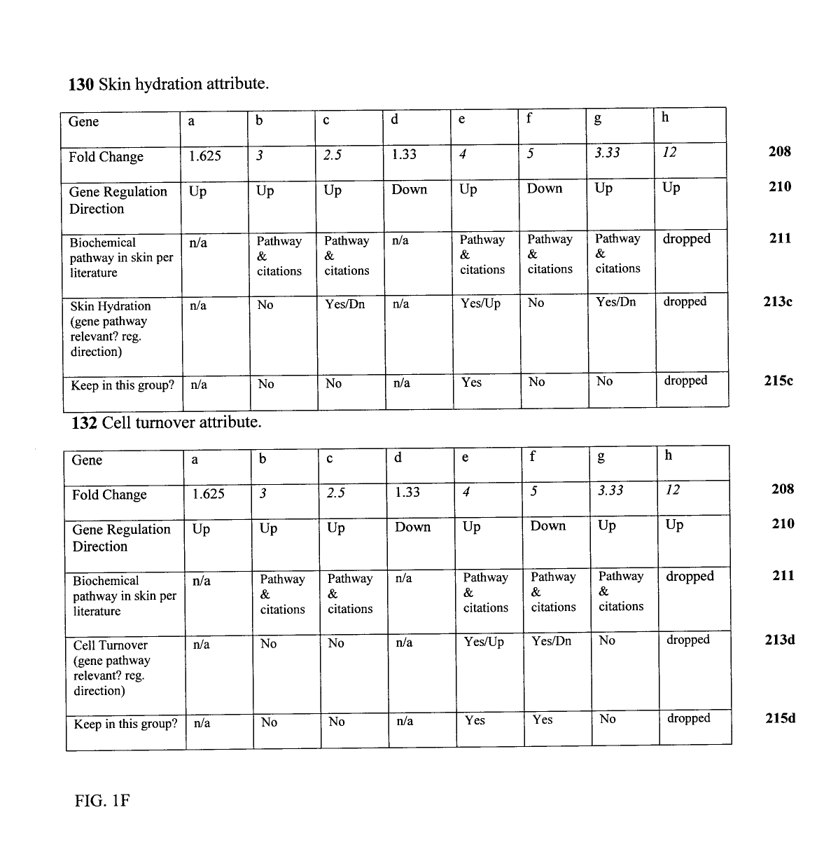

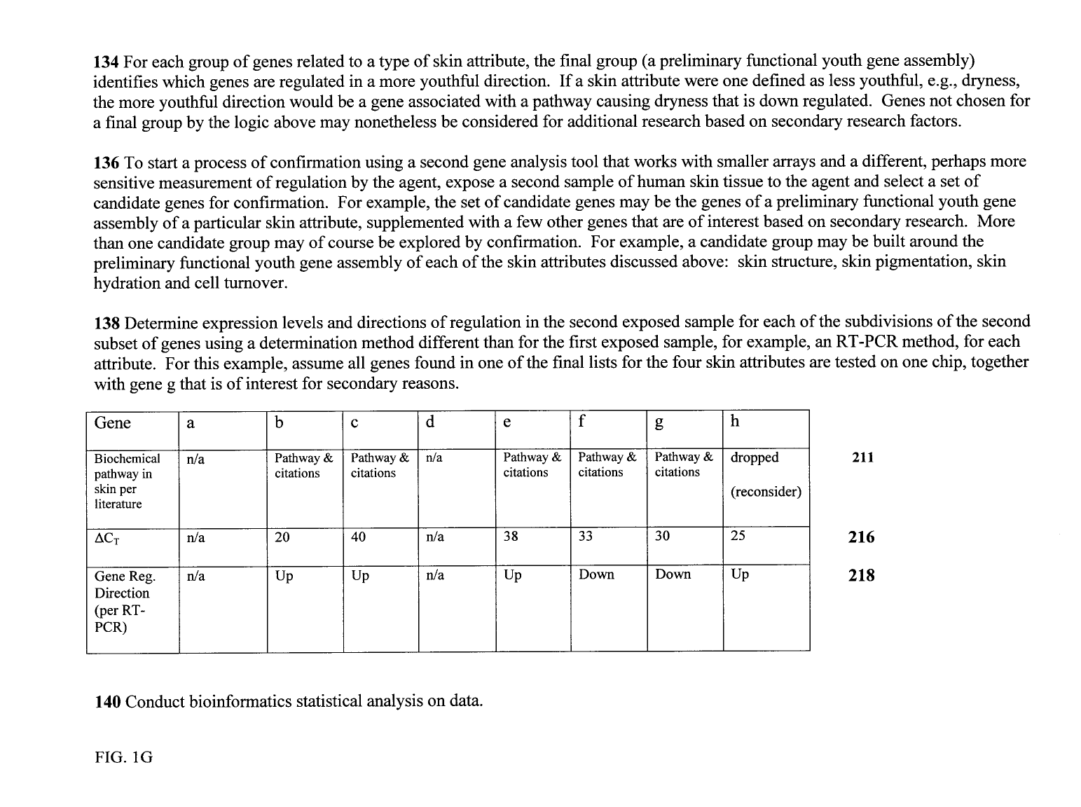

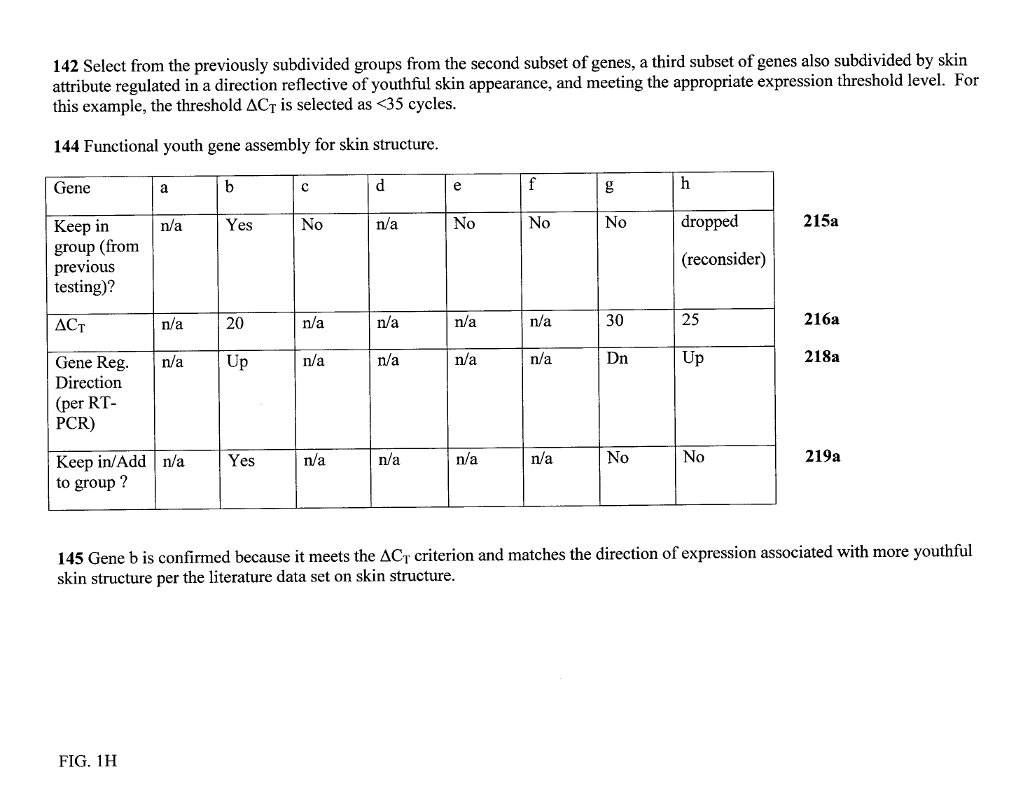

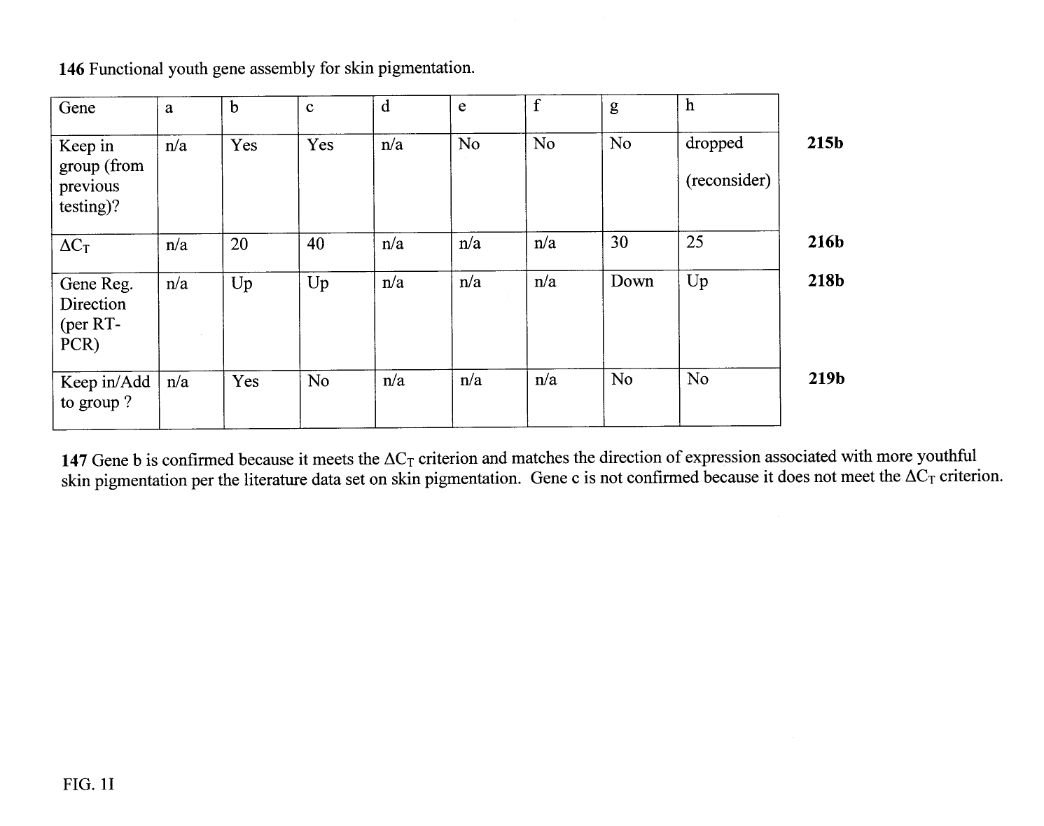

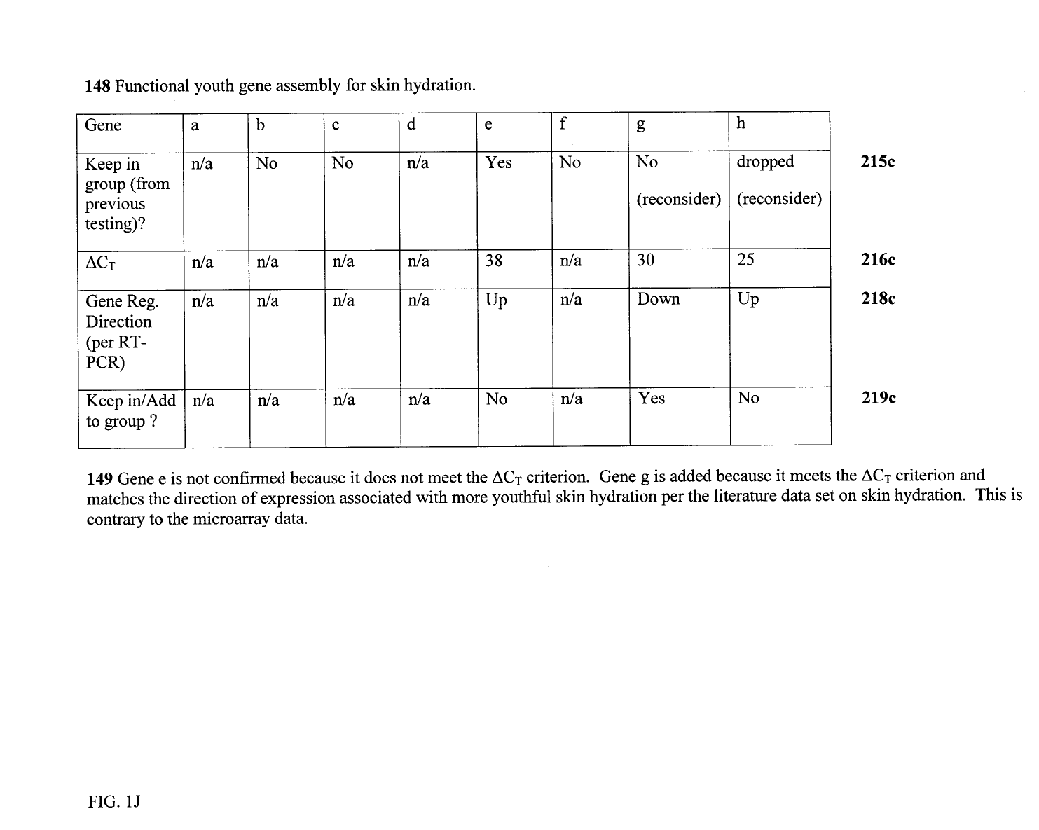

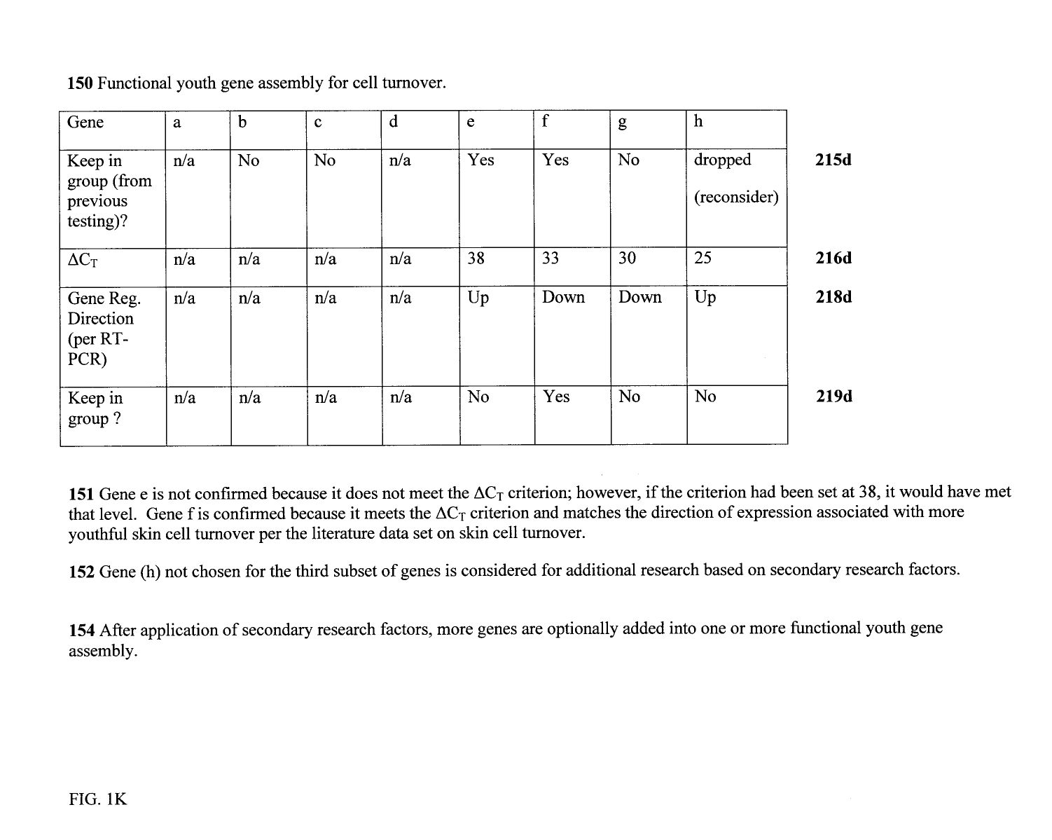

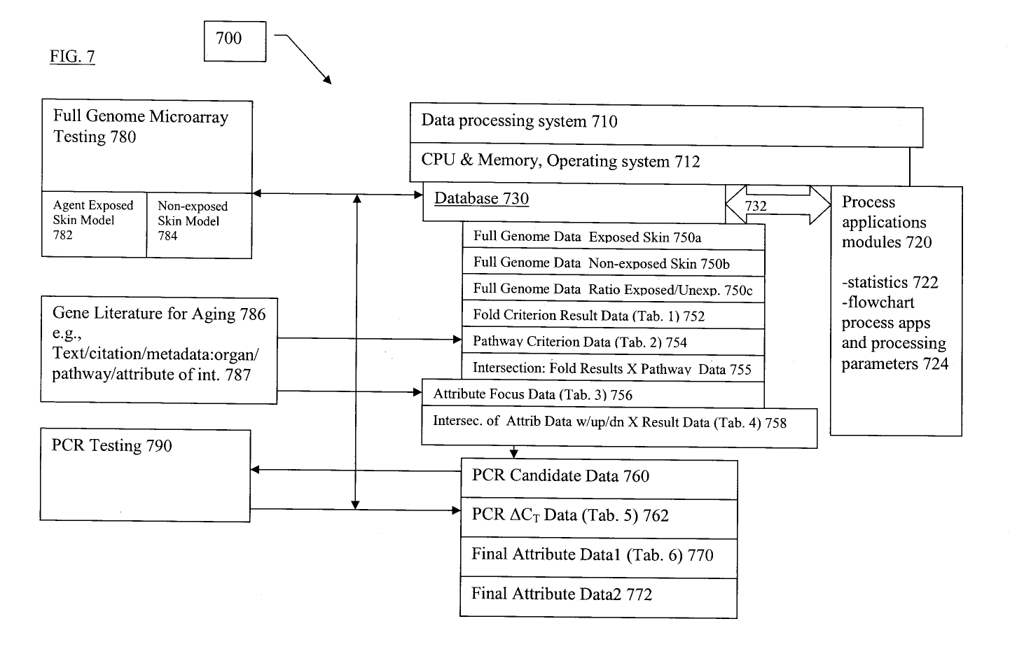

The array-based data is of sufficient volume that it is desirable (and likely necessary) to carry out the analysis that transforms a genome-wide set of data from microarray equipment into smaller, focused sets of data using a computer-based system. FIG. 7 shows a schematic diagram for a system 700, including data-processing components, for carrying out the method disclosed, including data sets developed and processed as the method proceeds. System 700 includes a data processing system 710 that may be implemented with a desktop computer, a cluster of computers, a group of computer resources in a cloud, a supercomputer or any other configuration of at least one CPU, memory and an operating system 712 that permits data from a full genome microarray 780 to be received in a database 730. The system 700 further includes various process application modules 720, including statistics modules 722 and other data processing modules and parameters 724 for performing steps outlined in the flowchart of FIGS. 1A-1K that carry out the data processing described in greater detail below. The database 730 receives from the microarray 780 raw or semiprocessed data that are produced in one or more runs of the microarray. This data may include data comprising a full genome data set 750a derived from a sample of agent-exposed tissue 782 and from a full genome control data set 750b derived from a sample of tissue not agent-exposed 784. These data will be subject to computations that produce a further data set 750c, with computed ratios by gene of levels of gene expression in exposed and unexposed tissue (782/784) as detected by microarray 780. The computations further include applying to this data set of ratios 750c a criterion for whether the level of gene expression associated with the agent, is significant, in either an up-regulated or a down-regulated direction. The criterion may be a selected and stored parameter in the applications modules 720, specifically in the data processing modules and parameters 724. Application of a stored “fold” criterion to the ratio data set 750c for the full genome yields a fold criterion data set 752, i.e., a data set listing genes found by testing to have a level of expression in the agent-exposed tissue that meet the fold criterion.

Also in the database 730 are data sets developed from literature 786 on the biological pathways that have been reported as associated with various genes. One data set 754 identifies genes reported as having biological pathways that are significant for skin. (The data set 754 may be derived from literature by automated keyword and/or metadata analysis of the text of scientific journals, patents or other sources reporting on activity of particular genes, including non-published studies, or may be built by the input of one or more scientific experts. For this purpose, it may be helpful to build a database of sources annotated with metadata 787 that permit ready identification of each gene associated with skin (or other organs) and, for genes associated with skin, as addressing biochemical pathways associated with particular skin attributes.) This data set may be used in an intersection analysis to identify the genes in a larger test result data set, for example the full genome microarray data sets 750a, 750b or the fold criterion result data set 752, that are related to skin and also meet the specified fold criterion that led to the fold criterion result data set 752, forming a new skin pathway intersection data set 755. Thus, the literature data set 754 may be used as a filter for the test data to provide a focus on genes for which data from the literature data set supports a pathway of interest in skin.

The data sets derived from literature can also have a narrower focus within the broader area of skin. For example, a skin attribute data set 756 may be derived that identifies genes reported as having biological pathways that are significant for a particular attribute of skin, such as skin structure or skin pigmentation. Such a data set may be used in an intersection analysis to identify the genes in a larger test result data set, for example the skin pathway intersection data set 755, to identify genes that are related to a specific skin structure and also meet the specified fold criterion that led to the fold criterion data set 752, forming a new skin attribute intersection data set 758. The skin attribute intersection data set 758 is then focused on genes related to one specific skin structure; an attribute data set 756 for another skin attribute, e.g., skin pigmentation, can lead to a different skin attribute intersection data set 758. At this level of focus, assuming the goal of focus is to identify genes associated with more youthful manifestations of a skin attribute, the skin attribute data sets also include up or down regulation coding. That is, if a gene is associated with a particular skin attribute, it may be reported as involved in either the up or down regulation of a pathway that either leads to more or less youthful appearance. To the extent a goal is to identify agents to influence biological pathways that enhance youthfulness, it is significant to identify both the genes that can be up-regulated to cause a more youthful state, as well as the genes that can be down-regulated to reduce action of a pathway that leads to less youthful state. Thus, the skin attribute data sets 756 are coded to identify for each gene, the up or down regulation of a pathway that either leads to more or less youthful appearance as to the particular skin attribute involved.

The results embodied in a skin attribute intersection data set 758 may be considered preliminary and will be considered more reliable if they can in some way be confirmed, or refined. The present system in one embodiment develops and analyzes further data to provide possible confirmation and refining. As seen in FIG. 7, a second instrument for determining levels of gene expression, such as an instrument 790 that relies on PCR techniques for determining expression levels may be employed. The candidate genes whose expression level is the focus for testing in this instrument are selected based on one or more of the skin attribute data sets 758. In one embodiment, the candidate genes identified in one of these sets 758 may be supplemented for second instrument testing with other candidate genes of interest, based on secondary research or simply because they are useful to provide reference values. In one embodiment, the other candidate genes of interest may be selected by searching in literature and in fold criterion result data 752 for genes associated with aging (although not specifically with skin aging) and showing up regulation of an anti-aging biochemical pathway. Thus, a PCR Candidate data set 760 is assembled to define the analytical focus of gene expression level testing in the PCR Testing instrument 790.

The test result data from the PCR Testing instrument 790 show levels of gene expression for the selected candidate genes when the same agent used to develop data in the upper portion of FIG. 7 is exposed to a new tissue sample. In one embodiment, this is the same type of skin tissue sample as used to develop data in microarray 780 in the upper portion of FIG. 7. The level of gene expression data, e.g., PCR ΔC_{T} data 762, are developed for each selected candidate gene. These data are statistically cleaned and processed, then correlated with a particular skin attribute intersection data set 758 having the same genes or with data for corresponding genes found in data sets 752 or 755. In some instances, for a particular candidate gene, a high level of expression in the same direction as was associated with more youthful appearance in the intersection data set 758 will confirm that the gene and its associated biological pathway(s) appears to be capable of agent stimulation for expression that leads to more youthful appearance as to a particular skin attribute. A set of such genes then may become part of a final functional youth gene assembly 770 for the skin attribute. A different set of candidate data 760 based on a different skin attribute intersection data set 758, and the set of PCR ΔC_{T} test data 762 developed for those candidates may become part of a final functional youth gene assembly 772 for a different skin attribute. Each such assembly would appear to provide a more useful and economic focus for further study than any whole genome study. The processes leading the various data sets identified in FIG. 7 are discussed in greater detail below.

The discussion below proceeds at two levels. At one level, with reference to FIGS. 1A-1K, a highly simplified example assuming a genome with only genes a-h and with entirely hypothetical data is used to show the process steps. At a second level, with reference to Tables 1-6, the same process is described, but Tables 1-6 show actual test data from lab testing using actual skin tissue samples and equipment that finds expression levels for actual genes that are included in the human genome and the literature data sets (754, 756) are derived from actual gene literature.

### Global Gene Expression Levels

In the present method and system, expression profiling for thousands of genes, substantially all of the genes of the human genome, is performed to determine a first set of expression levels of a plurality of genes in a first sample of agent-exposed human or human derived skin.

Genome refers to all nucleic acid sequences, coding and non-coding, present in each cell type of a subject. The term also includes all naturally occurring or induced variations of these sequences that may be present in a mutant or disease variant of any cell type, including, for example, tumor cells. Genomic DNA and genomic nucleic acids are thus nucleic acids isolated from a nucleus of one or more cells, and include nucleic acids derived from, isolated from, amplified from, or cloned from genomic DNA, as well as synthetic versions of all or any part of a genome.

For example, the human genome consists of approximately 3.0×10⁹ base pairs of DNA organized into 46 distinct chromosomes. The genome of a normal human diploid somatic cell consists of 22 pairs of autosomes (chromosomes 1 to 22) and either chromosomes X and Y (male) or a pair of chromosome Xs (female) for a total of 46 chromosomes.

In some embodiments, Affymetrix® DNA microarray technology is used for measuring global gene expression in human in vitro skin cultures. Microarrays are ideal for simultaneously measuring the effects of a test compound on the activity of thousands of genes in the human genome. (Microchip Methods in Diagnostics, vol. 509, chapter 3, Expression Profiling Using Affymetrix GeneChip Microarrays, Auer et al. (2009)).

For one embodiment, EpidermFT™ Skin Model (EFT-400) full thickness skin cultures (MatTek Corp, Ashland, Mass.) is used as a skin model. These cultures contain normal, human-derived epidermal keratinocytes from neonatal foreskin tissue and normal human-derived dermal fibroblasts, from mammary tissue. These cells are cultured to form a multilayered, highly differentiated model of the human dermis and epidermis. The model parallels human skin and is useful for in vitro testing, where a microarray is used to develop and collect data. Skin models typically contain human derived skin cells cultured to form a model of skin tissue. Generally, these skin models are referred to as “human equivalent skin tissue” or “human derived skin tissue”. Skin cells include keratinocytes, fibroblasts, adipocytes and melanocytes.

FIGS. 1A-1K show steps in a flowchart 100 with steps for choosing from the simplified hypothetical genes a-h, genes for a functional youth gene assembly. Reference numerals associated with method steps and tabular data on flowchart 100 appear in the description of the method below.

The steps begin with selecting an agent that is a candidate to help skin appearance 102 (or explore effects of skin aging). In some embodiments, the agent tested is salicin at a concentration of 0.5% salicin, available from Symrise Corporation (Teterborro, N.J.). The salicin is dissolved in water. An agent that has some known effects on skin aging may be useful for revealing gene-based effects, but other agents may be selected.

The first samples of the human equivalent skin tissue are exposed to the skin anti-aging agent 104. Untreated cultures, or human skin tissues not exposed to the agent, serve as controls or reference samples. (See FIG. 7 at 782, 784)

RNA is extracted from each of the human skin cells, or cultures, using an RNeasy® Fibrous kit (Qiagen, Valencia, Calif.) following the manufacturer's protocol. (RNeasy® Fibrous Tissue Handbook, November 2006). cDNA is synthesized from 100 ng of total RNA, and then converted to biotin-labeled amplified RNA (aRNA) using an Affymetrix GeneChip® 3′ IVT Express kit, according to the manufacturer's instructions. (Affymetrix User Manual GeneChip® 3′ IVT Express Kit (2008)).

In some embodiments, the samples are hybridized to Affymetrix GeneChip® HG U133 Plus 2.0 microarrays, washed, stained and scanned according to Affymetrix protocols. The microarray laser scanner measures fluorescence intensities of all of the transcripts on the gene chip; the fluorescence of each transcript is compared among each of the samples. (See FIG. 7 at 780).

Results from these experiments reveal that the skin anti-aging agent selected for exploration may have influenced the activity of any of more than 15,000 genes in the human genome.

The activity is measured by determining expression levels for genes in the first exposed tissue sample (test sample) 106. This involves reading the array that has a human equivalent tissue model exposed to the agent. This results in a set of data that is stored in database 730 of a data processing system 710 for implementing the method described herein, (See FIG. 7 at 750a).

To give a basis for comparison, the method uses a reference tissue that has not been exposed to the agent. The same skin model is used to provide data on expression levels when the agent is not present. The reference level is measured by determining expression levels for genes in the first unexposed sample (reference sample) 108. Again, this results in a set of data that is stored in database 730 of a data processing system 710 for implementing the method described herein. (See FIG. 7 at 750b).

The agent-exposed and reference data are analyzed to determine the affects of agent exposure. The expression level data for each gene of the test sample are compared with the corresponding data of the reference sample to obtain a ratio of the data 110. (See FIG. 7 at 750c). At least part of the gathering, storing and comparing of the expression level data is performed by a computer, such as the data processing system 710, with CPU 712 (see FIG. 7) for performing data access and storage and various computations specified by software modules corresponding to the functions occurring at various described steps of this method.

A bioinformatics statistical analysis is conducted on data 112 to identify and characterize candidate genes. A Parametric t-test with a Benjamini and Hochberg false discovery rate correction is the most common statistical parameter used for microarrays to identify genes with a statistically significant p value equal to or less than 0.05 and with a selected biological relevance level, e.g., a fold change of 2.0 and greater. To help organize the results, genes are either grouped by biological function or relation to a particular disease, depending on the objectives of the study.

### Selecting First Subset of Genes

Flowchart 100 in FIG. 1A has an illustration of a table of the extremely simplified hypothetical example set of genes (for simplicity identified as “a-h”) with rows showing expression levels in the exposed sample 202, expression levels in unexposed sample 204, and the ratio 206 of row 204 compared to row 202 (row 202÷ row 204=row 206). (The data in rows 202 and 204 are entirely hypothetical, for purposes of an example, and row 206 shows a computation from the hypothetical data.)

FIG. 1B shows an expanded table of the example set of genes. Row 208 shows the ratios of row 206 as fold changes, reflecting that genes may be up-regulated or down-regulated.

The raw dataset from the first subset of genes, row 208, is then filtered, using bioinformatics methods, to identify and characterize genes. In particular, the data is filtered to focus on genes with biologically relevant fold change values. In data from a typical microarray device, e.g., data from the Affymetrix testing, the up or down regulation of the gene is also identifiable. The row 210 shows the regulation direction of the hypothetical gene.

In some embodiments at least part of the analysis of data is performed by a computer. Statistical data analysis may be carried out using GeneSpring GX software (version 10). A parametric t-test with a Benjamini and Hochberg false discovery rate correction is performed to identify genes with a statistically significant p value equal to or less than 0.05. (Statistical analysis may be performed by one or more of the process application modules 720. (See FIG. 7). In one set of data collected, the 0.05% salicin treated cultures (N=7) are compared with untreated control cultures (N=4) after 24 hours of stimulation.

Returning to the simplified example, at step 114, the system selects a first subset of genes (b, c, e, f, g and h) with biologically relevant fold changes in the level of expression, for example, those calculated to be at least a two fold change. (See row 208). As noted with reference to rows 202, 204, 206, the ratio is measured from the first exposed sample (test sample), as compared to the first unexposed sample (reference sample). The method also involves identifying which genes are regulated in a particular direction (up or down regulated). (See row 210). In one embodiment, a ratio of data in row 202 to data in row 204, shows up-regulation if the ratio is greater than 1 and down-regulation is the ratio is less that 1.

As shown in row 206, example genes b, c, e, f, g and h have ratios of 3, 2.5, 4, 0.2, 3.33 and 12, respectively. Thus, with a fold change threshold selected at 2, the table in FIG. 1B shows the example genes b, c, e, f, g and h, meet the fold change criterion. This is the first subset as described for the hypothetical example.

In the test data from skin model experiments, fold changes of at least about 2.0, about 2.0, of at least about 3.0, between about 2.0 and about 4.0, between about 2.0 and about 7.0, and even between 2.0 and about 200.0 may be selected as biologically relevant. Fold change significance may vary based on the instrument used for testing, tissue sample and other factors. For Affymetrix DNA microarray data, using the specific statistically scientific parameter, a fold change of 2 or more is biologically significant. (The fold change criterion is a selectable parameter 724 in process application modules 720.)

As shown on row 206 of the table on FIG. 1B, example genes a and d have ratios of 1.625 (up), and 0.75 (down), respectively, or fold changes of 1.625 and 1.33, respectively, both below a fold change selection criterion of 2 for up-regulation or down-regulation. By comparison, for gene e, the expression level was higher in the exposed test sample than in the reference, so in comparison to the reference, this gene was up-regulated. The column “f” value would be a “down” regulation gene situation, with a fold change of 5 (a ratio of 2/10=0.2). Thus, gene fin the example also meets the fold change criterion. Genes not meeting the fold change criterion and not chosen for the first subset of genes (a and d, in the example) may be considered for additional research based on secondary research factors 116. (Secondary research factors are discussed below Table 6.)

Correspondingly, as will be seen below and in Table 1, the actual skin model data test results from the microarray instrument 780 were subjected to the same fold change criterion as in the simplified example; thus genes with fold changes reported below 2.0 were not included in any of the tables for skin model actual experimental results.

Table 1 below shows experimental data from a microarray for 0.05% salicin treated cultures (N=7) compared with untreated control cultures (N=4) corresponding to the simplified hypothetical data in the table of FIG. 1B. (See also, FIG. 7 at 752). This is a list of more than 2,300 genes tested by the Affymetrix microarray test methods with filtering of data as described in the example to focus on genes with biologically relevant fold change values. Table 1 thus shows the first subset for the microarray data.

TABLE 1

| | Fold | | | |
|---|---|---|---|---|
| Probe Set ID | change | Direction | Gene Symbol | Gene Title |
| 1556410_a_at | 24.649403 | up | KRTAP19-1 | keratin associated protein 19-1 |
| 210229_s_at | 16.55027 | up | CSF2 | colony stimulating factor 2 |
| | | | | (granulocyte-macrophage) |
| 205114_s_at | 15.552067 | up | CCL3 /// | chemokine (C-C motif) ligand 3 /// |
| | | | CCL3L1 /// | chemokine (C-C motif) ligand 3-like 1 |
| | | | CCL3L3 /// | /// chemokine (C-C motif) ligand 3-like |
| | | | LOC728830 | 3 /// similar to C-C motif chemokine 3- |
| | | | | like 1 precursor (Small-inducible |
| | | | | cytokine A3-like 1) (Tonsillar |
| | | | | lymphocyte LD78 beta protein) |
| | | | | (LD78-beta(1-70)) (G0/G1 switch |
| | | | | regulatory protein 19-2) (G0S19-2 |
| | | | | protein) (PAT 464.2) |
| 213418_at | 14.241181 | up | HSPA6 | heat shock 70 kDa protein 6 (HSP70B′) |
| 205943_at | 12.260297 | up | TDO2 | tryptophan 2,3-dioxygenase |
| 214038_at | 11.567539 | up | CCL8 | chemokine (C-C motif) ligand 8 |
| 207442_at | 11.09133 | up | CSF3 | colony stimulating factor 3 |
| | | | | (granulocyte) |
| 241031_at | 10.975465 | up | FAM148A | family with sequence similarity 148, |
| | | | | member A |
| 225207_at | 10.136719 | up | PDK4 | pyruvate dehydrogenase kinase, |
| | | | | isozyme 4 |
| 205931_s_at | 9.909108 | up | CREB5 | cAMP responsive element binding |
| | | | | protein 5 |
| 229228_at | 9.767824 | up | CREB5 | cAMP responsive element binding |
| | | | | protein 5 |
| 206924_at | 9.694227 | up | IL11 | interleukin 11 |
| 207526_s_at | 9.089992 | up | IL1RL1 | interleukin 1 receptor-like 1 |
| 206569_at | 8.736957 | up | IL24 | interleukin 24 |
| 207850_at | 8.702696 | up | CXCL3 | chemokine (C—X—C motif) ligand 3 |
| 206926_s_at | 7.970876 | up | IL11 | interleukin 11 |
| 206176_at | 7.765054 | up | BMP6 | bone morphogenetic protein 6 |
| 216248_s_at | 7.5919886 | up | NR4A2 | nuclear receptor subfamily 4, group A, |
| | | | | member 2 |
| 204622_x_at | 7.5477886 | up | NR4A2 | nuclear receptor subfamily 4, group A, |
| | | | | member 2 |
| 209774_x_at | 7.5460076 | up | CXCL2 | chemokine (C—X—C motif) ligand 2 |
| 233011_at | 7.322324 | up | ANXA1 | Annexin A1, mRNA (cDNA clone |
| | | | | MGC: 32774 IMAGE: 4662939) |
| 217388_s_at | 7.295115 | up | KYNU | kynureninase (L-kynurenine hydrolase) |
| 216979_at | 7.203941 | up | NR4A3 | nuclear receptor subfamily 4, group A, |
| | | | | member 3 |
| 204621_s_at | 7.06227 | up | NR4A2 | nuclear receptor subfamily 4, group A, |
| | | | | member 2 |
| 1554685_a_at | 7.039142 | up | KIAA1199 | KIAA1199 |
| 228501_at | 6.957408 | up | GALNTL2 | UDP-N-acetyl-alpha-D- |
| | | | | galactosamine:polypeptide N- |
| | | | | acetylgalactosaminyltransferase-like 2 |
| 235004_at | 6.917818 | up | RBM24 | RNA binding motif protein 24 |
| 202643_s_at | 6.915339 | up | TNFAIP3 | tumor necrosis factor, alpha-induced |
| | | | | protein 3 |
| 235086_at | 6.8579264 | up | THBS1 | (clone lambda-TS-33) thrombospondin |
| | | | | (THBS) mRNA, 5′ end |
| 206382_s_at | 6.826926 | up | BDNF | brain-derived neurotrophic factor |
| 1554997_a_at | 6.812711 | up | PTGS2 | prostaglandin-endoperoxide synthase 2 |
| | | | | (prostaglandin G/H synthase and |
| | | | | cyclooxygenase) |
| 237411_at | 6.7217474 | up | ADAMTS6 | ADAM metallopeptidase with |
| | | | | thrombospondin type 1 motif, 6 |
| 212942_s_at | 6.522843 | up | KIAA1199 | KIAA1199 |
| 230748_at | 6.4130154 | up | SLC16A6 | solute carrier family 16, member 6 |
| | | | | (monocarboxylic acid transporter 7) |
| 239367_at | 6.3149343 | up | BDNF | brain-derived neurotrophic factor |
| 204932_at | 6.287326 | up | TNFRSF11B | tumor necrosis factor receptor |
| | | | | superfamily, member 11b |
| 204933_s_at | 6.249607 | up | TNFRSF11B | tumor necrosis factor receptor |
| | | | | superfamily, member 11b |
| 236361_at | 6.174206 | up | GALNTL2 | UDP-N-acetyl-alpha-D- |
| | | | | galactosamine:polypeptide N- |
| | | | | acetylgalactosaminyltransferase-like 2 |
| 208075_s_at | 6.120267 | up | CCL7 | chemokine (C-C motif) ligand 7 |
| 226614_s_at | 6.0170407 | up | FAM167A | family with sequence similarity 167, |
| | | | | member A |
| 206137_at | 5.972218 | up | RIMS2 | regulating synaptic membrane |
| | | | | exocytosis 2 |
| 210133_at | 5.958449 | up | CCL11 | chemokine (C-C motif) ligand 11 |
| 1557257_at | 5.733714 | up | BCL10 | CDNA FLJ25924 fis, clone CBR05109 |
| 215078_at | 5.7122235 | up | SOD2 | superoxide dismutase 2, mitochondrial |
| 202644_s_at | 5.7050176 | up | TNFAIP3 | tumor necrosis factor, alpha-induced |
| | | | | protein 3 |
| 221577_x_at | 5.573193 | up | GDF15 | growth differentiation factor 15 |
| 206407_s_at | 5.516013 | up | CCL13 | chemokine (C-C motif) ligand 13 |
| 216598_s_at | 5.407172 | up | CCL2 | chemokine (C-C motif) ligand 2 |
| 203708_at | 5.313648 | up | PDE4B | phosphodiesterase 4B, cAMP-specific |
| | | | | (phosphodiesterase E4 dunce homolog, |
| | | | | Drosophila) |
| 242809_at | 5.274104 | up | IL1RL1 | ST2 protein |
| 209840_s_at | 5.160858 | up | LRRN3 | leucine rich repeat neuronal 3 |
| 204748_at | 5.148792 | up | PTGS2 | prostaglandin-endoperoxide synthase 2 |
| | | | | (prostaglandin G/H synthase and |
| | | | | cyclooxygenase) |
| 203645_s_at | 5.145445 | up | CD163 | CD163 molecule |
| 209841_s_at | 5.0964932 | up | LRRN3 | leucine rich repeat neuronal 3 |
| 207038_at | 5.0897655 | up | SLC16A6 | solute carrier family 16, member 6 |
| | | | | (monocarboxylic acid transporter 7) |
| 211302_s_at | 5.000998 | up | PDE4B | phosphodiesterase 4B, cAMP-specific |
| | | | | (phosphodiesterase E4 dunce homolog, |
| | | | | Drosophila) |
| 210663_s_at | 4.9557395 | up | KYNU | kynureninase (L-kynurenine hydrolase) |
| 215506_s_at | 4.9070177 | up | DIRAS3 | DIRAS family, GTP-binding RAS-like 3 |
| 220817_at | 4.90531 | up | TRPC4 | transient receptor potential cation |
| | | | | channel, subfamily C, member 4 |
| 215501_s_at | 4.860024 | up | DUSP10 | dual specificity phosphatase 10 |
| 209990_s_at | 4.7615633 | up | GABBR2 | gamma-aminobutyric acid (GABA) B |
| | | | | receptor, 2 |
| 203680_at | 4.729812 | up | PRKAR2B | protein kinase, cAMP-dependent, |
| | | | | regulatory, type II, beta |
| 207815_at | 4.719714 | up | PF4V1 | platelet factor 4 variant 1 |
| 206167_s_at | 4.7120314 | up | ARHGAP6 | Rho GTPase activating protein 6 |
| 210997_at | 4.711954 | up | HGF | hepatocyte growth factor (hepapoietin |
| | | | | A; scatter factor) |
| 213524_s_at | 4.7030506 | up | G0S2 | G0/G1switch 2 |
| 206950_at | 4.684502 | up | SCN9A | sodium channel, voltage-gated, type |
| | | | | IX, alpha |
| | | | | subunit |
| 215049_x_at | 4.532594 | up | CD163 | CD163 molecule |
| 224071_at | 4.5009127 | up | IL20 | interleukin 20 |
| 232017_at | 4.4948945 | up | TJP2 | tight junction protein 2 (zona |
| | | | | occludens 2) |
| 239461_at | 4.4746847 | up | GALNTL2 | UDP-N-acetyl-alpha-D- |
| | | | | galactosamine:polypeptide N- |
| | | | | acetylgalactosaminyltransferase-like 2 |
| 205681_at | 4.474304 | up | BCL2A1 | BCL2-related protein A1 |
| 201107_s_at | 4.4725666 | up | THBS1 | thrombospondin 1 |
| 203895_at | 4.4250426 | up | PLCB4 | phospholipase C, beta 4 |
| 212353_at | 4.4241323 | up | SULF1 | sulfatase 1 |
| 1569020_at | 4.418171 | up | NEDD9 | neural precursor cell expressed, |
| | | | | developmentally down-regulated 9 |
| 242329_at | 4.370621 | up | LOC401317 | hypothetical LOC401317 |
| 217999_s_at | 4.368414 | up | PHLDA1 | pleckstrin homology-like domain, |
| | | | | family A, member 1 |
| 220088_at | 4.3576527 | up | C5AR1 | complement component 5a receptor 1 |
| 213921_at | 4.35561 | up | SST | somatostatin |
| 210998_s_at | 4.350414 | up | HGF | hepatocyte growth factor (hepapoietin |
| | | | | A; scatter factor) |
| 205992_s_at | 4.32588 | up | IL15 | interleukin 15 |
| 242767_at | 4.3130975 | up | LMCD1 | CDNA FLJ52480 complete cds, highly |
| | | | | similar to LIM and cysteine-rich |
| | | | | domains protein 1 |
| 203817_at | 4.3071494 | up | GUCY1B3 | guanylate cyclase 1, soluble, beta 3 |
| 203304_at | 4.288485 | up | BAMBI | BMP and activin membrane-bound |
| | | | | inhibitor homolog (Xenopus laevis) |
| 237132_at | 4.2871304 | up | TJP2 | tight junction protein 2 (zona |
| | | | | occludens 2) |
| 207332_s_at | 4.2861505 | up | TFRC | transferrin receptor (p90, CD71) |
| 201860_s_at | 4.228699 | up | PLAT | plasminogen activator, tissue |
| 1552694_at | 4.2249846 | up | SLC2A13 | solute carrier family 2 (facilitated |
| | | | | glucose transporter), member 13 |
| 117_at | 4.210481 | up | HSPA6 | heat shock 70 kDa protein 6 (HSP70B′) |
| 207978_s_at | 4.165331 | up | NR4A3 | nuclear receptor subfamily 4, group A, |
| | | | | member 3 |
| 200664_s_at | 4.1382117 | up | DNAJB1 | DnaJ (Hsp40) homolog, subfamily B, |
| | | | | member 1 |
| 229160_at | 4.13802 | up | MUM1L1 | melanoma associated antigen (mutated) |
| | | | | 1-like 1 |
| 220818_s_at | 4.1121902 | up | TRPC4 | transient receptor potential cation |
| | | | | channel, subfamily C, member 4 |
| 210755_at | 4.1063404 | up | HGF | hepatocyte growth factor (hepapoietin |
| | | | | A; scatter factor) |
| 217371_s_at | 4.106031 | up | IL15 | interleukin 15 |
| 204160_s_at | 4.1003046 | up | ENPP4 | ectonucleotide |
| | | | | pyrophosphatase/phosphodiesterase 4 |
| | | | | (putative function) |
| 205476_at | 4.091709 | up | CCL20 | chemokine (C-C motif) ligand 20 |
| 203549_s_at | 4.0753236 | up | LPL | lipoprotein lipase |
| 234066_at | 4.0528307 | up | IL1RL1 | ST2 protein |
| 203896_s_at | 4.0160823 | up | PLCB4 | phospholipase C, beta 4 |
| 1556134_a_at | 3.9963338 | up | B3GNT5 | Homo sapiens, clone |
| | | | | IMAGE: 5122250, mRNA |
| 205410_s_at | 3.9841306 | up | ATP2B4 | ATPase, Ca++ transporting, plasma |
| | | | | membrane 4 |
| 210287_s_at | 3.9503276 | up | FLT1 | fms-related tyrosine kinase 1 (vascular |
| | | | | endothelial growth factor/vascular |
| | | | | permeability factor receptor) |
| 219778_at | 3.9186013 | up | ZFPM2 | zinc finger protein, multitype 2 |
| 204273_at | 3.9157705 | up | EDNRB | endothelin receptor type B |
| 208691_at | 3.9147308 | up | TFRC | transferrin receptor (p90, CD71) |
| 1556629_a_at | 3.913148 | up | SNAP25 | HUMSNAP25B(F) |
| 209960_at | 3.912294 | up | HGF | hepatocyte growth factor (hepapoietin |
| | | | | A; scatter factor) |
| 228962_at | 3.9099233 | up | PDE4D | phosphodiesterase 4D, cAMP-specific |
| | | | | (phosphodiesterase E3 dunce homolog, |
| | | | | Drosophila) |
| 1553133_at | 3.861553 | up | C9orf72 | chromosome 9 open reading frame 72 |
| 204421_s_at | 3.8445234 | up | FGF2 | fibroblast growth factor 2 (basic) |
| 229435_at | 3.841224 | up | GLIS3 | GLIS family zinc finger 3 |
| 214582_at | 3.8272183 | up | PDE3B | phosphodiesterase 3B, cGMP-inhibited |
| 229199_at | 3.8180137 | up | SCN9A | sodium channel, voltage-gated, type |
| | | | | IX, alpha subunit |
| 203548_s_at | 3.8146515 | up | LPL | lipoprotein lipase |
| 224219_s_at | 3.8127234 | up | TRPC4 | transient receptor potential cation |
| | | | | channel, subfamily C, member 4 |
| 202422_s_at | 3.7855804 | up | ACSL4 | acyl-CoA synthetase long-chain family |
| | | | | member 4 |
| 204385_at | 3.7706523 | up | KYNU | kynureninase (L-kynurenine hydrolase) |
| 206701_x_at | 3.767451 | up | EDNRB | endothelin receptor type B |
| 212099_at | 3.7642062 | up | RHOB | ras homolog gene family, member B |
| 227176_at | 3.7533486 | up | SLC2A13 | solute carrier family 2 (facilitated |
| | | | | glucose transporter), member 13 |
| 201044_x_at | 3.7260537 | up | DUSP1 | dual specificity phosphatase 1 |
| 205266_at | 3.7018826 | up | LIF | leukemia inhibitory factor (cholinergic |
| | | | | differentiation factor) |
| 221563_at | 3.6977322 | up | DUSP10 | dual specificity phosphatase 10 |
| 230258_at | 3.689173 | up | GLIS3 | GLIS family zinc finger 3 |
| 215910_s_at | 3.663163 | up | FNDC3A | fibronectin type III domain containing |
| | | | | 3A |
| 215033_at | 3.6403422 | up | TM4SF1 | transmembrane 4 L six family member 1 |
| 215966_x_at | 3.6169035 | up | GK3P | glycerol kinase 3 pseudogene |
| 1552695_a_at | 3.5807984 | up | SLC2A13 | solute carrier family 2 (facilitated |
| | | | | glucose transporter), member 13 |
| 204931_at | 3.5795379 | up | TCF21 | transcription factor 21 |
| 209959_at | 3.5782444 | up | NR4A3 | nuclear receptor subfamily 4, group A, |
| | | | | member 3 |
| 215034_s_at | 3.5756443 | up | TM4SF1 | transmembrane 4 L six family member 1 |
| 235591_at | 3.5697513 | up | SSTR1 | somatostatin receptor 1 |
| 202843_at | 3.5600665 | up | DNAJB9 | DnaJ (Hsp40) homolog, subfamily B, |
| | | | | member 9 |
| 231367_s_at | 3.558707 | up | LOC647131 | hypothetical LOC647131 |
| 202508_s_at | 3.5226371 | up | SNAP25 | synaptosomal-associated protein, |
| | | | | 25 kDa |
| 1569617_at | 3.5221984 | up | OSBP2 | CDNA clone IMAGE: 3632045 |
| 202672_s_at | 3.5205257 | up | ATF3 | activating transcription factor 3 |
| 210837_s_at | 3.520149 | up | PDE4D | phosphodiesterase 4D, cAMP-specific |
| | | | | (phosphodiesterase E3 dunce homolog, |
| | | | | Drosophila) |
| 218000_s_at | 3.5197904 | up | PHLDA1 | pleckstrin homology-like domain, |
| | | | | family A, member 1 |
| 214632_at | 3.510114 | up | NRP2 | neuropilin 2 |
| 215977_x_at | 3.509982 | up | GK | glycerol kinase |
| 219935_at | 3.4860334 | up | ADAMTS5 | ADAM metallopeptidase with |
| | | | | thrombospondin type 1 motif, 5 |
| 209101_at | 3.474422 | up | CTGF | connective tissue growth factor |
| 244804_at | 3.4737628 | up | SQSTM1 | Sequestosome 1 (SQSTM1), transcript |
| | | | | variant 1, mRNA |
| 212224_at | 3.462966 | up | ALDH1A1 | aldehyde dehydrogenase 1 family, |
| | | | | member A1 |
| 212354_at | 3.4603515 | up | SULF1 | sulfatase 1 |
| 204422_s_at | 3.455492 | up | FGF2 | fibroblast growth factor 2 (basic) |
| 229357_at | 3.450523 | up | ADAMTS5 | ADAM metallopeptidase with |
| | | | | thrombospondin type 1 motif, 5 |
| 209071_s_at | 3.4271708 | up | RGS5 | regulator of G-protein signaling 5 |
| 205088_at | 3.4216924 | up | MAMLD1 | mastermind-like domain containing 1 |
| 217998_at | 3.4190629 | up | LOC652993 | hypothetical LOC652993 /// pleckstrin |
| | | | /// PHLDA1 | homology-like domain, family A, |
| | | | | member 1 |
| 227361_at | 3.409933 | up | HS3ST3B1 | heparan sulfate (glucosamine) 3-O- |
| | | | | sulfotransferase 3B1 |
| 217997_at | 3.4090981 | up | PHLDA1 | pleckstrin homology-like domain, |
| | | | | family A, member 1 |
| 218177_at | 3.39026 | up | CHMP1B | chromatin modifying protein 1B |
| 204271_s_at | 3.385063 | up | EDNRB | endothelin receptor type B |
| 204491_at | 3.380295 | up | PDE4D | phosphodiesterase 4D, cAMP-specific |
| | | | | (phosphodiesterase E3 dunce homolog, |
| | | | | Drosophila) |
| 229273_at | 3.3785377 | up | SALL1 | sal-like 1 (Drosophila) |
| 206411_s_at | 3.3458714 | up | ABL2 | v-abl Abelson murine leukemia viral |
| | | | | oncogene homolog 2 (arg, Abelson- |
| | | | | related gene) |
| 219195_at | 3.344138 | up | PPARGC1A | peroxisome proliferator-activated |
| | | | | receptor gamma, coactivator 1 alpha |
| 231031_at | 3.3397727 | up | KGFLP2 | keratinocyte growth factor-like protein 2 |
| 206029_at | 3.3350718 | up | ANKRD1 | ankyrin repeat domain 1 (cardiac |
| | | | | muscle) |
| 211555_s_at | 3.3216224 | up | GUCY1B3 | guanylate cyclase 1, soluble, beta 3 |
| 217167_x_at | 3.3193831 | up | GK | glycerol kinase |
| 210511_s_at | 3.3132741 | up | INHBA | inhibin, beta A |
| 210836_x_at | 3.3034697 | up | PDE4D | phosphodiesterase 4D, cAMP-specific |
| | | | | (phosphodiesterase E3 dunce homolog, |
| | | | | Drosophila) |
| 207387_s_at | 3.2925398 | up | GK | glycerol kinase |
| 209961_s_at | 3.291533 | up | HGF | hepatocyte growth factor (hepapoietin |
| | | | | A; scatter factor) |
| 1554779_s_at | 3.281243 | up | PHLDB2 | pleckstrin homology-like domain, |
| | | | | family B, member 2 |
| 243357_at | 3.2709684 | up | NEGR1 | neuronal growth regulator 1 |
| 206893_at | 3.2649176 | up | SALL1 | sal-like 1 (Drosophila) |
| 1552632_a_at | 3.2559857 | up | ARSG | arylsulfatase G |
| 211844_s_at | 3.2455873 | up | NRP2 | neuropilin 2 |
| 212094_at | 3.234917 | up | PEG10 | paternally expressed 10 |
| 232235_at | 3.2294521 | up | DSEL | dermatan sulfate epimerase-like |
| 220416_at | 3.2220476 | up | ATP8B4 | ATPase, class I, type 8B, member 4 |
| 1555257_a_at | 3.2004068 | up | MYO3B | myosin IIIB |
| 225566_at | 3.1977339 | up | NRP2 | neuropilin 2 |
| 205207_at | 3.1970735 | up | IL6 | interleukin 6 (interferon, beta 2) |
| 240556_at | 3.1939862 | up | DCN | EST from clone 130486, 5′ end |
| 209387_s_at | 3.1869683 | up | TM4SF1 | transmembrane 4 L six family member 1 |
| 206025_s_at | 3.1820982 | up | TNFAIP6 | tumor necrosis factor, alpha-induced |
| | | | | protein 6 |
| 233126_s_at | 3.1724448 | up | OLAH | oleoyl-ACP hydrolase |
| 209386_at | 3.1714027 | up | TM4SF1 | transmembrane 4 L six family member 1 |
| 205782_at | 3.1584346 | up | FGF7 | fibroblast growth factor 7 (keratinocyte |
| | | | | growth factor) |
| 1553271_at | 3.1546502 | up | DIP2B | DIP2 disco-interacting protein 2 |
| | | | | homolog B (Drosophila) |
| 227487_s_at | 3.1483386 | up | SERPINE2 | Serpin peptidase inhibitor, clade E |
| | | | | (nexin, plasminogen activator inhibitor |
| | | | | type 1), member 2 (SERPINE2), |
| | | | | transcript variant 1, mRNA |
| 202558_s_at | 3.1297736 | up | HSPA13 | heat shock protein 70 kDa family, |
| | | | | member 13 |
| 219155_at | 3.1254086 | up | PITPNC1 | phosphatidylinositol transfer protein, |
| | | | | cytoplasmic 1 |
| 226814_at | 3.121966 | up | ADAMTS9 | ADAM metallopeptidase with |
| | | | | thrombospondin type 1 motif, 9 |
| 225946_at | 3.1174657 | up | RASSF8 | Ras association (RalGDS/AF-6) |
| | | | | domain family (N-terminal) member 8 |
| 1554163_at | 3.112223 | up | TWIST2 | twist homolog 2 (Drosophila) |
| 200800_s_at | 3.1119442 | up | HSPA1A /// | heat shock 70 kDa protein 1A /// heat |
| | | | HSPA1B | shock 70 kDa protein 1B |
| 201631_s_at | 3.1092095 | up | IER3 | immediate early response 3 |
| 202304_at | 3.109106 | up | FNDC3A | fibronectin type III domain containing |
| | | | | 3A |
| 223754_at | 3.1015599 | up | MGC13057 | hypothetical protein MGC13057 |
| 219975_x_at | 3.0908885 | up | OLAH | oleoyl-ACP hydrolase |
| 218546_at | 3.0864964 | up | C1orf115 | chromosome 1 open reading frame 115 |
| 205935_at | 3.0835295 | up | FOXF1 | forkhead box F1 |
| 229529_at | 3.0813122 | up | TCF21 | transcription factor 21 |
| 228490_at | 3.0776792 | up | ABHD2 | abhydrolase domain containing 2 |
| 206118_at | 3.0758493 | up | STAT4 | signal transducer and activator of |
| | | | | transcription 4 |
| 219279_at | 3.074901 | up | DOCK10 | dedicator of cytokinesis 10 |
| 222018_at | 3.0714924 | up | NACA /// | nascent polypeptide-associated |
| | | | NACA2 /// | complex alpha subunit /// nascent |
| | | | NACAP1 | polypeptide-associated complex alpha |
| | | | | subunit 2 /// nascent-polypeptide- |
| | | | | associated complex alpha polypeptide |
| | | | | pseudogene 1 |
| 225842_at | 3.0654018 | up | PHLDA1 | pleckstrin homology-like domain, |
| | | | | family A, member 1 |
| 1555167_s_at | 3.0645018 | up | NAMPT | nicotinamide |
| | | | | phosphoribosyltransferase |
| 201502_s_at | 3.0644495 | up | NFKBIA | nuclear factor of kappa light |
| | | | | polypeptide gene enhancer in B-cells |
| | | | | inhibitor, alpha |
| 228195_at | 3.054012 | up | MGC13057 | hypothetical protein MGC13057 |
| 209183_s_at | 3.0345566 | up | C10orf10 | chromosome 10 open reading frame 10 |
| 209833_at | 3.0264297 | up | CRADD | CASP2 and RIPK1 domain containing |
| | | | | adaptor with death domain |
| 204596_s_at | 3.0160112 | up | STC1 | stanniocalcin 1 |
| 204440_at | 3.0156825 | up | CD83 | CD83 molecule |
| 229461_x_at | 3.014515 | up | NEGR1 | neuronal growth regulator 1 |
| 201041_s_at | 3.0053132 | up | DUSP1 | dual specificity phosphatase 1 |
| 205356_at | 3.0039837 | up | USP13 | ubiquitin specific peptidase 13 |
| | | | | (isopeptidase T-3) |
| 216316_x_at | 2.9982364 | up | GK /// GK3P | glycerol kinase /// glycerol kinase 3 |
| | | | | pseudogene |
| 219257_s_at | 2.9839096 | up | SPHK1 | sphingosine kinase 1 |
| 205239_at | 2.983703 | up | AREG /// | amphiregulin /// amphiregulin B |
| | | | AREGB | |
| 225847_at | 2.9832149 | up | AADACL1 | arylacetamide deacetylase-like 1 |
| 214370_at | 2.9802194 | up | S100A8 | Calcium-binding protein in |
| | | | | macrophages (MRP-8) macrophage |
| | | | | migration inhibitory factor (MIF)- |
| | | | | related protein |
| 206756_at | 2.9774065 | up | CHST7 | carbohydrate (N-acetylglucosamine 6- |
| | | | | O) sulfotransferase 7 |
| 213325_at | 2.9673648 | up | PVRL3 | poliovirus receptor-related 3 |
| 220054_at | 2.967118 | up | IL23A | interleukin 23, alpha subunit p19 |
| 203372_s_at | 2.9669046 | up | SOCS2 | suppressor of cytokine signaling 2 |
| 231899_at | 2.9664657 | up | ZC3H12C | zinc finger CCCH-type containing 12C |
| 237215_s_at | 2.9501724 | up | TFRC | transferrin receptor (p90, CD71) |
| 227613_at | 2.935803 | up | ZNF331 | zinc finger protein 331 |
| 231944_at | 2.9320385 | up | ERO1LB | ERO1-like beta (S. cerevisiae), mRNA |
| | | | | (cDNA clone MGC: 26065 |
| | | | | IMAGE: 4829502) |
| 243296_at | 2.9062977 | up | NAMPT | G0S9 mRNA, instability elements |
| 207754_at | 2.9039783 | up | RASSF8 | Ras association (RalGDS/AF-6) |
| | | | | domain family (N-terminal) member 8 |
| 214681_at | 2.8969262 | up | GK | glycerol kinase |
| 225606_at | 2.8948586 | up | BCL2L11 | BCL2-like 11 (apoptosis facilitator) |
| 229225_at | 2.893143 | up | NRP2 | neuropilin 2 |
| 208962_s_at | 2.889333 | up | FADS1 | fatty acid desaturase 1 |
| 219926_at | 2.8884964 | up | POPDC3 | popeye domain containing 3 |
| 241611_s_at | 2.88746 | up | FNDC3A | fibronectin type III domain containing |
| | | | | 3A |
| 227235_at | 2.8838294 | up | GUCY1A3 | guanylate cyclase 1, soluble, alpha 3 |
| 241762_at | 2.8783326 | up | FBXO32 | F-box protein 32 (FBXO32), transcript |
| | | | | variant 2, mRNA |
| 236645_at | 2.8695679 | up | HBP1 | HMG-box transcription factor 1 |
| 206618_at | 2.864942 | up | IL18R1 | interleukin 18 receptor 1 |
| 223510_at | 2.864709 | up | NRP2 | neuropilin 2 |
| 208591_s_at | 2.851169 | up | PDE3B | phosphodiesterase 3B, cGMP-inhibited |
| 213103_at | 2.8436816 | up | STARD13 | StAR-related lipid transfer (START) |
| | | | | domain containing 13 |
| 224942_at | 2.8339565 | up | PAPPA | pregnancy-associated plasma protein |
| | | | | A, pappalysin 1 |
| 241763_s_at | 2.832837 | up | FBXO32 | F-box protein 32 (FBXO32), transcript |
| | | | | variant 2, mRNA |
| 229088_at | 2.831386 | up | ENPP1 | ectonucleotide |
| | | | | pyrophosphatase/phosphodiesterase 1 |
| 202779_s_at | 2.8305142 | up | LOC731049 | similar to Ubiquitin-conjugating |
| | | | /// UBE2S | enzyme E2S (Ubiquitin-conjugating |
| | | | | enzyme E2-24 kDa) (Ubiquitin-protein |
| | | | | ligase) (Ubiquitin carrier protein) (E2- |
| | | | | EPF5) /// ubiquitin-conjugating |
| | | | | enzyme E2S |
| 225142_at | 2.8252053 | up | JHDM1D | jumonji C domain containing histone |
| | | | | demethylase 1 homolog D (S. cerevisiae) |
| 232825_s_at | 2.8207173 | up | DSEL | dermatan sulfate epimerase-like |
| 212092_at | 2.8129215 | up | PEG10 | paternally expressed 10 |
| 1552508_at | 2.8024065 | up | KCNE4 | potassium voltage-gated channel, Isk- |
| | | | | related family, member 4 |
| 213931_at | 2.8021426 | up | ID2 /// ID2B | inhibitor of DNA binding 2, dominant |
| | | | | negative helix-loop-helix protein /// |
| | | | | inhibitor of DNA binding 2B, |
| | | | | dominant negative helix-loop-helix |
| | | | | protein (pseudogene) |
| 235996_at | 2.7879438 | up | RASSF8 | Ras association (RalGDS/AF-6) |
| | | | | domain family (N-terminal) member 8 |
| 204457_s_at | 2.7829573 | up | GAS1 | growth arrest-specific 1 |
| 207386_at | 2.7815282 | up | CYP7B1 | cytochrome P450, family 7, subfamily |
| | | | | B, polypeptide 1 |
| 209406_at | 2.773616 | up | BAG2 | BCL2-associated athanogene 2 |
| 207388_s_at | 2.7678204 | up | PTGES | prostaglandin E synthase |
| 204818_at | 2.7658327 | up | HSD17B2 | hydroxysteroid (17-beta) |
| | | | | dehydrogenase 2 |
| 201108_s_at | 2.7555368 | up | THBS1 | thrombospondin 1 |
| 224229_s_at | 2.7508733 | up | AKT3 | v-akt murine thymoma viral oncogene |
| | | | | homolog 3 (protein kinase B, gamma) |
| 235368_at | 2.7506857 | up | ADAMTS5 | ADAM metallopeptidase with |
| | | | | thrombospondin type 1 motif, 5 |
| 203827_at | 2.7443364 | up | WIPI1 | WD repeat domain, phosphoinositide |
| | | | | interacting 1 |
| 237867_s_at | 2.7432609 | up | PID1 | phosphotyrosine interaction domain |
| | | | | containing 1 |
| 206806_at | 2.7395847 | up | DGKI | diacylglycerol kinase, iota |
| 224941_at | 2.7371583 | up | PAPPA | pregnancy-associated plasma protein |
| | | | | A, pappalysin 1 |
| 231779_at | 2.7335684 | up | IRAK2 | interleukin-1 receptor-associated |
| | | | | kinase 2 |
| 204038_s_at | 2.7315595 | up | LPAR1 | lysophosphatidic acid receptor 1 |
| 201981_at | 2.7272985 | up | PAPPA | pregnancy-associated plasma protein |
| | | | | A, pappalysin 1 |
| 212374_at | 2.7260473 | up | FEM1B | fem-1 homolog b (C. elegans) |
| 216005_at | 2.7193272 | up | TNC | Tenascin |
| 229310_at | 2.719125 | up | KLHL29 | kelch-like 29 (Drosophila) |
| 211958_at | 2.7118793 | up | IGFBP5 | insulin-like growth factor binding |
| | | | | protein 5 |
| 214702_at | 2.7099462 | up | FN1 | fibronectin 1 |
| 224220_x_at | 2.7084796 | up | TRPC4 | transient receptor potential cation |
| | | | | channel, subfamily C, member 4 |
| 235723_at | 2.706666 | up | BNC2 | basonuclin 2 |
| 211965_at | 2.7050216 | up | ZFP36L1 | zinc finger protein 36, C3H type-like 1 |
| 225337_at | 2.704214 | up | ABHD2 | abhydrolase domain containing 2 |
| 209070_s_at | 2.700427 | up | RGS5 | regulator of G-protein signaling 5 |
| 1562606_a_at | 2.7000384 | up | LOC440028 | hypothetical gene supported by |
| | | | | BC040853 |
| 210764_s_at | 2.6922395 | up | CYR61 | cysteine-rich, angiogenic inducer, 61 |
| 222379_at | 2.6855981 | up | KCNE4 | potassium voltage-gated channel, Isk- |
| | | | | related family, member 4 |
| 205794_s_at | 2.6822994 | up | NOVA1 | neuro-oncological ventral antigen 1 |
| 207535_s_at | 2.6700058 | up | NFKB2 | nuclear factor of kappa light |
| | | | | polypeptide gene enhancer in B-cells 2 |
| | | | | (p49/p100) |
| 204015_s_at | 2.6682754 | up | DUSP4 | dual specificity phosphatase 4 |
| 217738_at | 2.6675475 | up | NAMPT | nicotinamide |
| | | | | phosphoribosyltransferase |
| 221569_at | 2.6674023 | up | AHI1 | Abelson helper integration site 1 |
| 236140_at | 2.6667895 | up | GCLM | glutamate-cysteine ligase, modifier |
| | | | | subunit |
| 1554462_a_at | 2.6664002 | up | DNAJB9 | DnaJ (Hsp40) homolog, subfamily B, |
| | | | | member 9 |
| 212344_at | 2.6652708 | up | SULF1 | sulfatase 1 |
| 202557_at | 2.6646934 | up | HSPA13 | heat shock protein 70 kDa family, |
| | | | | member 13 |
| 224940_s_at | 2.6638696 | up | PAPPA | pregnancy-associated plasma protein |
| | | | | A, pappalysin 1 |
| 220655_at | 2.6540391 | up | TNIP3 | TNFAIP3 interacting protein 3 |
| 216199_s_at | 2.6540227 | up | MAP3K4 | mitogen-activated protein kinase |
| | | | | kinase kinase 4 |
| 230206_at | 2.6534097 | up | DOCK5 | Dedicator of cytokinesis 5, mRNA |
| | | | | (cDNA clone IMAGE: 3347029) |
| 204557_s_at | 2.6526718 | up | DZIP1 | DAZ interacting protein 1 |
| 206390_x_at | 2.6515956 | up | PF4 | platelet factor 4 |
| 226322_at | 2.6468246 | up | TMTC1 | transmembrane and tetratricopeptide |
| | | | | repeat containing 1 |
| 203925_at | 2.6462543 | up | GCLM | glutamate-cysteine ligase, modifier |
| | | | | subunit |
| 220955_x_at | 2.644316 | up | RAB23 | RAB23, member RAS oncogene |
| | | | | family |
| 208322_s_at | 2.6438155 | up | ST3GAL1 | ST3 beta-galactoside alpha-2,3- |
| | | | | sialyltransferase 1 |
| 225033_at | 2.640394 | up | ST3GAL1 | ST3 beta-galactoside alpha-2,3- |
| | | | | sialyltransferase 1 |
| 208964_s_at | 2.6379926 | up | FADS1 | fatty acid desaturase 1 |
| 219134_at | 2.6371877 | up | ELTD1 | EGF, latrophilin and seven |
| | | | | transmembrane domain containing 1 |
| 232504_at | 2.6357627 | up | LOC285628 | hypothetical protein LOC285628 |
| 239163_at | 2.6356966 | up | UBE2B | ubiquitin-conjugating enzyme E2B |
| | | | | (RAD6 homolog) |
| 205924_at | 2.6338334 | up | RAB3B | RAB3B, member RAS oncogene |
| | | | | family |
| 225532_at | 2.6329355 | up | CABLES1 | Cdk5 and Abl enzyme substrate 1 |
| 228785_at | 2.6269226 | up | ZNF281 | Full length insert cDNA clone |
| | | | | ZE09A11 |
| 1554980_a_at | 2.623143 | up | ATF3 | activating transcription factor 3 |
| 212607_at | 2.6224685 | up | AKT3 | v-akt murine thymoma viral oncogene |
| | | | | homolog 3 (protein kinase B, gamma) |
| 227058_at | 2.6209402 | up | C13orf33 | chromosome 13 open reading frame 33 |
| 238649_at | 2.6207957 | up | PITPNC1 | phosphatidylinositol transfer protein, |
| | | | | cytoplasmic 1 |
| 242163_at | 2.6196032 | up | THRAP3 | thyroid hormone receptor associated |
| | | | | protein 3 |
| 229947_at | 2.618586 | up | PI15 | peptidase inhibitor 15 |
| 223264_at | 2.6175916 | up | MESDC1 | mesoderm development candidate 1 |
| 242283_at | 2.6143296 | up | C1orf67 /// | chromosome 1 open reading frame 67 |
| | | | DNAH14 | /// dynein, axonemal, heavy chain 14 |
| 202842_s_at | 2.612975 | up | DNAJB9 | DnaJ (Hsp40) homolog, subfamily B, |
| | | | | member 9 |
| 214472_at | 2.6077855 | up | HIST1H2AD | histone cluster 1, H2ad /// histone |
| | | | /// | cluster 1, H3a /// histone cluster 1, H3b |
| | | | HIST1H3A | /// histone cluster 1, H3c /// histone |
| | | | /// HIST1H3B | cluster 1, H3d /// histone cluster 1, H3e |
| | | | /// HIST1H3C | /// histone cluster 1, H3f /// histone |
| | | | /// | cluster 1, H3g /// histone cluster 1, H3h |
| | | | HIST1H3D | /// histone cluster 1, H3i /// histone |
| | | | /// HIST1H3E | cluster 1, H3j |
| | | | /// HIST1H3F | |
| | | | /// | |
| | | | HIST1H3G | |
| | | | /// | |
| | | | HIST1H3H | |
| | | | /// HIST1H3I | |
| | | | /// HIST1H3J | |
| 218980_at | 2.6065404 | up | FHOD3 | formin homology 2 domain containing 3 |
| 217590_s_at | 2.6019518 | up | TRPA1 | transient receptor potential cation |
| | | | | channel, subfamily A, member 1 |
| 211756_at | 2.5990396 | up | PTHLH | parathyroid hormone-like hormone |
| 227080_at | 2.5989056 | up | ZNF697 | zinc finger protein 697 |
| 220346_at | 2.5971677 | up | MTHFD2L | methylenetetrahydrofolate |
| | | | | dehydrogenase (NADP+ dependent) 2- |
| | | | | like |
| 221942_s_at | 2.5942783 | up | GUCY1A3 | guanylate cyclase 1, soluble, alpha 3 |
| 205825_at | 2.5902019 | up | PCSK1 | proprotein convertase subtilisin/kexin |
| | | | | type 1 |
| 222945_x_at | 2.5855827 | up | OLAH | oleoyl-ACP hydrolase |
| 226931_at | 2.5830119 | up | TMTC1 | transmembrane and tetratricopeptide |
| | | | | repeat containing 1 |
| 241765_at | 2.5736842 | up | CPM | carboxypeptidase M |
| 230237_at | 2.5733738 | up | ADCYAP1 | adenylate cyclase activating |
| | | | | polypeptide 1 (pituitary) |
| 209808_x_at | 2.5724812 | up | ING1 | inhibitor of growth family, member 1 |
| 227945_at | 2.5660486 | up | TBC1D1 | TBC1 (tre-2/USP6, BUB2, cdc16) |
| | | | | domain family, member 1 |
| 214446_at | 2.5570273 | up | ELL2 | elongation factor, RNA polymerase II, 2 |
| 221782_at | 2.5525873 | up | DNAJC10 | DnaJ (Hsp40) homolog, subfamily C, |
| | | | | member 10 |
| 223058_at | 2.5435784 | up | FAM107B | family with sequence similarity 107, |
| | | | | member B |
| 202213_s_at | 2.5383074 | up | CUL4B | cullin 4B |
| 237106_at | 2.5334878 | up | SLC11A2 | NRAMP2 |
| 235927_at | 2.5293667 | up | XPO1 | exportin 1 (CRM1 homolog, yeast) |
| 230494_at | 2.5260012 | up | SLC20A1 | Solute carrier family 20 (phosphate |
| | | | | transporter), member 1, mRNA (cDNA |
| | | | | clone MGC: 8767 IMAGE: 3918690) |
| 204897_at | 2.5254738 | up | PTGER4 | prostaglandin E receptor 4 (subtype |
| | | | | EP4) |
| 214469_at | 2.5204768 | up | HIST1H2AB | histone cluster 1, H2ab /// histone |
| | | | /// | cluster 1, H2ae |
| | | | HIST1H2AE | |
| 204472_at | 2.5198975 | up | GEM | GTP binding protein overexpressed in |
| | | | | skeletal muscle |
| 206026_s_at | 2.519742 | up | TNFAIP6 | tumor necrosis factor, alpha-induced |
| | | | | protein 6 |
| 210367_s_at | 2.5187645 | up | PTGES | prostaglandin E synthase |
| 217996_at | 2.5148838 | up | PHLDA1 | pleckstrin homology-like domain, |
| | | | | family A, member 1 |
| 227123_at | 2.5144148 | up | RAB3B | Small GTP binding protein RAB3B |
| | | | | (RAB3B) |
| 208885_at | 2.5127752 | up | LCP1 | lymphocyte cytosolic protein 1 (L- |
| | | | | plastin) |
| 214254_at | 2.5125573 | up | MAGEA4 | melanoma antigen family A, 4 |
| 226886_at | 2.512313 | up | GFPT1 | glutamine-fructose-6-phosphate |
| | | | | transaminase 1 |
| 205566_at | 2.5082593 | up | ABHD2 | abhydrolase domain containing 2 |
| 221781_s_at | 2.5082314 | up | DNAJC10 | DnaJ (Hsp40) homolog, subfamily C, |
| | | | | member 10 |
| 1554741_s_at | 2.5060341 | up | FGF7 /// | fibroblast growth factor 7 (keratinocyte |
| | | | KGFLP1 /// | growth factor) /// keratinocyte growth |
| | | | KGFLP2 | factor-like protein 1 /// keratinocyte |
| | | | | growth factor-like protein 2 |
| 206300_s_at | 2.5052757 | up | PTHLH | parathyroid hormone-like hormone |
| 206805_at | 2.5045469 | up | SEMA3A | sema domain, immunoglobulin domain |
| | | | | (Ig), short basic domain, secreted, |
| | | | | (semaphorin) 3A |
| 202052_s_at | 2.5033193 | up | RAI14 | retinoic acid induced 14 |
| 213993_at | 2.499253 | up | SPON1 | spondin 1, extracellular matrix protein |
| 209897_s_at | 2.4991436 | up | SLIT2 | slit homolog 2 (Drosophila) |
| 229530_at | 2.498387 | up | GUCY1A3 | guanylate cyclase 1, soluble, alpha 3 |
| 215285_s_at | 2.4979303 | up | PHTF1 | putative homeodomain transcription |
| | | | | factor 1 |
| 218615_s_at | 2.4974163 | up | TMEM39A | transmembrane protein 39A |
| 220615_s_at | 2.4965284 | up | FAR2 | fatty acyl CoA reductase 2 |
| 205303_at | 2.49271 | up | KCNJ8 | potassium inwardly-rectifying channel, |
| | | | | subfamily J, member 8 |
| 222735_at | 2.4891634 | up | TMEM38B | transmembrane protein 38B |
| 209501_at | 2.487513 | up | CDR2 | cerebellar degeneration-related protein |
| | | | | 2, 62 kDa |
| 205767_at | 2.486593 | up | EREG | epiregulin |
| 205830_at | 2.4858525 | up | CLGN | calmegin |
| 228128_x_at | 2.4817405 | up | PAPPA | pregnancy-associated plasma protein |
| | | | | A, pappalysin 1 |
| 229256_at | 2.4780662 | up | PGM2L1 | phosphoglucomutase 2-like 1 |
| 223059_s_at | 2.4761326 | up | FAM107B | family with sequence similarity 107, |
| | | | | member B |
| 228551_at | 2.473978 | up | DENND5B | DENN/MADD domain containing 5B |
| 205098_at | 2.4739516 | up | CCR1 | chemokine (C-C motif) receptor 1 |
| 220272_at | 2.4710832 | up | BNC2 | basonuclin 2 |
| 212975_at | 2.4708278 | up | DENND3 | DENN/MADD domain containing 3 |
| 222690_s_at | 2.469726 | up | TMEM39A | transmembrane protein 39A |
| 203239_s_at | 2.4657195 | up | CNOT3 | CCR4-NOT transcription complex, |
| | | | | subunit 3 |
| 203823_at | 2.4619107 | up | RGS3 | regulator of G-protein signaling 3 |
| 203373_at | 2.4602883 | up | SOCS2 | suppressor of cytokine signaling 2 |
| 210692_s_at | 2.456732 | up | SLC43A3 | solute carrier family 43, member 3 |
| 205139_s_at | 2.4541962 | up | UST | uronyl-2-sulfotransferase |
| 220145_at | 2.4529 | up | MAP9 | microtubule-associated protein 9 |
| 226743_at | 2.451173 | up | SLFN11 | schlafen family member 11 |
| 219093_at | 2.4472542 | up | PID1 | phosphotyrosine interaction domain |
| | | | | containing 1 |
| 213112_s_at | 2.4471107 | up | SQSTM1 | sequestosome 1 |
| 219228_at | 2.4418485 | up | ZNF331 | zinc finger protein 331 |
| 1554110_at | 2.4401536 | up | CDCP1 | CUB domain containing protein 1 |
| 217649_at | 2.4384716 | up | ZFAND5 | zinc finger, AN1-type domain 5 |
| 226804_at | 2.4369545 | up | FAM20A | family with sequence similarity 20, |
| | | | | member A |
| 243403_x_at | 2.4360526 | up | CPM | carboxypeptidase M |
| 214701_s_at | 2.4357622 | up | FN1 | fibronectin 1 |
| 207522_s_at | 2.434122 | up | ATP2A3 | ATPase, Ca++ transporting, ubiquitous |
| 219117_s_at | 2.4314268 | up | FKBP11 | FK506 binding protein 11, 19 kDa |
| 211840_s_at | 2.4287648 | up | PDE4D | phosphodiesterase 4D, cAMP-specific |
| | | | | (phosphodiesterase E3 dunce homolog, |
| | | | | Drosophila) |
| 239001_at | 2.428258 | up | MGST1 | Microsomal glutathione S-transferase 1 |
| | | | | (MGST1), transcript variant 1d, |
| | | | | mRNA |
| 219427_at | 2.4277742 | up | FAT4 | FAT tumor suppressor homolog 4 |
| | | | | (Drosophila) |
| 228540_at | 2.426083 | up | QKI | quaking homolog, KH domain RNA |
| | | | | binding (mouse) |
| 205547_s_at | 2.4258394 | up | TAGLN | transgelin |
| 214375_at | 2.425571 | up | LOC729222 | similar to mKIAA1230 protein /// |
| | | | /// PPFIBP1 | PTPRF interacting protein, binding |
| | | | | protein 1 (liprin beta 1) |
| 204037_at | 2.4254246 | up | LPAR1 | lysophosphatidic acid receptor 1 |
| 213704_at | 2.4229991 | up | RABGGTB | Rab geranylgeranyltransferase, beta |
| | | | | subunit |
| 219118_at | 2.422199 | up | FKBP11 | FK506 binding protein 11, 19 kDa |
| 225582_at | 2.4213138 | up | ITPRIP | inositol 1,4,5-triphosphate receptor |
| | | | | interacting protein |
| 229307_at | 2.4208326 | up | ANKRD28 | ankyrin repeat domain 28 |
| 214290_s_at | 2.4204948 | up | HIST2H2AA3 | histone cluster 2, H2aa3 /// histone |
| | | | /// | cluster 2, H2aa4 |
| | | | HIST2H2AA4 | |
| 201739_at | 2.4191535 | up | SGK1 | serum/glucocorticoid regulated kinase 1 |
| 239336_at | 2.4168572 | up | THBS1 | (clone lambda-TS-33) thrombospondin |
| | | | | (THBS) mRNA, 5′ end |
| 1559400_s_at | 2.4151716 | up | PAPPA | pregnancy-associated plasma protein |
| | | | | A, pappalysin 1 |
| 205066_s_at | 2.4135945 | up | ENPP1 | ectonucleotide |
| | | | | pyrophosphatase/phosphodiesterase 1 |
| 220034_at | 2.4113884 | up | IRAK3 | interleukin-1 receptor-associated |
| | | | | kinase 3 |
| 208963_x_at | 2.4111784 | up | FADS1 | fatty acid desaturase 1 |
| 213496_at | 2.4059057 | up | LPPR4 | plasticity related gene 1 |
| 205453_at | 2.4039452 | up | HOXB2 | homeobox B2 |
| 205119_s_at | 2.4022236 | up | FPR1 | formyl peptide receptor 1 |
| 232224_at | 2.4019115 | up | MASP1 | mannan-binding lectin serine peptidase |
| | | | | 1 (C4/C2 activating component of Ra- |
| | | | | reactive factor) |
| 202392_s_at | 2.40164 | up | PISD | phosphatidylserine decarboxylase |
| 212906_at | 2.4009886 | up | GRAMD1B | GRAM domain containing 1B, mRNA |
| | | | | (cDNA clone IMAGE: 3854666) |
| 218801_at | 2.4000664 | up | UGCGL2 | UDP-glucose ceramide |
| | | | | glucosyltransferase-like 2 |
| 212122_at | 2.3983467 | up | RHOQ | ras homolog gene family, member Q |
| 220153_at | 2.3949509 | up | ENTPD7 | ectonucleoside triphosphate |
| | | | | diphosphohydrolase 7 |
| 204036_at | 2.3905594 | up | LPAR1 | lysophosphatidic acid receptor 1 |
| 228461_at | 2.3892233 | up | SH3RF3 | SH3 domain containing ring finger 3 |
| 205304_s_at | 2.3868387 | up | KCNJ8 | potassium inwardly-rectifying channel, |
| | | | | subfamily J, member 8 |
| 212350_at | 2.386301 | up | TBC1D1 | TBC1 (tre-2/USP6, BUB2, cdc16) |
| | | | | domain family, member 1 |
| 205003_at | 2.3832471 | up | DOCK4 | dedicator of cytokinesis 4 |
| 235339_at | 2.376645 | up | SETDB2 | SET domain, bifurcated 2 |
| 220012_at | 2.3763833 | up | ERO1LB | ERO1-like beta (S. cerevisiae) |
| 44783_s_at | 2.3720322 | up | HEY1 | hairy/enhancer-of-split related with |
| | | | | YRPW motif 1 |
| 1554290_at | 2.3719432 | up | HERC3 | hect domain and RLD 3 |
| 1570351_at | 2.370291 | up | ADAMTS6 | ADAM metallopeptidase with |
| | | | | thrombospondin type 1 motif, 6 |
| 204222_s_at | 2.3661144 | up | GLIPR1 | GLI pathogenesis-related 1 |
| 222880_at | 2.3649035 | up | AKT3 | v-akt murine thymoma viral oncogene |
| | | | | homolog 3 (protein kinase B, gamma) |
| 235593_at | 2.3646154 | up | LOC100128821 | hypothetical protein LOC100128821 /// |
| | | | /// ZEB2 | zinc finger E-box binding homeobox 2 |
| 232263_at | 2.3646133 | up | SLC6A15 | solute carrier family 6 (neutral amino |
| | | | | acid transporter), member 15 |
| 1552701_a_at | 2.3634586 | up | CARD16 | caspase recruitment domain family, |
| | | | | member 16 |
| 224807_at | 2.3633232 | up | GRAMD1A | GRAM domain containing 1A |
| 204470_at | 2.362897 | up | CXCL1 | chemokine (C—X—C motif) ligand 1 |
| | | | | (melanoma growth stimulating activity, |
| | | | | alpha) |
| 230263_s_at | 2.3622723 | up | DOCK5 | Dedicator of cytokinesis 5, mRNA |
| | | | | (cDNA clone IMAGE: 3347029) |
| 213035_at | 2.3611116 | up | ANKRD28 | ankyrin repeat domain 28 |
| 202986_at | 2.3609338 | up | ARNT2 | aryl-hydrocarbon receptor nuclear |
| | | | | translocator 2 |
| 209437_s_at | 2.3600132 | up | SPON1 | spondin 1, extracellular matrix protein |
| 206613_s_at | 2.3585851 | up | TAF1A | TATA box binding protein (TBP)- |
| | | | | associated factor, RNA polymerase I, |
| | | | | A, 48 kDa |
| 242079_at | 2.357697 | up | RGS12 | regulator of G-protein signaling 12 |
| 211924_s_at | 2.3567264 | up | PLAUR | plasminogen activator, urokinase |
| | | | | receptor |
| 203836_s_at | 2.3556855 | up | MAP3K5 | mitogen-activated protein kinase |
| | | | | kinase kinase 5 |
| 1555471_a_at | 2.354568 | up | FMN2 | formin 2 |
| 201042_at | 2.3514817 | up | TGM2 | transglutaminase 2 (C polypeptide, |
| | | | | protein-glutamine-gamma- |
| | | | | glutamyltransferase) |
| 223217_s_at | 2.3498836 | up | NFKBIZ | nuclear factor of kappa light |
| | | | | polypeptide gene enhancer in B-cells |
| | | | | inhibitor, zeta |
| 1555103_s_at | 2.3495677 | up | FGF7 | fibroblast growth factor 7 (keratinocyte |
| | | | | growth factor) |
| 221815_at | 2.3482256 | up | ABHD2 | abhydrolase domain containing 2 |
| 1558143_a_at | 2.346621 | up | BCL2L11 | BCL2-like 11 (apoptosis facilitator) |
| 223774_at | 2.3389745 | up | SNHG12 | small nucleolar RNA host gene 12 |
| | | | | (non-protein coding) |
| 1553194_at | 2.3387334 | up | NEGR1 | neuronal growth regulator 1 |
| 231015_at | 2.3381798 | up | KLF15 | Kruppel-like factor 15 |
| 1552578_a_at | 2.337757 | up | MYO3B | myosin IIIB |
| 202340_x_at | 2.33773 | up | NR4A1 | nuclear receptor subfamily 4, group A, |
| | | | | member 1 |
| 202609_at | 2.334194 | up | EPS8 | epidermal growth factor receptor |
| | | | | pathway substrate 8 |
| 210941_at | 2.333396 | up | PCDH7 | protocadherin 7 |
| 203921_at | 2.331509 | up | CHST2 | carbohydrate (N-acetylglucosamine-6- |
| | | | | O) sulfotransferase 2 |
| 1562102_at | 2.3314924 | up | AKR1C1 | Aldo-keto reductase family 1, member |
| | | | | C1 (dihydrodiol dehydrogenase 1; 20- |
| | | | | alpha (3-alpha)-hydroxysteroid |
| | | | | dehydrogenase), mRNA (cDNA clone |
| | | | | MGC: 42600 IMAGE: 4825338) |
| 222305_at | 2.3291554 | up | HK2 | hexokinase 2 |
| 217310_s_at | 2.3269384 | up | FOXJ3 | forkhead box J3 |
| 205128_x_at | 2.326936 | up | PTGS1 | prostaglandin-endoperoxide synthase 1 |
| | | | | (prostaglandin G/H synthase and |
| | | | | cyclooxygenase) |
| 238989_at | 2.326773 | up | C1GALT1C1 | C1GALT1-specific chaperone 1 |
| 235770_at | 2.3237767 | up | MASP1 | mannan-binding lectin serine peptidase |
| | | | | 1 (C4/C2 activating component of Ra- |
| | | | | reactive factor) |
| 226103_at | 2.3234518 | up | NEXN | nexilin (F actin binding protein) |
| 220841_s_at | 2.3223662 | up | AHI1 | Abelson helper integration site 1 |
| 228817_at | 2.3209872 | up | ALG9 | asparagine-linked glycosylation 9, |
| | | | | alpha-1,2-mannosyltransferase |
| | | | | homolog (S. cerevisiae) |
| 227688_at | 2.318034 | up | LRCH2 | leucine-rich repeats and calponin |
| | | | | homology (CH) domain containing 2 |
| 236290_at | 2.3161294 | up | DOK6 | docking protein 6 |
| 222736_s_at | 2.3157086 | up | TMEM38B | transmembrane protein 38B |
| 229584_at | 2.3139408 | up | LRRK2 | leucine-rich repeat kinase 2 |
| 203414_at | 2.313451 | up | MMD | monocyte to macrophage |
| | | | | differentiation-associated |
| 207570_at | 2.312389 | up | SHOX | short stature homeobox |
| 205214_at | 2.3090763 | up | STK17B | serine/threonine kinase 17b |
| 207630_s_at | 2.3087645 | up | CREM | cAMP responsive element modulator |
| 207237_at | 2.3084404 | up | KCNA3 | potassium voltage-gated channel, |
| | | | | shaker-related subfamily, member 3 |
| 228699_at | 2.3063605 | up | NRP2 | Vascular endothelial cell growth factor |
| | | | | 165 receptor 2 (VEGF165R2) |
| 215813_s_at | 2.3061419 | up | PTGS1 | prostaglandin-endoperoxide synthase 1 |
| | | | | (prostaglandin G/H synthase and |
| | | | | cyclooxygenase) |
| 209180_at | 2.3054507 | up | RABGGTB | Rab geranylgeranyltransferase, beta |
| | | | | subunit |
| 218696_at | 2.3052793 | up | EIF2AK3 | eukaryotic translation initiation factor |
| | | | | 2-alpha kinase 3 |
| 223370_at | 2.302342 | up | PLEKHA3 | pleckstrin homology domain |
| | | | | containing, family A (phosphoinositide |
| | | | | binding specific) member 3 |
| 205100_at | 2.2997909 | up | GFPT2 | glutamine-fructose-6-phosphate |
| | | | | transaminase 2 |
| 212609_s_at | 2.2997367 | up | AKT3 | Clones 23920 and 23921 mRNA |
| | | | | sequence |
| 1555281_x_at | 2.2991943 | up | ARMC8 | armadillo repeat containing 8 |
| 204944_at | 2.2989373 | up | PTPRG | protein tyrosine phosphatase, receptor |
| | | | | type, G |
| 229438_at | 2.2988055 | up | LOC100132244 | CDNA: FLJ22487 fis, clone |
| | | | | HRC10931 |
| 212902_at | 2.298442 | up | SEC24A | SEC24 family, member A (S. cerevisiae) |
| 224826_at | 2.2983568 | up | RP5- | hypothetical protein KIAA1434 |
| | | | 1022P6.2 | |
| 218772_x_at | 2.296167 | up | TMEM38B | transmembrane protein 38B |
| 241372_at | 2.2959862 | up | ZC3H6 | zinc finger CCCH-type containing 6 |
| 229555_at | 2.295903 | up | GALNT5 | UDP-N-acetyl-alpha-D- |
| | | | | galactosamine:polypeptide N- |
| | | | | acetylgalactosaminyltransferase 5 |
| | | | | (GalNAc-T5) |
| 202375_at | 2.295754 | up | SEC24D | SEC24 family, member D (S. cerevisiae) |
| 1559582_at | 2.2931254 | up | RHOQ | ras homolog gene family, member Q |
| 1555279_at | 2.29071 | up | ARMC8 | armadillo repeat containing 8 |
| 203475_at | 2.2897353 | up | CYP19A1 | cytochrome P450, family 19, |
| | | | | subfamily A, polypeptide 1 |
| 204984_at | 2.2885683 | up | GPC4 | glypican 4 |
| 1560007_at | 2.28589 | up | LOC645984 | hypothetical LOC645984 |
| 235236_at | 2.2848015 | up | LOC100131897 | Uncharacterized protein |
| | | | | LOC100131897 (LOC100131897), |
| | | | | mRNA |
| 1559060_a_at | 2.2843337 | up | FNIP1 | MRNA; cDNA DKFZp451A064 (from |
| | | | | clone DKFZp451A064) |
| 204089_x_at | 2.283146 | up | MAP3K4 | mitogen-activated protein kinase |
| | | | | kinase kinase 4 |
| 227621_at | 2.2817085 | up | WTAP | Wilms tumor 1 associated protein |
| | | | | (WTAP), transcript variant 2, mRNA |
| 203498_at | 2.2808936 | up | RCAN2 | regulator of calcineurin 2 |
| 230207_s_at | 2.2794304 | up | DOCK5 | Dedicator of cytokinesis 5, mRNA |
| | | | | (cDNA clone IMAGE: 3347029) |
| 213425_at | 2.2791493 | up | WNT5A | wingless-type MMTV integration site |
| | | | | family, member 5A |
| 220745_at | 2.2776432 | up | IL19 | interleukin 19 |
| 235338_s_at | 2.2773378 | up | SETDB2 | SET domain, bifurcated 2 |
| 200790_at | 2.2756164 | up | ODC1 | ornithine decarboxylase 1 |
| 218280_x_at | 2.2754815 | up | HIST2H2AA3 | histone cluster 2, H2aa3 /// histone |
| | | | /// | cluster 2, H2aa4 |
| | | | HIST2H2AA4 | |
| 204720_s_at | 2.2753987 | up | DNAJC6 | DnaJ (Hsp40) homolog, subfamily C, |
| | | | | member 6 |
| 202820_at | 2.2720664 | up | AHR | aryl hydrocarbon receptor |
| 228423_at | 2.270422 | up | MAP9 | microtubule-associated protein 9 |
| 203424_s_at | 2.2697344 | up | IGFBP5 | insulin-like growth factor binding |
| | | | | protein 5 |
| 221918_at | 2.2681808 | up | PCTK2 | PCTAIRE protein kinase 2 |
| 238877_at | 2.2665446 | up | EYA4 | eyes absent homolog 4 (Drosophila) |
| 202721_s_at | 2.2655363 | up | GFPT1 | glutamine-fructose-6-phosphate |
| | | | | transaminase 1 |
| 213159_at | 2.265123 | up | PCNX | pecanex homolog (Drosophila) |
| 203837_at | 2.2618065 | up | MAP3K5 | mitogen-activated protein kinase |
| | | | | kinase kinase 5 |
| 244808_at | 2.2614517 | up | GRAMD1A | GRAM domain containing 1A, mRNA |
| | | | | (cDNA clone IMAGE: 5921205) |
| 222802_at | 2.2602103 | up | EDN1 | endothelin 1 |
| 202722_s_at | 2.2597966 | up | GFPT1 | glutamine-fructose-6-phosphate |
| | | | | transaminase 1 |
| 228368_at | 2.2592928 | up | ARHGAP20 | Rho GTPase activating protein 20 |
| 228653_at | 2.2589445 | up | SAMD5 | sterile alpha motif domain containing 5 |
| 224455_s_at | 2.2587087 | up | ADPGK | ADP-dependent glucokinase |
| 1570515_a_at | 2.258047 | up | FILIP1 | filamin A interacting protein 1 |
| 44790_s_at | 2.257748 | up | C13orf18 /// | chromosome 13 open reading frame 18 |
| | | | LOC728970 | /// hypothetical LOC728970 |
| 213836_s_at | 2.2555509 | up | WIPI1 | WD repeat domain, phosphoinositide |
| | | | | interacting 1 |
| 225589_at | 2.254314 | up | SH3RF1 | SH3 domain containing ring finger 1 |
| 209545_s_at | 2.2526133 | up | RIPK2 | receptor-interacting serine-threonine |
| | | | | kinase 2 |
| 1556113_at | 2.2524776 | up | DKFZp451A211 | DKFZp451A211 protein |
| 206249_at | 2.2523835 | up | MAP3K13 | mitogen-activated protein kinase |
| | | | | kinase kinase 13 |
| 244852_at | 2.2514307 | up | DSEL | dermatan sulfate epimerase-like |
| 203980_at | 2.2506835 | up | FABP4 | fatty acid binding protein 4, adipocyte |
| 212585_at | 2.2501442 | up | OSBPL8 | oxysterol binding protein-like 8 |
| 210845_s_at | 2.249447 | up | PLAUR | plasminogen activator, urokinase |
| | | | | receptor |
| 223463_at | 2.2488134 | up | RAB23 | RAB23, member RAS oncogene |
| | | | | family |
| 209204_at | 2.2469199 | up | LMO4 | LIM domain only 4 |
| 227445_at | 2.2429285 | up | ZNF689 | zinc finger protein 689 |
| 223222_at | 2.2426856 | up | SLC25A19 | solute carrier family 25 (mitochondrial |
| | | | | thiamine pyrophosphate carrier), |
| | | | | member 19 |
| 209277_at | 2.2425933 | up | TFPI2 | tissue factor pathway inhibitor 2 |
| 205138_s_at | 2.241066 | up | UST | uronyl-2-sulfotransferase |
| 219390_at | 2.240858 | up | FKBP14 | FK506 binding protein 14, 22 kDa |
| 223315_at | 2.240703 | up | NTN4 | netrin 4 |
| 213309_at | 2.2406566 | up | PLCL2 | phospholipase C-like 2 |
| 218541_s_at | 2.2391245 | up | C8orf4 | chromosome 8 open reading frame 4 |
| 210510_s_at | 2.2383678 | up | NRP1 | neuropilin 1 |
| 212915_at | 2.2363226 | up | PDZRN3 | PDZ domain containing ring finger 3 |
| 226890_at | 2.2358947 | up | WDR35 | WD repeat domain 35 |
| 224523_s_at | 2.2345538 | up | C3orf26 | chromosome 3 open reading frame 26 |
| 213029_at | 2.2345178 | up | NFIB | nuclear factor I/B |
| 204597_x_at | 2.2341132 | up | STC1 | stanniocalcin 1 |
| 204595_s_at | 2.2336535 | up | STC1 | stanniocalcin 1 |
| 210875_s_at | 2.233045 | up | ZEB1 | zinc finger E-box binding homeobox 1 |
| 210257_x_at | 2.231 | up | CUL4B | cullin 4B |
| 230123_at | 2.2295592 | up | NECAP2 | NECAP endocytosis associated 2 |
| 229588_at | 2.229439 | up | DNAJC10 | DnaJ (Hsp40) homolog, subfamily C, |
| | | | | member 10 |
| 220092_s_at | 2.2293727 | up | ANTXR1 | anthrax toxin receptor 1 |
| 213417_at | 2.228594 | up | TBX2 | T-box 2 |
| 228986_at | 2.2255878 | up | OSBPL8 | oxysterol binding protein-like 8 |
| 201531_at | 2.2251139 | up | ZFP36 | zinc finger protein 36, C3H type, |
| | | | | homolog (mouse) |
| 210896_s_at | 2.224665 | up | ASPH | aspartate beta-hydroxylase |
| 222619_at | 2.2228825 | up | ZNF281 | zinc finger protein 281 |
| 220254_at | 2.2226748 | up | LRP12 | low density lipoprotein-related protein |
| | | | | 12 |
| 219910_at | 2.220998 | up | FICD | FIC domain containing |
| 227351_at | 2.2207675 | up | C16orf52 | chromosome 16 open reading frame 52 |
| 218401_s_at | 2.2176473 | up | ZNF281 | zinc finger protein 281 |
| 201289_at | 2.2144566 | up | CYR61 | cysteine-rich, angiogenic inducer, 61 |
| 202543_s_at | 2.2127845 | up | GMFB | glia maturation factor, beta |
| 213113_s_at | 2.211486 | up | SLC43A3 | solute carrier family 43, member 3 |
| 226337_at | 2.2105165 | up | SCYL1BP1 | SCY1-like 1 binding protein 1 |
| 221958_s_at | 2.209569 | up | GPR177 | G protein-coupled receptor 177 |
| 219985_at | 2.2090566 | up | HS3ST3A1 | heparan sulfate (glucosamine) 3-O- |
| | | | | sulfotransferase 3A1 |
| 223866_at | 2.2079794 | up | ARMC2 | armadillo repeat containing 2 |
| 221978_at | 2.207626 | up | HLA-F | major histocompatibility complex, |
| | | | | class I, F |
| 240728_at | 2.20726 | up | PLCB4 | Phospholipase C beta 4 (PLCB4) |
| 213338_at | 2.2054002 | up | TMEM158 | transmembrane protein 158 |
| 204151_x_at | 2.205384 | up | AKR1C1 | aldo-keto reductase family 1, member |
| | | | | C1 (dihydrodiol dehydrogenase 1; 20- |
| | | | | alpha (3-alpha)-hydroxysteroid |
| | | | | dehydrogenase) |
| 1554414_a_at | 2.2053685 | up | OSGIN2 | oxidative stress induced growth |
| | | | | inhibitor family member 2 |
| 209708_at | 2.2048678 | up | MOXD1 | monooxygenase, DBH-like 1 |
| 229868_s_at | 2.2048671 | up | GDF15 | Macrophage inhibitory cytokine-1 |
| | | | | (MIC-1) |
| 230031_at | 2.2040954 | up | HSPA5 | heat shock 70 kDa protein 5 (glucose- |
| | | | | regulated protein, 78 kDa) |
| 203857_s_at | 2.203801 | up | PDIA5 | protein disulfide isomerase family A, |
| | | | | member 5 |
| 239286_at | 2.2035995 | up | CDH11 | cadherin 11, type 2, OB-cadherin |
| | | | | (osteoblast) |
| 225174_at | 2.2034101 | up | DNAJC10 | DnaJ (Hsp40) homolog, subfamily C, |
| | | | | member 10 |
| 235706_at | 2.202396 | up | CPM | carboxypeptidase M |
| 205495_s_at | 2.2022398 | up | GNLY | granulysin |
| 235550_at | 2.2019966 | up | MAP9 | microtubule-associated protein 9 |
| 210275_s_at | 2.2013817 | up | ZFAND5 | zinc finger, AN1-type domain 5 |
| 216218_s_at | 2.2008579 | up | PLCL2 | phospholipase C-like 2 |
| 202888_s_at | 2.2007616 | up | ANPEP | alanyl (membrane) aminopeptidase |
| 210191_s_at | 2.199756 | up | PHTF1 | putative homeodomain transcription |
| | | | | factor 1 |
| 209781_s_at | 2.199483 | up | KHDRBS3 | KH domain containing, RNA binding, |
| | | | | signal transduction associated 3 |
| 1554036_at | 2.1965137 | up | ZBTB24 | zinc finger and BTB domain |
| | | | | containing 24 |
| 238669_at | 2.1943119 | up | PTGS1 | prostaglandin-endoperoxide synthase 1 |
| | | | | (prostaglandin G/H synthase and |
| | | | | cyclooxygenase) |
| 213036_x_at | 2.191737 | up | ATP2A3 | ATPase, Ca++ transporting, ubiquitous |
| 227220_at | 2.1902707 | up | NFXL1 | nuclear transcription factor, X-box |
| | | | | binding-like 1 |
| 236129_at | 2.1839883 | up | GALNT5 | CDNA FLJ75131 complete cds, highly |
| | | | | similar to Homo sapiens UDP-N- |
| | | | | acetyl-alpha-D- |
| | | | | galactosamine:polypeptide N- |
| | | | | acetylgalactosaminyltransferase 5 |
| | | | | (GalNAc-T5) (GALNT5), mRNA |
| 1555724_s_at | 2.1835961 | up | TAGLN | transgelin |
| 208510_s_at | 2.1819134 | up | PPARG | peroxisome proliferator-activated |
| | | | | receptor gamma |
| 229271_x_at | 2.1817298 | up | COL11A1 | collagen, type XI, alpha 1 |
| 219631_at | 2.1808488 | up | LRP12 | low density lipoprotein-related protein |
| | | | | 12 |
| 218193_s_at | 2.1798851 | up | GOLT1B | golgi transport 1 homolog B (S. cerevisiae) |
| 227660_at | 2.1793392 | up | ANTXR1 | anthrax toxin receptor 1 |
| 1552309_a_at | 2.176896 | up | NEXN | nexilin (F actin binding protein) |
| 214061_at | 2.1762218 | up | WDR67 | WD repeat domain 67 |
| 227294_at | 2.1758094 | up | ZNF689 | zinc finger protein 689 |
| 223296_at | 2.175538 | up | SLC25A33 | solute carrier family 25, member 33 |
| 203425_s_at | 2.1755157 | up | IGFBP5 | insulin-like growth factor binding |
| | | | | protein 5 |
| 226001_at | 2.1747112 | up | KLHL5 | kelch-like 5 (Drosophila) |
| 206445_s_at | 2.1745844 | up | PRMT1 | protein arginine methyltransferase 1 |
| 213988_s_at | 2.1742477 | up | SAT1 | spermidine/spermine N1- |
| | | | | acetyltransferase 1 |
| 214657_s_at | 2.1736887 | up | NCRNA00084 | non-protein coding RNA 84 |
| 210350_x_at | 2.1736152 | up | ING1 | inhibitor of growth family, member 1 |
| 204556_s_at | 2.1734612 | up | DZIP1 | DAZ interacting protein 1 |
| 207345_at | 2.170217 | up | FST | follistatin |
| 220301_at | 2.1696754 | up | CCDC102B | coiled-coil domain containing 102B |
| 203574_at | 2.169319 | up | NFIL3 | nuclear factor, interleukin 3 regulated |
| 215058_at | 2.1687279 | up | DENND5B | DENN/MADD domain containing 5B |
| 238497_at | 2.1683004 | up | TMEM136 | transmembrane protein 136 |
| 239468_at | 2.167167 | up | MKX | mohawk homeobox |
| 1554474_a_at | 2.1654644 | up | MOXD1 | monooxygenase, DBH-like 1 |
| 205991_s_at | 2.1652021 | up | PRRX1 | paired related homeobox 1 |
| 219500_at | 2.1651778 | up | CLCF1 | cardiotrophin-like cytokine factor 1 |
| 207829_s_at | 2.1641598 | up | BNIP1 | BCL2/adenovirus E1B 19 kDa |
| | | | | interacting protein 1 |
| 37226_at | 2.1634657 | up | BNIP1 | BCL2/adenovirus E1B 19 kDa |
| | | | | interacting protein 1 |
| 209850_s_at | 2.160621 | up | CDC42EP2 | CDC42 effector protein (Rho GTPase |
| | | | | binding) 2 |
| 217741_s_at | 2.1604116 | up | ZFAND5 | zinc finger, AN1-type domain 5 |
| 1554140_at | 2.1590114 | up | WDR78 | WD repeat domain 78 |
| 205110_s_at | 2.1575923 | up | FGF13 | fibroblast growth factor 13 |
| 63825_at | 2.157092 | up | ABHD2 | abhydrolase domain containing 2 |
| 228702_at | 2.1557388 | up | FLJ43663 | hypothetical LOC378805 |
| 226800_at | 2.1555407 | up | EFCAB7 | EF-hand calcium binding domain 7 |
| 202952_s_at | 2.155327 | up | ADAM12 | ADAM metallopeptidase domain 12 |
| 217739_s_at | 2.155086 | up | NAMPT | nicotinamide |
| | | | | phosphoribosyltransferase |
| 225185_at | 2.1548986 | up | MRAS | muscle RAS oncogene homolog |
| 214211_at | 2.1543927 | up | FTH1 | ferritin, heavy polypeptide 1 |
| 235311_at | 2.15348 | up | FKBP14 | FK506 binding protein 14, 22 kDa, |
| | | | | mRNA (cDNA clone MGC: 12218 |
| | | | | IMAGE: 4042173) |
| 225871_at | 2.153166 | up | STEAP2 | six transmembrane epithelial antigen of |
| | | | | the prostate 2 |
| 1561042_at | 2.1507823 | up | ITGB1 | integrin, beta 1 (fibronectin receptor, |
| | | | | beta polypeptide, antigen CD29 |
| | | | | includes MDF2, MSK12) |
| 204790_at | 2.1506758 | up | SMAD7 | SMAD family member 7 |
| 203518_at | 2.1503856 | up | LYST | lysosomal trafficking regulator |
| 208415_x_at | 2.1496897 | up | ING1 | inhibitor of growth family, member 1 |
| 230291_s_at | 2.1495125 | up | NFIB | HMGIC/NFIB fusion protein |
| | | | | (HMGIC/NFIB) |
| 213032_at | 2.146494 | up | NFIB | nuclear factor I/B |
| 214913_at | 2.1450849 | up | ADAMTS3 | ADAM metallopeptidase with |
| | | | | thrombospondin type 1 motif, 3 |
| 209436_at | 2.1450522 | up | SPON1 | spondin 1, extracellular matrix protein |
| 210355_at | 2.1439502 | up | PTHLH | parathyroid hormone-like hormone |
| 225436_at | 2.1422665 | up | FAM108C1 | family with sequence similarity 108, |
| | | | | member C1 |
| 1554602_at | 2.1414428 | up | RBM8A | RNA binding motif protein 8A |
| 204420_at | 2.1413243 | up | FOSL1 | FOS-like antigen 1 |
| 204742_s_at | 2.1387715 | up | PDS5B | PDS5, regulator of cohesion |
| | | | | maintenance, homolog B (S. cerevisiae) |
| 235182_at | 2.138126 | up | ISM1 | isthmin 1 homolog (zebrafish) |
| 203740_at | 2.1379848 | up | MPHOSPH6 | M-phase phosphoprotein 6 |
| 215997_s_at | 2.1378837 | up | CUL4B | cullin 4B |
| 219558_at | 2.1378727 | up | ATP13A3 | ATPase type 13A3 |
| 223758_s_at | 2.137832 | up | GTF2H2 | general transcription factor IIH, |
| | | | | polypeptide 2, 44 kDa |
| 241986_at | 2.1363626 | up | BMPER | BMP binding endothelial regulator |
| 232267_at | 2.135431 | up | GPR133 | G protein-coupled receptor 133 |
| 210841_s_at | 2.132714 | up | NRP2 | neuropilin 2 |
| 229623_at | 2.1318607 | up | FLJ12993 | Hypothetical LOC441027 (FLJ12993), |
| | | | | mRNA |
| 224461_s_at | 2.13164 | up | AIFM2 | apoptosis-inducing factor, |
| | | | | mitochondrion-associated, 2 |
| 219774_at | 2.1300507 | up | CCDC93 | coiled-coil domain containing 93 |
| 229306_at | 2.129455 | up | FAM148B | Family with sequence similarity 148, |
| | | | | member B (FAM148B), mRNA |
| 226142_at | 2.1290789 | up | GLIPR1 | GLI pathogenesis-related 1 |
| 210424_s_at | 2.1265538 | up | GOLGA8A | golgi autoantigen, golgin subfamily a, |
| | | | /// | 8A /// golgi autoantigen, golgin |
| | | | GOLGA8B | subfamily a, 8B |
| 232004_at | 2.1262782 | up | HNRNPR | heterogeneous nuclear |
| | | | | ribonucleoprotein R |
| 228950_s_at | 2.1261652 | up | GPR177 | G protein-coupled receptor 177 |
| 239415_at | 2.1261606 | up | MAP9 | microtubule-associated protein 9 |
| 213093_at | 2.125206 | up | PRKCA | protein kinase C, alpha |
| 213033_s_at | 2.1251779 | up | NFIB | nuclear factor I/B |
| 218574_s_at | 2.1227338 | up | LMCD1 | LIM and cysteine-rich domains 1 |
| 201467_s_at | 2.1219635 | up | NQO1 | NAD(P)H dehydrogenase, quinone 1 |
| 224978_s_at | 2.1217666 | up | USP36 | ubiquitin specific peptidase 36 |
| 232311_at | 2.1214871 | up | B2M | Beta 2-mu = beta 2-microglobulin |
| | | | | [human, SK-MEL-33 cells, mRNA |
| | | | | Mutant, 433 nt] |
| 211981_at | 2.121121 | up | COL4A1 | collagen, type IV, alpha 1 |
| 219471_at | 2.1198602 | up | C13orf18 /// | chromosome 13 open reading frame 18 |
| | | | LOC728970 | /// hypothetical LOC728970 |
| 214014_at | 2.119122 | up | CDC42EP2 | CDC42 effector protein (Rho GTPase |
| | | | | binding) 2 |
| 235019_at | 2.1169195 | up | CPM | carboxypeptidase M |
| 218810_at | 2.1168468 | up | ZC3H12A | zinc finger CCCH-type containing 12A |
| 210519_s_at | 2.116195 | up | NQO1 | NAD(P)H dehydrogenase, quinone 1 |
| 219790_s_at | 2.1150982 | up | NPR3 | natriuretic peptide receptor |
| | | | | C/guanylate cyclase C (atrionatriuretic |
| | | | | peptide receptor C) |
| 215012_at | 2.1141691 | up | ZNF451 | zinc finger protein 451 |
| 203853_s_at | 2.114138 | up | GAB2 | GRB2-associated binding protein 2 |
| 229553_at | 2.1131918 | up | PGM2L1 | phosphoglucomutase 2-like 1 |
| 203066_at | 2.1125245 | up | GALNAC4S- | B cell RAG associated protein |
| | | | 6ST | |
| 221881_s_at | 2.1089115 | up | CLIC4 | chloride intracellular channel 4 |
| 229414_at | 2.1082094 | up | PITPNC1 | phosphatidylinositol transfer protein, |
| | | | | cytoplasmic 1 |
| 228082_at | 2.1080198 | up | ASAM | adipocyte-specific adhesion molecule |
| 225212_at | 2.1078928 | up | SLC25A25 | solute carrier family 25 (mitochondrial |
| | | | | carrier; phosphate carrier), member 25 |
| 204115_at | 2.1078105 | up | GNG11 | guanine nucleotide binding protein (G |
| | | | | protein), gamma 11 |
| 229194_at | 2.1072176 | up | PCGF5 | polycomb group ring finger 5 |
| 219283_at | 2.107142 | up | C1GALT1C1 | C1GALT1-specific chaperone 1 |
| 215785_s_at | 2.1066854 | up | CYFIP2 | cytoplasmic FMR1 interacting protein 2 |
| 217202_s_at | 2.1064858 | up | GLUL | glutamate-ammonia ligase (glutamine |
| | | | | synthetase) |
| 228949_at | 2.1053102 | up | GPR177 | G protein-coupled receptor 177 |
| 226136_at | 2.10407 | up | GLIPR1 | GLI pathogenesis-related 1 |
| 225590_at | 2.1038868 | up | SH3RF1 | SH3 domain containing ring finger 1 |
| 226160_at | 2.1030507 | up | H6PD | hexose-6-phosphate dehydrogenase |
| | | | | (glucose 1-dehydrogenase) |
| 236154_at | 2.1026752 | up | QKI | CDNA FLJ39382 fis, clone |
| | | | | PERIC2000473 |
| 225034_at | 2.099384 | up | ST3GAL1 | ST3 beta-galactoside alpha-2,3- |
| | | | | sialyltransferase 1 |
| 202724_s_at | 2.0982356 | up | FOXO1 | forkhead box O1 |
| 1559477_s_at | 2.0980742 | up | MEIS1 | Meis homeobox 1 |
| 226731_at | 2.0977373 | up | PELO | Pelota major mRNA, complete cds; |
| | | | | alternatively spliced |
| 220253_s_at | 2.0964284 | up | LRP12 | low density lipoprotein-related protein |
| | | | | 12 |
| 1555270_a_at | 2.0956323 | up | WFS1 | Wolfram syndrome 1 (wolframin) |
| 219054_at | 2.0956144 | up | C5orf23 | chromosome 5 open reading frame 23 |
| 1553962_s_at | 2.0940528 | up | RHOB | ras homolog gene family, member B |
| 201468_s_at | 2.0929265 | up | NQO1 | NAD(P)H dehydrogenase, quinone 1 |
| 211725_s_at | 2.0922117 | up | BID | BH3 interacting domain death agonist |
| 210665_at | 2.091109 | up | TFPI | tissue factor pathway inhibitor |
| | | | | (lipoprotein-associated coagulation |
| | | | | inhibitor) |
| 243797_at | 2.089624 | up | STK17B | serine/threonine kinase 17b |
| 204456_s_at | 2.0887516 | up | GAS1 | growth arrest-specific 1 |
| 243631_at | 2.0873725 | up | LOC727815 | hypothetical LOC727815 |
| 204396_s_at | 2.0871058 | up | GRK5 | G protein-coupled receptor kinase 5 |
| 213994_s_at | 2.0860326 | up | SPON1 | spondin 1, extracellular matrix protein |
| 213790_at | 2.0849094 | up | ADAM12 | ADAM metallopeptidase domain 12 |
| 244752_at | 2.0830863 | up | ZNF438 | zinc finger protein 438 |
| 212554_at | 2.0824976 | up | CAP2 | CAP, adenylate cyclase-associated |
| | | | | protein, 2 (yeast) |
| 239108_at | 2.081294 | up | FAR2 | Fatty acyl CoA reductase 2, mRNA |
| | | | | (cDNA clone MGC: 22328 |
| | | | | IMAGE: 4732586) |
| 219284_at | 2.0798855 | up | HSPBAP1 | HSPB (heat shock 27 kDa) associated |
| | | | | protein 1 |
| 221586_s_at | 2.0788653 | up | E2F5 | E2F transcription factor 5, p130- |
| | | | | binding |
| 218665_at | 2.0779874 | up | FZD4 | frizzled homolog 4 (Drosophila) |
| 240395_at | 2.077514 | up | LOC100128727 | hypothetical LOC100128727 |
| 224583_at | 2.0774055 | up | COTL1 | coactosin-like 1 (Dictyostelium) |
| 203426_s_at | 2.0749454 | up | IGFBP5 | insulin-like growth factor binding |
| | | | | protein 5 |
| 238893_at | 2.0748096 | up | LOC338758 | hypothetical protein LOC338758 |
| 212298_at | 2.072682 | up | NRP1 | neuropilin 1 |
| 210145_at | 2.071591 | up | PLA2G4A | phospholipase A2, group IVA |
| | | | | (cytosolic, calcium-dependent) |
| 229404_at | 2.0715733 | up | TWIST2 | twist homolog 2 (Drosophila) |
| 205659_at | 2.0714095 | up | HDAC9 | histone deacetylase 9 |
| 213173_at | 2.071243 | up | PCNX | pecanex homolog (Drosophila) |
| 229287_at | 2.0710356 | up | PCNX | pecanex homolog (Drosophila) |
| 227027_at | 2.0672464 | up | GFPT1 | glutamine-fructose-6-phosphate |
| | | | | transaminase 1 |
| 205290_s_at | 2.064931 | up | BMP2 | bone morphogenetic protein 2 |
| 227658_s_at | 2.0645716 | up | PLEKHA3 | pleckstrin homology domain |
| | | | | containing, family A (phosphoinositide |
| | | | | binding specific) member 3 |
| 209324_s_at | 2.0643253 | up | RGS16 | regulator of G-protein signaling 16 |
| 209699_x_at | 2.0639524 | up | AKR1C2 | aldo-keto reductase family 1, member |
| | | | | C2 (dihydrodiol dehydrogenase 2; bile |
| | | | | acid binding protein; 3-alpha |
| | | | | hydroxysteroid dehydrogenase, type |
| | | | | III) |
| 221059_s_at | 2.063764 | up | COTL1 | coactosin-like 1 (Dictyostelium) |
| 219374_s_at | 2.0623355 | up | ALG9 | asparagine-linked glycosylation 9, |
| | | | | alpha-1,2-mannosyltransferase |
| | | | | homolog (S. cerevisiae) |
| 229942_at | 2.0615158 | up | BNC2 | basonuclin 2 |
| 212120_at | 2.0612128 | up | RHOQ | ras homolog gene family, member Q |
| 242281_at | 2.060927 | up | GLUL | glutamate-ammonia ligase (glutamine |
| | | | | synthetase) |
| 202214_s_at | 2.0583868 | up | CUL4B | cullin 4B |
| 201150_s_at | 2.0579739 | up | TIMP3 | TIMP metallopeptidase inhibitor 3 |
| 203927_at | 2.0568886 | up | NFKBIE | nuclear factor of kappa light |
| | | | | polypeptide gene enhancer in B-cells |
| | | | | inhibitor, epsilon |
| 220980_s_at | 2.0566983 | up | ADPGK | ADP-dependent glucokinase |
| 212454_x_at | 2.053964 | up | HNRPDL | HnRNP JKTBP |
| 235391_at | 2.0537112 | up | FAM92A1 | family with sequence similarity 92, |
| | | | | member A1 |
| 202784_s_at | 2.053589 | up | NNT | nicotinamide nucleotide |
| | | | | transhydrogenase |
| 203810_at | 2.0534089 | up | DNAJB4 | DnaJ (Hsp40) homolog, subfamily B, |
| | | | | member 4 |
| 222731_at | 2.0529802 | up | ZDHHC2 | zinc finger, DHHC-type containing 2 |
| 219682_s_at | 2.051939 | up | TBX3 | T-box 3 |
| 200755_s_at | 2.0517964 | up | CALU | calumenin |
| 210198_s_at | 2.051592 | up | PLP1 | proteolipid protein 1 |
| 203294_s_at | 2.0499084 | up | LMAN1 | lectin, mannose-binding, 1 |
| 210243_s_at | 2.0492911 | up | B4GALT3 | UDP-Gal:betaGlcNAc beta 1,4- |
| | | | | galactosyltransferase, polypeptide 3 |
| 201772_at | 2.0489492 | up | AZIN1 | antizyme inhibitor 1 |
| 218513_at | 2.046744 | up | C4orf43 | chromosome 4 open reading frame 43 |
| 212110_at | 2.0458658 | up | SLC39A14 | solute carrier family 39 (zinc |
| | | | | transporter), member 14 |
| 244246_at | 2.0455189 | up | MIPOL1 | mirror-image polydactyly 1 |
| 235359_at | 2.0454898 | up | LRRC33 | leucine rich repeat containing 33 |
| 211952_at | 2.0442946 | up | IPO5 | importin 5 |
| 239261_s_at | 2.0437918 | up | CORIN | corin, serine peptidase |
| 40560_at | 2.0437784 | up | TBX2 | T-box 2 |
| 231824_at | 2.0437446 | up | LARP2 | La ribonucleoprotein domain family, |
| | | | | member 2 |
| 238417_at | 2.0436635 | up | PGM2L1 | phosphoglucomutase 2-like 1 |
| 235061_at | 2.0422926 | up | PPM1K | protein phosphatase 1K (PP2C domain |
| | | | | containing) |
| 214077_x_at | 2.0414855 | up | MEIS3P1 | Meis homeobox 3 pseudogene 1 |
| 212977_at | 2.039824 | up | CXCR7 | chemokine (C—X—C motif) receptor 7 |
| 218178_s_at | 2.0365124 | up | CHMP1B | chromatin modifying protein 1B |
| 202581_at | 2.0352576 | up | HSPA1A /// | heat shock 70 kDa protein 1A /// heat |
| | | | HSPA1B | shock 70 kDa protein 1B |
| 239781_at | 2.034794 | up | hCG_1815504 | hCG1815504 |
| 204845_s_at | 2.0343878 | up | ENPEP | glutamyl aminopeptidase |
| | | | | (aminopeptidase A) |
| 221207_s_at | 2.033731 | up | NBEA | neurobeachin |
| 242814_at | 2.033107 | up | SERPINB9 | serpin peptidase inhibitor, clade B |
| | | | | (ovalbumin), member 9 |
| 225793_at | 2.0320432 | up | LIX1L | Lix1 homolog (mouse)-like |
| 211467_s_at | 2.0315952 | up | NFIB | nuclear factor I/B |
| 205499_at | 2.0314283 | up | SRPX2 | sushi-repeat-containing protein, X- |
| | | | | linked 2 |
| 202906_s_at | 2.0313315 | up | NBN | nibrin |
| 222343_at | 2.0312388 | up | BCL2L11 | BCL2-like 11 (apoptosis facilitator) |
| 204719_at | 2.03084 | up | ABCA8 | ATP-binding cassette, sub-family A |
| | | | | (ABC1), member 8 |
| 244128_x_at | 2.0301714 | up | GLIS1 | GLIS family zinc finger 1 |
| 202014_at | 2.0300674 | up | PPP1R15A | protein phosphatase 1, regulatory |
| | | | | (inhibitor) subunit 15A |
| 210001_s_at | 2.028387 | up | SOCS1 | suppressor of cytokine signaling 1 |
| 203184_at | 2.0282688 | up | FBN2 | fibrillin 2 |
| 203835_at | 2.0257106 | up | LRRC32 | leucine rich repeat containing 32 |
| 213435_at | 2.0254192 | up | SATB2 | SATB homeobox 2 |
| 203001_s_at | 2.025328 | up | STMN2 | stathmin-like 2 |
| 216594_x_at | 2.0242531 | up | AKR1C1 | aldo-keto reductase family 1, member |
| | | | | C1 (dihydrodiol dehydrogenase 1; 20- |
| | | | | alpha (3-alpha)-hydroxysteroid |
| | | | | dehydrogenase) |
| 221062_at | 2.0242138 | up | HS3ST3B1 | heparan sulfate (glucosamine) 3-O- |
| | | | | sulfotransferase 3B1 |
| 221685_s_at | 2.0240426 | up | CCDC99 | coiled-coil domain containing 99 |
| 210007_s_at | 2.0238538 | up | GPD2 | glycerol-3-phosphate dehydrogenase 2 |
| | | | | (mitochondrial) |
| 209676_at | 2.0226731 | up | TFPI | tissue factor pathway inhibitor |
| | | | | (lipoprotein-associated coagulation |
| | | | | inhibitor) |
| 219921_s_at | 2.0220945 | up | DOCK5 | dedicator of cytokinesis 5 |
| 212558_at | 2.0209737 | up | SPRY1 | sprouty homolog 1, antagonist of FGF |
| | | | | signaling (Drosophila) |
| 228728_at | 2.0184631 | up | C7orf58 | chromosome 7 open reading frame 58 |
| 1557258_a_at | 2.018125 | up | BCL10 | CDNA FLJ25924 fis, clone CBR05109 |
| 213302_at | 2.017492 | up | PFAS | phosphoribosylformylglycinamidine |
| | | | | synthase |
| 209289_at | 2.0158818 | up | NFIB | nuclear factor I/B |
| 230559_x_at | 2.0148904 | up | FGD4 | FYVE, RhoGEF and PH domain |
| | | | | containing 4 |
| 220093_at | 2.0146642 | up | ANTXR1 | anthrax toxin receptor 1 |
| 214587_at | 2.0145714 | up | COL8A1 | collagen, type VIII, alpha 1 |
| 213359_at | 2.013772 | up | HNRNPD | HnRNP-C like protein |
| 200648_s_at | 2.0120223 | up | GLUL | glutamate-ammonia ligase (glutamine |
| | | | | synthetase) |
| 241902_at | 2.0113673 | up | MKX | mohawk homeobox |
| 204114_at | 2.0113 | up | NID2 | nidogen 2 (osteonidogen) |
| 225385_s_at | 2.0110688 | up | HNRPLL | heterogeneous nuclear |
| | | | | ribonucleoprotein L-like |
| 227983_at | 2.0109632 | up | RILPL2 | Rab interacting lysosomal protein-like 2 |
| 203194_s_at | 2.0106804 | up | NUP98 | nucleoporin 98 kDa |
| 1567107_s_at | 2.0090067 | up | TPM4 | tropomyosin 4 |
| 201941_at | 2.0086033 | up | CPD | carboxypeptidase D |
| 202710_at | 2.0079546 | up | BET1 | blocked early in transport 1 homolog |
| | | | | (S. cerevisiae) |
| 202908_at | 2.0073936 | up | WFS1 | Wolfram syndrome 1 (wolframin) |
| 202302_s_at | 2.0070374 | up | RSRC2 | arginine/serine-rich coiled-coil 2 |
| 210839_s_at | 2.0057895 | up | ENPP2 | ectonucleotide |
| | | | | pyrophosphatase/phosphodiesterase 2 |
| 216235_s_at | 2.0052087 | up | EDNRA | endothelin receptor type A |
| 218995_s_at | 2.0046716 | up | EDN1 | endothelin 1 |
| 238049_at | 2.0043075 | up | GRAMD3 | GRAM domain containing 3 |
| 1554334_a_at | 2.0030234 | up | DNAJA4 | DnaJ (Hsp40) homolog, subfamily A, |
| | | | | member 4 |
| 202804_at | 2.0030096 | up | ABCC1 | ATP-binding cassette, sub-family C |
| | | | | (CFTR/MRP), member 1 |
| 219872_at | 2.0028145 | up | C4orf18 | chromosome 4 open reading frame 18 |
| 237056_at | 2.0025263 | up | INSC | inscuteable homolog (Drosophila) |
| 209290_s_at | 2.0020652 | up | NFIB | nuclear factor I/B |
| 203404_at | 2.0020292 | up | ARMCX2 | armadillo repeat containing, X-linked 2 |
| 200799_at | 2.0019038 | up | HSPA1A /// | heat shock 70 kDa protein 1A /// heat |
| | | | HSPA1B | shock 70 kDa protein 1B |
| 226025_at | 2.0013952 | up | ANKRD28 | ankyrin repeat domain 28 |
| 207710_at | 200.50969 | down | LCE2B | late cornified envelope 2B |
| 221470_s_at | 100.54604 | down | IL1F7 | interleukin 1 family, member 7 (zeta) |
| 211548_s_at | 92.36748 | down | HPGD | hydroxyprostaglandin dehydrogenase |
| | | | | 15-(NAD) |
| 1553081_at | 88.25469 | down | WFDC12 | WAP four-disulfide core domain 12 |
| 203914_x_at | 78.77154 | down | HPGD | hydroxyprostaglandin dehydrogenase |
| | | | | 15-(NAD) |
| 231930_at | 71.26495 | down | ELMOD1 | ELMO/CED-12 domain containing 1 |
| 1560531_at | 71.235 | down | LCE1B | late cornified envelope 1B |
| 203913_s_at | 67.80365 | down | HPGD | hydroxyprostaglandin dehydrogenase |
| | | | | 15-(NAD) |
| 206643_at | 55.867226 | down | HAL | histidine ammonia-lyase |
| 211549_s_at | 53.149216 | down | HPGD | hydroxyprostaglandin dehydrogenase |
| | | | | 15-(NAD) |
| 240420_at | 52.46811 | down | AADACL2 | arylacetamide deacetylase-like 2 |
| 209309_at | 51.06968 | down | AZGP1 | alpha-2-glycoprotein 1, zinc-binding |
| 1569410_at | 45.91465 | down | FLG2 | filaggrin family member 2 |
| 239787_at | 41.795918 | down | KCTD4 | potassium channel tetramerisation |
| | | | | domain containing 4 |
| 216935_at | 40.454136 | down | C1orf46 | chromosome 1 open reading frame 46 |
| 1553602_at | 33.973278 | down | MUCL1 | mucin-like 1 |
| 207720_at | 32.353355 | down | LOR | loricrin |
| 220625_s_at | 31.788977 | down | ELF5 | E74-like factor 5 (ets domain |
| | | | | transcription factor) |
| 223720_at | 25.761408 | down | SPINK7 | serine peptidase inhibitor, Kazal type 7 |
| | | | | (putative) |
| 241412_at | 25.479097 | down | BTC | betacellulin |
| 224555_x_at | 21.945213 | down | IL1F7 | interleukin 1 family, member 7 (zeta) |
| 235514_at | 21.287827 | down | ASPRV1 | aspartic peptidase, retroviral-like 1 |
| 237974_at | 21.200142 | down | ABHD12B | abhydrolase domain containing 12B |
| 1552532_a_at | 20.645466 | down | ATP6V1C2 | ATPase, H+ transporting, lysosomal |
| | | | | 42 kDa, V1 subunit C2 |
| 226188_at | 20.39768 | down | HSPC159 | galectin-related protein |
| 202688_at | 20.340603 | down | TNFSF10 | tumor necrosis factor (ligand) |
| | | | | superfamily, member 10 |
| 202687_s_at | 20.30895 | down | TNFSF10 | tumor necrosis factor (ligand) |
| | | | | superfamily, member 10 |
| 205159_at | 20.243301 | down | CSF2RB | colony stimulating factor 2 receptor, |
| | | | | beta, low-affinity (granulocyte- |
| | | | | macrophage) |
| 1553534_at | 20.000898 | down | NLRP10 | NLR family, pyrin domain containing |
| | | | | 10 |
| 211712_s_at | 19.846176 | down | ANXA9 | annexin A9 |
| 228766_at | 19.823524 | down | CD36 | CD36 molecule (thrombospondin |
| | | | | receptor) |
| 219476_at | 19.65667 | down | C1orf116 | chromosome 1 open reading frame 116 |
| 206295_at | 19.309084 | down | IL18 | interleukin 18 (interferon-gamma- |
| | | | | inducing factor) |
| 220635_at | 19.078548 | down | PSORS1C2 | psoriasis susceptibility 1 candidate 2 |
| 223816_at | 18.854237 | down | SLC46A2 | solute carrier family 46, member 2 |
| 240512_x_at | 18.449383 | down | KCTD4 | potassium channel tetramerisation |
| | | | | domain containing 4 |
| 1552544_at | 18.410263 | down | SERPINA12 | serpin peptidase inhibitor, clade A |
| | | | | (alpha-1 antiproteinase, antitrypsin), |
| | | | | member 12 |
| 207908_at | 18.13667 | down | KRT2 | keratin 2 |
| 202454_s_at | 17.877632 | down | ERBB3 | v-erb-b2 erythroblastic leukemia viral |
| | | | | oncogene homolog 3 (avian) |
| 233488_at | 17.474066 | down | RNASE7 | ribonuclease, RNase A family, 7 |
| 225667_s_at | 17.434992 | down | FAM84A | family with sequence similarity 84, |
| | | | | member A |
| 234331_s_at | 17.273813 | down | FAM84A | family with sequence similarity 84, |
| | | | | member A |
| 227717_at | 17.084187 | down | FLJ41603 | FLJ41603 protein |
| 203608_at | 17.013868 | down | ALDH5A1 | aldehyde dehydrogenase 5 family, |
| | | | | member A1 |
| 227241_at | 16.848194 | down | MUC15 | mucin 15, cell surface associated |
| 209604_s_at | 16.352425 | down | GATA3 | GATA binding protein 3 |
| 204733_at | 16.28207 | down | KLK6 | kallikrein-related peptidase 6 |
| 1564307_a_at | 15.90927 | down | A2ML1 | alpha-2-macroglobulin-like 1 |
| 209602_s_at | 15.703371 | down | GATA3 | GATA binding protein 3 |
| 1554195_a_at | 15.667729 | down | C5orf46 | chromosome 5 open reading frame 46 |
| 222484_s_at | 15.34712 | down | CXCL14 | chemokine (C—X—C motif) ligand 14 |
| 1554252_a_at | 15.340231 | down | LASS3 | LAG1 homolog, ceramide synthase 3 |
| 203798_s_at | 15.19176 | down | VSNL1 | visinin-like 1 |
| 232170_at | 15.098177 | down | S100A7A | S100 calcium binding protein A7A |
| 209493_at | 15.042625 | down | PDZD2 | PDZ domain containing 2 |
| 219795_at | 15.010469 | down | SLC6A14 | solute carrier family 6 (amino acid |
| | | | | transporter), member 14 |
| 213135_at | 14.684513 | down | TIAM1 | T-cell lymphoma invasion and |
| | | | | metastasis 1 |
| 237120_at | 14.594361 | down | KRT77 | keratin 77 |
| 226789_at | 14.518254 | down | LOC647121 | embigin homolog (mouse) pseudogene |
| 1555773_at | 13.957752 | down | BPIL2 | bactericidal/permeability-increasing |
| | | | | protein-like 2 |
| 205439_at | 13.706697 | down | GSTT2 | glutathione S-transferase theta 2 |
| 219695_at | 13.657985 | down | SMPD3 | sphingomyelin phosphodiesterase 3, |
| | | | | neutral membrane (neutral |
| | | | | sphingomyelinase II) |
| 213933_at | 13.583514 | down | PTGER3 | prostaglandin E receptor 3 (subtype |
| | | | | EP3) |
| 214329_x_at | 13.48385 | down | TNFSF10 | tumor necrosis factor (ligand) |
| | | | | superfamily, member 10 |
| 218454_at | 13.372534 | down | FLJ22662 | hypothetical protein FLJ22662 |
| 213780_at | 13.321 | down | TCHH | trichohyalin |
| 231867_at | 13.271721 | down | ODZ2 | odz, odd Oz/ten-m homolog 2 |
| | | | | (Drosophila) |
| 227238_at | 13.252493 | down | MUC15 | mucin 15, cell surface associated |
| 243386_at | 13.026348 | down | CASZ1 | castor zinc finger 1 |
| 207324_s_at | 12.947083 | down | DSC1 | desmocollin 1 |
| 232165_at | 12.401502 | down | EPPK1 | epiplakin 1 |
| 204393_s_at | 12.384473 | down | ACPP | acid phosphatase, prostate |
| 1552797_s_at | 12.167754 | down | PROM2 | prominin 2 |
| 219681_s_at | 12.068543 | down | RAB11FIP1 | RAB11 family interacting protein 1 |
| | | | | (class I) |
| 219115_s_at | 12.066238 | down | IL20RA | interleukin 20 receptor, alpha |
| 203797_at | 12.020528 | down | VSNL1 | visinin-like 1 |
| 1569886_a_at | 12.018516 | down | GLB1L3 | galactosidase, beta 1-like 3 |
| 212531_at | 12.014198 | down | LCN2 | lipocalin 2 |
| 200965_s_at | 11.87853 | down | ABLIM1 | actin binding LIM protein 1 |
| 206177_s_at | 11.859509 | down | ARG1 | arginase, liver |
| 227449_at | 11.784953 | down | EPHA4 | EPH receptor A4 |
| 205108_s_at | 11.751713 | down | APOB | apolipoprotein B (including Ag(x) |
| | | | | antigen) |
| 210096_at | 11.683115 | down | CYP4B1 | cytochrome P450, family 4, subfamily |
| | | | | B, polypeptide 1 |
| 217014_s_at | 11.667914 | down | AZGP1 /// | alpha-2-glycoprotein 1, zinc-binding /// |
| | | | AZGP1P1 | alpha-2-glycoprotein 1, zinc-binding |
| | | | | pseudogene 1 |
| 1564333_a_at | 11.665463 | down | PSAPL1 | prosaposin-like 1 |
| 222891_s_at | 11.499077 | down | BCL11A | B-cell CLL/lymphoma 11A (zinc |
| | | | | finger protein) |
| 224262_at | 11.446743 | down | IL1F10 | interleukin 1 family, member 10 (theta) |
| 238778_at | 11.35283 | down | MPP7 | membrane protein, palmitoylated 7 |
| | | | | (MAGUK p55 subfamily member 7) |
| 219895_at | 11.33746 | down | FAM70A | family with sequence similarity 70, |
| | | | | member A |
| 210020_x_at | 11.268426 | down | CALML3 | calmodulin-like 3 |
| 210085_s_at | 11.268161 | down | ANXA9 | annexin A9 |
| 207367_at | 11.168736 | down | ATP12A | ATPase, H+/K+ transporting, |
| | | | | nongastric, alpha polypeptide |
| 207008_at | 11.107841 | down | IL8RB | interleukin 8 receptor, beta |
| 206193_s_at | 11.105947 | down | CDSN | corneodesmosin |
| 218963_s_at | 10.944303 | down | KRT23 | keratin 23 (histone deacetylase |
| | | | | inducible) |
| 206192_at | 10.920915 | down | CDSN | corneodesmosin |
| 207254_at | 10.88806 | down | SLC15A1 | solute carrier family 15 (oligopeptide |
| | | | | transporter), member 1 |
| 220724_at | 10.756084 | down | FLJ21511 | hypothetical protein FLJ21511 |
| 219858_s_at | 10.733388 | down | MFSD6 | major facilitator superfamily domain |
| | | | | containing 6 |
| 236119_s_at | 10.709725 | down | SPRR2G | small proline-rich protein 2G |
| 232164_s_at | 10.52053 | down | EPPK1 | epiplakin 1 |
| 207381_at | 10.421057 | down | ALOX12B | arachidonate 12-lipoxygenase, 12R |
| | | | | type |
| 229764_at | 10.407254 | down | TPRG1 | tumor protein p63 regulated 1 |
| 218002_s_at | 10.403229 | down | CXCL14 | chemokine (C—X—C motif) ligand 14 |
| 227752_at | 10.369038 | down | SPTLC3 | serine palmitoyltransferase, long chain |
| | | | | base subunit 3 |
| 206385_s_at | 10.252723 | down | ANK3 | ankyrin 3, node of Ranvier (ankyrin G) |
| 236471_at | 10.205697 | down | NFE2L3 | nuclear factor (erythroid-derived 2)- |
| | | | | like 3 |
| 205513_at | 10.141289 | down | TCN1 | transcobalamin I (vitamin B12 binding |
| | | | | protein, R binder family) |
| 219313_at | 10.072841 | down | GRAMD1C | GRAM domain containing 1C |
| 228575_at | 10.042443 | down | IL20RB | interleukin 20 receptor beta |
| 227177_at | 9.9979105 | down | CORO2A | coronin, actin binding protein, 2A |
| 220090_at | 9.935171 | down | CRNN | cornulin |
| 1556793_a_at | 9.737699 | down | FAM83C | family with sequence similarity 83, |
| | | | | member C |
| 222242_s_at | 9.652021 | down | KLK5 | kallikrein-related peptidase 5 |
| 242204_at | 9.497831 | down | WFDC5 | WAP four-disulfide core domain 5 |
| 228523_at | 9.446722 | down | NANOS1 | nanos homolog 1 (Drosophila) |
| 226185_at | 9.266244 | down | CDS1 | CDP-diacylglycerol synthase |
| | | | | (phosphatidate cytidylyltransferase) 1 |
| 237690_at | 9.185177 | down | GPR115 | G protein-coupled receptor 115 |
| 214071_at | 9.122895 | down | MPPE1 | MRNA; cDNA DKFZp686K2379 |
| | | | | (from clone DKFZp686K2379) |
| 225540_at | 9.112212 | down | MAP2 | microtubule-associated protein 2 |
| 227209_at | 9.101238 | down | CNTN1 | Contactin 2 precursor (CNTN1) |
| 220624_s_at | 9.078715 | down | ELF5 | E74-like factor 5 (ets domain |
| | | | | transcription factor) |
| 243871_at | 9.058688 | down | LOC100130476 | PREDICTED: Homo sapiens similar to |
| | | | | hCG2036711 (LOC100130476), |
| | | | | mRNA |
| 242998_at | 8.8784485 | down | RDH12 | retinol dehydrogenase 12 (all-trans/9- |
| | | | | cis/11-cis) |
| 219995_s_at | 8.86155 | down | ZNF750 | zinc finger protein 750 |
| 225846_at | 8.854407 | down | RBM35A | RNA binding motif protein 35A |
| 227747_at | 8.839586 | down | MPZL3 | myelin protein zero-like 3 |
| 213056_at | 8.795081 | down | FRMD4B | FERM domain containing 4B |
| 204702_s_at | 8.784606 | down | NFE2L3 | nuclear factor (erythroid-derived 2)- |
| | | | | like 3 |
| 238017_at | 8.7703905 | down | SDR16C5 | short chain dehydrogenase/reductase |
| | | | | family 16C, member 5 |
| 220414_at | 8.748902 | down | CALML5 | calmodulin-like 5 |
| 225792_at | 8.72326 | down | HOOK1 | hook homolog 1 (Drosophila) |
| 209442_x_at | 8.710819 | down | ANK3 | ankyrin 3, node of Ranvier (ankyrin G) |
| 204469_at | 8.700343 | down | PTPRZ1 | protein tyrosine phosphatase, receptor- |
| | | | | type, Z polypeptide 1 |
| 226213_at | 8.644065 | down | ERBB3 | v-erb-b2 erythroblastic leukemia viral |
| | | | | oncogene homolog 3 (avian) |
| 205220_at | 8.635616 | down | GPR109B | G protein-coupled receptor 109B |
| 219369_s_at | 8.634883 | down | OTUB2 | OTU domain, ubiquitin aldehyde |
| | | | | binding 2 |
| 206595_at | 8.616346 | down | CST6 | cystatin E/M |
| 1556361_s_at | 8.6023035 | down | ANKRD13C | ankyrin repeat domain 13C |
| 1559226_x_at | 8.588551 | down | LCE1E | late cornified envelope 1E |
| 243582_at | 8.550055 | down | SH3RF2 | SH3 domain containing ring finger 2 |
| 221666_s_at | 8.532053 | down | PYCARD | PYD and CARD domain containing |
| 204455_at | 8.512845 | down | DST | dystonin |
| 201348_at | 8.504907 | down | GPX3 | glutathione peroxidase 3 (plasma) |
| 212538_at | 8.472947 | down | DOCK9 | dedicator of cytokinesis 9 |
| 211361_s_at | 8.422554 | down | SERPINB13 | serpin peptidase inhibitor, clade B |
| | | | | (ovalbumin), member 13 |
| 205969_at | 8.273929 | down | AADAC | arylacetamide deacetylase (esterase) |
| 220013_at | 8.225659 | down | ABHD9 | abhydrolase domain containing 9 |
| 219756_s_at | 8.173803 | down | POF1B | premature ovarian failure, 1B |
| 230349_at | 8.170966 | down | XKRX | XK, Kell blood group complex |
| | | | | subunit-related, X-linked |
| 238654_at | 8.165287 | down | LOC147645 | hypothetical protein LOC147645 |
| 220723_s_at | 8.1235 | down | FLJ21511 | hypothetical protein FLJ21511 |
| 225325_at | 8.100503 | down | MFSD6 | major facilitator superfamily domain |
| | | | | containing 6 |
| 236514_at | 8.063193 | down | ACOT8 | HIV-Nef associated acyl CoA |
| | | | | thioesterase (hNAACTE) |
| 224650_at | 7.978321 | down | MAL2 | mal, T-cell differentiation protein 2 |
| 205709_s_at | 7.964628 | down | CDS1 | CDP-diacylglycerol synthase |
| | | | | (phosphatidate cytidylyltransferase) 1 |
| 215704_at | 7.9460135 | down | FLG | PLAC2 placenta-specific 2 (non- |
| | | | | protein coding) |
| 223544_at | 7.941029 | down | TMEM79 | transmembrane protein 79 |
| 243722_at | 7.872192 | down | PYDC1 | PYD (pyrin domain) containing 1 |
| 206642_at | 7.8642535 | down | DSG1 | desmoglein 1 |
| 204952_at | 7.8289723 | down | LYPD3 | LY6/PLAUR domain containing 3 |
| 217087_at | 7.8114066 | down | C1orf68 | chromosome 1 open reading frame 68 |
| 222549_at | 7.7580996 | down | CLDN1 | claudin 1 |
| 229385_s_at | 7.6575327 | down | PLAC2 | placenta-specific 2 (non-protein |
| | | | | coding) |
| 219267_at | 7.655961 | down | GLTP | glycolipid transfer protein |
| 217272_s_at | 7.6130595 | down | SERPINB13 | serpin peptidase inhibitor, clade B |
| | | | | (ovalbumin), member 13 |
| 1555383_a_at | 7.5732036 | down | POF1B | premature ovarian failure, 1B |
| 238028_at | 7.5382996 | down | LOC100128918 | hypothetical protein LOC100128918 |
| 1552502_s_at | 7.5355606 | down | RHBDL2 | rhomboid, veinlet-like 2 (Drosophila) |
| 205568_at | 7.4951296 | down | AQP9 | aquaporin 9 |
| 205442_at | 7.491568 | down | MFAP3L | microfibrillar-associated protein 3-like |
| 227955_s_at | 7.4095197 | down | EFNA5 | ephrin-A5 |
| 210833_at | 7.343155 | down | PTGER3 | prostaglandin E receptor 3 (subtype |
| | | | | EP3) |
| 212543_at | 7.342191 | down | AIM1 | absent in melanoma 1 |
| 218186_at | 7.3316717 | down | RAB25 | RAB25, member RAS oncogene |
| | | | | family |
| 219403_s_at | 7.320557 | down | HPSE | heparanase |
| 210074_at | 7.306036 | down | CTSL2 | cathepsin L2 |
| 219850_s_at | 7.2735214 | down | EHF | ets homologous factor |
| 228708_at | 7.271517 | down | RAB27B | Small GTP-binding protein Rab27b |
| 227202_at | 7.2712536 | down | CNTN1 | Contactin 2 precursor (CNTN1) |
| 228538_at | 7.258556 | down | ZNF662 | zinc finger protein 662 |
| 232158_x_at | 7.256812 | down | NPAL1 | NIPA-like domain containing 1 |
| 226803_at | 7.2564917 | down | CHMP4C | chromatin modifying protein 4C |
| 206488_s_at | 7.236786 | down | CD36 | CD36 molecule (thrombospondin |
| | | | | receptor) |
| 218035_s_at | 7.19059 | down | RBM47 | RNA binding motif protein 47 |
| 205185_at | 7.1835594 | down | SPINK5 | serine peptidase inhibitor, Kazal type 5 |
| 206115_at | 7.160374 | down | EGR3 | early growth response 3 |
| 221854_at | 7.152312 | down | PKP1 | plakophilin 1 (ectodermal |
| | | | | dysplasia/skin fragility syndrome) |
| 239770_at | 7.1368814 | down | FAM62C | family with sequence similarity 62 (C2 |
| | | | | domain containing), member C |
| 214091_s_at | 7.1271777 | down | GPX3 | glutathione peroxidase 3 (plasma) |
| 218764_at | 7.120812 | down | PRKCH | protein kinase C, eta |
| 214536_at | 7.1027665 | down | SLURP1 | secreted LY6/PLAUR domain |
| | | | | containing 1 |
| 222496_s_at | 7.0082264 | down | RBM47 | RNA binding motif protein 47 |
| 232056_at | 7.0050087 | down | SCEL | sciellin |
| 217496_s_at | 6.950076 | down | IDE | insulin-degrading enzyme |
| 215465_at | 6.9448395 | down | ABCA12 | ATP-binding cassette, sub-family A |
| | | | | (ABC1), member 12 |
| 229070_at | 6.942199 | down | C6orf105 | chromosome 6 open reading frame 105 |
| 208937_s_at | 6.8969717 | down | ID1 | inhibitor of DNA binding 1, dominant |
| | | | | negative helix-loop-helix protein |
| 219630_at | 6.8924527 | down | PDZK1IP1 | PDZK1 interacting protein 1 |
| 1553454_at | 6.8833647 | down | RPTN | repetin |
| 1553589_a_at | 6.879349 | down | PDZK1IP1 | PDZK1 interacting protein 1 |
| 219388_at | 6.834852 | down | GRHL2 | grainyhead-like 2 (Drosophila) |
| 207326_at | 6.8310184 | down | BTC | betacellulin |
| 228038_at | 6.8282814 | down | SOX2 | SRY (sex determining region Y)-box 2 |
| 36499_at | 6.82723 | down | CELSR2 | cadherin, EGF LAG seven-pass G-type |
| | | | | receptor 2 (flamingo homolog, |
| | | | | Drosophila) |
| 231148_at | 6.815531 | down | IGFL2 | IGF-like family member 2 |
| 213611_at | 6.770561 | down | AQP5 | aquaporin 5 |
| 231849_at | 6.6379747 | down | KRT80 | keratin 80 |
| 226535_at | 6.6363 | down | ITGB6 | integrin, beta 6 |
| 204942_s_at | 6.622183 | down | ALDH3B2 | aldehyde dehydrogenase 3 family, |
| | | | | member B2 |
| 203178_at | 6.615657 | down | GATM | glycine amidinotransferase (L- |
| | | | | arginine:glycine amidinotransferase) |
| 1557136_at | 6.601717 | down | ATP13A4 | ATPase type 13A4 |
| 213927_at | 6.5928116 | down | MAP3K9 | mitogen-activated protein kinase |
| | | | | kinase kinase 9 |
| 220945_x_at | 6.560972 | down | MANSC1 | MANSC domain containing 1 |
| 206125_s_at | 6.5505314 | down | KLK8 | kallikrein-related peptidase 8 |
| 202712_s_at | 6.507372 | down | CKMT1A /// | creatine kinase, mitochondrial 1A /// |
| | | | CKMT1B /// | creatine kinase, mitochondrial 1B /// |
| | | | LOC100133623 | similar to Creatine kinase, ubiquitous |
| | | | | mitochondrial precursor (U-MtCK) |
| | | | | (Mia-CK) (Acidic-type mitochondrial |
| | | | | creatine kinase) |
| 1561225_at | 6.4982934 | down | LOC338579 | hypothetical protein LOC338579 |
| 224189_x_at | 6.491583 | down | EHF | ets homologous factor |
| 211788_s_at | 6.4853163 | down | TREX2 | three prime repair exonuclease 2 |
| 203180_at | 6.4791126 | down | ALDH1A3 | aldehyde dehydrogenase 1 family, |
| | | | | member A3 |
| 1555890_at | 6.4574604 | down | OR2A20P /// | olfactory receptor, family 2, subfamily |
| | | | OR2A9P | A, member 20 pseudogene /// olfactory |
| | | | | receptor, family 2, subfamily A, |
| | | | | member 9 pseudogene |
| 226482_s_at | 6.4535813 | down | hCG_20857 | thiosulfate sulfurtransferase KAT, |
| | | | /// RP11- | putative /// KAT protein |
| | | | 544M22.4 | |
| 208156_x_at | 6.4456053 | down | EPPK1 | epiplakin 1 |
| 228948_at | 6.4166746 | down | EPHA4 | EPH receptor A4 |
| 223658_at | 6.402442 | down | KCNK6 | potassium channel, subfamily K, |
| | | | | member 6 |
| 230179_at | 6.3794336 | down | LOC285812 | hypothetical protein LOC285812 |
| 1554253_a_at | 6.3690333 | down | LASS3 | LAG1 homolog, ceramide synthase 3 |
| 240304_s_at | 6.356037 | down | TMC5 | transmembrane channel-like 5 |
| 207109_at | 6.272226 | down | POU2F3 | POU class 2 homeobox 3 |
| 206114_at | 6.2673965 | down | EPHA4 | EPH receptor A4 |
| 219232_s_at | 6.265491 | down | EGLN3 | egl nine homolog 3 (C. elegans) |
| 41469_at | 6.259633 | down | PI3 | peptidase inhibitor 3, skin-derived |
| 238710_at | 6.2384377 | down | TMEM86A | transmembrane protein 86A |
| 202193_at | 6.232698 | down | LIMK2 | LIM domain kinase 2 |
| 220664_at | 6.2243595 | down | SPRR2C | small proline-rich protein 2C |
| | | | | (pseudogene) |
| 203021_at | 6.1999464 | down | SLPI | secretory leukocyte peptidase inhibitor |
| 210461_s_at | 6.1930823 | down | ABLIM1 | actin binding LIM protein 1 |
| 210015_s_at | 6.163196 | down | MAP2 | microtubule-associated protein 2 |
| 206392_s_at | 6.1626806 | down | RARRES1 | retinoic acid receptor responder |
| | | | | (tazarotene induced) 1 |
| 212570_at | 6.1608458 | down | ENDOD1 | endonuclease domain containing 1 |
| 214070_s_at | 6.1544147 | down | ATP10B | ATPase, class V, type 10B |
| 226863_at | 6.136124 | down | FAM110C | family with sequence similarity 110, |
| | | | | member C |
| 207192_at | 6.1150155 | down | DNASE1L2 | deoxyribonuclease I-like 2 |
| 215125_s_at | 6.1119647 | down | UGT1A1 /// | UDP glucuronosyltransferase 1 family, |
| | | | UGT1A10 /// | polypeptide A1 /// UDP |
| | | | UGT1A3 /// | glucuronosyltransferase 1 family, |
| | | | UGT1A4 /// | polypeptide A10 /// UDP |
| | | | UGT1A5 /// | glucuronosyltransferase 1 family, |
| | | | UGT1A6 /// | polypeptide A3 /// UDP |
| | | | UGT1A7 /// | glucuronosyltransferase 1 family, |
| | | | UGT1A8 /// | polypeptide A4 /// UDP |
| | | | UGT1A9 | glucuronosyltransferase 1 family, |
| | | | | polypeptide A5 /// UDP |
| | | | | glucuronosyltransferase 1 family, |
| | | | | polypeptide A6 /// UDP |
| | | | | glucuronosyltransferase 1 family, |
| | | | | polypeptide A7 /// UDP |
| | | | | glucuronosyltransferase 1 family, |
| | | | | polypeptide A8 /// UDP |
| | | | | glucuronosyltransferase 1 family, |
| | | | | polypeptide A9 |
| 1554921_a_at | 6.0999765 | down | SCEL | sciellin |
| 225645_at | 6.0821557 | down | EHF | Ets homologous factor, mRNA (cDNA |
| | | | | clone MGC: 47678 IMAGE: 6055934) |
| 219936_s_at | 6.0806403 | down | GPR87 | G protein-coupled receptor 87 |
| 219532_at | 6.073317 | down | ELOVL4 | elongation of very long chain fatty |
| | | | | acids (FEN1/Elo2, SUR4/Elo3, yeast)- |
| | | | | like 4 |
| 216258_s_at | 6.0713954 | down | SERPINB13 | serpin peptidase inhibitor, clade B |
| | | | | (ovalbumin), member 13 |
| 203328_x_at | 6.0695624 | down | IDE | insulin-degrading enzyme |
| 230323_s_at | 6.0668006 | down | TMEM45B | transmembrane protein 45B |
| 227180_at | 6.0664496 | down | ELOVL7 | ELOVL family member 7, elongation |
| | | | | of long chain fatty acids (yeast) |
| 219497_s_at | 6.0622134 | down | BCL11A | B-cell CLL/lymphoma 11A (zinc |
| | | | | finger protein) |
| 212560_at | 6.057562 | down | SORL1 | sortilin-related receptor, L(DLR class) |
| | | | | A repeats-containing |
| 216074_x_at | 6.0346746 | down | WWC1 | WW and C2 domain containing 1 |
| 210117_at | 6.022336 | down | SPAG1 | sperm associated antigen 1 |
| 221872_at | 5.9994154 | down | RARRES1 | retinoic acid receptor responder |
| | | | | (tazarotene induced) 1 |
| 238096_at | 5.9605427 | down | LOC284023 | hypothetical protein LOC284023 |
| 209603_at | 5.944856 | down | GATA3 | GATA binding protein 3 |
| 215554_at | 5.9089704 | down | GPLD1 | glycosylphosphatidylinositol specific |
| | | | | phospholipase D1 |
| 203691_at | 5.895547 | down | PI3 | peptidase inhibitor 3, skin-derived |
| 210130_s_at | 5.889496 | down | TM7SF2 | transmembrane 7 superfamily member 2 |
| 1553989_a_at | 5.889157 | down | ATP6V1C2 | ATPase, H+ transporting, lysosomal |
| | | | | 42 kDa, V1 subunit C2 |
| 235230_at | 5.864316 | down | PLCXD2 | phosphatidylinositol-specific |
| | | | | phospholipase C, X domain containing 2 |
| 1554897_s_at | 5.809435 | down | RHBDL2 | rhomboid, veinlet-like 2 (Drosophila) |
| 236172_at | 5.80707 | down | LTB4R | leukotriene B4 receptor |
| 228865_at | 5.8040857 | down | C1orf116 | chromosome 1 open reading frame 116 |
| 223659_at | 5.8040533 | down | TMPRSS13 | transmembrane protease, serine 13 |
| 205778_at | 5.800858 | down | KLK7 | kallikrein-related peptidase 7 |
| 219529_at | 5.7376733 | down | CLIC3 | chloride intracellular channel 3 |
| 216615_s_at | 5.662501 | down | HTR3A | 5-hydroxytryptamine (serotonin) |
| | | | | receptor 3A |
| 238567_at | 5.6408434 | down | SGPP2 | sphingosine-1-phosphate phosphotase 2 |
| 206165_s_at | 5.6202765 | down | CLCA2 | chloride channel regulator 2 |
| 206008_at | 5.6143503 | down | TGM1 | transglutaminase 1 (K polypeptide |
| | | | | epidermal type I, protein-glutamine- |
| | | | | gamma-glutamyltransferase) |
| 211906_s_at | 5.612926 | down | SERPINB4 | serpin peptidase inhibitor, clade B |
| | | | | (ovalbumin), member 4 |
| 235955_at | 5.6074624 | down | MARVELD2 | MARVEL domain containing 2 |
| 206214_at | 5.594604 | down | PLA2G7 | phospholipase A2, group VII (platelet- |
| | | | | activating factor acetylhydrolase, |
| | | | | plasma) |
| 202286_s_at | 5.5906916 | down | TACSTD2 | tumor-associated calcium signal |
| | | | | transducer 2 |
| 206164_at | 5.5694885 | down | CLCA2 | chloride channel regulator 2 |
| 239381_at | 5.5625143 | down | KLK7 | kallikrein-related peptidase 7 |
| 227014_at | 5.5577726 | down | ASPHD2 | aspartate beta-hydroxylase domain |
| | | | | containing 2 |
| 208539_x_at | 5.553559 | down | SPRR2B | small proline-rich protein 2B |
| 222847_s_at | 5.53052 | down | EGLN3 | egl nine homolog 3 (C. elegans) |
| 227450_at | 5.5204797 | down | ERP27 | endoplasmic reticulum protein 27 kDa |
| 225615_at | 5.499722 | down | IFFO2 | intermediate filament family orphan 2 |
| 219461_at | 5.4821987 | down | PAK6 | p21 protein (Cdc42/Rac)-activated |
| | | | | kinase 6 |
| 206683_at | 5.4811287 | down | ZNF165 | zinc finger protein 165 |
| 1555310_a_at | 5.4699154 | down | PAK6 | p21 protein (Cdc42/Rac)-activated |
| | | | | kinase 6 |
| 242828_at | 5.4434 | down | FIGN | fidgetin |
| 224329_s_at | 5.4422174 | down | CNFN | cornifelin |
| 219998_at | 5.4138575 | down | HSPC159 | galectin-related protein |
| 33767_at | 5.4136043 | down | NEFH | neurofilament, heavy polypeptide |
| 205363_at | 5.4128237 | down | BBOX1 | butyrobetaine (gamma), 2-oxoglutarate |
| | | | | dioxygenase (gamma-butyrobetaine |
| | | | | hydroxylase) 1 |
| 211597_s_at | 5.409704 | down | HOPX | HOP homeobox |
| 203962_s_at | 5.383551 | down | NEBL | nebulette |
| 206032_at | 5.376959 | down | DSC3 | desmocollin 3 |
| 227570_at | 5.3740797 | down | TMEM86A | transmembrane protein 86A |
| 226926_at | 5.3653264 | down | DMKN | dermokine |
| 202191_s_at | 5.363796 | down | GAS7 | growth arrest-specific 7 |
| 206482_at | 5.34657 | down | PTK6 | PTK6 protein tyrosine kinase 6 |
| 223611_s_at | 5.3417473 | down | LNX1 | ligand of numb-protein X 1 |
| 204379_s_at | 5.287966 | down | FGFR3 | fibroblast growth factor receptor 3 |
| 212992_at | 5.2794547 | down | AHNAK2 | AHNAK nucleoprotein 2 |
| 217528_at | 5.258966 | down | CLCA2 | chloride channel regulator 2 |
| 236534_at | 5.2476907 | down | BNIPL | BCL2/adenovirus E1B 19 kD |
| | | | | interacting protein like |
| 201131_s_at | 5.239927 | down | CDH1 | cadherin 1, type 1, E-cadherin |
| | | | | (epithelial) |
| 235099_at | 5.228119 | down | CMTM8 | CKLF-like MARVEL transmembrane |
| | | | | domain containing 8 |
| 226755_at | 5.2196016 | down | LOC642587 | CDNA FLJ33794 fis, clone |
| | | | | CTONG1000009 |
| 235146_at | 5.2194705 | down | TMCC3 | transmembrane and coiled-coil domain |
| | | | | family 3 |
| 1554593_s_at | 5.2057705 | down | SLC1A6 | solute carrier family 1 (high affinity |
| | | | | aspartate/glutamate transporter), |
| | | | | member 6 |
| 205832_at | 5.1992483 | down | CPA4 | carboxypeptidase A4 |
| 213085_s_at | 5.183778 | down | WWC1 | WW and C2 domain containing 1 |
| 229518_at | 5.179313 | down | FAM46B | family with sequence similarity 46, |
| | | | | member B |
| 226177_at | 5.172866 | down | GLTP | glycolipid transfer protein |
| 200862_at | 5.170659 | down | DHCR24 | 24-dehydrocholesterol reductase |
| 212242_at | 5.147954 | down | TUBA4A | tubulin, alpha 4a |
| 242103_at | 5.142739 | down | TMEM86A | transmembrane protein 86A |
| 206166_s_at | 5.137547 | down | CLCA2 | chloride channel regulator 2 |
| 210715_s_at | 5.117244 | down | SPINT2 | serine peptidase inhibitor, Kunitz type, 2 |
| 1554912_at | 5.1171923 | down | FAM62C | family with sequence similarity 62 (C2 |
| | | | | domain containing), member C |
| 1553077_at | 5.116311 | down | SDR9C7 | short chain dehydrogenase/reductase |
| | | | | family 9C, member 7 |
| 206628_at | 5.108023 | down | SLC5A1 | solute carrier family 5 (sodium/glucose |
| | | | | cotransporter), member 1 |
| 228698_at | 5.09854 | down | SOX7 | SRY (sex determining region Y)-box 7 |
| 220161_s_at | 5.089994 | down | EPB41L4B | erythrocyte membrane protein band 4.1 |
| | | | | like 4B |
| 202421_at | 5.073653 | down | IGSF3 | immunoglobulin superfamily, member 3 |
| 238909_at | 5.0720587 | down | S100A10 | Calpactin I light chain, 5′UTR region |
| 225299_at | 5.0482273 | down | MYO5B | myosin VB |
| 235141_at | 5.0444136 | down | MARVELD2 | MARVEL domain containing 2 |
| 201236_s_at | 5.0438776 | down | BTG2 | BTG family, member 2 |
| 1555382_at | 5.0425205 | down | POF1B | premature ovarian failure, 1B |
| 1557094_at | 5.0291085 | down | LOC653110 | hypothetical LOC653110 |
| 201243_s_at | 5.024576 | down | ATP1B1 | ATPase, Na+/K+ transporting, beta 1 |
| | | | | polypeptide |
| 207114_at | 5.0127378 | down | LY6G6C | lymphocyte antigen 6 complex, locus |
| | | | | G6C |
| 227676_at | 4.999608 | down | FAM3D | family with sequence similarity 3, |
| | | | | member D |
| 219680_at | 4.998101 | down | NLRX1 | NLR family member X1 |
| 202295_s_at | 4.9733486 | down | CTSH | cathepsin H |
| 206561_s_at | 4.97185 | down | AKR1B10 | aldo-keto reductase family 1, member |
| | | | | B10 (aldose reductase) |
| 218717_s_at | 4.965496 | down | LEPREL1 | leprecan-like 1 |
| 209212_s_at | 4.9630527 | down | KLF5 | Kruppel-like factor 5 (intestinal) |
| 207414_s_at | 4.9624557 | down | PCSK6 | proprotein convertase subtilisin/kexin |
| | | | | type 6 |
| 242271_at | 4.962056 | down | SLC26A9 | solute carrier family 26, member 9 |
| 216918_s_at | 4.960929 | down | DST | dystonin |
| 204855_at | 4.9595795 | down | SERPINB5 | serpin peptidase inhibitor, clade B |
| | | | | (ovalbumin), member 5 |
| 1553929_at | 4.9470873 | down | ACER1 | alkaline ceramidase 1 |
| 203961_at | 4.92486 | down | NEBL | nebulette |
| 205807_s_at | 4.9016848 | down | TUFT1 | tuftelin 1 |
| 203453_at | 4.897337 | down | SCNN1A | sodium channel, nonvoltage-gated 1 |
| | | | | alpha |
| 222383_s_at | 4.875127 | down | ALOXE3 | arachidonate lipoxygenase 3 |
| 1552319_a_at | 4.8667984 | down | KLK8 | kallikrein-related peptidase 8 |
| 216733_s_at | 4.8367143 | down | GATM | glycine amidinotransferase (L- |
| | | | | arginine:glycine amidinotransferase) |
| 208153_s_at | 4.819246 | down | FAT2 | FAT tumor suppressor homolog 2 |
| | | | | (Drosophila) |
| 1559224_at | 4.81139 | down | LCE1E | late cornified envelope 1E |
| 222892_s_at | 4.811261 | down | TMEM40 | transmembrane protein 40 |
| 213992_at | 4.8003573 | down | COL4A6 | collagen, type IV, alpha 6 |
| 206023_at | 4.7886114 | down | NMU | neuromedin U |
| 214734_at | 4.7853513 | down | EXPH5 | exophilin 5 |
| 60474_at | 4.7850847 | down | FERMT1 | fermitin family homolog 1 |
| | | | | (Drosophila) |
| 219498_s_at | 4.7693014 | down | BCL11A | B-cell CLL/lymphoma 11A (zinc |
| | | | | finger protein) |
| 59625_at | 4.763774 | down | NOL3 | nucleolar protein 3 (apoptosis repressor |
| | | | | with CARD domain) |
| 202179_at | 4.7527394 | down | BLMH | bleomycin hydrolase |
| 236128_at | 4.73937 | down | ZNF91 | zinc finger protein 91 |
| 203642_s_at | 4.735424 | down | COBLL1 | COBL-like 1 |
| 205011_at | 4.726828 | down | VWA5A | von Willebrand factor A domain |
| | | | | containing 5A |
| 203407_at | 4.7218986 | down | PPL | periplakin |
| 205590_at | 4.7012725 | down | RASGRP1 | RAS guanyl releasing protein 1 |
| | | | | (calcium and DAG-regulated) |
| 222603_at | 4.700272 | down | ERMP1 | endoplasmic reticulum |
| | | | | metallopeptidase 1 |
| 203779_s_at | 4.6950703 | down | MPZL2 | myelin protein zero-like 2 |
| 210834_s_at | 4.694923 | down | PTGER3 | prostaglandin E receptor 3 (subtype |
| | | | | EP3) |
| 223484_at | 4.6944065 | down | C15orf48 | chromosome 15 open reading frame 48 |
| 206033_s_at | 4.676169 | down | DSC3 | desmocollin 3 |
| 232306_at | 4.6732864 | down | CDH26 | cadherin-like 26 |
| 215808_at | 4.67278 | down | KLK10 | kallikrein-related peptidase 10 |
| 202411_at | 4.671116 | down | IFI27 | interferon, alpha-inducible protein 27 |
| 207802_at | 4.665757 | down | CRISP3 | cysteine-rich secretory protein 3 |
| 221667_s_at | 4.658692 | down | HSPB8 | heat shock 22 kDa protein 8 |
| 219087_at | 4.6529336 | down | ASPN | asporin |
| 218741_at | 4.6508865 | down | CENPM | centromere protein M |
| 203741_s_at | 4.6428866 | down | ADCY7 | adenylate cyclase 7 |
| 203074_at | 4.63983 | down | ANXA8 /// | annexin A8 /// annexin A8-like 1 /// |
| | | | ANXA8L1 /// | annexin A8-like 2 |
| | | | ANXA8L2 | |
| 201286_at | 4.6295676 | down | SDC1 | syndecan 1 |
| 231733_at | 4.617893 | down | CARD18 | caspase recruitment domain family, |
| | | | | member 18 |
| 209873_s_at | 4.6149898 | down | PKP3 | plakophilin 3 |
| 212573_at | 4.6098948 | down | ENDOD1 | endonuclease domain containing 1 |
| 244780_at | 4.60221 | down | SGPP2 | sphingosine-1-phosphate phosphotase 2 |
| 225177_at | 4.5943675 | down | RAB11FIP1 | RAB11 family interacting protein 1 |
| | | | | (class I) |
| 223322_at | 4.591433 | down | RASSF5 | Ras association (RalGDS/AF-6) |
| | | | | domain family member 5 |
| 227309_at | 4.5890346 | down | YOD1 | YOD1 OTU deubiquinating enzyme 1 |
| | | | | homolog (S. cerevisiae) |
| 206515_at | 4.587165 | down | CYP4F3 | cytochrome P450, family 4, subfamily |
| | | | | F, polypeptide 3 |
| 204995_at | 4.5868692 | down | CDK5R1 | cyclin-dependent kinase 5, regulatory |
| | | | | subunit 1 (p35) |
| 202826_at | 4.585347 | down | SPINT1 | serine peptidase inhibitor, Kunitz type 1 |
| 205651_x_at | 4.5728 | down | RAPGEF4 | Rap guanine nucleotide exchange |
| | | | | factor (GEF) 4 |
| 222746_s_at | 4.5700636 | down | BSPRY | B-box and SPRY domain containing |
| 219722_s_at | 4.555332 | down | GDPD3 | glycerophosphodiester |
| | | | | phosphodiesterase domain containing 3 |
| 205538_at | 4.549461 | down | CORO2A | coronin, actin binding protein, 2A |
| 210619_s_at | 4.5378346 | down | HYAL1 | hyaluronoglucosaminidase 1 |
| 204990_s_at | 4.5270753 | down | ITGB4 | integrin, beta 4 |
| 227204_at | 4.515133 | down | PARD6G | par-6 partitioning defective 6 homolog |
| | | | | gamma (C. elegans) |
| 238063_at | 4.513157 | down | TMEM154 | transmembrane protein 154 |
| 219358_s_at | 4.5108666 | down | ADAP2 | ArfGAP with dual PH domains 2 |
| 206276_at | 4.498863 | down | LY6D | lymphocyte antigen 6 complex, locus D |
| 209792_s_at | 4.4899607 | down | KLK10 | kallikrein-related peptidase 10 |
| 208892_s_at | 4.489426 | down | DUSP6 | dual specificity phosphatase 6 |
| 206265_s_at | 4.488534 | down | GPLD1 | glycosylphosphatidylinositol specific |
| | | | | phospholipase D1 |
| 235852_at | 4.484227 | down | STON2 | CDNA FLJ37480 fis, clone |
| | | | | BRAWH2013866, highly similar to |
| | | | | Homo sapiens stonin 2 mRNA |
| 221107_at | 4.484099 | down | CHRNA9 | cholinergic receptor, nicotinic, alpha 9 |
| 219691_at | 4.478258 | down | SAMD9 | sterile alpha motif domain containing 9 |
| 226499_at | 4.476055 | down | NRARP | MRNA full length insert cDNA clone |
| | | | | EUROIMAGE 1499812 |
| 227556_at | 4.4707904 | down | NME7 | non-metastatic cells 7, protein |
| | | | | expressed in (nucleoside-diphosphate |
| | | | | kinase) |
| 1558846_at | 4.46551 | down | PNLIPRP3 | pancreatic lipase-related protein 3 |
| 215425_at | 4.46542 | down | BTG3 | BTG family, member 3 |
| 226226_at | 4.4539366 | down | TMEM45B | transmembrane protein 45B |
| 204204_at | 4.4521403 | down | SLC31A2 | solute carrier family 31 (copper |
| | | | | transporters), member 2 |
| 208893_s_at | 4.4509206 | down | DUSP6 | dual specificity phosphatase 6 |
| 204029_at | 4.4487 | down | CELSR2 | cadherin, EGF LAG seven-pass G-type |
| | | | | receptor 2 (flamingo homolog, |
| | | | | Drosophila) |
| 201656_at | 4.4418707 | down | ITGA6 | integrin, alpha 6 |
| 206034_at | 4.426019 | down | SERPINB8 | serpin peptidase inhibitor, clade B |
| | | | | (ovalbumin), member 8 |
| 206391_at | 4.4257607 | down | RARRES1 | retinoic acid receptor responder |
| | | | | (tazarotene induced) 1 |
| 228531_at | 4.4111724 | down | SAMD9 | sterile alpha motif domain containing 9 |
| 203638_s_at | 4.4092565 | down | FGFR2 | fibroblast growth factor receptor 2 |
| 206264_at | 4.4085407 | down | GPLD1 | glycosylphosphatidylinositol specific |
| | | | | phospholipase D1 |
| 201015_s_at | 4.407748 | down | JUP | junction plakoglobin |
| 226029_at | 4.401417 | down | VANGL2 | vang-like 2 (van gogh, Drosophila) |
| 202053_s_at | 4.3994246 | down | ALDH3A2 | aldehyde dehydrogenase 3 family, |
| | | | | member A2 |
| 205675_at | 4.3889413 | down | MTTP | microsomal triglyceride transfer |
| | | | | protein |
| 203917_at | 4.3878665 | down | CXADR | coxsackie virus and adenovirus |
| | | | | receptor |
| 1560250_s_at | 4.384614 | down | LOC284242 | hypothetical protein LOC284242 |
| 201242_s_at | 4.3839946 | down | ATP1B1 | ATPase, Na+/K+ transporting, beta 1 |
| | | | | polypeptide |
| 203126_at | 4.3744597 | down | IMPA2 | inositol(myo)-1(or 4)- |
| | | | | monophosphatase 2 |
| 219412_at | 4.3728795 | down | RAB38 | RAB38, member RAS oncogene |
| | | | | family |
| 220124_at | 4.371851 | down | GAN | gigaxonin |
| 211067_s_at | 4.3444204 | down | GAS7 | growth arrest-specific 7 |
| 220066_at | 4.3412647 | down | NOD2 | nucleotide-binding oligomerization |
| | | | | domain containing 2 |
| 230188_at | 4.3192954 | down | ICHTHYIN | ichthyin protein |
| 202504_at | 4.3078084 | down | TRIM29 | tripartite motif-containing 29 |
| 1569144_a_at | 4.3075566 | down | C9orf169 /// | chromosome 9 open reading frame 169 |
| | | | LOC100130547 | /// hypothetical protein LOC100130547 |
| 226733_at | 4.2923603 | down | PFKFB2 | 6-phosphofructo-2-kinase/fructose-2,6- |
| | | | | biphosphatase 2 |
| 222829_s_at | 4.292116 | down | IL20RA | interleukin 20 receptor, alpha |
| 229720_at | 4.2816586 | down | BAG1 | BCL2-associated athanogene |
| 219395_at | 4.2743106 | down | RBM35B | RNA binding motif protein 35B |
| 208891_at | 4.2635026 | down | DUSP6 | dual specificity phosphatase 6 |
| 203585_at | 4.2597046 | down | ZNF185 | zinc finger protein 185 (LIM domain) |
| 221690_s_at | 4.2536693 | down | NLRP2 | NLR family, pyrin domain containing 2 |
| 231270_at | 4.2472186 | down | CA13 | carbonic anhydrase XIII |
| 244692_at | 4.2340446 | down | CYP4F22 | cytochrome P450, family 4, subfamily |
| | | | | F, polypeptide 22 |
| 220413_at | 4.2331395 | down | SLC39A2 | solute carrier family 39 (zinc |
| | | | | transporter), member 2 |
| 202289_s_at | 4.229875 | down | TACC2 | transforming, acidic coiled-coil |
| | | | | containing protein 2 |
| 211362_s_at | 4.220602 | down | SERPINB13 | serpin peptidase inhibitor, clade B |
| | | | | (ovalbumin), member 13 |
| 203327_at | 4.2173615 | down | IDE | insulin-degrading enzyme |
| 1555173_at | 4.212528 | down | STX19 | syntaxin 19 |
| 213924_at | 4.212505 | down | MPPE1 | MRNA; cDNA DKFZp686K2379 |
| | | | | (from clone DKFZp686K2379) |
| 204484_at | 4.199939 | down | PIK3C2B | phosphoinositide-3-kinase, class 2, |
| | | | | beta polypeptide |
| 226907_at | 4.199478 | down | PPP1R14C | protein phosphatase 1, regulatory |
| | | | | (inhibitor) subunit 14C |
| 205863_at | 4.1991963 | down | S100A12 | S100 calcium binding protein A12 |
| 222881_at | 4.1936674 | down | HPSE | heparanase |
| 201884_at | 4.1872177 | down | CEACAM5 | carcinoembryonic antigen-related cell |
| | | | | adhesion molecule 5 |
| 228570_at | 4.181567 | down | BTBD11 | BTB (POZ) domain containing 11 |
| 210138_at | 4.179159 | down | RGS20 | regulator of G-protein signaling 20 |
| 228010_at | 4.178918 | down | PPP2R2C | protein phosphatase 2 (formerly 2A), |
| | | | | regulatory subunit B, gamma isoform |
| 232181_at | 4.1646137 | down | LOC153346 | hypothetical protein LOC153346 |
| 41660_at | 4.1577396 | down | CELSR1 | cadherin, EGF LAG seven-pass G-type |
| | | | | receptor 1 (flamingo homolog, |
| | | | | Drosophila) |
| 227197_at | 4.152967 | down | SGEF | Src homology 3 domain-containing |
| | | | | guanine nucleotide exchange factor |
| 225822_at | 4.1515694 | down | TMEM125 | transmembrane protein 125 |
| 219474_at | 4.142456 | down | C3orf52 | chromosome 3 open reading frame 52 |
| 205900_at | 4.1411896 | down | KRT1 | keratin 1 |
| 203367_at | 4.1374893 | down | DUSP14 | dual specificity phosphatase 14 |
| 225671_at | 4.136382 | down | SPNS2 | spinster homolog 2 (Drosophila) |
| 55081_at | 4.132561 | down | MICALL1 | MICAL-like 1 |
| 223832_s_at | 4.123751 | down | CAPNS2 | calpain, small subunit 2 |
| 214490_at | 4.1185994 | down | ARSF | arylsulfatase F |
| 210372_s_at | 4.1101165 | down | TPD52L1 | tumor protein D52-like 1 |
| 209863_s_at | 4.1084065 | down | TP63 | tumor protein p63 |
| 210297_s_at | 4.1008377 | down | MSMB | microseminoprotein, beta- |
| 224210_s_at | 4.0987024 | down | PXMP4 | peroxisomal membrane protein 4, |
| | | | | 24 kDa |
| 221245_s_at | 4.0908217 | down | FZD5 | frizzled homolog 5 (Drosophila) |
| 205977_s_at | 4.087086 | down | EPHA1 | EPH receptor A1 |
| 230563_at | 4.0841923 | down | RASGEF1A | RasGEF domain family, member 1A |
| 226272_at | 4.0820107 | down | RCAN3 | RCAN family member 3 |
| 204004_at | 4.077613 | down | PAWR | PRKC, apoptosis, WT1, regulator |
| 205534_at | 4.076567 | down | PCDH7 | protocadherin 7 |
| 239272_at | 4.074428 | down | MMP28 | matrix metallopeptidase 28 |
| 204300_at | 4.0636954 | down | PET112L | PET112-like (yeast) |
| 235879_at | 4.043492 | down | MBNL1 | MBNL protein |
| 1554179_s_at | 4.0318265 | down | LYNX1 | Ly6/neurotoxin 1 |
| 235272_at | 4.0244374 | down | SBSN | suprabasin |
| 235085_at | 4.018871 | down | PRAGMIN | homolog of rat pragma of Rnd2 |
| 213954_at | 4.013676 | down | FAM169A | family with sequence similarity 169, |
| | | | | member A |
| 226490_at | 4.012788 | down | NHSL1 | NHS-like 1 |
| 211002_s_at | 4.012248 | down | TRIM29 | tripartite motif-containing 29 |
| 212706_at | 3.9973824 | down | LOC100132214 | similar to HSPC047 protein /// similar |
| | | | /// | to RAS p21 protein activator 4 /// |
| | | | LOC100133005 | similar to HSPC047 protein /// RAS |
| | | | /// | p21 protein activator 4 |
| | | | LOC100134722 | |
| | | | /// RASA4 | |
| 225354_s_at | 3.9922047 | down | SH3BGRL2 | SH3 domain binding glutamic acid- |
| | | | | rich protein like 2 |
| 205190_at | 3.9871655 | down | PLS1 | plastin 1 (I isoform) |
| 202054_s_at | 3.9822264 | down | ALDH3A2 | aldehyde dehydrogenase 3 family, |
| | | | | member A2 |
| 232449_at | 3.9744756 | down | BCO2 | beta-carotene oxygenase 2 |
| 218342_s_at | 3.9690635 | down | ERMP1 | endoplasmic reticulum |
| | | | | metallopeptidase 1 |
| 206043_s_at | 3.963122 | down | ATP2C2 | ATPase, Ca++ transporting, type 2C, |
| | | | | member 2 |
| 206284_x_at | 3.962128 | down | CLTB | clathrin, light chain (Lcb) |
| 209126_x_at | 3.952012 | down | KRT6B | keratin 6B |
| 1553213_a_at | 3.9390984 | down | KRT78 | keratin 78 |
| 210413_x_at | 3.938409 | down | SERPINB3 /// | serpin peptidase inhibitor, clade B |
| | | | SERPINB4 | (ovalbumin), member 3 /// serpin |
| | | | | peptidase inhibitor, clade B |
| | | | | (ovalbumin), member 4 |
| 223895_s_at | 3.9289596 | down | EPN3 | epsin 3 |
| 202546_at | 3.9224277 | down | VAMP8 | vesicle-associated membrane protein 8 |
| | | | | (endobrevin) |
| 206453_s_at | 3.921748 | down | NDRG2 | NDRG family member 2 |
| 205020_s_at | 3.9133503 | down | ARL4A | ADP-ribosylation factor-like 4A |
| 213279_at | 3.907437 | down | DHRS1 | dehydrogenase/reductase (SDR family) |
| | | | | member 1 |
| 214838_at | 3.9040148 | down | SFT2D2 | SFT2 domain containing 2 |
| 210102_at | 3.8968651 | down | VWA5A | von Willebrand factor A domain |
| | | | | containing 5A |
| 225687_at | 3.8950646 | down | FAM83D | family with sequence similarity 83, |
| | | | | member D |
| 1553695_a_at | 3.8867338 | down | NLRX1 | NLR family member X1 |
| 226064_s_at | 3.8825026 | down | DGAT2 | diacylglycerol O-acyltransferase |
| | | | | homolog 2 (mouse) |
| 205773_at | 3.8760667 | down | CPEB3 | cytoplasmic polyadenylation element |
| | | | | binding protein 3 |
| 223232_s_at | 3.8725953 | down | CGN | cingulin |
| 209570_s_at | 3.870752 | down | D4S234E | DNA segment on chromosome 4 |
| | | | | (unique) 234 expressed sequence |
| 224806_at | 3.8689046 | down | TRIM25 | tripartite motif-containing 25 |
| 1554648_a_at | 3.8646846 | down | DUOXA1 | dual oxidase maturation factor 1 |
| 213501_at | 3.8544102 | down | ACOX1 | acyl-Coenzyme A oxidase 1, palmitoyl |
| 220786 s_at | 3.844398 | down | SLC38A4 | solute carrier family 38, member 4 |
| 216060_s_at | 3.8402324 | down | DAAM1 | dishevelled associated activator of |
| | | | | morphogenesis 1 |
| 224435_at | 3.8286057 | down | C10orf57 /// | chromosome 10 open reading frame 57 |
| | | | C10orf58 | /// chromosome 10 open reading frame |
| | | | | 58 |
| 202489_s_at | 3.826976 | down | FXYD3 | FXYD domain containing ion transport |
| | | | | regulator 3 |
| 228124_at | 3.8211079 | down | ABHD12 | abhydrolase domain containing 12 |
| 223748_at | 3.8204598 | down | SLC4A11 | solute carrier family 4, sodium borate |
| | | | | transporter, member 11 |
| 225301_s_at | 3.8147264 | down | MYO5B | myosin VB |
| 205030_at | 3.803794 | down | FABP7 | fatty acid binding protein 7, brain |
| 203997_at | 3.7991052 | down | PTPN3 | protein tyrosine phosphatase, non- |
| | | | | receptor type 3 |
| 206409_at | 3.792147 | down | TIAM1 | T-cell lymphoma invasion and |
| | | | | metastasis 1 |
| 230464_at | 3.7890499 | down | S1PR5 | sphingosine-1-phosphate receptor 5 |
| 217080_s_at | 3.788767 | down | HOMER2 | homer homolog 2 (Drosophila) |
| 228596_at | 3.7859466 | down | LOC728377 | similar to rho guanine nucleotide |
| | | | | exchange factor 5 |
| 219121_s_at | 3.7856553 | down | RBM35A | RNA binding motif protein 35A |
| 206605_at | 3.7852893 | down | P11 | 26 serine protease |
| 218796_at | 3.7840903 | down | FERMT1 | fermitin family homolog 1 |
| | | | | (Drosophila) |
| 206004_at | 3.7830596 | down | TGM3 | transglutaminase 3 (E polypeptide, |
| | | | | protein-glutamine-gamma- |
| | | | | glutamyltransferase) |
| 204519_s_at | 3.7812307 | down | PLLP | plasma membrane proteolipid |
| | | | | (plasmolipin) |
| 203128_at | 3.7796383 | down | SPTLC2 | serine palmitoyltransferase, long chain |
| | | | | base subunit 2 |
| 231875_at | 3.7795398 | down | KIF21A | kinesin family member 21A |
| 212096_s_at | 3.7734342 | down | MTUS1 | mitochondrial tumor suppressor 1 |
| 226003_at | 3.7700498 | down | KIF21A | kinesin family member 21A |
| 200832_s_at | 3.7699654 | down | SCD | stearoyl-CoA desaturase (delta-9- |
| | | | | desaturase) |
| 208614_s_at | 3.7652352 | down | FLNB | filamin B, beta (actin binding protein |
| | | | | 278) |
| 35820_at | 3.763104 | down | GM2A | GM2 ganglioside activator |
| 235048_at | 3.7585585 | down | FAM169A | family with sequence similarity 169, |
| | | | | member A |
| 202540_s_at | 3.7533727 | down | HMGCR | 3-hydroxy-3-methylglutaryl-Coenzyme |
| | | | | A reductase |
| 210375_at | 3.7446036 | down | PTGER3 | prostaglandin E receptor 3 (subtype |
| | | | | EP3) |
| 219010_at | 3.7444217 | down | C1orf106 | chromosome 1 open reading frame 106 |
| 226129_at | 3.7394128 | down | FAM83H | family with sequence similarity 83, |
| | | | | member H |
| 232151_at | 3.7381117 | down | MACC1 | metastasis associated in colon cancer 1 |
| 236313_at | 3.736902 | down | CDKN2B | cyclin-dependent kinase inhibitor 2B |
| | | | | (p15, inhibits CDK4) |
| 227962_at | 3.7365305 | down | ACOX1 | acyl-Coenzyme A oxidase 1, palmitoyl |
| 235626_at | 3.7264879 | down | CAMK1D | calcium/calmodulin-dependent protein |
| | | | | kinase ID |
| 1559190_s_at | 3.7257981 | down | RDH13 | CDNA PSEC0082 fis, clone |
| | | | | NT2RP2004966, highly similar to |
| | | | | Retinol dehydrogenase 13 (EC 1.1.1.—) |
| 227735_s_at | 3.7186708 | down | C10orf99 | chromosome 10 open reading frame 99 |
| 227109_at | 3.7156994 | down | CYP2R1 | cytochrome P450, family 2, subfamily |
| | | | | R, polypeptide 1 |
| 242773_at | 3.714043 | down | SLC5A1 | solute carrier family 5 (sodium/glucose |
| | | | | cotransporter), member 1 |
| 223541_at | 3.713034 | down | HAS3 | hyaluronan synthase 3 |
| 1553057_at | 3.7106702 | down | SERPINB12 | serpin peptidase inhibitor, clade B |
| | | | | (ovalbumin), member 12 |
| 214279_s_at | 3.7052615 | down | NDRG2 | NDRG family member 2 |
| 221566_s_at | 3.7041054 | down | NOL3 | nucleolar protein 3 (apoptosis repressor |
| | | | | with CARD domain) |
| 219496_at | 3.6988554 | down | ANKRD57 | ankyrin repeat domain 57 |
| 225806_at | 3.6983435 | down | JUB | jub, ajuba homolog (Xenopus laevis) |
| 204765_at | 3.6892798 | down | ARHGEF5 | Rho guanine nucleotide exchange |
| | | | | factor (GEF) 5 |
| 207430 s_at | 3.6888688 | down | MSMB | microseminoprotein, beta- |
| 226382_at | 3.6853971 | down | LOC283070 | hypothetical protein LOC283070 |
| 201693_s_at | 3.6807284 | down | EGR1 | early growth response 1 |
| 206277_at | 3.679081 | down | P2RY2 | purinergic receptor P2Y, G-protein |
| | | | | coupled, 2 |
| 219821_s_at | 3.6787474 | down | GFOD1 | glucose-fructose oxidoreductase |
| | | | | domain containing 1 |
| 212737_at | 3.6714 | down | GM2A | GM2 ganglioside activator |
| 209719_x_at | 3.6645396 | down | SERPINB3 | serpin peptidase inhibitor, clade B |
| | | | | (ovalbumin), member 3 |
| 205172_x_at | 3.6626048 | down | CLTB | clathrin, light chain (Lcb) |
| 1553764_a_at | 3.662437 | down | JUB | jub, ajuba homolog (Xenopus laevis) |
| 204351_at | 3.6600852 | down | S100P | S100 calcium binding protein P |
| 203148_s_at | 3.6579587 | down | TRIM14 | tripartite motif-containing 14 |
| 209000_s_at | 3.6571388 | down | 40064 | septin 8 |
| 205786_s_at | 3.6454937 | down | ITGAM | integrin, alpha M (complement |
| | | | | component 3 receptor 3 subunit) |
| 223298_s_at | 3.6409454 | down | NT5C3 | 5′-nucleotidase, cytosolic III |
| 1568868_at | 3.6400533 | down | CYP27C1 | cytochrome P450, family 27, |
| | | | | subfamily C, polypeptide 1 |
| 210544_s_at | 3.6329489 | down | ALDH3A2 | aldehyde dehydrogenase 3 family, |
| | | | | member A2 |
| 212314_at | 3.628236 | down | KIAA0746 /// | KIAA0746 protein /// serine |
| | | | SERINC2 | incorporator 2 |
| 206421_s_at | 3.622729 | down | SERPINB7 | serpin peptidase inhibitor, clade B |
| | | | | (ovalbumin), member 7 |
| 202539_s_at | 3.6214926 | down | HMGCR | 3-hydroxy-3-methylglutaryl-Coenzyme |
| | | | | A reductase |
| 1553211_at | 3.6197479 | down | ANKFN1 | ankyrin-repeat and fibronectin type III |
| | | | | domain containing 1 |
| 202761_s_at | 3.6197405 | down | SYNE2 | spectrin repeat containing, nuclear |
| | | | | envelope 2 |
| 205783_at | 3.6192052 | down | KLK13 | kallikrein-related peptidase 13 |
| 225502_at | 3.607785 | down | DOCK8 | dedicator of cytokinesis 8 |
| 216905_s_at | 3.6036117 | down | ST14 | suppression of tumorigenicity 14 |
| | | | | (colon carcinoma) |
| 221779_at | 3.6017182 | down | MICALL1 | MICAL-like 1 |
| 204734_at | 3.6002238 | down | KRT15 | keratin 15 |
| 231732_at | 3.594498 | down | SMPD3 | sphingomyelin phosphodiesterase 3, |
| | | | | neutral membrane (neutral |
| | | | | sphingomyelinase II) |
| 220030_at | 3.5835576 | down | STYK1 | serine/threonine/tyrosine kinase 1 |
| 39248_at | 3.5794623 | down | AQP3 | aquaporin 3 (Gill blood group) |
| 222876_s_at | 3.5791473 | down | ADAP2 | ArfGAP with dual PH domains 2 |
| 204636_at | 3.5764592 | down | COL17A1 | collagen, type XVII, alpha 1 |
| 210553_x_at | 3.5725062 | down | PCSK6 | proprotein convertase subtilisin/kexin |
| | | | | type 6 |
| 225864_at | 3.5664208 | down | FAM84B | family with sequence similarity 84, |
| | | | | member B |
| 209211_at | 3.5630605 | down | KLF5 | Kruppel-like factor 5 (intestinal) |
| 1552777_a_at | 3.5581756 | down | RAET1E | retinoic acid early transcript 1E |
| 239547_at | 3.5570898 | down | HS3ST6 | heparan sulfate (glucosamine) 3-O- |
| | | | | sulfotransferase 6 |
| 224839_s_at | 3.5547016 | down | GPT2 | glutamic pyruvate transaminase |
| | | | | (alanine aminotransferase) 2 |
| 1553364_at | 3.5530663 | down | PNPLA1 | patatin-like phospholipase domain |
| | | | | containing 1 |
| 235117_at | 3.5501385 | down | CHAC2 | ChaC, cation transport regulator |
| | | | | homolog 2 (E. coli) |
| 222354_at | 3.5409195 | down | F11R | F11 receptor |
| 219976_at | 3.54039 | down | HOOK1 | hook homolog 1 (Drosophila) |
| 211372_s_at | 3.5362017 | down | IL1R2 | interleukin 1 receptor, type II |
| 219316_s_at | 3.5322564 | down | FLVCR2 | feline leukemia virus subgroup C |
| | | | | cellular receptor family, member 2 |
| 224646_x_at | 3.5317452 | down | H19 | H19, imprinted maternally expressed |
| | | | | transcript (non-protein coding) |
| 65438_at | 3.5268037 | down | KIAA1609 | KIAA1609 |
| 226666_at | 3.5185058 | down | DAAM1 | dishevelled associated activator of |
| | | | | morphogenesis 1 |
| 219597_s_at | 3.5163934 | down | DUOX1 | dual oxidase 1 |
| 205421_at | 3.5158527 | down | SLC22A3 | solute carrier family 22 (extraneuronal |
| | | | | monoamine transporter), member 3 |
| 206714_at | 3.5117545 | down | ALOX15B | arachidonate 15-lipoxygenase, type B |
| 219752_at | 3.5069795 | down | RASAL1 | RAS protein activator like 1 (GAP1 |
| | | | | like) |
| 203256_at | 3.5057578 | down | CDH3 | cadherin 3, type 1, P-cadherin |
| | | | | (placental) |
| 207558_s_at | 3.5035906 | down | PITX2 | paired-like homeodomain 2 |
| 231969_at | 3.4996755 | down | STOX2 | storkhead box 2 |
| 205249_at | 3.4971406 | down | EGR2 | early growth response 2 (Krox-20 |
| | | | | homolog, Drosophila) |
| 1563900_at | 3.4969769 | down | FAM83B | family with sequence similarity 83, |
| | | | | member B |
| 210026_s_at | 3.474502 | down | CARD10 | caspase recruitment domain family, |
| | | | | member 10 |
| 225095_at | 3.4744895 | down | SPTLC2 | KIAA0526 protein |
| 1553333_at | 3.474137 | down | C1orf161 | chromosome 1 open reading frame 161 |
| 239710_at | 3.4720347 | down | FIGN | fidgetin |
| 203780_at | 3.4676228 | down | MPZL2 | myelin protein zero-like 2 |
| 221843_s_at | 3.4610984 | down | KIAA1609 | KIAA1609 |
| 208191_x_at | 3.4576283 | down | PSG4 | pregnancy specific beta-1-glycoprotein 4 |
| 201428_at | 3.4539902 | down | CLDN4 | claudin 4 |
| 229296_at | 3.4521377 | down | LOC100128501 | hypothetical protein LOC100128501 |
| 211043_s_at | 3.446346 | down | CLTB | clathrin, light chain (Lcb) |
| 218432_at | 3.4458005 | down | FBXO3 | F-box protein 3 |
| 201005_at | 3.4436064 | down | CD9 | CD9 molecule |
| 210868_s_at | 3.4410589 | down | ELOVL6 | ELOVL family member 6, elongation |
| | | | | of long chain fatty acids (FEN1/Elo2, |
| | | | | SUR4/Elo3-like, yeast) |
| 227276_at | 3.4398205 | down | PLXDC2 | plexin domain containing 2 |
| 213820_s_at | 3.4357908 | down | STARD5 | StAR-related lipid transfer (START) |
| | | | | domain containing 5 |
| 209301_at | 3.4329016 | down | CA2 | carbonic anhydrase II |
| 204941_s_at | 3.4287107 | down | ALDH3B2 | aldehyde dehydrogenase 3 family, |
| | | | | member B2 |
| 223199_at | 3.4269447 | down | MKNK2 | MAP kinase interacting |
| | | | | serine/threonine kinase 2 |
| 224327_s_at | 3.4269319 | down | DGAT2 | diacylglycerol O-acyltransferase |
| | | | | homolog 2 (mouse) |
| 235678_at | 3.4267802 | down | GM2A | GM2 ganglioside activator |
| 236225_at | 3.426428 | down | GGT6 | gamma-glutamyltransferase 6 |
| 221567_at | 3.4161565 | down | NOL3 | nucleolar protein 3 (apoptosis repressor |
| | | | | with CARD domain) |
| 204546_at | 3.4134994 | down | KIAA0513 | KIAA0513 |
| 204058_at | 3.4133024 | down | ME1 | malic enzyme 1, NADP(+)-dependent, |
| | | | | cytosolic |
| 210065_s_at | 3.4122272 | down | UPK1B | uroplakin 1B |
| 1557165_s_at | 3.4083939 | down | KLHL18 | kelch-like 18 (Drosophila) |
| 205014_at | 3.40602 | down | FGFBP1 | fibroblast growth factor binding |
| | | | | protein 1 |
| 221669_s_at | 3.4037082 | down | ACAD8 | acyl-Coenzyme A dehydrogenase |
| | | | | family, member 8 |
| 225525_at | 3.4010828 | down | CTA- | KIAA1671 protein /// hypothetical |
| | | | 221G9.4 /// | protein LOC100131004 |
| | | | LOC100131004 | |
| 231118_at | 3.3923123 | down | ANKRD35 | ankyrin repeat domain 35 |
| 229337_at | 3.3913348 | down | USP2 | ubiquitin specific peptidase 2 |
| 219670_at | 3.3814268 | down | BEND5 | BEN domain containing 5 |
| 205048_s_at | 3.3812659 | down | PSPH | phosphoserine phosphatase |
| 1554062_at | 3.3779898 | down | XG | Xg blood group |
| 222866_s_at | 3.372543 | down | FLVCR2 | feline leukemia virus subgroup C |
| | | | | cellular receptor family, member 2 |
| 219429_at | 3.369738 | down | FA2H | fatty acid 2-hydroxylase |
| 232500_at | 3.36646 | down | C20orf74 | chromosome 20 open reading frame 74 |
| 209600_s_at | 3.3606262 | down | ACOX1 | acyl-Coenzyme A oxidase 1, palmitoyl |
| 236213_at | 3.3596435 | down | LOC100130885 | hypothetical protein LOC100130885 |
| 225536_at | 3.354181 | down | TMEM54 | transmembrane protein 54 |
| 201534_s_at | 3.353898 | down | UBL3 | ubiquitin-like 3 |
| 228729_at | 3.349102 | down | CCNB1 | cyclin B1 |
| 223839_s_at | 3.3401856 | down | SCD /// | stearoyl-CoA desaturase (delta-9- |
| | | | SCDOS | desaturase) /// stearoyl-CoA desaturase |
| | | | | opposite strand |
| 243611_at | 3.3352149 | down | MICALCL | MICAL C-terminal like |
| 211382_s_at | 3.335143 | down | TACC2 | transforming, acidic coiled-coil |
| | | | | containing protein 2 |
| 220149_at | 3.3329418 | down | C2orf54 | chromosome 2 open reading frame 54 |
| 227856_at | 3.3276808 | down | C4orf32 | chromosome 4 open reading frame 32 |
| 228469_at | 3.3271294 | down | PPID | Cyclophilin-40 |
| 230518_at | 3.3258517 | down | MPZL2 | myelin protein zero-like 2 |
| 209558_s_at | 3.322049 | down | HIP1R | huntingtin interacting protein 1 related |
| 227015_at | 3.3209336 | down | ASPHD2 | aspartate beta-hydroxylase domain |
| | | | | containing 2 |
| 201287_s_at | 3.32023 | down | SDC1 | syndecan 1 |
| 205470_s_at | 3.3177328 | down | KLK11 | kallikrein-related peptidase 11 |
| 209569_x_at | 3.3130386 | down | D4S234E | DNA segment on chromosome 4 |
| | | | | (unique) 234 expressed sequence |
| 228640_at | 3.3072002 | down | PCDH7 | protocadherin 7 |
| 225001_at | 3.305868 | down | RAB3D | RAB3D, member RAS oncogene |
| | | | | family |
| 232082_x_at | 3.3027992 | down | SPRR3 | small proline-rich protein 3 |
| 223694_at | 3.3023326 | down | TRIM7 | tripartite motif-containing 7 |
| 215393_s_at | 3.3011584 | down | COBLL1 | COBL-like 1 |
| 229114_at | 3.3010995 | down | GAB1 | GRB2-associated binding protein 1 |
| 235405_at | 3.2997503 | down | GSTA4 | glutathione S-transferase alpha 4 |
| 232090_at | 3.2993267 | down | LOC100128178 | similar to hCG2041313 |
| 228256_s_at | 3.2980163 | down | EPB41L4A | erythrocyte membrane protein band 4.1 |
| | | | | like 4A |
| 227134_at | 3.2938037 | down | SYTL1 | synaptotagmin-like 1 |
| 218150_at | 3.2934453 | down | ARL5A | ADP-ribosylation factor-like 5A |
| 205403_at | 3.2927191 | down | IL1R2 | interleukin 1 receptor, type II |
| 231771_at | 3.2887797 | down | GJB6 | gap junction protein, beta 6, 30 kDa |
| 227782_at | 3.2884097 | down | ZBTB7C | zinc finger and BTB domain |
| | | | | containing 7C |
| 227461_at | 3.287732 | down | STON2 | stonin 2 |
| 203509_at | 3.2846527 | down | SORL1 | sortilin-related receptor, L(DLR class) |
| | | | | A repeats-containing |
| 223168_at | 3.2830467 | down | RHOU | ras homolog gene family, member U |
| 209631_s_at | 3.2649126 | down | GPR37 | G protein-coupled receptor 37 |
| | | | | (endothelin receptor type B-like) |
| 205029_s_at | 3.2629297 | down | FABP7 | fatty acid binding protein 7, brain |
| 226649_at | 3.2610345 | down | PANK1 | pantothenate kinase 1 |
| 227889_at | 3.25648 | down | LPCAT2 | lysophosphatidylcholine |
| | | | | acyltransferase 2 |
| 213094_at | 3.2563322 | down | GPR126 | G protein-coupled receptor 126 |
| 244261_at | 3.2521746 | down | IL28RA | interleukin 28 receptor, alpha |
| | | | | (interferon, lambda receptor) |
| 201340_s_at | 3.2456722 | down | ENC1 | ectodermal-neural cortex (with BTB- |
| | | | | like domain) |
| 217974_at | 3.2413647 | down | TM7SF3 | transmembrane 7 superfamily member 3 |
| 223631_s_at | 3.2411804 | down | C19orf33 | chromosome 19 open reading frame 33 |
| 230765_at | 3.2385905 | down | KIAA1239 | KIAA1239 |
| 1552566_at | 3.2320094 | down | BTBD16 | BTB (POZ) domain containing 16 |
| 225613_at | 3.2306297 | down | LOC100128443 | hypothetical protein LOC100128443 /// |
| | | | /// MAST4 | microtubule associated |
| | | | | serine/threonine kinase family member 4 |
| 205776_at | 3.222692 | down | FMO5 | flavin containing monooxygenase 5 |
| 203007_x_at | 3.2214603 | down | LYPLA1 | lysophospholipase I |
| 229103_at | 3.2176034 | down | WNT3 | wingless-type MMTV integration site |
| | | | | family, member 3 |
| 224367_at | 3.2115495 | down | BEX2 | brain expressed X-linked 2 |
| 225834_at | 3.2113602 | down | FAM72A /// | family with sequence similarity 72, |
| | | | FAM72B /// | member A /// family with sequence |
| | | | GCUD2 | similarity 72, member B /// gastric |
| | | | | cancer up-regulated-2 |
| 230266_at | 3.2071729 | down | RAB7B | RAB7B, member RAS oncogene |
| | | | | family |
| 206059_at | 3.2045915 | down | ZNF91 | zinc finger protein 91 |
| 225807_at | 3.1980147 | down | JUB | jub, ajuba homolog (Xenopus laevis) |
| 203786_s_at | 3.196283 | down | TPD52L1 | tumor protein D52-like 1 |
| 1554895_a_at | 3.1930003 | down | RHBDL2 | rhomboid, veinlet-like 2 (Drosophila) |
| 203665_at | 3.1928537 | down | HMOX1 | heme oxygenase (decycling) 1 |
| 204363_at | 3.1915643 | down | F3 | coagulation factor III (thromboplastin, |
| | | | | tissue factor) |
| 238164_at | 3.190472 | down | USP6NL | USP6 N-terminal like |
| 212322_at | 3.1838899 | down | SGPL1 | sphingosine-1-phosphate lyase 1 |
| 1566766_a_at | 3.1814005 | down | MACC1 | metastasis associated in colon cancer 1 |
| 207126_x_at | 3.1803102 | down | UGT1A1 /// | UDP glucuronosyltransferase 1 family, |
| | | | UGT1A10 /// | polypeptide A1 /// UDP |
| | | | UGT1A4 /// | glucuronosyltransferase 1 family, |
| | | | UGT1A6 /// | polypeptide A10 /// UDP |
| | | | UGT1A8 /// | glucuronosyltransferase 1 family, |
| | | | UGT1A9 | polypeptide A4 /// UDP |
| | | | | glucuronosyltransferase 1 family, |
| | | | | polypeptide A6 /// UDP |
| | | | | glucuronosyltransferase 1 family, |
| | | | | polypeptide A8 /// UDP |
| | | | | glucuronosyltransferase 1 family, |
| | | | | polypeptide A9 |
| 222223_s_at | 3.176874 | down | IL1F5 | interleukin 1 family, member 5 (delta) |
| 239694_at | 3.174275 | down | TRIM7 | tripartite motif-containing 7 |
| 206969_at | 3.1713967 | down | KRT34 | keratin 34 |
| 207540_s_at | 3.1631026 | down | SYK | spleen tyrosine kinase |
| 235857_at | 3.157146 | down | KCTD11 | potassium channel tetramerisation |
| | | | | domain containing 11 |
| 205829_at | 3.1498919 | down | HSD17B1 | hydroxysteroid (17-beta) |
| | | | | dehydrogenase 1 |
| 209720_s_at | 3.1471493 | down | SERPINB3 | serpin peptidase inhibitor, clade B |
| | | | | (ovalbumin), member 3 |
| 226698_at | 3.1410942 | down | FCHSD1 | FCH and double SH3 domains 1 |
| 203147_s_at | 3.140271 | down | TRIM14 | tripartite motif-containing 14 |
| 214036_at | 3.1385825 | down | EFNA5 | ephrin-A5 |
| 1558378_a_at | 3.1375017 | down | AHNAK2 | AHNAK nucleoprotein 2 |
| 203887_s_at | 3.1370428 | down | THBD | thrombomodulin |
| 205015_s_at | 3.1335502 | down | TGFA | transforming growth factor, alpha |
| 218756_s_at | 3.1297085 | down | DHRS11 | dehydrogenase/reductase (SDR family) |
| | | | | member 11 |
| 204256_at | 3.1214519 | down | ELOVL6 | ELOVL family member 6, elongation |
| | | | | of long chain fatty acids (FEN1/Elo2, |
| | | | | SUR4/Elo3-like, yeast) |
| 206912_at | 3.120563 | down | FOXE1 | forkhead box E1 (thyroid transcription |
| | | | | factor 2) |
| 209885_at | 3.1201258 | down | RHOD | ras homolog gene family, member D |
| 229638_at | 3.1183026 | down | IRX3 | iroquois homeobox 3 |
| 220249_at | 3.116939 | down | HYAL4 | hyaluronoglucosaminidase 4 |
| 210347_s_at | 3.1119802 | down | BCL11A | B-cell CLL/lymphoma 11A (zinc |
| | | | | finger protein) |
| 224480_s_at | 3.1057024 | down | AGPAT9 | 1-acylglycerol-3-phosphate O- |
| | | | | acyltransferase 9 |
| 236359_at | 3.10485 | down | SCN4B | sodium channel, voltage-gated, type |
| | | | | IV, beta |
| 229374_at | 3.1005714 | down | EPHA4 | EPH receptor A4 |
| 226617_at | 3.0999246 | down | ARL5A | ADP-ribosylation factor-like 5A |
| 221856_s_at | 3.0998528 | down | FAM63A | family with sequence similarity 63, |
| | | | | member A |
| 238720_at | 3.0991333 | down | OMG | OMGP mRNA for oligodendrocyte- |
| | | | | myelin glycoprotein |
| 214295_at | 3.0939116 | down | KIAA0485 | hypothetical LOC57235 |
| 218951_s_at | 3.0916145 | down | PLCXD1 | phosphatidylinositol-specific |
| | | | | phospholipase C, X domain containing 1 |
| 223574_x_at | 3.0913527 | down | PPP2R2C | protein phosphatase 2 (formerly 2A), |
| | | | | regulatory subunit B, gamma isoform |
| 224685_at | 3.0898316 | down | MLLT4 | myeloid/lymphoid or mixed-lineage |
| | | | | leukemia (trithorax homolog, |
| | | | | Drosophila); translocated to, 4 |
| 231778_at | 3.089387 | down | DLX3 | distal-less homeobox 3 |
| 229290_at | 3.0886714 | down | DAPL1 | death associated protein-like 1 |
| 218829_s_at | 3.0885017 | down | CHD7 | chromodomain helicase DNA binding |
| | | | | protein 7 |
| 223894_s_at | 3.0799034 | down | AKTIP | AKT interacting protein |
| 226187_at | 3.0795898 | down | CDS1 | CDP-diacylglycerol synthase |
| | | | | (phosphatidate cytidylyltransferase) 1 |
| 227944_at | 3.077466 | down | PTPN3 | protein tyrosine phosphatase, non- |
| | | | | receptor type 3 |
| 241813_at | 3.0757666 | down | MBD1 | methyl-CpG binding domain protein 1 |
| 203287_at | 3.0720277 | down | LAD1 | ladinin 1 |
| 229546_at | 3.0575104 | down | LOC653602 | hypothetical LOC653602 |
| 218849_s_at | 3.0525377 | down | PPP1R13L | protein phosphatase 1, regulatory |
| | | | | (inhibitor) subunit 13 like |
| 1558281_a_at | 3.0518956 | down | TMEM184A | transmembrane protein 184A |
| 224579_at | 3.0447147 | down | SLC38A1 | solute carrier family 38, member 1 |
| 222830_at | 3.0440314 | down | GRHL1 | grainyhead-like 1 (Drosophila) |
| 209605_at | 3.037887 | down | TST | thiosulfate sulfurtransferase |
| | | | | (rhodanese) |
| 206632_s_at | 3.0369 | down | APOBEC3B | apolipoprotein B mRNA editing |
| | | | | enzyme, catalytic polypeptide-like 3B |
| 213030_s_at | 3.0361433 | down | PLXNA2 | plexin A2 |
| 213787_s_at | 3.0355 | down | EBP | emopamil binding protein (sterol |
| | | | | isomerase) |
| 212095_s_at | 3.0337477 | down | MTUS1 | mitochondrial tumor suppressor 1 |
| 1564308_a_at | 3.0335965 | down | MPP7 | membrane protein, palmitoylated 7 |
| | | | | (MAGUK p55 subfamily member 7) |
| 204059_s_at | 3.028911 | down | ME1 | malic enzyme 1, NADP(+)-dependent, |
| | | | | cytosolic |
| 218922_s_at | 3.0278475 | down | LASS4 | LAG1 homolog, ceramide synthase 4 |
| 219990_at | 3.021536 | down | E2F8 | E2F transcription factor 8 |
| 244758_at | 3.021281 | down | SCAND3 | SCAN domain containing 3 |
| 201427_s_at | 3.011953 | down | SEPP1 | selenoprotein P, plasma, 1 |
| 237899_at | 3.0117605 | down | LOC729994 | hypothetical LOC729994 |
| 233814_at | 3.0052407 | down | EFNA5 | Receptor tyrosine kinase ligand LERK- |
| | | | | 7 precursor (EPLG7) |
| 226829_at | 3.0025802 | down | AFAP1L2 | actin filament associated protein 1-like 2 |
| 203397_s_at | 3.002098 | down | GALNT3 | UDP-N-acetyl-alpha-D- |
| | | | | galactosamine:polypeptide N- |
| | | | | acetylgalactosaminyltransferase 3 |
| | | | | (GalNAc-T3) |
| 222809_x_at | 3.0019972 | down | C14orf65 | chromosome 14 open reading frame 65 |
| 234725_s_at | 3.0007038 | down | SEMA4B | sema domain, immunoglobulin domain |
| | | | | (Ig), transmembrane domain (TM) and |
| | | | | short cytoplasmic domain, |
| | | | | (semaphorin) 4B |
| 203560_at | 2.9994648 | down | GGH | gamma-glutamyl hydrolase (conjugase, |
| | | | | folylpolygammaglutamyl hydrolase) |
| 233565_s_at | 2.9988334 | down | SDCBP2 | syndecan binding protein (syntenin) 2 |
| 1553212_at | 2.9964101 | down | KRT78 | keratin 78 |
| 204135_at | 2.9959981 | down | FILIP1L | filamin A interacting protein 1-like |
| 223278_at | 2.995849 | down | GJB2 | gap junction protein, beta 2, 26 kDa |
| 227385_at | 2.9950788 | down | PPAPDC2 | phosphatidic acid phosphatase type 2 |
| | | | | domain containing 2 |
| 216347_s_at | 2.9949014 | down | PPP1R13B | protein phosphatase 1, regulatory |
| | | | | (inhibitor) subunit 13B |
| 203359_s_at | 2.9933915 | down | MYCBP | c-myc binding protein |
| 242064_at | 2.9917743 | down | SDK2 | sidekick homolog 2 (chicken) |
| 1553114_a_at | 2.9870312 | down | PTK6 | PTK6 protein tyrosine kinase 6 |
| 236266 at | 2.985503 | down | RORA | Hypothetical protein LOC283666, |
| | | | | mRNA (cDNA clone |
| | | | | IMAGE: 4750925) |
| 242317_at | 2.9845624 | down | HIGD1A | HIG1 domain family, member 1A |
| 209203_s_at | 2.984112 | down | BICD2 | bicaudal D homolog 2 (Drosophila) |
| 226245_at | 2.9801052 | down | KCTD1 | potassium channel tetramerisation |
| | | | | domain containing 1 |
| 214765_s_at | 2.9758816 | down | NAAA | N-acylethanolamine acid amidase |
| 223216_x_at | 2.9703996 | down | ZNF395 | zinc finger protein 395 |
| 221215_s_at | 2.9702475 | down | RIPK4 | receptor-interacting serine-threonine |
| | | | | kinase 4 |
| 221081_s_at | 2.9699113 | down | DENND2D | DENN/MADD domain containing 2D |
| 202154_x_at | 2.9669414 | down | TUBB3 | tubulin, beta 3 |
| 208126_s_at | 2.9666116 | down | CYP2C18 | cytochrome P450, family 2, subfamily |
| | | | | C, polypeptide 18 |
| 213476_x_at | 2.966195 | down | TUBB3 | tubulin, beta 3 |
| 203722_at | 2.9630923 | down | ALDH4A1 | aldehyde dehydrogenase 4 family, |
| | | | | member A1 |
| 227135_at | 2.9602783 | down | NAAA | N-acylethanolamine acid amidase |
| 214705_at | 2.9595757 | down | INADL | InaD-like (Drosophila) |
| 211985_s_at | 2.9585447 | down | CALM1 /// | calmodulin 1 (phosphorylase kinase, |
| | | | CALM2 /// | delta) /// calmodulin 2 (phosphorylase |
| | | | CALM3 | kinase, delta) /// calmodulin 3 |
| | | | | (phosphorylase kinase, delta) |
| 238513_at | 2.9496446 | down | PRRG4 | Proline rich Gla (G-carboxyglutamic |
| | | | | acid) 4 (transmembrane), mRNA |
| | | | | (cDNA clone MGC: 19793 |
| | | | | IMAGE: 3841745) |
| 243837_x_at | 2.947061 | down | LOC100128500 | hypothetical protein LOC100128500 |
| 218677_at | 2.9463606 | down | S100A14 | S100 calcium binding protein A14 |
| 218900_at | 2.9451632 | down | CNNM4 | cyclin M4 |
| 220266_s_at | 2.9415443 | down | KLF4 | Kruppel-like factor 4 (gut) |
| 204341_at | 2.9409094 | down | TRIM16 | tripartite motif-containing 16 |
| 230769_at | 2.939519 | down | DENND2C | DENN/MADD domain containing 2C |
| 232693_s_at | 2.9363637 | down | FBXO16 /// | F-box protein 16 /// zinc finger protein |
| | | | ZNF395 | 395 |
| 226246_at | 2.9359393 | down | KCTD1 | potassium channel tetramerisation |
| | | | | domain containing 1 |
| 226421 at | 2.933277 | down | AMMECR1 | Alport syndrome, mental retardation, |
| | | | | midface hypoplasia and elliptocytosis |
| | | | | chromosomal region gene 1 |
| 225655_at | 2.9276872 | down | UHRF1 | ubiquitin-like with PHD and ring |
| | | | | finger domains 1 |
| 222717_at | 2.926197 | down | SDPR | serum deprivation response |
| | | | | (phosphatidylserine binding protein) |
| 235871_at | 2.9253323 | down | LIPH | lipase, member H |
| 223339_at | 2.9252036 | down | ATPIF1 | ATPase inhibitory factor 1 |
| 223233_s_at | 2.9242868 | down | CGN | cingulin |
| 225051_at | 2.9183056 | down | EPB41 | erythrocyte membrane protein band 4.1 |
| | | | | (elliptocytosis 1, RH-linked) |
| 209372_x_at | 2.914044 | down | TUBB2A /// | tubulin, beta 2A /// tubulin, beta 2B |
| | | | TUBB2B | |
| 224495_at | 2.9137444 | down | TMEM107 | transmembrane protein 107 |
| 218722_s_at | 2.9108763 | down | CCDC51 | coiled-coil domain containing 51 |
| 228221_at | 2.9098802 | down | SLC44A3 | solute carrier family 44, member 3 |
| 207955_at | 2.907911 | down | CCL27 | chemokine (C-C motif) ligand 27 |
| 206094_x_at | 2.9065409 | down | UGT1A1 /// | UDP glucuronosyltransferase 1 family, |
| | | | UGT1A10 /// | polypeptide A1 /// UDP |
| | | | UGT1A3 /// | glucuronosyltransferase 1 family, |
| | | | UGT1A4 /// | polypeptide A10 /// UDP |
| | | | UGT1A5 /// | glucuronosyltransferase 1 family, |
| | | | UGT1A6 /// | polypeptide A3 /// UDP |
| | | | UGT1A7 /// | glucuronosyltransferase 1 family, |
| | | | UGT1A8 /// | polypeptide A4 /// UDP |
| | | | UGT1A9 | glucuronosyltransferase 1 family, |
| | | | | polypeptide A5 /// UDP |
| | | | | glucuronosyltransferase 1 family, |
| | | | | polypeptide A6 /// UDP |
| | | | | glucuronosyltransferase 1 family, |
| | | | | polypeptide A7 /// UDP |
| | | | | glucuronosyltransferase 1 family, |
| | | | | polypeptide A8 /// UDP |
| | | | | glucuronosyltransferase 1 family, |
| | | | | polypeptide A9 |
| 231830_x_at | 2.902842 | down | RAB11FIP1 | RAB11 family interacting protein 1 |
| | | | | (class I) |
| 1554966_a_at | 2.9024658 | down | FILIP1L | filamin A interacting protein 1-like |
| 226959_at | 2.9023726 | down | LOC283070 | CDNA FLJ40058 fis, clone |
| | | | | TCOLN1000180 |
| 206400_at | 2.9014132 | down | LGALS7 /// | lectin, galactoside-binding, soluble, 7 |
| | | | LGALS7B | /// lectin, galactoside-binding, soluble, |
| | | | | 7B |
| 1554246_at | 2.8979917 | down | C1orf210 | chromosome 1 open reading frame 210 |
| 227736_at | 2.8947847 | down | C10orf99 | chromosome 10 open reading frame 99 |
| 221123_x_at | 2.8909688 | down | ZNF395 | zinc finger protein 395 |
| 222890_at | 2.8908694 | down | CCDC113 | coiled-coil domain containing 113 |
| 208190_s_at | 2.890325 | down | LSR | lipolysis stimulated lipoprotein |
| | | | | receptor |
| 229396_at | 2.8896506 | down | OVOL1 | ovo-like 1(Drosophila) |
| 227034_at | 2.888465 | down | ANKRD57 | ankyrin repeat domain 57 |
| 219648_at | 2.8866937 | down | MREG | melanoregulin |
| 218792_s_at | 2.8844924 | down | BSPRY | B-box and SPRY domain containing |
| 204542_at | 2.8817751 | down | ST6GALNAC2 | ST6 (alpha-N-acetyl-neuraminyl-2,3- |
| | | | | beta-galactosyl-1,3)-N- |
| | | | | acetylgalactosaminide alpha-2,6- |
| | | | | sialyltransferase 2 |
| 238962_at | 2.877806 | down | ZNF681 | zinc finger protein 681 |
| 227964_at | 2.877196 | down | FRMD8 | FERM domain containing 8 |
| 238964_at | 2.8724544 | down | FIGN | fidgetin |
| 201564_s_at | 2.8720455 | down | FSCN1 | fascin homolog 1, actin-bundling |
| | | | | protein (Strongylocentrotus |
| | | | | purpuratus) |
| 38340_at | 2.8708196 | down | HIP1R | huntingtin interacting protein 1 related |
| 218032_at | 2.8703704 | down | SNN | stannin |
| 204547_at | 2.8703067 | down | RAB40B | RAB40B, member RAS oncogene |
| | | | | family |
| 213506_at | 2.8534102 | down | F2RL1 | coagulation factor II (thrombin) |
| | | | | receptor-like 1 |
| 235095_at | 2.851771 | down | CCDC64B | coiled-coil domain containing 64B |
| 202962_at | 2.8498514 | down | KIF13B | kinesin family member 13B |
| 200606_at | 2.8482468 | down | DSP | desmoplakin |
| 220578_at | 2.8452191 | down | ADAMTSL4 | ADAMTS-like 4 |
| 218789_s_at | 2.840476 | down | C11orf71 | chromosome 11 open reading frame 71 |
| 242093_at | 2.8397858 | down | SYTL5 | synaptotagmin-like 5 |
| 228975_at | 2.8345933 | down | SP6 | Sp6 transcription factor |
| 1569555_at | 2.832668 | down | GDA | guanine deaminase |
| 201694_s_at | 2.8325536 | down | EGR1 | early growth response 1 |
| 210128_s_at | 2.8322942 | down | LTB4R | leukotriene B4 receptor |
| 213805_at | 2.8317797 | down | ABHD5 | abhydrolase domain containing 5 |
| 238762_at | 2.8312757 | down | MTHFD2L | methylenetetrahydrofolate |
| | | | | dehydrogenase (NADP+ dependent) 2- |
| | | | | like |
| 1558111_at | 2.8277514 | down | MBNL1 | muscleblind-like (Drosophila) |
| 223000_s_at | 2.8271255 | down | F11R | F11 receptor |
| 227998_at | 2.8236406 | down | S100A16 | S100 calcium binding protein A16 |
| 238206_at | 2.8226795 | down | RXFP1 | relaxin/insulin-like family peptide |
| | | | | receptor 1 |
| 1552648_a_at | 2.82174 | down | TNFRSF10A | tumor necrosis factor receptor |
| | | | | superfamily, member 10a |
| 235850_at | 2.8215306 | down | WDR5B | WD repeat domain 5B |
| 200636_s_at | 2.816918 | down | PTPRF | protein tyrosine phosphatase, receptor |
| | | | | type, F |
| 218779_x_at | 2.813133 | down | EPS8L1 | EPS8-like 1 |
| 220456_at | 2.811995 | down | SPTLC3 | serine palmitoyltransferase, long chain |
| | | | | base subunit 3 |
| 210652_s_at | 2.8102167 | down | TTC39A | tetratricopeptide repeat domain 39A |
| 239853_at | 2.809029 | down | KLC3 | kinesin light chain 3 |
| 205765_at | 2.8077855 | down | CYP3A5 | cytochrome P450, family 3, subfamily |
| | | | | A, polypeptide 5 |
| 1552620_at | 2.8073084 | down | SPRR4 | small proline-rich protein 4 |
| 225779_at | 2.8072405 | down | SLC27A4 | solute carrier family 27 (fatty acid |
| | | | | transporter), member 4 |
| 203699_s_at | 2.806607 | down | DIO2 | deiodinase, iodothyronine, type II |
| 231810_at | 2.8063636 | down | BRI3BP | Cervical cancer oncogene binding |
| | | | | protein |
| 221893_s_at | 2.7909083 | down | ADCK2 | aarF domain containing kinase 2 |
| 209260_at | 2.7901292 | down | SFN | stratifin |
| 205054_at | 2.7879531 | down | NEB | nebulin |
| 213050_at | 2.7861958 | down | COBL | cordon-bleu homolog (mouse) |
| 218028_at | 2.7856884 | down | ELOVL1 | elongation of very long chain fatty |
| | | | | acids (FEN1/Elo2, SUR4/Elo3, yeast)- |
| | | | | like 1 |
| 224496_s_at | 2.7819924 | down | TMEM107 | transmembrane protein 107 |
| 1553031_at | 2.7813523 | down | GPR115 | G protein-coupled receptor 115 |
| 223497_at | 2.7785342 | down | FAM135A | family with sequence similarity 135, |
| | | | | member A |
| 204856_at | 2.7779574 | down | B3GNT3 | UDP-GlcNAc:betaGal beta-1,3-N- |
| | | | | acetylglucosaminyltransferase 3 |
| 220911_s_at | 2.7676141 | down | KIAA1305 | KIAA1305 |
| 219696_at | 2.7632923 | down | DENND1B | DENN/MADD domain containing 1B |
| 1559096_x_at | 2.758654 | down | FBXO9 | F-box protein 9 |
| 204976_s_at | 2.7562022 | down | AMMECR1 | Alport syndrome, mental retardation, |
| | | | | midface hypoplasia and elliptocytosis |
| | | | | chromosomal region gene 1 |
| 218149_s_at | 2.7463727 | down | ZNF395 | zinc finger protein 395 |
| 229513_at | 2.7461805 | down | STRBP | Chromosome 9 open reading frame 45, |
| | | | | mRNA (cDNA clone MGC: 45613 |
| | | | | IMAGE: 2989018) |
| 235605_at | 2.7404695 | down | CASZ1 | castor zinc finger 1 |
| 213201_s_at | 2.7397995 | down | TNNT1 | troponin T type 1 (skeletal, slow) |
| 204141_at | 2.738532 | down | TUBB2A | tubulin, beta 2A |
| 230398_at | 2.7380996 | down | TNS4 | tensin 4 |
| 238013_at | 2.7366548 | down | PLEKHA2 | pleckstrin homology domain |
| | | | | containing, family A (phosphoinositide |
| | | | | binding specific) member 2 |
| 204532_x_at | 2.7337391 | down | UGT1A1 /// | UDP glucuronosyltransferase 1 family, |
| | | | UGT1A10 /// | polypeptide A1 /// UDP |
| | | | UGT1A4 /// | glucuronosyltransferase 1 family, |
| | | | UGT1A6 /// | polypeptide A10 /// UDP |
| | | | UGT1A8 /// | glucuronosyltransferase 1 family, |
| | | | UGT1A9 | polypeptide A4 /// UDP |
| | | | | glucuronosyltransferase 1 family, |
| | | | | polypeptide A6 /// UDP |
| | | | | glucuronosyltransferase 1 family, |
| | | | | polypeptide A8 /// UDP |
| | | | | glucuronosyltransferase 1 family, |
| | | | | polypeptide A9 |
| 204554_at | 2.7311485 | down | PPP1R3D | protein phosphatase 1, regulatory |
| | | | | (inhibitor) subunit 3D |
| 205552_s_at | 2.7279844 | down | OAS1 | 2′,5′-oligoadenylate synthetase 1, |
| | | | | 40/46 kDa |
| 228565_at | 2.7276409 | down | KIAA1804 | mixed lineage kinase 4 |
| 228155_at | 2.7267504 | down | C10orf57 /// | chromosome 10 open reading frame 57 |
| | | | C10orf58 | /// chromosome 10 open reading frame |
| | | | | 58 |
| 223427_s_at | 2.7255745 | down | EPB41L4B | erythrocyte membrane protein band 4.1 |
| | | | | like 4B |
| 204867_at | 2.7246413 | down | GCHFR | GTP cyclohydrolase I feedback |
| | | | | regulator |
| 221802_s_at | 2.723564 | down | KIAA1598 | KIAA1598 |
| 231807_at | 2.7220626 | down | KIAA1217 | KIAA1217 |
| 205818_at | 2.7216518 | down | DBC1 | deleted in bladder cancer 1 |
| 226106_at | 2.7186909 | down | RNF141 | ring finger protein 141 |
| 205251_at | 2.718256 | down | PER2 | period homolog 2 (Drosophila) |
| 201249_at | 2.7168527 | down | SLC2A1 | solute carrier family 2 (facilitated |
| | | | | glucose transporter), member 1 |
| 215891_s_at | 2.712683 | down | GM2A | GM2 ganglioside activator |
| 206102_at | 2.7067106 | down | GINS1 | GINS complex subunit 1 (Psf1 |
| | | | | homolog) |
| 205676_at | 2.704932 | down | CYP27B1 | cytochrome P450, family 27, |
| | | | | subfamily B, polypeptide 1 |
| 230475_at | 2.704732 | down | C15orf59 | chromosome 15 open reading frame 59 |
| 1554878_a_at | 2.7038002 | down | ABCD3 | ATP-binding cassette, sub-family D |
| | | | | (ALD), member 3 |
| 226968_at | 2.7027438 | down | KIF1B | kinesin family member 1B |
| 229997_at | 2.7027018 | down | VANGL1 | vang-like 1 (van gogh, Drosophila) |
| 222833_at | 2.6967285 | down | LPCAT2 | lysophosphatidylcholine |
| | | | | acyltransferase 2 |
| 202449_s_at | 2.6946986 | down | RXRA | retinoid X receptor, alpha |
| 222912_at | 2.6944733 | down | ARRB1 | arrestin, beta 1 |
| 1555292_at | 2.6942291 | down | FAM40B | family with sequence similarity 40, |
| | | | | member B |
| 240303_at | 2.6937366 | down | TMC5 | Transmembrane channel-like 5, mRNA |
| | | | | (cDNA clone IMAGE: 5265527) |
| 203641_s_at | 2.6928594 | down | COBLL1 | COBL-like 1 |
| 208999_at | 2.6917293 | down | 40064 | septin 8 |
| 210716_s_at | 2.6889458 | down | CLIP1 | CAP-GLY domain containing linker |
| | | | | protein 1 |
| 204503_at | 2.6869638 | down | EVPL | envoplakin |
| 220658_s_at | 2.6862872 | down | ARNTL2 | aryl hydrocarbon receptor nuclear |
| | | | | translocator-like 2 |
| 57163_at | 2.6833892 | down | ELOVL1 | elongation of very long chain fatty |
| | | | | acids (FEN1/Elo2, SUR4/Elo3, yeast)- |
| | | | | like 1 |
| 230630_at | 2.682703 | down | AK3L1 /// | adenylate kinase 3-like 1 /// adenylate |
| | | | AK3L2 | kinase 3-like 2 |
| 226690_at | 2.681126 | down | ADCYAP1R1 | CDNA FLJ39226 fis, clone |
| | | | | OCBBF2007232 |
| 218845_at | 2.680393 | down | DUSP22 | dual specificity phosphatase 22 |
| 212702_s_at | 2.680374 | down | BICD2 | bicaudal D homolog 2 (Drosophila) |
| 224516_s_at | 2.67937 | down | CXXC5 | CXXC finger 5 |
| 201732_s_at | 2.6788018 | down | CLCN3 | chloride channel 3 |
| 239273_s_at | 2.6781023 | down | MMP28 | matrix metallopeptidase 28 |
| 205739_x_at | 2.678095 | down | ZNF107 | zinc finger protein 107 |
| 225597_at | 2.6771371 | down | SLC45A4 | solute carrier family 45, member 4 |
| 204430_s_at | 2.6757636 | down | SLC2A5 | solute carrier family 2 (facilitated |
| | | | | glucose/fructose transporter), member 5 |
| 212921_at | 2.6731923 | down | SMYD2 | SET and MYND domain containing 2 |
| 242123_at | 2.6718855 | down | PAQR7 | progestin and adipoQ receptor family |
| | | | | member VII |
| 227271_at | 2.6716337 | down | FGF11 | fibroblast growth factor 11 |
| 225987_at | 2.669063 | down | STEAP4 | STEAP family member 4 |
| 209790_s_at | 2.6684937 | down | CASP6 | caspase 6, apoptosis-related cysteine |
| | | | | peptidase |
| 222477_s_at | 2.6653984 | down | TM7SF3 | transmembrane 7 superfamily member 3 |
| 203411_s_at | 2.663246 | down | LMNA | lamin A/C |
| 224580_at | 2.660365 | down | SLC38A1 | solute carrier family 38, member 1 |
| 205569_at | 2.6560183 | down | LAMP3 | lysosomal-associated membrane |
| | | | | protein 3 |
| 236496_at | 2.653787 | down | DEGS2 | degenerative spermatocyte homolog 2, |
| | | | | lipid desaturase (Drosophila) |
| 231775_at | 2.652064 | down | TNFRSF10A | tumor necrosis factor receptor |
| | | | | superfamily, member 10a |
| 211950_at | 2.6516557 | down | UBR4 | ubiquitin protein ligase E3 component |
| | | | | n-recognin 4 |
| 207431_s_at | 2.6450002 | down | DEGS1 | degenerative spermatocyte homolog 1, |
| | | | | lipid desaturase (Drosophila) |
| 200635_s_at | 2.6411211 | down | PTPRF | protein tyrosine phosphatase, receptor |
| | | | | type, F |
| 230252_at | 2.640716 | down | LPAR5 | lysophosphatidic acid receptor 5 |
| 226584_s_at | 2.6402125 | down | FAM110A | family with sequence similarity 110, |
| | | | | member A |
| 233955_x_at | 2.6379757 | down | CXXC5 | CXXC finger 5 |
| 213624_at | 2.63438 | down | SMPDL3A | sphingomyelin phosphodiesterase, |
| | | | | acid-like 3A |
| 212449_s_at | 2.632708 | down | LYPLA1 | lysophospholipase I |
| 231755_at | 2.6293309 | down | IL1F8 | interleukin 1 family, member 8 (eta) |
| 216836_s_at | 2.6269796 | down | ERBB2 | v-erb-b2 erythroblastic leukemia viral |
| | | | | oncogene homolog 2, |
| | | | | neuro/glioblastoma derived oncogene |
| | | | | homolog (avian) |
| 212086_x_at | 2.6258197 | down | LMNA | lamin A/C |
| 211984_at | 2.623374 | down | CALM1 /// | calmodulin 1 (phosphorylase kinase, |
| | | | CALM2 /// | delta) /// calmodulin 2 (phosphorylase |
| | | | CALM3 | kinase, delta) /// calmodulin 3 |
| | | | | (phosphorylase kinase, delta) |
| 229522_at | 2.6224053 | down | SDR42E1 | short chain dehydrogenase/reductase |
| | | | | family 42E, member 1 |
| 229545_at | 2.6210556 | down | FERMT1 | fermitin family homolog 1 |
| | | | | (Drosophila) |
| 218373_at | 2.6198244 | down | AKTIP | AKT interacting protein |
| 209502_s_at | 2.6195574 | down | BAIAP2 | BAI1-associated protein 2 |
| 1554541_a_at | 2.6188276 | down | GPRIN2 | G protein regulated inducer of neurite |
| | | | | outgrowth 2 |
| 228067_at | 2.6184916 | down | C2orf55 | chromosome 2 open reading frame 55 |
| 218174_s_at | 2.6181328 | down | C10orf57 | chromosome 10 open reading frame 57 |
| 234931_at | 2.6159503 | down | AYP1p1 | AYP1 pseudogene 1 |
| 1553906_s_at | 2.6134384 | down | FGD2 | FYVE, RhoGEF and PH domain |
| | | | | containing 2 |
| 232893_at | 2.6126134 | down | LMBRD2 | LMBR1 domain containing 2 |
| 215380_s_at | 2.6118267 | down | GGCT | gamma-glutamyl cyclotransferase |
| 1552691_at | 2.6064768 | down | ARL11 | ADP-ribosylation factor-like 11 |
| 209930_s_at | 2.603701 | down | NFE2 | nuclear factor (erythroid-derived 2), |
| | | | | 45 kDa |
| 225327_at | 2.6030378 | down | KIAA1370 | KIAA1370 |
| 226198_at | 2.602333 | down | TOM1L2 | target of myb1-like 2 (chicken) |
| 210872_x_at | 2.6014338 | down | GAS7 | growth arrest-specific 7 |
| 200831_s_at | 2.6004403 | down | SCD | stearoyl-CoA desaturase (delta-9- |
| | | | | desaturase) |
| 227701_at | 2.5987177 | down | C10orf118 | chromosome 10 open reading frame |
| | | | | 118 |
| 230986_at | 2.5968971 | down | KLF8 | CDNA selection clone ADS40 |
| 220944_at | 2.5951362 | down | PGLYRP4 | peptidoglycan recognition protein 4 |
| 225611_at | 2.5939965 | down | LOC100128443 | hypothetical protein LOC100128443 /// |
| | | | /// MAST4 | microtubule associated |
| | | | | serine/threonine kinase family member 4 |
| 237159_x_at | 2.5931563 | down | AP1S3 | adaptor-related protein complex 1, |
| | | | | sigma 3 subunit |
| 208977_x_at | 2.5907562 | down | TUBB2C | tubulin, beta 2C |
| 202735_at | 2.5905764 | down | EBP | emopamil binding protein (sterol |
| | | | | isomerase) |
| 203535_at | 2.590224 | down | S100A9 | S100 calcium binding protein A9 |
| 221698_s_at | 2.5864825 | down | CLEC7A | C-type lectin domain family 7, member A |
| 202596_at | 2.5864198 | down | ENSA | endosulfine alpha |
| 229776_at | 2.5856552 | down | SLCO3A1 | solute carrier organic anion transporter |
| | | | | family, member 3A1 |
| 218802_at | 2.5770578 | down | CCDC109B | coiled-coil domain containing 109B |
| 219230_at | 2.5765135 | down | TMEM100 | transmembrane protein 100 |
| 226769_at | 2.5724943 | down | FIBIN | fin bud initiation factor homolog |
| | | | | (zebrafish) |
| 211661_x_at | 2.5699801 | down | PTAFR | platelet-activating factor receptor |
| 218556_at | 2.5698657 | down | ORMDL2 | ORM1-like 2 (S. cerevisiae) |
| 209218_at | 2.5663185 | down | SQLE | squalene epoxidase |
| 1557828_a_at | 2.564371 | down | C5orf28 | CDNA FLJ36759 fis, clone |
| | | | | UTERU2018566 |
| 207169_x_at | 2.5612175 | down | DDR1 | discoidin domain receptor tyrosine |
| | | | | kinase 1 |
| 213174_at | 2.5579307 | down | TTC9 | tetratricopeptide repeat domain 9 |
| 236760_at | 2.5566704 | down | AMMECR1 | Alport syndrome, mental retardation, |
| | | | | midface hypoplasia and elliptocytosis |
| | | | | chromosomal region, gene 1, mRNA |
| | | | | (cDNA clone IMAGE: 5405764) |
| 224008_s_at | 2.5530894 | down | KCNK7 | potassium channel, subfamily K, |
| | | | | member 7 |
| 203970_s_at | 2.546217 | down | PEX3 | peroxisomal biogenesis factor 3 |
| 239533_at | 2.539709 | down | GPR155 | G protein-coupled receptor 155 |
| 202814_s_at | 2.5392075 | down | HEXIM1 | hexamethylene bis-acetamide inducible 1 |
| 212479_s_at | 2.5390153 | down | RMND5A | required for meiotic nuclear division 5 |
| | | | | homolog A (S. cerevisiae) |
| 208188_at | 2.5387063 | down | KRT9 | keratin 9 |
| 218888_s_at | 2.537066 | down | NETO2 | neuropilin (NRP) and tolloid (TLL)- |
| | | | | like 2 |
| 202081_at | 2.5366287 | down | IER2 | immediate early response 2 |
| 1569065_s_at | 2.5342972 | down | C15orf62 | chromosome 15 open reading frame 62 |
| 238687_x_at | 2.531887 | down | ZNF770 | zinc finger protein 770 |
| 214639_s_at | 2.5304656 | down | HOXA1 | homeobox A1 |
| 200864_s_at | 2.5266373 | down | RAB11A | RAB11A, member RAS oncogene |
| | | | | family |
| 221044_s_at | 2.5264642 | down | TRIM34 /// | tripartite motif-containing 34 /// |
| | | | TRIM6 /// | tripartite motif-containing 6 /// TRIM6- |
| | | | TRIM6- | TRIM34 readthrough transcript |
| | | | TRIM34 | |
| 226002_at | 2.5262494 | down | GAB1 | GRB2-associated binding protein 1 |
| 218237_s_at | 2.5249395 | down | SLC38A1 | solute carrier family 38, member 1 |
| 228320_x_at | 2.5236611 | down | CCDC64 | coiled-coil domain containing 64 |
| 218094_s_at | 2.5231547 | down | DBNDD2 /// | dysbindin (dystrobrevin binding |
| | | | SYS1- | protein 1) domain containing 2 /// |
| | | | DBNDD2 | SYS1-DBNDD2 readthrough transcript |
| 204451_at | 2.5215912 | down | FZD1 | frizzled homolog 1 (Drosophila) |
| 237030_at | 2.5207937 | down | ACPP | acid phosphatase, prostate |
| 203215_s_at | 2.5199451 | down | MYO6 | myosin VI |
| 204334_at | 2.5181212 | down | KLF7 | Kruppel-like factor 7 (ubiquitous) |
| 221610_s_at | 2.5177839 | down | STAP2 | signal transducing adaptor family |
| | | | | member 2 |
| 224414_s_at | 2.5176234 | down | CARD6 | caspase recruitment domain family, |
| | | | | member 6 |
| 219428_s_at | 2.5164838 | down | PXMP4 | peroxisomal membrane protein 4, |
| | | | | 24 kDa |
| 223805_at | 2.5157578 | down | OSBPL6 | oxysterol binding protein-like 6 |
| 208534_s_at | 2.515689 | down | RASA4 /// | RAS p21 protein activator 4 /// RAS |
| | | | RASA4P | p21 protein activator 4 pseudogene |
| 222725_s_at | 2.5121596 | down | PALMD | palmdelphin |
| 207847_s_at | 2.5091352 | down | MUC1 | mucin 1, cell surface associated |
| 212882_at | 2.5058832 | down | KLHL18 | kelch-like 18 (Drosophila) |
| 204653_at | 2.5022001 | down | TFAP2A | transcription factor AP-2 alpha |
| | | | | (activating enhancer binding protein 2 |
| | | | | alpha) |
| 219518_s_at | 2.5002704 | down | ELL3 | elongation factor RNA polymerase II- |
| | | | | like 3 |
| 201939_at | 2.4999027 | down | PLK2 | polo-like kinase 2 (Drosophila) |
| 210582_s_at | 2.499334 | down | LIMK2 | LIM domain kinase 2 |
| 237252_at | 2.4946501 | down | THBD | thrombomodulin |
| 201349_at | 2.490851 | down | SLC9A3R1 | solute carrier family 9 |
| | | | | (sodium/hydrogen exchanger), member |
| | | | | 3 regulator 1 |
| 203216_s_at | 2.4889913 | down | MYO6 | myosin VI |
| 213256_at | 2.4881077 | down | 39875 | membrane-associated ring finger |
| | | | | (C3HC4) 3 |
| 204989_s_at | 2.4878535 | down | ITGB4 | integrin, beta 4 |
| 213154_s_at | 2.484296 | down | BICD2 | bicaudal D homolog 2 (Drosophila) |
| 214058_at | 2.4831367 | down | MYCL1 | v-myc myelocytomatosis viral |
| | | | | oncogene homolog 1, lung carcinoma |
| | | | | derived (avian) |
| 220638_s_at | 2.4813514 | down | CBLC | Cas-Br-M (murine) ecotropic retroviral |
| | | | | transforming sequence c |
| 218180_s_at | 2.4810977 | down | EPS8L2 | EPS8-like 2 |
| 235333_at | 2.480436 | down | B4GALT6 | UDP-Gal:betaGlcNAc beta 1,4- |
| | | | | galactosyltransferase, polypeptide 6 |
| 203632_s_at | 2.4795673 | down | GPRC5B | G protein-coupled receptor, family C, |
| | | | | group 5, member B |
| 229568_at | 2.4786081 | down | MOBKL2B | MOB1, Mps One Binder kinase |
| | | | | activator-like 2B (yeast) |
| 206653_at | 2.4773498 | down | POLR3G | polymerase (RNA) III (DNA directed) |
| | | | | polypeptide G (32 kD) |
| 201432_at | 2.4770842 | down | CAT | catalase |
| 224810_s_at | 2.4760013 | down | ANKRD13A | ankyrin repeat domain 13A |
| 1555942_a_at | 2.4756498 | down | LOC642587 | CDNA FLJ33794 fis, clone |
| | | | | CTONG1000009 |
| 213726_x_at | 2.4728134 | down | TUBB2C | tubulin, beta 2C |
| 242197_x_at | 2.4713676 | down | CD36 | CD36 antigen |
| 223394_at | 2.4701357 | down | SERTAD1 | SERTA domain containing 1 |
| 201641_at | 2.4696527 | down | BST2 | bone marrow stromal cell antigen 2 |
| 213288_at | 2.4695723 | down | MBOAT2 | membrane bound O-acyltransferase |
| | | | | domain containing 2 |
| 221841_s_at | 2.467558 | down | KLF4 | Kruppel-like factor 4 (gut) |
| 227970_at | 2.4662564 | down | GPR157 | G protein-coupled receptor 157 |
| 222996_s_at | 2.4653852 | down | CXXC5 | CXXC finger 5 |
| 209566_at | 2.4633334 | down | INSIG2 | insulin induced gene 2 |
| 214696_at | 2.4627934 | down | C17orf91 | chromosome 17 open reading frame 91 |
| 214234_s_at | 2.4618382 | down | CYP3A5 | cytochrome P450, family 3, subfamily |
| | | | | A, polypeptide 5 |
| 223471_at | 2.4607677 | down | RAB3IP | RAB3A interacting protein (rabin3) |
| 234305_s_at | 2.4569333 | down | GSDMC | gasdermin C |
| 216388_s_at | 2.4562252 | down | LTB4R | leukotriene B4 receptor |
| 227624_at | 2.4547067 | down | TET2 | tet oncogene family member 2 |
| 223279_s_at | 2.453393 | down | UACA | uveal autoantigen with coiled-coil |
| | | | | domains and ankyrin repeats |
| 40016_g_at | 2.452864 | down | LOC100128443 | hypothetical protein LOC100128443 /// |
| | | | /// MAST4 | microtubule associated |
| | | | | serine/threonine kinase family member 4 |
| 201341_at | 2.4487681 | down | ENC1 | ectodermal-neural cortex (with BTB- |
| | | | | like domain) |
| 242722_at | 2.4461777 | down | LMO7 | LIM domain 7 |
| 224301_x_at | 2.4447956 | down | H2AFJ | H2A histone family, member J |
| 225521_at | 2.4442914 | down | ANAPC7 | anaphase promoting complex subunit 7 |
| 206355_at | 2.442836 | down | GNAL | guanine nucleotide binding protein (G |
| | | | | protein), alpha activating activity |
| | | | | polypeptide, olfactory type |
| 219296_at | 2.4395428 | down | ZDHHC13 | zinc finger, DHHC-type containing 13 |
| 200752_s_at | 2.4395049 | down | CAPN1 | calpain 1, (mu/I) large subunit |
| 209031_at | 2.4321353 | down | CADM1 | cell adhesion molecule 1 |
| 216641_s_at | 2.4319324 | down | LAD1 | ladinin 1 |
| 209758_s_at | 2.4311805 | down | MFAP5 | microfibrillar associated protein 5 |
| 228892_at | 2.4310536 | down | SH3RF2 | SH3 domain containing ring finger 2 |
| 223681_s_at | 2.4298599 | down | INADL | InaD-like (Drosophila) |
| 200768_s_at | 2.428787 | down | MAT2A | methionine adenosyltransferase II, |
| | | | | alpha |
| 207455_at | 2.4276183 | down | P2RY1 | purinergic receptor P2Y, G-protein |
| | | | | coupled, 1 |
| 220403_s_at | 2.4254568 | down | P53AIP1 | p53-regulated apoptosis-inducing |
| | | | | protein 1 |
| 229800_at | 2.4252849 | down | DCLK1 | KIAA0369 gene |
| 1555404_a_at | 2.4229205 | down | DUOXA1 | dual oxidase maturation factor 1 |
| 1558834_s_at | 2.4209795 | down | C1orf62 | chromosome 1 open reading frame 62 |
| 209291_at | 2.4183009 | down | ID4 | inhibitor of DNA binding 4, dominant |
| | | | | negative helix-loop-helix protein |
| 210831_s_at | 2.4178216 | down | PTGER3 | prostaglandin E receptor 3 (subtype |
| | | | | EP3) |
| 1559571_a_at | 2.4145973 | down | ATP13A4 | ATPase type 13A4 |
| 231928_at | 2.4141552 | down | HES2 | hairy and enhancer of split 2 |
| | | | | (Drosophila) |
| 202005_at | 2.41391 | down | ST14 | suppression of tumorigenicity 14 |
| | | | | (colon carcinoma) |
| 212406_s_at | 2.413782 | down | PCMTD2 | protein-L-isoaspartate (D-aspartate) O- |
| | | | | methyltransferase domain containing 2 |
| 218826_at | 2.4101262 | down | SLC35F2 | solute carrier family 35, member F2 |
| 212707_s_at | 2.4100726 | down | LOC100133005 | similar to RAS p21 protein activator 4 |
| | | | /// | /// similar to HSPC047 protein /// RAS |
| | | | LOC100134722 | p21 protein activator 4 /// RAS p21 |
| | | | /// RASA4 | protein activator 4 pseudogene |
| | | | /// RASA4P | |
| 208779_x_at | 2.4082775 | down | DDR1 | discoidin domain receptor tyrosine |
| | | | | kinase 1 |
| 206873_at | 2.4066236 | down | CA6 | carbonic anhydrase VI |
| 221750_at | 2.4064143 | down | HMGCS1 | 3-hydroxy-3-methylglutaryl-Coenzyme |
| | | | | A synthase 1 (soluble) |
| 212321_at | 2.405509 | down | SGPL1 | sphingosine-1-phosphate lyase 1 |
| 201535_at | 2.4043798 | down | UBL3 | ubiquitin-like 3 |
| 231804_at | 2.4037292 | down | RXFP1 | relaxin/insulin-like family peptide |
| | | | | receptor 1 |
| 200703_at | 2.4029148 | down | DYNLL1 | dynein, light chain, LC8-type 1 |
| 205968_at | 2.402016 | down | KCNS3 | potassium voltage-gated channel, |
| | | | | delayed-rectifier, subfamily S, member 3 |
| 219856_at | 2.4011526 | down | C1orf116 | chromosome 1 open reading frame 116 |
| 217979_at | 2.400637 | down | TSPAN13 | tetraspanin 13 |
| 218739_at | 2.3995485 | down | ABHD5 | abhydrolase domain containing 5 |
| 33646_g_at | 2.3972998 | down | GM2A | GM2 ganglioside activator |
| 211071_s_at | 2.3966746 | down | MLLT11 | myeloid/lymphoid or mixed-lineage |
| | | | | leukemia (trithorax homolog, |
| | | | | Drosophila); translocated to, 11 |
| 1569157_s_at | 2.3959668 | down | ZNF846 | zinc finger protein 846 |
| 222536_s_at | 2.3936675 | down | ZNF395 | zinc finger protein 395 |
| 213051_at | 2.39318 | down | ZC3HAV1 | zinc finger CCCH-type, antiviral 1 |
| 227335_at | 2.3928032 | down | DIDO1 | death inducer-obliterator 1 |
| 223265_at | 2.3924136 | down | SH3BP5L | SH3-binding domain protein 5-like |
| 218902_at | 2.390648 | down | NOTCH1 | Notch homolog 1, translocation- |
| | | | | associated (Drosophila) |
| 201975_at | 2.38955 | down | CLIP1 | CAP-GLY domain containing linker |
| | | | | protein 1 |
| 203515_s_at | 2.388306 | down | PMVK | phosphomevalonate kinase |
| 202016_at | 2.3868265 | down | MEST | mesoderm specific transcript homolog |
| | | | | (mouse) |
| 213280_at | 2.38649 | down | GARNL4 | GTPase activating Rap/RanGAP |
| | | | | domain-like 4 |
| 209424_s_at | 2.381329 | down | AMACR | alpha-methylacyl-CoA racemase |
| 202720_at | 2.3791854 | down | TES | testis derived transcript (3 LIM |
| | | | | domains) |
| 219272_at | 2.3777165 | down | TRIM62 | tripartite motif-containing 62 |
| 223454_at | 2.3772516 | down | CXCL16 | chemokine (C—X—C motif) ligand 16 |
| 205073_at | 2.3759983 | down | CYP2J2 | cytochrome P450, family 2, subfamily |
| | | | | J, polypeptide 2 |
| 227263_at | 2.3751676 | down | C8orf58 | chromosome 8 open reading frame 58 |
| 227725_at | 2.3737078 | down | ST6GALNAC1 | ST6 (alpha-N-acetyl-neuraminyl-2,3- |
| | | | | beta-galactosyl-1,3)-N- |
| | | | | acetylgalactosaminide alpha-2,6- |
| | | | | sialyltransferase 1 |
| 212830_at | 2.3727896 | down | MEGF9 | multiple EGF-like-domains 9 |
| 201733_at | 2.3707964 | down | CLCN3 | chloride channel 3 |
| 224836_at | 2.368579 | down | TP53INP2 | tumor protein p53 inducible nuclear |
| | | | | protein 2 |
| 213562_s_at | 2.367167 | down | SQLE | squalene epoxidase |
| 230076_at | 2.3642602 | down | PITPNM3 | PITPNM family member 3 |
| 227880_s_at | 2.363816 | down | LOC100132969 | hypothetical protein LOC100132969 /// |
| | | | /// | transmembrane protein 185A |
| | | | TMEM185A | |
| 226980_at | 2.362776 | down | DEPDC1B | DEP domain containing 1B |
| 224622_at | 2.3615942 | down | TBC1D14 | TBC1 domain family, member 14 |
| 231861_at | 2.3615434 | down | LRP10 | low density lipoprotein receptor-related |
| | | | | protein 10 |
| 230864_at | 2.3605864 | down | MGC42105 | serine/threonine-protein kinase NIM1 |
| 227868_at | 2.3588343 | down | LOC154761 | hypothetical LOC154761 |
| 228647_at | 2.357218 | down | LOC100049716 | hypothetical protein LOC100049716 |
| 223289_s_at | 2.356582 | down | USP38 | ubiquitin specific peptidase 38 |
| 218816_at | 2.3564343 | down | LRRC1 | leucine rich repeat containing 1 |
| 235068_at | 2.355547 | down | ZDHHC21 | zinc finger, DHHC-type containing 21 |
| 212861_at | 2.3544407 | down | MFSD5 | major facilitator superfamily domain |
| | | | | containing 5 |
| 227404_s_at | 2.3532495 | down | EGR1 | Putative zinc finger protein mRNA, 3′ |
| | | | | flank |
| 202669_s_at | 2.3530884 | down | EFNB2 | ephrin-B2 |
| 202506_at | 2.3506389 | down | SSFA2 | sperm specific antigen 2 |
| 228123_s_at | 2.3497157 | down | ABHD12 | abhydrolase domain containing 12 |
| 239598_s_at | 2.3495939 | down | LPCAT2 | lysophosphatidylcholine |
| | | | | acyltransferase 2 |
| 201734_at | 2.3489254 | down | CLCN3 | Chloride channel protein 3 (CLCN3) |
| 220599_s_at | 2.3469906 | down | CARD14 | caspase recruitment domain family, |
| | | | | member 14 |
| 202341_s_at | 2.346867 | down | TRIM2 | tripartite motif-containing 2 |
| 225099_at | 2.3461185 | down | FBXO45 | F-box protein 45 |
| 225634_at | 2.3442137 | down | ZC3HAV1 | zinc finger CCCH-type, antiviral 1 |
| 204675_at | 2.341739 | down | SRD5A1 | steroid-5-alpha-reductase, alpha |
| | | | | polypeptide 1 (3-oxo-5 alpha-steroid |
| | | | | delta 4-dehydrogenase alpha 1) |
| 222774_s_at | 2.3412967 | down | NETO2 | neuropilin (NRP) and tolloid (TLL)- |
| | | | | like 2 |
| 207708_at | 2.3381183 | down | ALOXE3 | arachidonate lipoxygenase 3 |
| 226599_at | 2.3369987 | down | FHDC1 | FH2 domain containing 1 |
| 235475_at | 2.3346572 | down | LOC100129720 | CDNA clone IMAGE: 5302722 |
| 231211_s_at | 2.333402 | down | LOC541469 | hypothetical protein LOC541469 |
| 202950_at | 2.3329318 | down | CRYZ | crystallin, zeta (quinone reductase) |
| 223340_at | 2.3317142 | down | ATL1 | atlastin GTPase 1 |
| 236207_at | 2.3308241 | down | SSFA2 | sperm specific antigen 2 |
| 225245_x_at | 2.328221 | down | H2AFJ | H2A histone family, member J |
| 1554122_a_at | 2.3248432 | down | HSD17B12 | hydroxysteroid (17-beta) |
| | | | | dehydrogenase 12 |
| 222853_at | 2.3233314 | down | FLRT3 | fibronectin leucine rich transmembrane |
| | | | | protein 3 |
| 227163_at | 2.3232682 | down | GSTO2 | glutathione S-transferase omega 2 |
| 205774_at | 2.317882 | down | F12 | coagulation factor XII (Hageman |
| | | | | factor) |
| 45288_at | 2.317875 | down | ABHD6 /// | abhydrolase domain containing 6 /// |
| | | | LOC643635 | similar to DEAD/H (Asp-Glu-Ala- |
| | | | | Asp/His) box polypeptide 11 |
| 201371_s_at | 2.3138196 | down | CUL3 | cullin 3 |
| 214355_x_at | 2.3121972 | down | CTAGE4 /// | CTAGE family, member 4 /// CTAGE |
| | | | LOC100142659 | family member /// similar to CTAGE6 |
| | | | /// | |
| | | | LOC441294 | |
| 235987_at | 2.3110762 | down | PRKXP1 | protein kinase, X-linked, pseudogene 1 |
| 200813_s_at | 2.3108816 | down | PAFAH1B1 | platelet-activating factor |
| | | | | acetylhydrolase, isoform Ib, alpha |
| | | | | subunit 45 kDa |
| 214651_s_at | 2.3090641 | down | HOXA9 | homeobox A9 |
| 201790_s_at | 2.3061028 | down | DHCR7 | 7-dehydrocholesterol reductase |
| 1557458_s_at | 2.3032465 | down | SHB | Src homology 2 domain containing |
| | | | | adaptor protein B |
| 202748_at | 2.302949 | down | GBP2 | guanylate binding protein 2, interferon- |
| | | | | inducible |
| 229492_at | 2.300059 | down | VANGL1 | vang-like 1 (van gogh, Drosophila) |
| 225108_at | 2.2977571 | down | AGPS | alkylglycerone phosphate synthase |
| 238638_at | 2.2977488 | down | SLC37A2 | solute carrier family 37 (glycerol-3- |
| | | | | phosphate transporter), member 2 |
| 223594_at | 2.294716 | down | TMEM117 | transmembrane protein 117 |
| 225973_at | 2.294342 | down | TAP2 | transporter 2, ATP-binding cassette, |
| | | | | sub-family B (MDR/TAP) |
| 217118_s_at | 2.2925084 | down | C22orf9 | chromosome 22 open reading frame 9 |
| 210383_at | 2.2922642 | down | SCN1A | sodium channel, voltage-gated, type I, |
| | | | | alpha subunit |
| 208866_at | 2.291897 | down | CSNK1A1 | casein kinase 1, alpha 1 |
| 223349_s_at | 2.2913735 | down | BOK | BCL2-related ovarian killer |
| 207911_s_at | 2.287757 | down | TGM5 | transglutaminase 5 |
| 212989_at | 2.2870367 | down | SGMS1 | sphingomyelin synthase 1 |
| 225517_at | 2.2865238 | down | ZNF770 | zinc finger protein 770 |
| 235606_at | 2.2857234 | down | LOC344595 | hypothetical LOC344595 |
| 214235_at | 2.285186 | down | CYP3A5 | cytochrome P450, family 3, subfamily |
| | | | | A, polypeptide 5 |
| 213430_at | 2.283703 | down | RUFY3 | RUN and FYVE domain containing 3 |
| 219528_s_at | 2.2819934 | down | BCL11B | B-cell CLL/lymphoma 11B (zinc |
| | | | | finger protein) |
| 1563805_a_at | 2.281725 | down | FAM83C | family with sequence similarity 83, |
| | | | | member C |
| 213546_at | 2.276899 | down | DKFZP586I1420 | hypothetical protein DKFZp586I1420 |
| 206263_at | 2.274217 | down | FMO4 | flavin containing monooxygenase 4 |
| 225954_s_at | 2.2734365 | down | MIDN | midnolin |
| 215103_at | 2.2728736 | down | CYP2C18 | cytochrome P450, family 2, subfamily |
| | | | | C, polypeptide 18 |
| 227228_s_at | 2.2723181 | down | CCDC88C | coiled-coil domain containing 88C |
| 220318_at | 2.2720828 | down | EPN3 | epsin 3 |
| 227981_at | 2.271106 | down | CYB561D1 | cytochrome b-561 domain containing 1 |
| 202562_s_at | 2.269635 | down | C14orf1 | chromosome 14 open reading frame 1 |
| 202788_at | 2.269064 | down | MAPKAPK3 | mitogen-activated protein kinase- |
| | | | | activated protein kinase 3 |
| 231166_at | 2.2656186 | down | GPR155 | G protein-coupled receptor 155 |
| 1555716_a_at | 2.2642384 | down | CXADR | coxsackie virus and adenovirus |
| | | | | receptor |
| 220966_x_at | 2.2637703 | down | ARPC5L | actin related protein 2/3 complex, |
| | | | | subunit 5-like |
| 232034_at | 2.2627313 | down | LOC203274 | CDNA FLJ31544 fis, clone |
| | | | | NT2RI2000865 |
| 226021_at | 2.2623584 | down | RDH10 | retinol dehydrogenase 10 (all-trans) |
| 209608_s_at | 2.2597988 | down | ACAT2 | acetyl-Coenzyme A acetyltransferase 2 |
| 212978_at | 2.2586613 | down | LRRC8B | leucine rich repeat containing 8 family, |
| | | | | member B |
| 223288_at | 2.2585614 | down | USP38 | ubiquitin specific peptidase 38 |
| 1555905_a_at | 2.2584996 | down | C3orf23 | chromosome 3 open reading frame 23 |
| 205109_s_at | 2.2583284 | down | ARHGEF4 | Rho guanine nucleotide exchange |
| | | | | factor (GEF) 4 |
| 204465_s_at | 2.2553155 | down | INA | internexin neuronal intermediate |
| | | | | filament protein, alpha |
| 209679_s_at | 2.2552056 | down | LOC57228 | small trans-membrane and |
| | | | | glycosylated protein |
| 217856_at | 2.2544947 | down | RBM8A | RNA binding motif protein 8A |
| 218261_at | 2.253576 | down | AP1M2 | adaptor-related protein complex 1, mu |
| | | | | 2 subunit |
| 231990_at | 2.251771 | down | USP15 | ubiquitin specific peptidase 15 |
| 210933_s_at | 2.2504241 | down | FSCN1 | fascin homolog 1, actin-bundling |
| | | | | protein (Strongylocentrotus |
| | | | | purpuratus) |
| 209872_s_at | 2.249547 | down | PKP3 | plakophilin 3 |
| 205602_x_at | 2.2482867 | down | PSG7 | pregnancy specific beta-1-glycoprotein 7 |
| 228851_s_at | 2.2482166 | down | ENSA | endosulfine alpha |
| 238846_at | 2.2471364 | down | TNFRSF11A | tumor necrosis factor receptor |
| | | | | superfamily, member 11a, NFKB |
| | | | | activator |
| 1553059_at | 22462165 | down | PGLYRP3 | peptidoglycan recognition protein 3 |
| 215513_at | 2.2452734 | down | HYMAI | hydatidiform mole associated and |
| | | | | imprinted (non-protein coding) |
| 231062_at | 2.2450545 | down | LOC100129122 | Clone IMAGE: 1257951, mRNA |
| | | | | sequence |
| 204761_at | 2.244495 | down | USP6NL | USP6 N-terminal like |
| 219517_at | 2.2439237 | down | ELL3 | elongation factor RNA polymerase II- |
| | | | | like 3 |
| 201791_s_at | 2.242235 | down | DHCR7 | 7-dehydrocholesterol reductase |
| 210058_at | 2.2416518 | down | MAPK13 | mitogen-activated protein kinase 13 |
| 225342_at | 2.2410355 | down | AK3L1 /// | adenylate kinase 3-like 1 /// adenylate |
| | | | AK3L2 | kinase 3-like 2 |
| 210059_s_at | 2.2387981 | down | MAPK13 | mitogen-activated protein kinase 13 |
| 206149_at | 2.236605 | down | CHP2 | calcineurin B homologous protein 2 |
| 201566_x_at | 2.2364604 | down | ID2 | inhibitor of DNA binding 2, dominant |
| | | | | negative helix-loop-helix protein |
| 223101_s_at | 2.2357152 | down | ARPC5L | actin related protein 2/3 complex, |
| | | | | subunit 5-like |
| 209905_at | 2.2349339 | down | HOXA9 | homeobox A9 |
| 227383_at | 2.2334142 | down | LOC727820 | hypothetical protein LOC727820 |
| 219330_at | 2.2328598 | down | VANGL1 | vang-like 1 (van gogh, Drosophila) |
| 232103_at | 2.229358 | down | BPNT1 | 3′(2′), 5′-bisphosphate nucleotidase 1 |
| 225300_at | 2.2289581 | down | C15orf23 | chromosome 15 open reading frame 23 |
| 229596_at | 2.2280157 | down | AMDHD1 | amidohydrolase domain containing 1 |
| 232138_at | 2.2262597 | down | MBNL2 | Muscleblind-like 2 (Drosophila), |
| | | | | mRNA (cDNA clone |
| | | | | IMAGE: 4157895) |
| 207236_at | 2.2259998 | down | ZNF345 | zinc finger protein 345 |
| 227224_at | 2.2250893 | down | RALGPS2 | Ral GEF with PH domain and SH3 |
| | | | | binding motif 2 |
| 221896_s_at | 2.224972 | down | HIGD1A | HIG1 domain family, member 1A |
| 200637_s_at | 2.2237906 | down | PTPRF | protein tyrosine phosphatase, receptor |
| | | | | type, F |
| 214798_at | 2.2234876 | down | ATP2C2 | ATPase, Ca++ transporting, type 2C, |
| | | | | member 2 |
| 226915_s_at | 2.223121 | down | ARPC5L | actin related protein 2/3 complex, |
| | | | | subunit 5-like |
| 213533 at | 2.219701 | down | D4S234E | DNA segment on chromosome 4 |
| | | | | (unique) 234 expressed sequence |
| 213577_at | 2.218055 | down | SQLE | squalene epoxidase |
| 219956_at | 2.2179477 | down | GALNT6 | UDP-N-acetyl-alpha-D- |
| | | | | galactosamine:polypeptide N- |
| | | | | acetylgalactosaminyltransferase 6 |
| | | | | (GalNAc-T6) |
| 200632_s_at | 2.2170808 | down | NDRG1 | N-myc downstream regulated 1 |
| 202488_s_at | 2.2170005 | down | FXYD3 | FXYD domain containing ion transport |
| | | | | regulator 3 |
| 202967_at | 2.2159603 | down | GSTA4 | glutathione S-transferase alpha 4 |
| 209727_at | 2.2159023 | down | GM2A | GM2 ganglioside activator |
| 203081_at | 2.2149336 | down | CTNNBIP1 | catenin, beta interacting protein 1 |
| 1555416_a_at | 2.214861 | down | ALOX15B | arachidonate 15-lipoxygenase, type B |
| 236725_at | 2.2132413 | down | WWC1 | WW and C2 domain containing 1 |
| 225726_s_at | 2.213236 | down | PLEKHH1 | pleckstrin homology domain |
| | | | | containing, family H (with MyTH4 |
| | | | | domain) member 1 |
| 203625_x_at | 2.21156 | down | SKP2 | CDNA: FLJ22571 fis, clone HSI02239 |
| 206417_at | 2.211349 | down | CNGA1 | cyclic nucleotide gated channel alpha 1 |
| 207602_at | 2.2072852 | down | TMPRSS11D | transmembrane protease, serine 11D |
| 202951_at | 2.2066703 | down | STK38 | serine/threonine kinase 38 |
| 220985_s_at | 2.2064672 | down | RNF170 | ring finger protein 170 |
| 205092_x_at | 2.2053156 | down | ZBTB1 | zinc finger and BTB domain |
| | | | | containing 1 |
| 226844_at | 2.2050638 | down | MOBKL2B | MOB1, Mps One Binder kinase |
| | | | | activator-like 2B (yeast) |
| 226448_at | 2.2045517 | down | FAM89A | family with sequence similarity 89, |
| | | | | member A |
| 227997_at | 2.2044806 | down | IL17RD | interleukin 17 receptor D |
| 220225_at | 2.203769 | down | IRX4 | iroquois homeobox 4 |
| 40524_at | 2.2021623 | down | PTPN21 | protein tyrosine phosphatase, non- |
| | | | | receptor type 21 |
| 202725_at | 2.199355 | down | POLR2A | polymerase (RNA) II (DNA directed) |
| | | | | polypeptide A, 220 kDa |
| 207065_at | 2.198705 | down | KRT75 | keratin 75 |
| 224746_at | 2.1967638 | down | KIAA1522 | KIAA1522 |
| 202330_s_at | 2.196532 | down | UNG | uracil-DNA glycosylase |
| 201735_s_at | 2.1951425 | down | CLCN3 | chloride channel 3 |
| 212848_s_at | 2.1947465 | down | C9orf3 | chromosome 9 open reading frame 3 |
| 209389_x_at | 2.194297 | down | DBI | diazepam binding inhibitor (GABA |
| | | | | receptor modulator, acyl-Coenzyme A |
| | | | | binding protein) |
| 217188_s_at | 2.19428 | down | C14orf1 | chromosome 14 open reading frame 1 |
| 232843_s_at | 2.193926 | down | DOCK8 | dedicator of cytokinesis 8 |
| 210749_x_at | 2.1906087 | down | DDR1 | discoidin domain receptor tyrosine |
| | | | | kinase 1 |
| 1569385_s_at | 2.1899562 | down | TET2 | tet oncogene family member 2 |
| 218640_s_at | 2.1887794 | down | PLEKHF2 | pleckstrin homology domain |
| | | | | containing, family F (with FYVE |
| | | | | domain) member 2 |
| 205535_s_at | 2.1884425 | down | PCDH7 | protocadherin 7 |
| 203395_s_at | 2.1880076 | down | HES1 | hairy and enhancer of split 1, |
| | | | | (Drosophila) |
| 225927_at | 2.1876142 | down | MAP3K1 | mitogen-activated protein kinase |
| | | | | kinase kinase 1 |
| 44120_at | 2.1861672 | down | ADCK2 | aarF domain containing kinase 2 |
| 225367_at | 2.1861136 | down | PGM2 | phosphoglucomutase 2 |
| 202178_at | 2.1859634 | down | PRKCZ | protein kinase C, zeta |
| 205964_at | 2.1812165 | down | ZNF426 | zinc finger protein 426 |
| 224799_at | 2.1811337 | down | NDFIP2 | Nedd4 family interacting protein 2 |
| 209163_at | 2.1807954 | down | CYB561 | cytochrome b-561 |
| 228762_at | 2.1806395 | down | LFNG | LFNG O-fucosylpeptide 3-beta-N- |
| | | | | acetylglucosaminyltransferase |
| 214786_at | 2.180576 | down | MAP3K1 | mitogen-activated protein kinase |
| | | | | kinase kinase 1 |
| 239693_at | 2.1804395 | down | SNX24 | Sorting nexin SNX24 (SNX24) |
| 225100_at | 2.176683 | down | FBXO45 | F-box protein 45 |
| 212175_s_at | 2.1766407 | down | AK2 | adenylate kinase 2 |
| 217744_s_at | 2.1757214 | down | PERP | PERP, TP53 apoptosis effector |
| 212831_at | 2.1755953 | down | MEGF9 | multiple EGF-like-domains 9 |
| 223075_s_at | 2.168681 | down | AIF1L | allograft inflammatory factor 1-like |
| 216699_s_at | 2.168667 | down | KLK1 | kallikrein 1 |
| 244546_at | 2.1682029 | down | CYCS | cytochrome c, somatic |
| 213365_at | 2.1674378 | down | ERI2 | exoribonuclease 2 |
| 201215_at | 2.1666453 | down | PLS3 | plastin 3 (T isoform) |
| 203881_s_at | 2.165027 | down | DMD | dystrophin |
| 208854_s_at | 2.1649718 | down | STK24 | serine/threonine kinase 24 (STE20 |
| | | | | homolog, yeast) |
| 212944_at | 2.1649513 | down | SLC5A3 | solute carrier family 5 (sodium/myo- |
| | | | | inositol cotransporter), member 3 |
| 205416_s_at | 2.1644816 | down | ATXN3 | ataxin 3 |
| 228378_at | 2.164461 | down | C12orf29 | chromosome 12 open reading frame 29 |
| 219229_at | 2.1620889 | down | SLCO3A1 | solute carrier organic anion transporter |
| | | | | family, member 3A1 |
| 218657_at | 2.1619468 | down | RAPGEFL1 | Rap guanine nucleotide exchange |
| | | | | factor (GEF)-like 1 |
| 204005_s_at | 2.161023 | down | PAWR | PRKC, apoptosis, WT1, regulator |
| 208345_s_at | 2.1601677 | down | POU3F1 | POU class 3 homeobox 1 |
| 209733_at | 2.160098 | down | LOC286440 | hypothetical protein LOC286440 |
| 225136_at | 2.1593971 | down | PLEKHA2 | pleckstrin homology domain |
| | | | | containing, family A (phosphoinositide |
| | | | | binding specific) member 2 |
| 201565_s_at | 2.1581004 | down | ID2 | inhibitor of DNA binding 2, dominant |
| | | | | negative helix-loop-helix protein |
| 235148_at | 2.1576118 | down | KRTCAP3 | keratinocyte associated protein 3 |
| 201820_at | 2.1536007 | down | KRT5 | keratin 5 |
| 226269_at | 2.1534703 | down | GDAP1 | ganglioside-induced differentiation- |
| | | | | associated protein 1 |
| 218705_s_at | 2.1520202 | down | SNX24 | sorting nexin 24 |
| 235896_s_at | 2.1518188 | down | SMCR7 | Smith-Magenis syndrome chromosome |
| | | | | region, candidate 7 |
| 1559078_at | 2.1510541 | down | BCL11A | CDNA FLJ58516 complete cds, highly |
| | | | | similar to B-cell lymphoma/leukemia |
| | | | | 11A |
| 223214_s_at | 2.1498406 | down | ZHX1 | zinc fingers and homeoboxes 1 |
| 229515_at | 2.149676 | down | PAWR | CDNA clone IMAGE: 3892559 |
| 209815_at | 2.14831 | down | PTCH1 | patched homolog 1 (Drosophila) |
| 227478_at | 2.1481898 | down | SETBP1 | SET binding protein 1 |
| 238686_at | 2.1473422 | down | FBXO3 | F-box protein 3 |
| 202838_at | 2.1467884 | down | FUCA1 | fucosidase, alpha-L-1, tissue |
| 209598_at | 2.1460617 | down | PNMA2 | paraneoplastic antigen MA2 |
| 214975_s_at | 2.1457093 | down | MTMR1 | myotubularin related protein 1 |
| 203276_at | 2.1456895 | down | LMNB1 | lamin B1 |
| 203431_s_at | 2.1429577 | down | RICS | Rho GTPase-activating protein |
| 1554445_at | 2.1428692 | down | ZNF85 | zinc finger protein 85 |
| 208051_s_at | 2.141477 | down | PAIP1 | poly(A) binding protein interacting |
| | | | | protein 1 |
| 225282_at | 2.140211 | down | SMAP2 | small ArfGAP2 |
| 238451_at | 2.1400113 | down | MPP7 | membrane protein, palmitoylated 7 |
| | | | | (MAGUK p55 subfamily member 7) |
| 201079_at | 2.1399426 | down | SYNGR2 | synaptogyrin 2 |
| 225878_at | 2.139625 | down | KIF1B | kinesin family member 1B |
| 213693_s_at | 2.138388 | down | MUC1 | mucin 1, cell surface associated |
| 218421_at | 2.137857 | down | CERK | ceramide kinase |
| 219090_at | 2.1377168 | down | SLC24A3 | solute carrier family 24 |
| | | | | (sodium/potassium/calcium |
| | | | | exchanger), member 3 |
| 226071_at | 2.1368642 | down | ADAMTSL4 | ADAMTS-like 4 |
| 219410_at | 2.1362329 | down | TMEM45A | transmembrane protein 45A |
| 235200_at | 2.1350272 | down | ZNF561 | zinc finger protein 561 |
| 219459_at | 2.1346684 | down | POLR3B | polymerase (RNA) III (DNA directed) |
| | | | | polypeptide B |
| 225313_at | 2.1338372 | down | C20orf177 | chromosome 20 open reading frame |
| | | | | 177 |
| 243963_at | 2.133164 | down | SDCCAG8 | Serologically defined colon cancer |
| | | | | antigen 8, mRNA (cDNA clone |
| | | | | IMAGE: 5301251) |
| 210958_s_at | 2.1311638 | down | LOC100128443 | hypothetical protein LOC100128443 /// |
| | | | /// MAST4 | microtubule associated |
| | | | | serine/threonine kinase family member 4 |
| 210609_s_at | 2.129718 | down | TP53I3 | tumor protein p53 inducible protein 3 |
| 209949_at | 2.1277685 | down | NCF2 | neutrophil cytosolic factor 2 |
| 224055_x_at | 2.1269937 | down | KCNK7 | potassium channel, subfamily K, |
| | | | | member 7 |
| 232127_at | 2.1264925 | down | CLCN5 | chloride channel 5 |
| 223455_at | 2.126378 | down | TCHP | trichoplein, keratin filament binding |
| 209512_at | 2.1262662 | down | HSDL2 | hydroxysteroid dehydrogenase like 2 |
| 207206_s_at | 2.1244593 | down | ALOX12 | arachidonate 12-lipoxygenase |
| 1558924_s_at | 2.1232033 | down | CLIP1 | CAP-GLY domain containing linker |
| | | | | protein 1 |
| 216268_s_at | 2.1227787 | down | JAG1 | jagged 1 (Alagille syndrome) |
| 215100_at | 2.1208134 | down | C6orf105 | chromosome 6 open reading frame 105 |
| 229685_at | 2.1203196 | down | LOC100134937 | hypothetical LOC100134937 |
| 202263_at | 2.1194017 | down | CYB5R1 | cytochrome b5 reductase 1 |
| 212136_at | 2.1178362 | down | ATP2B4 | ATPase, Ca++ transporting, plasma |
| | | | | membrane 4 |
| 225308_s_at | 2.11776 | down | TANC1 | tetratricopeptide repeat, ankyrin repeat |
| | | | | and coiled-coil containing 1 |
| 224865_at | 2.117143 | down | FAR1 | fatty acyl CoA reductase 1 |
| 228954_at | 2.1161342 | down | LYSMD4 | LysM, putative peptidoglycan-binding, |
| | | | | domain containing 4 |
| 1563088_a_at | 2.1154768 | down | LOC284837 | hypothetical protein LOC284837 |
| 222849_s_at | 2.1153836 | down | SCRN3 | secernin 3 |
| 1568815_a_at | 2.1149223 | down | DDX50 | DEAD (Asp-Glu-Ala-Asp) box |
| | | | | polypeptide 50 |
| 1554608_at | 2.1143405 | down | TGOLN2 | trans-golgi network protein 2 |
| 206857_s_at | 2.1133657 | down | FKBP1B | FK506 binding protein 1B, 12.6 kDa |
| 213764_s_at | 2.1121294 | down | MFAP5 | microfibrillar associated protein 5 |
| 218559_s_at | 2.1115992 | down | MAFB | v-maf musculoaponeurotic |
| | | | | fibrosarcoma oncogene homolog B |
| | | | | (avian) |
| 203609_s_at | 2.1106138 | down | ALDH5A1 | aldehyde dehydrogenase 5 family, |
| | | | | member A1 |
| 214605_x_at | 2.1089497 | down | GPR1 | G protein-coupled receptor 1 |
| 203888_at | 2.1086705 | down | THBD | thrombomodulin |
| 215549_x_at | 2.1083808 | down | CTAGE4 | CTAGE family, member 4 |
| 241990_at | 2.1072762 | down | RHOV | ras homolog gene family, member V |
| 229955_at | 2.1066082 | down | FBXO3 | F-box protein 3, mRNA (cDNA clone |
| | | | | IMAGE: 5296662) |
| 222396_at | 2.10537 | down | HN1 | hematological and neurological |
| | | | | expressed 1 |
| 211203_s_at | 2.1050365 | down | CNTN1 | contactin 1 |
| 209234_at | 2.104905 | down | KIF1B | kinesin family member 1B |
| 1007_s_at | 2.10379 | down | DDR1 | discoidin domain receptor tyrosine |
| | | | | kinase 1 |
| 205909_at | 2.103594 | down | POLE2 | polymerase (DNA directed), epsilon 2 |
| | | | | (p59 subunit) |
| 205248_at | 2.1027164 | down | DOPEY2 | dopey family member 2 |
| 229958_at | 2.102053 | down | CLN8 | ceroid-lipofuscinosis, neuronal 8 |
| | | | | (epilepsy, progressive with mental |
| | | | | retardation) |
| 222256_s_at | 2.1013858 | down | JMJD7 /// | jumonji domain containing 7 /// |
| | | | JMJD7- | JMJD7-PLA2G4B readthrough |
| | | | PLA2G4B /// | transcript /// phospholipase A2, group |
| | | | PLA2G4B | IVB (cytosolic) |
| 212089_at | 2.1000233 | down | LMNA | lamin A/C |
| 206429_at | 2.0988882 | down | F2RL1 | coagulation factor II (thrombin) |
| | | | | receptor-like 1 |
| 1559094_at | 2.098726 | down | FBXO9 | F-box protein 9 |
| 238697_at | 2.0983906 | down | NCRNA00086 | Hypothetical protein MGC39606, |
| | | | | mRNA (cDNA clone MGC: 33489 |
| | | | | IMAGE: 4813443) |
| 222904_s_at | 2.096099 | down | TMC5 | transmembrane channel-like 5 |
| 225182_at | 2.0955298 | down | TMEM50B | transmembrane protein 50B |
| 203575_at | 2.0948935 | down | CSNK2A2 | casein kinase 2, alpha prime |
| | | | | polypeptide |
| 211922_s_at | 2.094567 | down | CAT | catalase |
| 1553982_a_at | 2.0945106 | down | RAB7B | RAB7B, member RAS oncogene |
| | | | | family |
| 201839_s_at | 2.0932202 | down | EPCAM | epithelial cell adhesion molecule |
| 47560_at | 2.0923448 | down | LPHN1 | latrophilin 1 |
| 212186_at | 2.0908222 | down | ACACA | acetyl-Coenzyme A carboxylase alpha |
| 219187_at | 2.0901995 | down | FKBPL | FK506 binding protein like |
| 241946_at | 2.0896347 | down | ZDHHC21 | zinc finger, DHHC-type containing 21 |
| 1553021_s_at | 2.0895314 | down | BICD2 | bicaudal D homolog 2 (Drosophila) |
| 203002_at | 2.0893335 | down | AMOTL2 | angiomotin like 2 |
| 203865_s_at | 2.08908 | down | ADARB1 | adenosine deaminase, RNA-specific, |
| | | | | B1 (RED1 homolog rat) |
| 200696_s_at | 2.0890284 | down | GSN | gelsolin (amyloidosis, Finnish type) |
| 204967_at | 2.0882254 | down | SHROOM2 | shroom family member 2 |
| 202894_at | 2.0872073 | down | EPHB4 | EPH receptor B4 |
| 1558292_s_at | 2.0845475 | down | PIGW | phosphatidylinositol glycan anchor |
| | | | | biosynthesis, class W |
| 235567_at | 2.0833743 | down | RORA | Hypothetical protein LOC283666, |
| | | | | mRNA (cDNA clone |
| | | | | IMAGE: 4750925) |
| 212991_at | 2.0831015 | down | FBXO9 | F-box protein 9 |
| 216718_at | 2.0828507 | down | C1orf46 | chromosome 1 open reading frame 46 |
| 1558152_at | 2.081475 | down | LOC100131262 | hypothetical LOC100131262 |
| 225211_at | 2.08033 | down | PVRL1 | poliovirus receptor-related 1 |
| | | | | (herpesvirus entry mediator C) |
| 202987_at | 2.0792933 | down | TRAF3IP2 | TRAF3 interacting protein 2 |
| 222236_s_at | 2.0789578 | down | ASAP3 | ArfGAP with SH3 domain, ankyrin |
| | | | | repeat and PH domain 3 |
| 239230_at | 2.0784454 | down | HES5 | hairy and enhancer of split 5 |
| | | | | (Drosophila) |
| 202501_at | 2.0782366 | down | MAPRE2 | microtubule-associated protein, RP/EB |
| | | | | family, member 2 |
| 242255_at | 2.0781825 | down | LOC100130837 | CDNA clone IMAGE: 4799914 |
| 202587_s_at | 2.0776887 | down | AK1 | adenylate kinase 1 |
| 201032_at | 2.0772772 | down | BLCAP | bladder cancer associated protein |
| 202850_at | 2.076881 | down | ABCD3 | ATP-binding cassette, sub-family D |
| | | | | (ALD), member 3 |
| 225618_at | 2.0766106 | down | ARHGAP27 | Rho GTPase activating protein 27 |
| 203666_at | 2.074363 | down | CXCL12 | chemokine (C—X—C motif) ligand 12 |
| | | | | (stromal cell-derived factor 1) |
| 218148_at | 2.0743272 | down | CENPT | centromere protein T |
| 203777_s_at | 2.0739517 | down | RPS6KB2 | ribosomal protein S6 kinase, 70 kDa, |
| | | | | polypeptide 2 |
| 226929_at | 2.0733016 | down | MTHFR | 5,10-methylenetetrahydrofolate |
| | | | | reductase (NADPH) |
| 225998_at | 2.0714586 | down | GAB1 | GRB2-associated binding protein 1 |
| 222843_at | 2.0714424 | down | FIGNL1 | fidgetin-like 1 |
| 225598_at | 2.0712779 | down | SLC45A4 | solute carrier family 45, member 4 |
| 223213_s_at | 2.0709321 | down | ZHX1 | zinc fingers and homeoboxes 1 |
| 39729_at | 2.0675848 | down | PRDX2 | peroxiredoxin 2 |
| 235174_s_at | 2.0673625 | down | LOC100128822 | hypothetical protein LOC100128822 |
| 219184_x_at | 2.0669382 | down | TIMM22 | translocase of inner mitochondrial |
| | | | | membrane 22 homolog (yeast) |
| 207023_x_at | 2.066204 | down | KRT10 | keratin 10 |
| 227739_at | 2.0635765 | down | LOC648245 | hypothetical LOC648245 |
| 218021_at | 2.0624394 | down | DHRS4 /// | dehydrogenase/reductase (SDR family) |
| | | | DHRS4L2 | member 4 /// dehydrogenase/reductase |
| | | | | (SDR family) member 4 like 2 |
| 202342_s_at | 2.061941 | down | TRIM2 | tripartite motif-containing 2 |
| 203332_s_at | 2.060985 | down | INPP5D | inositol polyphosphate-5-phosphatase, |
| | | | | 145 kDa |
| 227255_at | 2.058867 | down | PDIK1L | PDLIM1 interacting kinase 1 like |
| 219968_at | 2.0585458 | down | ZNF589 | zinc finger protein 589 |
| 210045_at | 2.058517 | down | IDH2 | isocitrate dehydrogenase 2 (NADP+), |
| | | | | mitochondrial |
| 211070_x_at | 2.0563624 | down | DBI | diazepam binding inhibitor (GABA |
| | | | | receptor modulator, acyl-Coenzyme A |
| | | | | binding protein) |
| 200653_s_at | 2.0558417 | down | CALM1 /// | calmodulin 1 (phosphorylase kinase, |
| | | | CALM2 /// | delta) /// calmodulin 2 (phosphorylase |
| | | | CALM3 | kinase, delta) /// calmodulin 3 |
| | | | | (phosphorylase kinase, delta) |
| 200884_at | 2.0550563 | down | CKB | creatine kinase, brain |
| 227384_s_at | 2.054656 | down | LOC727820 | hypothetical protein LOC727820 |
| 201409_s_at | 2.0540502 | down | PPP1CB | protein phosphatase 1, catalytic |
| | | | | subunit, beta isoform |
| 205184_at | 2.0536096 | down | GNG4 | guanine nucleotide binding protein (G |
| | | | | protein), gamma 4 |
| 1554600_s_at | 2.0529914 | down | LMNA | lamin A/C |
| 201474_s_at | 2.051571 | down | ITGA3 | integrin, alpha 3 (antigen CD49C, |
| | | | | alpha 3 subunit of VLA-3 receptor) |
| 204633_s_at | 2.0505228 | down | RPS6KA5 | ribosomal protein S6 kinase, 90 kDa, |
| | | | | polypeptide 5 |
| 212443_at | 2.0503902 | down | NBEAL2 | neurobeachin-like 2 |
| 210608_s_at | 2.0497348 | down | FUT2 | fucosyltransferase 2 (secretor status |
| | | | | included) |
| 1557036_at | 2.0495107 | down | ZBTB1 | Zinc finger and BTB domain |
| | | | | containing 1 (ZBTB1), transcript |
| | | | | variant 1, mRNA |
| 207950_s_at | 2.0490613 | down | ANK3 | ankyrin 3, node of Ranvier (ankyrin G) |
| 41858_at | 2.0485191 | down | FRAG1 | FGF receptor activating protein 1 |
| 234513_at | 2.0481179 | down | ELOVL3 | elongation of very long chain fatty |
| | | | | acids (FEN1/Elo2, SUR4/Elo3, yeast)- |
| | | | | like 3 |
| 223312_at | 2.0474238 | down | C2orf7 | chromosome 2 open reading frame 7 |
| 204137_at | 2.045931 | down | GPR137B | G protein-coupled receptor 137B |
| 229732_at | 2.0447505 | down | ZNF823 | zinc finger protein 823 |
| 206600_s_at | 2.0443823 | down | LOC100133772 | similar to MCT /// solute carrier family |
| | | | /// | 16, member 5 (monocarboxylic acid |
| | | | SLC16A5 | transporter 6) |
| 209099_x_at | 2.0436616 | down | JAG1 | jagged 1 (Alagille syndrome) |
| 221868_at | 2.043276 | down | PAIP2B | poly(A) binding protein interacting |
| | | | | protein 2B |
| 202790_at | 2.0431542 | down | CLDN7 | claudin 7 |
| 227272_at | 2.0418959 | down | C15orf52 | chromosome 15 open reading frame 52 |
| 200950_at | 2.041765 | down | ARPC1A | actin related protein 2/3 complex, |
| | | | | subunit 1A, 41 kDa |
| 226413_at | 2.0407355 | down | LOC400027 | hypothetical gene supported by |
| | | | | BC047417 |
| 202039_at | 2.0400221 | down | MYO18A /// | myosin XVIIIA /// TGFB1-induced |
| | | | TIAF1 | anti-apoptotic factor 1 |
| 233528_s_at | 2.0383992 | down | LOC652968 | hypothetical protein LOC652968 |
| 211993_at | 2.036943 | down | WNK1 | WNK lysine deficient protein kinase 1 |
| 222668_at | 2.0368426 | down | KCTD15 | potassium channel tetramerisation |
| | | | | domain containing 15 |
| 204718_at | 2.0357015 | down | EPHB6 | EPH receptor B6 |
| 202192_s_at | 2.0352569 | down | GAS7 | growth arrest-specific 7 |
| 208652_at | 2.0350847 | down | PPP2CA | protein phosphatase 2 (formerly 2A), |
| | | | | catalytic subunit, alpha isoform |
| 226388_at | 2.0340674 | down | TCEA3 | transcription elongation factor A (SII), 3 |
| 226104_at | 2.033753 | down | RNF170 | ring finger protein 170 |
| 224160_s_at | 2.0337167 | down | ACAD9 | acyl-Coenzyme A dehydrogenase |
| | | | | family, member 9 |
| 209125_at | 2.033617 | down | KRT6A | keratin 6A |
| 209529_at | 2.0331414 | down | PPAP2C | phosphatidic acid phosphatase type 2C |
| 226860_at | 2.0323775 | down | TMEM19 | transmembrane protein 19 |
| 226644_at | 2.0322123 | down | MIB2 | mindbomb homolog 2 (Drosophila) |
| 203747_at | 2.0315192 | down | AQP3 | aquaporin 3 (Gill blood group) |
| 1553960_at | 2.0313072 | down | SNX21 | sorting nexin family member 21 |
| 65630_at | 2.0311613 | down | TMEM80 | transmembrane protein 80 |
| 218171_at | 2.030586 | down | VPS4B | vacuolar protein sorting 4 homolog B |
| | | | | (S. cerevisiae) |
| 236863_at | 2.0298839 | down | C17orf67 | chromosome 17 open reading frame 67 |
| 205293_x_at | 2.0297394 | down | BAIAP2 | BAI1-associated protein 2 |
| 231115_at | 2.0290804 | down | POLH | polymerase (DNA directed), eta |
| 209563_x_at | 2.0285957 | down | CALM1 /// | calmodulin 1 (phosphorylase kinase, |
| | | | CALM2 /// | delta) /// calmodulin 2 (phosphorylase |
| | | | CALM3 | kinase, delta) /// calmodulin 3 |
| | | | | (phosphorylase kinase, delta) |
| 205668_at | 2.0280306 | down | LY75 | lymphocyte antigen 75 |
| 204347_at | 2.025962 | down | AK3L1 /// | adenylate kinase 3-like 1 /// adenylate |
| | | | AK3L2 | kinase 3-like 2 |
| 222482_at | 2.0252454 | down | LOC100131851 | hypothetical protein LOC100131851 /// |
| | | | /// | hypothetical protein LOC100134497 /// |
| | | | LOC100134497 | similar to single stranded DNA binding |
| | | | /// | protein 3 /// hypothetical LOC646674 |
| | | | LOC401002 | /// single stranded DNA binding |
| | | | /// | protein 3 |
| | | | LOC646674 | |
| | | | /// SSBP3 | |
| 220056_at | 2.023061 | down | IL22RA1 | interleukin 22 receptor, alpha 1 |
| 211986_at | 2.022835 | down | AHNAK | AHNAK nucleoprotein |
| 1555097_a_at | 2.0222125 | down | PTGFR | prostaglandin F receptor (FP) |
| 225684_at | 2.0216835 | down | FAM33A | family with sequence similarity 33, |
| | | | | member A |
| 209426_s_at | 2.0213833 | down | AMACR | alpha-methylacyl-CoA racemase |
| 203528_at | 2.0211873 | down | SEMA4D | sema domain, immunoglobulin domain |
| | | | | (Ig), transmembrane domain (TM) and |
| | | | | short cytoplasmic domain, |
| | | | | (semaphorin) 4D |
| 222895_s_at | 2.021075 | down | BCL11B | B-cell CLL/lymphoma 11B (zinc |
| | | | | finger protein) |
| 230836_at | 2.0209422 | down | ST8SIA4 | ST8 alpha-N-acetyl-neuraminide |
| | | | | alpha-2,8-sialyltransferase 4 |
| 224443_at | 2.0197344 | down | C1orf97 | chromosome 1 open reading frame 97 |
| 217845_x_at | 2.0170114 | down | HIGD1A | HIG1 domain family, member 1A |
| 1555964_at | 2.0155482 | down | ARL17 /// | ADP-ribosylation factor-like 17 /// |
| | | | ARL17P1 | ADP-ribosylation factor-like 17 |
| | | | | pseudogene 1 |
| 204061_at | 2.0146167 | down | PRKX | protein kinase, X-linked |
| 213572_s_at | 2.0136144 | down | SERPINB1 | serpin peptidase inhibitor, clade B |
| | | | | (ovalbumin), member 1 |
| 218218_at | 2.0132878 | down | APPL2 | adaptor protein, phosphotyrosine |
| | | | | interaction, PH domain and leucine |
| | | | | zipper containing 2 |
| 1554588_a_at | 2.0109353 | down | TTC30B | tetratricopeptide repeat domain 30B |
| 201563_at | 2.0097337 | down | SORD | sorbitol dehydrogenase |
| 39313_at | 2.0081642 | down | WNK1 | KIAA0344 gene |
| 225551_at | 2.0059721 | down | C1orf71 | chromosome 1 open reading frame 71 |
| 52285_f_at | 2.0037622 | down | CEP76 | centrosomal protein 76 kDa |
| 207593_at | 2.003508 | down | ABCG4 | ATP-binding cassette, sub-family G |
| | | | | (WHITE), member 4 |
| 212115_at | 2.0030887 | down | HN1L | hematological and neurological |
| | | | | expressed 1-like |
| 224605_at | 2.0020971 | down | C4orf3 | chromosome 4 open reading frame 3 |
| 1558882_at | 2.0020864 | down | LOC401233 | similar to HIV TAT specific factor 1; |
| | | | | cofactor required for Tat activation of |
| | | | | HIV-1 transcription |
| 217752_s_at | 2.001705 | down | CNDP2 | CNDP dipeptidase 2 (metallopeptidase |
| | | | | M20 family) |
| 204241_at | 2.0012212 | down | ACOX3 | acyl-Coenzyme A oxidase 3, pristanoyl |
| 219929_s_at | 2.0005429 | down | ZFYVE21 | zinc finger, FYVE domain containing |
| | | | | 21 |

Salicin 0.5% vs Untreated Control
24 hr treatment
N = 7 Salicin 0.5%, N = 4 Control
RMA normalization (performed on all 42 chips together)
t-test with Benjamini and Hochberg FDR correction
p-value cut-off: 0.05
Fold change cutoff: 2.0

### Selecting Second Subset of Genes

Because of the large amount of data associated with the gene expression level changes shown in Table 1, it is desirable to provide a further focus on genes associated with skin and aging. In one embodiment, further data sets extracted from the literature identify genes associated with physical skin aging attributes based on current knowledge of the biochemical pathways in skin to define functional youth gene assemblies. Such an assembly may then provide a gene expression focus for future further work on the same agent used to get the initial full genome data set or for other agents. For this next step, the method first uses a data set that identifies particular genes associated with biochemical pathways in skin 118 (see FIG. 1C).

Data about the biochemical pathways of genes are available from many sources of scientific literature, including databases of journal articles or from available unpublished data. To make it more useful in the present system, data collected may be supplemented with metadata classifying the conclusions reached in terminology or coding that clearly associates genes with skin or particular skin attributes. (See FIG. 7 at 786). In some embodiments, research is done on biochemical pathways of the skin for any of the genes from Table 1. (See also, FIG. 7 at 752). In some embodiments the biochemical pathway associated with physical appearance of skin aging comprises at least one of skin structural protein synthesis, degradation and maintenance, extracellular matrix assembly, cellular differentiation, skin barrier component synthesis, skin barrier integrity, water regulation, or regulation of melanin production and control. Structural protein synthesis includes, for example, elastin formation, keratinocyte differentiation and collagen production. Skin barrier component synthesis includes, for example, hyaluronic acid synthesis and lipid synthesis. Regulation of melanin production and control includes, for example, UV induced pigmentation and inhibition of tyrosinase. The data set on biochemical pathways associated with the physical appearance of skin aging known for selected genes is preferably collected and stored in database 730 in a format that promotes an intersection analysis with the data of Table 1. This can be done by building a table of all genes known to be associated with a biochemical pathway associated with the physical appearance of skin aging and finding its intersection with Table 1, or by starting the literature search with the genes in Table 1, which have already met the fold change criterion.

However approached, the intersection analysis of this step reduces the data of Table 1, by selecting from the first subset of genes a second subset of genes associated with the selected, identified biochemical pathways associated with the physical appearance of skin aging. With reference to the simplified example of FIG. 1C of the method, the method selects from genes (b, c, e, f, g and h) based on a biochemical pathway data set derived from review of scientific literature 120. For example, if in the biochemical pathway data set the gene “h” has no apparent association with a biochemical pathway associated with the physical appearance of skin aging, it may be excluded from the second subset at this point. The resulting genes in the second subset in the simplified example would be (b, c, e, f and g) as shown at 211. These genes correspond to hypothetical genes found in the intersection analysis to have a biochemical pathway associated with the physical appearance of skin aging.

The genes in the second subset for actual test results are categorized according to biological function or association with a plurality of biochemical pathways associated with the physical appearance of skin aging. For the experimental data for 0.05% salicin treated cultures (N=7) compared with untreated control cultures (N=4), Table 2 (see also, FIG. 7 at 754) shows a list of genes derived from Table 1, by selecting from Table 1 approximately 200 genes identified in a data set by their relationship to human skin in the scientific literature.

For each gene listed in Table 2, a reference is provided which discusses the mechanism of action/biochemical pathway of the gene. The references are incorporated herein by reference.

TABLE 2

| | Fold | | Gene | |
|---|---|---|---|---|
| Probe Set ID | change | Direction | Symbol | Reference |
| 225337_at | 2.704214 | up | ABHD2 | Genes to Cells (2009) 14, 407-424 |
| 200965_s_at | 11.87853 | down | ABLIM1 | Exp Gerontol. 2006 Apr; 41(4): 387-97 |
| 202422_s_at | 3.7855804 | up | ACSL4 | Arch Biochem Biophys. 1987 |
| | | | | Sep; 257(2): 302-14. |
| 214913_at | 2.1450849 | up | ADAMTS3 | J Biol Chem. 2001 Aug |
| | | | | 24; 276(34): 31502-9 |
| 202053_s_at | 4.3994246 | down | ALDH3A2 | Mol Genet Metab. 2007 Jan; 90(1): 1-9. |
| 1555416_a_at | 2.214861 | down | ALOX15B | Journal of Investigative Dermatology |
| | | | | (1991) 97, 291-297; |
| | | | | doi:10.1111/1523-1747 |
| 203747_at | 2.0315192 | down | AQP3 | Biol Cell. 2005 Jul; 97(7): 479-86 |
| 228082_at | 2.1080198 | up | ASAM | Am J Respir Cell Mol Biol. 2007 |
| | | | | Aug; 37(2): 169-85 |
| 1554980_a_at | 2.623143 | up | ATF3 | Pollack BP, Exp Dermatol.Activating |
| | | | | transcription factor 3 (ATF3) |
| | | | | expression is increased in erythema |
| | | | | multiforme and is regulated by IFN- |
| | | | | gamma in human keratinocytes |
| 205410_s_at | 3.9841306 | up | ATP2B4 | (2008) Lamellar Bodies of Human |
| | | | | Epidermis, Molecular & Cellular |
| | | | | Proteomics 7.11, 2151-2175 |
| 1558143_a_at | 2.346621 | up | BCL2L11 | J Med Invest. 2008 Aug; 55(3-4): 204-10 |
| 206176_at | 7.765054 | up | BMP6 | Exp Cell Res. 2001 Feb |
| | | | | 15; 263(2): 265-73 |
| 209563_x_at | 2.0285957 | down | CALM1 /// | Arch Dermatol Res. |
| | | | CALM2 /// | 1993; 285(5): 310-1 |
| | | | CALM3 | |
| 210020_x_at | 11.268426 | down | CALML3 | Arch Dermatol Res. |
| | | | | 1993; 285(5): 310-1 |
| 220414_at | 8.748902 | down | CALML5 | Arch Dermatol Res. |
| | | | | 1993; 285(5): 310-1 |
| 209790_s_at | 2.6684937 | down | CASP6 | Fa Yi Xue Za Zhi. 2007 |
| | | | | Oct; 23(5): 325-7, 331 |
| 211922_s_at | 2.094567 | down | CAT | Journal of Investigative Dermatology |
| | | | | (2006) 126, 182-190 |
| 206407_s_at | 5.516013 | up | CCL13 | J Allergy Clin Immunol. 2009 |
| | | | | Oct; 124(4): 753-60.e1. |
| 216598_s_at | 5.407172 | up | CCL2 | J Allergy Clin Immunol. 2009 |
| | | | | Oct; 124(4): 753-60.e1. |
| 206193_s_at | 11.105947 | down | CDSN | FASEB J. 1996 Jun; 10(8): 871-81. |
| 222549_at | 7.7580996 | down | CLDN1 | J Drugs Dermatol. 2007 Jun; 6(6 |
| | | | | Suppl): s20-4. |
| 201428_at | 3.4539902 | down | CLDN4 | Skin Pharmacol Physiol. |
| | | | | 2006; 19(2): 71-7. Epub 2006 May 9 |
| 205830_at | 2.4858525 | up | CLGN | Cell. 2008 Apr 18; 133(2): 223-34 |
| 224329_s_at | 5.4422174 | down | CNFN | Biochem Biophys Res Commun. |
| | | | | 2004 Jun 11; 318(4): 803-13 |
| 204636_at | 3.5764592 | down | COL17A1 | http://ghr.nlm.nih.gov/gene=col17a1 |
| 211981_at | 2.121121 | up | COL4A1 | Matrix Biol. 1998 Aug; 17(4): 279-91. |
| 213992_at | 4.8003573 | down | COL4A6 | The Journal of Cell Biology, Vol |
| | | | | 130, 1219-1229, Copyright © 1995 |
| 205931_s_at | 9.909108 | up | CREB5 | FEBS Lett. 2002 Jul 31; 524(1-3): |
| | | | | 193-8 |
| 229228_at | 9.767824 | up | CREB5 | FEBS Lett. 2002 Jul 31; 524(1-3): |
| | | | | 193-8 |
| 210229_s_at | 16.55027 | up | CSF2 | Thromb Haemost. 2004 |
| | | | | Aug; 92(2): 262-74 |
| 207442_at | 11.09133 | up | CSF3 | J Dermatol Sci. 2001 Apr; 25(3): 179-88 |
| 209774_x_at | 7.5460076 | up | CXCL2 | J Invest Dermatol. 2007 |
| | | | | May; 127(5): 1264-6 |
| 210764_s_at | 2.6922395 | up | CYR61 | J Biomed Sci. 2002 Jan-Feb; 9(1): 59-67. |
| 206806_at | 2.7395847 | up | DGKI | PNAS May 24, 2005 vol. 102 no. 21 |
| | | | | 7595-7600 |
| 203810_at | 2.0534089 | up | DNAJB4 | Photodermatol Photoimmunol |
| | | | | Photomed. 2004 Jun; 20(3): 129-37 |
| 202843_at | 3.5600665 | up | DNAJB9 | Mol Cancer Ther. 2008 |
| | | | | Aug; 7(8): 2319-27. |
| 221782_at | 2.5525873 | up | DNAJC10 | Photodermatol Photoimmunol |
| | | | | Photomed. 2004 Jun; 20(3): 129-37 |
| 207324_s_at | 12.947083 | down | DSC1 | FASEB J. 1996 Jun; 10(8): 871-81. |
| 206033_s_at | 4.676169 | down | DSC3 | FASEB J. 1996 Jun; 10(8): 871-81. |
| 244852_at | 2.2514307 | up | DSEL | Apr. 10, 2009 The Journal of |
| | | | | Biological Chemistry, 284, 9788-9795 |
| 200606_at | 2.8482468 | down | DSP | FASEB J. 1996 Jun; 10(8): 871-81. |
| 201044_x_at | 3.7260537 | up | DUSP1 | United States Patent Application |
| | | | | 20060116319 |
| 204273_at | 3.9157705 | up | EDNRB | Clinics in Dermatology, Volume 23, |
| | | | | Issue 1, January-February 2005, |
| | | | | Pages 56-67 |
| 214446_at | 2.5570273 | up | ELL2 | DNA Repair (Amst). 2007 Jun |
| | | | | 1; 6(6): 841-51. Epub 2007 Mar 19 |
| 234513_at | 2.0481179 | down | ELOVL3 | J Biol Chem. 2004 Feb |
| | | | | 13; 279(7): 5621-9. Epub 2003 Oct 27 |
| 204256_at | 3.1214519 | down | ELOVL6 | Prog Lipid Res. 2006 |
| | | | | May; 45(3): 237-49 |
| 201839_s_at | 2.0932202 | down | EPCAM | PLoS Genet. 2009 |
| | | | | Jul; 5(7): e1000563. Epub 2009 Jul 17 |
| 232165_at | 12.401502 | down | EPPK1 | FASEB J. 1996 Jun; 10(8): 871-81. |
| 202609_at | 2.334194 | up | EPS8 | Am J Pathol. 2000 Jul; 157(1): 59-68. |
| 205767_at | 2.486593 | up | EREG | J Biol Chem. 2000 Feb |
| | | | | 25; 275(8): 5748-53. |
| 206429_at | 2.0988882 | down | F2RL1 | Proc Assoc Am Physicians. 1997 |
| | | | | Mar; 109(2): 190-207 |
| 203980_at | 2.2506835 | up | FABP4 | FEBS Lett. 2009 Apr |
| | | | | 17; 583(8): 1319-22. |
| 208962_s_at | 2.889333 | up | FADS1 | Journal of Investigative Dermatology |
| | | | | 129, 2795-2804 (December 2009) |
| 203184_at | 2.0282688 | up | FBN2 | J Invest Dermatol. 1996 |
| | | | | May; 106(5): 1090-5 |
| 227271_at | 2.6716337 | down | FGF11 | Journal of Endocrinology (2005) |
| | | | | 186, 273-289 DOI: |
| | | | | 10.1677/joe.1.06055 |
| 205110_s_at | 2.1575923 | up | FGF13 | J Invest Dermatol. 2004 |
| | | | | May; 122(5): 1084-90. |
| 204421_s_at | 3.8445234 | up | FGF2 | Int J Cosmet Sci. 2009 |
| | | | | Dec; 31(6): 419-26. |
| 1555103_s_at | 2.3495677 | up | FGF7 | J Dermatol Sci. 2009 May; 54(2): 106-13. |
| 205782_at | 3.1584346 | up | FGF7 | J Dermatol Sci. 2009 May; 54(2): 106-13. |
| 1554741_s_at | 2.5060341 | up | FGF7 /// | Huang, T.-J., Lee, C.-J., Expression of |
| | | | KGFLP1 /// | keratinocyte growth factor (KGF) is |
| | | | KGFLP2 | regulated by two KGF-like proteins, |
| | | | | KGFLP1 and KGFLP2. Submitted |
| | | | | APR-2002 |
| 205014_at | 3.40602 | down | FGFBP1 | J Biol Chem. 2000 Apr |
| | | | | 14; 275(15): 10802-11. |
| 204379_s_at | 5.287966 | down | FGFR3 | J. Clin. Invest. 116: 8 |
| | | | | doi:10.1172/JCI28163 |
| 204135_at | 2.9959981 | down | FILIP1L | Nature Genetics 33, 487-491 |
| | | | | (2003) Published online: March 2003;\| |
| | | | | doi:10.1038/ng1119 |
| 1569410_at | 45.91465 | down | FLG2 | PLoS One. 2009; 4(4): e5227. |
| 210287_s_at | 3.9503276 | up | FLT1 | J Invest Dermatol. 2007 |
| | | | | Oct; 127(10): 2445-52. |
| 214702_at | 2.7099462 | up | FN1 | Forensic Science International, |
| | | | | Volume 126, Issue 2, Page 118 |
| 215910_s_at | 3.663163 | up | FNDC3A | FEBS Lett. 1994 Apr 18; 343(1): 47-50 |
| 202724_s_at | 2.0982356 | up | FOXO1 | J Investig Dermatol Symp Proc. 2009 |
| | | | | Aug; 14(1): 60-2. |
| 209602_s_at | 15.703371 | down | GATA3 | BMC Genomics. 2009 Sep 7; 10: 417. |
| 221577_x_at | 5.573193 | up | GDF15 | Oncogene. 2008 Jan 17; 27(4): 409-20. |
| 235405_at | 3.2997503 | down | GSTA4 | Int J Biochem Cell Biol. 1995 |
| | | | | Mar; 27(3): 271-7 |
| 225245_x_at | 2.328221 | down | H2AFJ | Exp Gerontol. 1999 Sep; 34(6): 741-54 |
| 206643_at | 55.867226 | down | HAL | J Dermatol Sci. 2008 Jun; 50(3): 209-15. |
| | | | | Epub 2008 Feb 15. |
| 223541_at | 3.713034 | down | HAS3 | Arch Dermatol Res. 2006 |
| | | | | Nov; 298(6): 273-82 |
| 210998_s_at | 4.350414 | up | HGF | Cytokine. 2000 Jun; 12(6): 780-5. |
| 221750_at | 2.4064143 | down | HMGCS1 | Clin Chim Acta. 1988 Jan |
| | | | | 15; 171(1): 95-101 |
| 222881_at | 4.1936674 | down | HPSE | Journal of Investigative Dermatology |
| | | | | (2001) 117, 1266-1273 |
| 227361_at | 3.409933 | up | HS3ST3B1 | Biochem Biophys Res Commun. |
| | | | | 2008 Aug 8; 372(4): 681-7 |
| 202558_s_at | 3.1297736 | up | HSPA13 | Cell Stress Chaperones. 2009 |
| | | | | Jan; 14(1): 1-21. |
| 200800_s_at | 3.1119442 | up | HSPA1A /// | Cell Stress Chaperones. 2009 |
| | | | HSPA1B | Jan; 14(1): 1-21. |
| 213418_at | 14.241181 | up | HSPA6 | Cell Stress Chaperones. 2009 Oct 7. |
| 221667_s_at | 4.658692 | down | HSPB8 | Oncogene. 2007 May |
| | | | | 24; 26(24): 3521-31 |
| 219284_at | 2.0798855 | up | HSPBAP1 | Br J Dermatol. 1995 |
| | | | | Aug; 133(2): 194-202 |
| 210619_s_at | 4.5378346 | down | HYAL1 | Journal of Investigative Dermatology |
| | | | | (2007) 127, 512-513 |
| 220249_at | 3.116939 | down | HYAL4 | J Invest Dermatol. 1994 |
| | | | | Mar; 102(3): 385-9 |
| 202411_at | 4.671116 | down | IFI27 | Journal of Investigative Dermatology |
| | | | | (2004) 122, 717-721 |
| 211958_at | 2.7118793 | up | IGFBP5 | Biochem Soc Trans. 2009 Aug; 37(Pt4): |
| | | | | 882-5. |
| 206924_at | 9.694227 | up | IL11 | J Allergy Clin Immunol. 2003 |
| | | | | Apr; 111(4): 875-81 |
| 205992_s_at | 4.32588 | up | IL15 | J Immunol. 1995 Nov 1; 155(9): 4492-6. |
| 206295_at | 19.309084 | down | IL18 | Arch Dermatol Res. 2001 |
| | | | | Jul; 293(7): 325-33 |
| 206618_at | 2.864942 | up | IL18R1 | Autoimmun Rev. 2009 Sep; 9(1): 45-8 |
| 220745_at | 2.2776432 | up | IL19 | J Invest Dermatol. 2003 |
| | | | | Dec; 121(6): 1306-11. |
| 221470_s_at | 100.54604 | down | IL1F7 | Wound Repair Regen. 2008 Jul-Aug; |
| | | | | 16(4): 534-41. |
| 205403_at | 3.2927191 | down | IL1R2 | Journal of Investigative Dermatology |
| | | | | (1996) 106, 1102-1107; |
| | | | | doi:10.1111/1523-1747 |
| 207526_s_at | 9.089992 | up | IL1RL1 | Hum Mol Genet. 2005 Oct |
| | | | | 1; 14(19): 2919-27 |
| 219115_s_at | 12.066238 | down | IL20RA | Genes Immun. 2008 Jul; 9(5): 445-51. |
| 228575_at | 10.042443 | down | IL20RB | Genes Immun. 2008 Jul; 9(5): 445-51. |
| 220056_at | 2.023061 | down | IL22RA1 | Clin Exp Immunol. 2007 December; |
| | | | | 150(3): 407-415. doi: |
| | | | | 10.1111/j.1365-2249.2007.03511.x |
| 220054_at | 2.967118 | up | IL23A | Genes Immun. 2009 Apr; 10(3): 201-9 |
| 206569_at | 8.736957 | up | IL24 | Cytokine. 2008 Jan; 41(1): 16-23 |
| 205207_at | 3.1970735 | up | IL6 | Journal of Investigative Dermatology |
| | | | | (2004) 123, 124-131 |
| 207008_at | 11.107841 | down | IL8RB | Lab Invest 2000, 80: 595-604 |
| 201474_s_at | 2.051571 | down | ITGA3 | J Clin Invest. 2008 Mar; 118(3): 965-74 |
| 201656_at | 4.4418707 | down | ITGA6 | J Cell Biol. 1996 July 2; 134(2): |
| | | | | 559-572 |
| 1561042_at | 2.1507823 | up | ITGB1 | The Journal of Immunology, Vol |
| | | | | 154, Issue 11 6058-6064 |
| 204990_s_at | 4.5270753 | down | ITGB4 | J Cell Biol. 1996 July 2; 134(2): |
| | | | | 559-572 |
| 231031_at | 3.3397727 | up | KGFLP2 | Huang, T.-J., Lee, C.-J., Expression of |
| | | | | keratinocyte growth factor (KGF) is |
| | | | | regulated by two KGF-like proteins, |
| | | | | KGFLP1 and KGFLP2. Submitted |
| | | | | APR-2002 |
| 231015_at | 2.3381798 | up | KLF15 | The Journal of Clinical |
| | | | | Endocrinology & Metabolism Vol. |
| | | | | 94, No. 7 2594-2601 |
| 221841_s_at | 2.467558 | down | KLF4 | Acta Biochim Biophys Sin |
| | | | | (Shanghai). 2008 Jul; 40(7): 554-64 |
| 216699_s_at | 2.168667 | down | KLK1 | Arch Dermatol Res. |
| | | | | 1980; 267(3): 301-11 |
| 215808_at | 4.67278 | down | KLK10 | Journal of Investigative Dermatology |
| | | | | (2005) 125, 1182-1189; |
| | | | | doi:10.1111/j.0022- |
| | | | | 202X.2005.23933.x |
| 205470_s_at | 3.3177328 | down | KLK11 | Journal of Investigative Dermatology |
| | | | | (2005) 125, 1182-1189; |
| | | | | doi:10.1111/j.0022- |
| | | | | 202X.2005.23933.x |
| 205783_at | 3.6192052 | down | KLK13 | Journal of Investigative Dermatology |
| | | | | (2005) 125, 1182-1189; |
| | | | | doi:10.1111/j.0022- |
| | | | | 202X.2005.23933.x |
| 204733_at | 16.28207 | down | KLK6 | J Biol Chem. 2007 Feb |
| | | | | 23; 282(8): 5834-41. |
| 1552319_a_at | 4.8667984 | down | KLK8 | Journal of Investigative Dermatology |
| | | | | (2005) 125, 1182-1189; |
| | | | | doi:10.1111/j.0022- |
| | | | | 202X.2005.23933.x |
| 205900_at | 4.1411896 | down | KRT1 | Gene Expr Patterns. 2005 |
| | | | | Aug; 5(6): 801-8 |
| 204734_at | 3.6002238 | down | KRT15 | J Invest Dermatol. 1999 |
| | | | | Mar; 112(3): 362-9 |
| 218963_s_at | 10.944303 | down | KRT23 | Molecular Oncology, Volume 1, |
| | | | | Issue 2, Pages 181-195 |
| 201820_at | 2.1536007 | down | KRT5 | Acta Derm Venereol. 2004; 84(1): 18-22 |
| 209125_at | 2.033617 | down | KRT6A | http://ghr.nlm.nih.gov/gene=krt6a |
| 208188_at | 2.5387063 | down | KRT9 | Journal of Investigative Dermatology |
| | | | | (1994) 103, 474-477; |
| | | | | doi:10.1111/1523-1747 |
| 1556410_a_at | 24.649403 | up | KRTAP19-1 | J Biol Chem. 2002 Dec |
| | | | | 13; 277(50): 48993-9002 |
| 235148_at | 2.1576118 | down | KRTCAP3 | Br J Dermatol. 2003 Apr; 148(4): 654-64. |
| 1554252_a_at | 15.340231 | down | LASS3 | (2003) J. Biol. Chem. 278, 43452-43459 |
| 218922_s_at | 3.0278475 | down | LASS4 | Molecular Endocrinology |
| | | | | 22(11): 2407-2419 |
| 1560531_at | 71.235 | down | LCE1B | Journal of Investigative Dermatology |
| | | | | (2005) 124, 1062-1070 |
| 1559224_at | 4.81139 | down | LCE1E | Journal of Investigative Dermatology |
| | | | | (2005) 124, 1062-1070; |
| | | | | doi:10.1111/j.0022- |
| | | | | 202X.2005.23699.x |
| 207710_at | 200.50969 | down | LCE2B | Journal of Investigative Dermatology |
| | | | | (2005) 124, 1062-1070 |
| 212531_at | 12.014198 | down | LCN2 | Journal of Investigative Dermatology |
| | | | | (2006) 126, 510-512. |
| | | | | doi:10.1038/sj.jid.5700035 |
| 205266_at | 3.7018826 | up | LIF | J Invest Dermatol. 2001 |
| | | | | Mar; 116(3): 476-8. |
| 207720_at | 32.353355 | down | LOR | Int Arch Allergy Immunol. |
| | | | | 2010; 151(1): 28-37. |
| 204038_s_at | 2.7315595 | up | LPAR1 | J Invest Dermatol. 2005 |
| | | | | Sep; 125(3): 421-7. |
| 203549_s_at | 4.0753236 | up | LPL | Diabetes. 2001 Jul; 50(7): 1643-53 |
| 216388_s_at | 2.4562252 | down | LTB4R | J Invest Dermatol. 2009 |
| | | | | Dec; 129(12): 2854-60 |
| 206249_at | 2.2523835 | up | MAP3K13 | The EMBO Journal (2006) 25, 5105-5116 |
| 216199_s_at | 2.6540227 | up | MAP3K4 | Biochem J. 2006 Jun 1; 396(2): 307-16. |
| 203836_s_at | 2.3556855 | up | MAP3K5 | r J Immunol. 2005 Jun; 35(6): 1886-95. |
| 213764_s_at | 2.1121294 | down | MFAP5 | J Biol Chem. 1996 Jan |
| | | | | 12; 271(2): 1096-103 |
| 239273_s_at | 2.6781023 | down | MMP28 | Matrix Biol. 2009 Mar; 28(2): 74-83. |
| | | | | Epub 2009 Jan 20 |
| 219648_at | 2.8866937 | down | MREG | J Biol Chem. 2009 Apr |
| | | | | 17; 284(16): 10877-89 |
| 1552578_a_at | 2.337757 | up | MYO3B | Journal of Biomedical Science |
| | | | | (2007) 14: 87-105 |
| 217738_at | 2.6675475 | up | NAMPT | Dec. 3, 2004 The Journal of |
| | | | | Biological Chemistry, 279, 50754-50763. |
| 201502_s_at | 3.0644495 | up | NFKBIA | Photochem Photobiol. 1995 |
| | | | | Sep; 62(3): 463-8. |
| 223217_s_at | 2.3498836 | up | NFKBIZ | J Biol Chem. 2004 Dec |
| | | | | 31; 279(53): 55493-8 |
| 202340_x_at | 2.33773 | up | NR4A1 | J Biol Chem. 2008 May |
| | | | | 2; 283(18): 12564-70 |
| 216248_s_at | 7.5919886 | up | NR4A2 | J Invest Dermatol. 2008 |
| | | | | Feb; 128(2): 300-10 |
| 216979_at | 7.203941 | up | NR4A3 | J Biol Chem. 2008 May |
| | | | | 2; 283(18): 12564-70 |
| 225566_at | 3.1977339 | up | NRP2 | Proc Natl Acad Sci USA. 2001 Oct |
| | | | | 23; 98(22): 12677-82. |
| 208591_s_at | 2.851169 | up | PDE3B | Med Hypotheses. 2009 |
| | | | | Jul; 73(1): 118-9. |
| 203708_at | 5.313648 | up | PDE4B | Int Immunopharmacol. 2009 |
| | | | | Jan; 9(1): 55-62. |
| 217997_at | 3.4090981 | up | PHLDA1 | J Clin Invest. 2006 Jan; 116(1): 249-60. |
| 209873_s_at | 4.6149898 | down | PKP3 | FASEB J. 1996 Jun; 10(8): 871-81. |
| 201860_s_at | 4.228699 | up | PLAT | J Am Podiatr Med Assoc. 2008 Sep-Oct; |
| | | | | 98(5): 345-52 |
| 210845_s_at | 2.249447 | up | PLAUR | Dev Biol. 2008 May 1; 317(1): 187-95. |
| 203896_s_at | 4.0160823 | up | PLCB4 | Journal of Investigative Dermatology |
| | | | | (1997) 108, 748-752 |
| 213093_at | 2.125206 | up | PRKCA | Exp Dermatol. 2002 Feb; 11(1): 25-33. |
| 220635_at | 19.078548 | down | PSORS1C2 | Journal of Investigative Dermatology |
| | | | | (2007) 127, 1605-1614 |
| 213933_at | 13.583514 | down | PTGER3 | J Allergy Clin Immunol. 2009 |
| | | | | Oct; 124(4): 809-18.e2. |
| 204897_at | 2.5254738 | up | PTGER4 | Nat Med. 2003 Jun; 9(6): 744-9 |
| 207388_s_at | 2.7678204 | up | PTGES | Arthritis Rheum. 2007 |
| | | | | Nov; 56(11): 3564-74. |
| 205128_x_at | 2.326936 | up | PTGS1 | www.jimmunol.org/cgi/reprint/173/2/1321.pdf |
| 1554997_a_at | 6.812711 | up | PTGS2 | J Lipid Res. 2006 May; 47(5): 921-30 |
| 204944_at | 2.2989373 | up | PTPRG | J Biol Chem. 2006 Sep |
| | | | | 15; 281(37): 27389-97 |
| 212099_at | 3.7642062 | up | RHOB | J Biol Chem. 2005 Dec |
| | | | | 30; 280(52): 43257-63. |
| 214370_at | 2.9802194 | up | S100A8 | Arch Dermatol Res. 2009 |
| | | | | Aug; 301(7): 523-9. Epub 2009 May |
| | | | | 23. |
| 200832_s_at | 3.7699654 | down | SCD | Life Sciences Volume 84, Issues 3-4, |
| | | | | 16 Jan. 2009, Pages 119-124 |
| 1554921_a_at | 6.0999765 | down | SCEL | Journal of Investigative Dermatology |
| | | | | (2003) 121, 781-785; |
| | | | | doi:10.1046/j.1523- |
| | | | | 1747.2003.12539.x |
| 229199_at | 3.8180137 | up | SCN9A | J Invest Dermatol. 2005 |
| | | | | Jun; 124(6): 1333-8. |
| 201287_s_at | 3.32023 | down | SDC1 | Arch Dermatol Res. 2008 August; |
| | | | | 300(7): 393-395 |
| 211906_s_at | 5.612926 | down | SERPINB4 | Clin Exp Allergy. 2005 |
| | | | | Oct; 35(10): 1327-33. |
| 227487_s_at | 3.1483386 | up | SERPINE2 | J Cell Sci. 2008 May 1; 121(Pt9): |
| | | | | 1435-43. |
| 212321_at | 2.405509 | down | SGPL1 | Cell Signal. 2007 Apr; 19(4): 748-60 |
| 238567_at | 5.6408434 | down | SGPP2 | Cell Signal. 2007 Apr; 19(4): 748-60. |
| 230494_at | 2.5260012 | up | SLC20A1 | Comp Funct Genomics. 2003 |
| | | | | February; 4(1): 47-55. doi: |
| | | | | 10.1002/cfg.239 |
| 223222_at | 2.2426856 | up | SLC25A19 | Cell Mol Life Sci. 2005 Oct; 62(19-20): |
| | | | | 2204-27 |
| 204790_at | 2.1506758 | up | SMAD7 | Developmental Cell, Volume 11, |
| | | | | Issue 3, Pages 301-312 |
| 219695_at | 13.657985 | down | SMPD3 | Arch Dermatol Res. 2009 |
| | | | | Sep; 301(8): 587-94. |
| 1553960_at | 2.0313072 | down | SNX21 | Proc Natl Acad Sci USA. 2001 Jul |
| | | | | 17; 98(15): 8756-61 |
| 215078_at | 5.7122235 | up | SOD2 | J Cell Physiol. 1995 Dec; 165(3): 576-87. |
| 202826_at | 4.585347 | down | SPINT1 | Am J Pathol. 2008 Nov; 173(5): 1464-75. |
| 209437_s_at | 2.3600132 | up | SPON1 | Nature Genetics 38, 1304-1309 (1 |
| | | | | Nov. 2006)\| |
| | | | | doi:10.1038/ng1907 |
| 208539_x_at | 5.553559 | down | SPRR2B | Journal of Investigative Dermatology |
| | | | | (2005) 125, 1286-1301 |
| 213921_at | 4.35561 | up | SST | Acta Derm Venereol. 1994 |
| | | | | Mar; 74(2): 106-9 |
| 213103_at | 2.8436816 | up | STARD13 | Journal of Cell Science 118, 2791-2801 |
| | | | | (2005) |
| 213820_s_at | 3.4357908 | down | STARD5 | J Biol Chem. 2003 Jun |
| | | | | 20; 278(25): 22183-6 |
| 212353_at | 4.4241323 | up | SULF1 | Development. 2007 |
| | | | | Sep; 134(18): 3327-38 |
| 205547_s_at | 2.4258394 | up | TAGLN | Wound Repair and Regeneration, |
| | | | | Volume 14, Issue 4 (p 463-470) |
| 205015_s_at | 3.1335502 | down | TGFA | J Invest Dermatol. 1990 Jun; 94(6 |
| | | | | Suppl): 164S-170S |
| 201042_at | 2.3514817 | up | TGM2 | JPET December 2006 vol. 319 no. 3 |
| | | | | 1162-1171 |
| 203887_s_at | 3.1370428 | down | THBD | Br J Dermatol. 1996 |
| | | | | Aug; 135(2): 187-93 |
| 201150_s_at | 2.0579739 | up | TIMP3 | J Invest Dermatol. 1998 |
| | | | | Apr; 110(4): 416-21 |
| 209387_s_at | 3.1869683 | up | TM4SF1 | J Cell Sci. 2008 Mar 1; 121(Pt5): |
| | | | | 685-94. Epub 2008 Feb 12. |
| 216005_at | 2.7193272 | up | TNC | J Cell Physiol. 2006 Mar; 206(3): 718-27. |
| 202643_s_at | 6.915339 | up | TNFAIP3 | FEBS Lett. 2003 Feb 11; 536(1-3): |
| | | | | 135-40 |
| 206025_s_at | 3.1820982 | up | TNFAIP6 | Am J Pathol. 2009 Nov; 175(5): 1915-28. |
| 204932_at | 6.287326 | up | INFRSF11B | United States Patent Application |
| | | | | 20030175710 |
| 202687_s_at | 20.30895 | down | TNFSF10 | Gene. 2004 Oct 27; 341: 199-207. |
| 230398_at | 2.7380996 | down | TNS4 | Journal of Investigative Dermatology |
| | | | | (2008) 128, 783-790. |
| | | | | doi:10.1038/sj.jid.5700969 |
| 224220_x_at | 2.7084796 | up | TRPC4 | Neurosci Lett. 2003 Dec |
| | | | | 26; 353(3): 189-92. |
| 217979_at | 2.400637 | down | TSPAN13 | Mol Cell Biol. 2004 Jul; 24(13): 5978-88 |
| 204141_at | 2.738532 | down | TUBB2A | Am J Dermatopathol. 1990 |
| | | | | Feb; 12(1): 17-24 |
| 202154_x_at | 2.9669414 | down | TUBB3 | Am J Dermatopathol. 1990 |
| | | | | Feb; 12(1): 17-24 |
| 218810_at | 2.1168468 | up | ZC3H12A | The American Journal of Pathology, |
| | | | | Vol. 171, No. 1, July 2007 |

Salicin 0.5% vs Untreated Control
24 hr treatment
N = 7 Salicin 0.5%, N = 4 Control
RMA normalization (performed on all 42 chips together)
t-test with Benjamini and Hochberg FDR correction
p-value cut-off: 0.05
Fold change cutoff: 2.0

Genes not chosen for the second subset of genes may nonetheless be considered for additional research based on secondary research factors 122. For example, a hypothetical gene may have interesting aging-related pathways, not yet associated with skin, or in the case of hypothetical gene h, a gene may have a high fold value.

### Grouping Second Subset of Genes with Attributes of Skin

Applying to Table 1 the data set specifying the associations with biochemical pathways with the physical appearance of skin aging is a meaningful focusing of the data, which results in the list of Table 2 (See FIG. 7 at 754). Even the shorter list of about 200 genes in Table 2 can benefit from further focus. In particular, it has been found useful to further focus on particular skin attributes that are associated with skin aging.

Returning to the flowchart of FIGS. 1A-1K for the simplified example, for one or more skin attributes of interest, data is derived from the literature that identifies with each skin attribute the genes and their biochemical pathways that are recognized as affecting the skin attribute 123. Thus, FIG. 1D shows a step of selecting from the second subset of hypothetical genes, which have biologically relevant fold changes and are identified with biochemical pathways with the physical appearance of skin aging, further subsets or groups associated with a particular skin attribute 124.

According to the method, genes of the second subset are further processed into a plurality of subsets (potentially overlapping) within the second subset by categorizing or associating each gene by an association with one or more skin attribute(s). (Gene b is in the subset of skin structure and also in the subset of skin pigmentation). For example, the genes from the second subset may be transformed into skin attribute subsets, each associated with a particular physical sign of skin aging and appearance, listed as follows in FIGS. 1E and 1F:

Skin structure attribute 126.

Skin pigmentation attribute 128.

Skin hydration attribute 130.

Cell turnover attribute 132.

Turning to the second subset as defined for the experimental data listed in Table 2, the use of skin attribute data sets is further explained. A data set identifying the relationship between a particular skin attribute and particular genes is developed based on the literature or on available unpublished data. The data set identifies biochemical pathways of the physical appearance of skin aging known to be associated with one of the genes of Table 2 and a particular skin attribute; it is preferably collected and stored in a format that promotes an intersection analysis with the data of Table 2. This can be done by building a table of all genes known to be associated with a biochemical pathway of the physical appearance of skin aging and a particular skin attribute of interest and finding its intersection with Table 2. In some embodiments, the genes involved in various biochemical pathways related to the particular attribute “skin structure” are chosen for analysis. Some of the biochemical pathways of interest for this skin attribute include skin structural proteins synthesis, degradation and maintenance and extracellular matrix assembly. However, other skin attributes and associated pathways may also be of interest.

The attributes skin structure, pigmentation, cell turnover and hydration are described below. One can derive from the literature for each attribute its own subset of genes associated with that attribute, which permits development of a data set identifying the relationship between each of these particular skin attributes and particular genes. However, a gene may be associated with more than one skin attribute. A brief discussion of general principles of skin aging is a useful preface to a discussion of skin attributes.

The stratum corneum is the layer of the skin that forms the top surface layer and serves to protect the skin while controlling moisture and the flow of substances in and out of the skin. As this barrier function is broken down, the skin suffers damaging effects, thus further contributing to premature aging. These damaging effects causing premature aging of the skin are a concern for many individuals wishing to maintain healthy, youthful looking and feeling skin.

Aging can occur from biological processes or environmental factors, and in some cases environmental factors that impact biological processes. These factors alone and in combination contribute to aging appearance and are responsible for the decline in skin health and function. Biological aging, which is intrinsic, is the result of changes, often genetically determined, that occur naturally within the body. Environmental aging, which is extrinsic, is the result of free radical damage generated by accumulated exposure to sunlight (photoaging), pollution, or cigarette smoke. Also, lifestyle choices like diet, sleep, and stress can affect how quickly one appears to age.

Whether from biological or environmental sources, the appearance of aging results from several mechanisms of action or biochemical pathways. For example, a loss of skin structure, a slowing skin cell turnover, pigmentation changes or a decrease in skin hydration.

One can group many of the detectable/sensible changes that occur with skin aging into four major skin attributes, skin structure, pigmentation, cell turnover and hydration. By defining these attributes and developing metrics for them, based where possible on instrumentation that makes the metrics more objective, research on interventions can be given focus. For example, with a chosen attribute and one or more genes and one or more biochemical pathways associated with it, the research can focus on particular parts of a biochemical pathway that can be enhanced to encourage the biochemical pathways that produce a more youthful version of that attribute or on inhibiting a biochemical pathway that produces a less youthful version of that attribute. It is believed that the biochemical pathways associated with genes can be regulated by many different factors. The focus on particular genes, particular biochemical pathways associated with the genes and particular skin attributes associated with those pathways may permit identifying an “intervention” where a specific technology can target the gene expression activity for a particular skin attribute to reflect a more youthful gene expression profile, ultimately influencing the physical appearance of the skin as it ages.

Functional youth gene assembly, refers to a group of genes, encompassing one or more mechanisms of aging, addressable for functional restoration or stabilization of a more youthful state in the skin. Each functional youth gene assembly may focus on a particular skin attribute that has youthful and non-youthful states. A functional youth gene assembly could also apply to characteristics in other tissues and organs.

By extension, a “youth gene family” is composed of a related group of functional youth gene assemblies and would address multiple (or all) the significant attributes of aging for skin (or another tissue or organ, such as adipose tissue, heart, brain, skeletal muscle, etc.).

Once it is defined, one can examine a functional youth gene assembly for a specific tissue associated with a specific function. For example, in skin, the youth gene family may comprise functional youth gene assemblies for skin pigmentation, structure, hydration and cell turnover. The skin is an easily accessible organ with easily measured or observed aging attributes. Therefore, one can readily examine manifestations of changed expression levels of a functional youth gene assembly for a specific attribute of skin.

This approach is part of an overall strategy to slow down the physical manifestation of the aging process in skin by developing a composition that addresses several genetic mechanisms of aging simultaneously, i.e., through actions targeted to expression levels of the members of functional youth gene assemblies, instead of in-depth analysis of an individual gene. The following focuses on four skin attributes for which it is useful to define a functional youth gene assembly.

A. Skin Structure

The skin structure group has genes that have biochemical pathways associated with the physical appearance of skin aging that include for example, skin structural proteins synthesis, degradation and maintenance and extracellular matrix assembly. Examples of skin structural protein synthesis include elastin formation, keratinocyte differentiation and collagen production.

Younger skin has the ability to balance damage and repair to collagen, a structural protein in the skin. This balance keeps skin looking smooth and wrinkle free. During the aging process, skin begins to lose this balance. Less and less collagen is created and more enzymes are produced which break down this protein resulting in lines and wrinkles. Increasing the production of structural proteins promotes youthful looking skin, whereas inhibiting the production of enzymes that break down the proteins in the skin is also beneficial.

B. Pigmentation

The pigmentation group has genes that have biochemical pathways associated with the physical appearance of skin aging that include for example, regulation of melanin production and control. All normal human skin contains chromophores that give the skin a characteristic coloration. The color of the skin is mostly due to melanin, eumelanin, hemoglobin and, to some degree, collagen and elastin. The primary function of pigmentation is the absorption of short wavelength light capable of damaging structural components in the deeper layers of the skin and the nuclear and mitochondrial DNA of keratinocytes, melanocytes, fibroblasts, lipocytes, Langerhans cells, other immune system cells and neural cells in the skin.

Aside from a sallow appearance, seen in thinner, lighter skinned individuals with poor circulation, around the globe the overall color of the skin does not reflect aging. However, across cultures the irregular distribution of skin color, sometimes called dispigmentation, is a key attribute that characterizes older skin and presents in the form of ephelides, letingines and hyperpigmented or hypopigmented scars.

The biochemical changes in the skin that drive an irregular pigmentation pattern can be grouped into several categories. Increased growth factor signaling by melanocyte-stimulating hormone and related cytokines, increased tyrosinase, an enzyme converting tyrosine to DOPA and on to melanin, and increased amounts of melanin transferred from the melanocytes to the keratinocytes.

During the aging process, melanocytes can cluster together. These clusters of melanocytes can then become overly active resulting in areas of hyperpigmentation, known as age spots or discoloration.

C. Cell Turnover

The cell turnover group has genes that have biochemical pathways associated with the physical appearance of skin aging that include for example, cellular differentiation. During the aging process, the outer layers of the skin do not slough off as they once did. The adhesion of these skin cells result in rough skin texture and dull, lifeless looking skin. When cell renewal slows with age, dead skin cells build up along the pores of the skin. This build up increases the appearance of pores, making them look larger than in youthful looking skin.

By increasing the cell renewal process, younger, healthier skin cells surface, promoting a smoother skin texture. When skin looks smoother, it reflects light more uniformly. Smoother skin appears more radiant and bright. Increasing cell renewal also stimulates a healthy exfoliation that results in the appearance of tighter pores.

D. Hydration

The skin hydration group has genes that have biochemical pathways associated with the physical appearance of skin aging that include for example, skin barrier component synthesis, skin barrier integrity and water regulation. Skin barrier component synthesis includes, for example, hyaluronic acid synthesis and lipid synthesis.

Moisture-binding glycosaminoglycans (GAG) found within the extracellular skin matrix play a role in the hydration and moisture levels within the skin. Ample moisturization within the extracellular matrix is a factor that helps maintain the strength and integrity of the structural proteins. Many GAGs are too large to enter through the epidermis, but there are ingredients that have been shown to increase GAG production.

Each skin attribute may be negatively impacted by one or more mechanisms that contribute to aging in the skin, or may be positively impacted by a mechanism that preserves youthful appearance. If biochemical processes affecting each attribute and the gene expression profile driving those biochemical pathways or processes can be addressed (up-regulated or down-regulated), a skin state more characteristic of a younger individual is established.

Referring to the simplified example of FIGS. 1D-1F, the processing of steps 124, 126, 128, 130, 132 begins by focusing on one or more particular skin attributes. For each skin attribute, from the literature a set of genes may be found that has a biochemical pathway relevant to the skin attribute. With sufficient knowledge of the pathway, it can be determined from the literature what the function of the pathway is and thus, whether that pathway will assist a more youthful state of the skin attribute of interest if the pathway is up-regulated or down regulated. The data on a skin attribute and genes with a biochemical pathway relevant to it may be assembled as a dataset for that skin attribute. FIGS. 1D-1F show for the simplified genome (a-h) one format. For each of genes a-h in the table at step 120 and for each skin attribute, a row of data can show which genes have association with the skin attribute of interest and show a direction of regulation of the gene expression associated with a more youthful appearance. This is shown in the table at step 123 in one row for each of the skin attributes. (See rows 213a-213d). As will be seen, these data sets set up the intersection processing for individual skin attributes that leads to the tables at steps 126, 128, 130, and 132.

Correspondingly, for the microarray data in Table 2, from the genes in Table 2 a data set can be prepared that show association for a chosen skin attribute of interest, which of these genes has a pathway relevant to the skin attribute of interest and from the function of that pathway can be determined a direction of regulation of the gene associated with a more youthful appearance. The association of a gene and its pathway and one or more skin attributes may come from published or private research. Table 3 shows a dataset taken form the genes in Table 2, and identifying those genes with a pathway relevant to the skin attribute “skin structure”. For each gene in the list (of Table 3) there is a column entry in which a function relative to the skin attribute appears. The resulting skin attribute data set may be input into a database 730, by a suitable process application module that stores and accesses such data and uses it for processing as contemplated by steps 124, 126, 128, 130, 132 in the hypothetical example.

The intersection analysis for the microarray data in Table 3 is similar to that shown in steps 124, 126, 128, 130, 132 (which deal with all four skin attributes identified above; for the data in Table 2, there is only one example skin attribute addressed in Table 3). The skin attribute data set of Table 3 may be processed to derive a skin attribute subset for a preliminary functional gene assembly. As the data of Table 2 is processed by intersection with a skin attribute data set designating genes with an association with a skin attributes of interest, it becomes necessary to consider for each gene the literature-reported regulation direction for the more youthful state of the skin attribute of interest. If a gene happens to have an association with more than one attribute, then a regulation direction for each skin attribute is identified in the particular skin attribute data set; up-regulation of a gene might be favorable for one skin attribute and down-regulation of the gene favorable for a different attribute.

Referring to again to FIG. 1D of the simplified example, the table at 123, rows 213a-d show a simplified hypothetical example of how the datasets on genes of interest may reflect how the hypothetical genes are associated with a particular skin attribute and whether a pathway identified with the gene has an up or down regulation relative to a youthful direction for the particular skin attribute. Thus, for the “skin structure” attribute, genes b and f are shown as having an up-regulation association with a more youthful appearance as to that attribute. For “skin pigmentation”, genes b and c are shown as having an up-regulation association with more youthful appearance as to that attribute. For “skin hydration”, genes c and g are shown as having a down-regulation association, and gene e is shown as having an up-regulation association with more youthful appearance as to that attribute. For cell turnover, gene e is shown as having an up-regulation association and gene f is shown as having a down-regulation association with more youthful appearance as to that attribute.

As for the microarray data, Table 3, shows a data set comprising genes from Table 2 for which the literature shows a connection to the skin structure attribute, including a function with respect to skin structure which will determine the more youthful regulation direction. Once the more youthful regulation direction is specified for a gene, the data set of Table 3 can be used for intersection processing to find a skin attribute subset list for the gene assembly for the skin structure attribute.

TABLE 3

| Probe | Fold | | Gene | | | |
|---|---|---|---|---|---|---|
| Set ID | change | Direction | Symbol | Gene Title | Function | Reference |
| 213992_at | 4.8003573 | down | COL4A6 | tumor | Structural | The Journal of |
| | | | | necrosis | protein in | Cell Biology, Vol |
| | | | | factor, alpha- | skin | 130, 1219-1229, |
| | | | | induced | | Copyright © 1995 |
| | | | | protein 9 | | |
| 201286_at | 4.6295676 | down | SDC1 | tumor | cell- | Arch Dermatol |
| | | | | necrosis | extracellular | Res. 2008 |
| | | | | factor, alpha- | matrix | August; 300(7): |
| | | | | induced | interactions | 393-395 |
| | | | | protein 8 | | |
| 205900_at | 4.1411896 | down | KRT1 | tumor | Epidermal | Gene Expr |
| | | | | necrosis | structural | Patterns. 2005 |
| | | | | factor, alpha- | protein | August; 5(6): 801-8 |
| | | | | induced | | |
| | | | | protein 7 | | |
| 239272_at | 4.074428 | down | MMP28 | tumor | Destroys | Matrix Biol. 2009 |
| | | | | necrosis | Collagen | March; 28(2): 74-83. |
| | | | | factor, alpha- | | Epub 2009 Jan. 20 |
| | | | | induced | | |
| | | | | protein 6 | | |
| 209126_x_at | 3.952012 | down | KRT6B | tumor | Epidermal | Acta Derm |
| | | | | necrosis | structural | Venereol 2004; |
| | | | | factor, alpha- | protein | 84: 18-22 |
| | | | | induced | | |
| | | | | protein 5 | | |
| 204734_at | 3.736902 | down | KRT15 | tumor | Epidermal | J Invest |
| | | | | necrosis | structural | Dermatol. 1999 |
| | | | | factor, alpha- | protein | March; 112(3): |
| | | | | induced | | 362-9 |
| | | | | protein 4 | | |
| 204636_at | 3.736902 | down | COL17A1 | tumor | Structural | http://ghr.nlm.nih. |
| | | | | necrosis | protein in | gov/gene=col17a1 |
| | | | | factor, alpha- | the skin | |
| | | | | induced | | |
| | | | | protein 3 | | |
| 204135_at | 3.736902 | down | FILIP1L | tumor | Cytoskeleton | Nature Genetics |
| | | | | necrosis | remodelling | 33, 487-491 |
| | | | | factor, alpha- | | (2003) Published |
| | | | | induced | | online: March |
| | | | | protein 2 | | 2003; \| |
| | | | | | | doi: 10.1038/ng1119 |
| 200606_at | 3.736902 | down | DSP | tumor | Cellular | FASEB J. 1996 |
| | | | | necrosis | Adhesion | June; 10(8): 871-81. |
| | | | | factor, alpha- | protein | |
| | | | | induced | | |
| | | | | protein 1 | | |
| 208188_at | 3.736902 | down | KRT9 | tumor | structural | Mech Ageing |
| | | | | necrosis | protein | Dev. 2008 |
| | | | | factor, alpha- | | October; 129(10): |
| | | | | induced | | 563-71. Epub 2008 |
| | | | | protein 0 | | Jun. 3. |
| 201820_at | 3.736902 | down | KRT5 | tumor | Structural | Mech Ageing |
| | | | | necrosis | protein | Dev. 2008 |
| | | | | factor, alpha- | | October; 129(10): |
| | | | | induced | | 563-71. Epub 2008 |
| | | | | protein 1 | | Jun. 3. |
| 211922_s_at | 3.736902 | down | CAT | tumor | Protects | Journal of |
| | | | | necrosis | from UV | Investigative |
| | | | | factor, alpha- | damage | Dermatology |
| | | | | induced | | (2006) 126, 182- |
| | | | | protein 2 | | 190 |
| 202643_s_at | 6.915339 | up | TNFAIP3 | tumor | Negative | FEBS Lett. 2003 |
| | | | | necrosis | Feedback | Feb. 11; 536(1- |
| | | | | factor, alpha- | of NFKA | 3):135-40 |
| | | | | induced | (aging | |
| | | | | protein 3 | marker) | |
| 215078_at | 5.7122235 | up | SOD2 | superoxide | Protects | Journal of |
| | | | | dismutase 2, | from UV | Investigative |
| | | | | mitochondrial | damage | Dermatology |
| | | | | | | (1994) 102, 476- |
| | | | | | | 480 |
| 215910_s_at | 3.663163 | up | FNDC3A | fibronectin | Collagen | FEBS Lett. 1994 |
| | | | | type III | Assembly | Apr. 18; 343(1): |
| | | | | domain | | 47-50 |
| | | | | containing 3A | | |
| 204422_s_at | 3.455492 | up | FGF2 | fibroblast | Inhibits | Arteriosclerosis, |
| | | | | growth factor | matrix | Thrombosis, and |
| | | | | 2 (basic) | collagen | Vascular Biology. |
| | | | | | synthesis | 1993; 13: 680-686 |
| 216005_at | 2.7193272 | up | TNC | Tenascin | ECM | Dev Dyn. 2000 |
| | | | | | Remodelling | June; 218(2): 235- |
| | | | | | | 59. |
| 211958_at | 2.7118793 | up | IGFBP5 | insulin-like | Collagen | Arthritis Rheum. |
| | | | | growthfactor | Deposition | 2006 September; |
| | | | | binding | | 54(9): 3001-10 |
| | | | | protein 5 | | |
| 214702_at | 2.7099462 | up | FN1 | fibronectin 1 | Structural | Exp Cell Res. |
| | | | | | protein | 1990 November; |
| | | | | | | 191(1): 8-13 |
| 206026_s_at | 2.519742 | up | TNFAIP6 | tumor | Induces | BMC Immunol. |
| | | | | necrosis | MMP's | 2009 Mar. 19; |
| | | | | factor, alpha- | | 10:15 |
| | | | | induced | | |
| | | | | protein 6 | | |
| 204790_at | 2.1506758 | up | SMAD7 | SMAD | Negative | J Biol Chem. |
| | | | | family | regulator of | 2005 Mar. 4; |
| | | | | member 7 | collagen | 280(9): 8079-85. |
| | | | | | synthesis | Epub 2004 Dec. 3 |
| 213093_at | 2.125206 | up | PRKCA | protein kinase | Stimulates | Free Radic Biol |
| | | | | C, alpha | Collagenase | Med. 1999 October; |
| | | | | | | 27(7-8): 729-37 |
| 202724_s_at | 2.0982356 | up | FOXO1 | forkhead box | Induces | Am J Physiol Cell |
| | | | | O1 | Collagen | Physiol 292: |
| | | | | | Synthesis | C850-C856, 2007 |
| 201150_s_at | 2.0579739 | up | TIMP3 | TIMP | Inhibits | J Invest |
| | | | | metallopepti- | MMP's | Dermatol. 1998 |
| | | | | daseinhibitor 3 | | April;110(4): 416-21 |
| 203184_at | 2.0282688 | up | FBN2 | fibrillin 2 | Elastin | J Invest |
| | | | | | Fibre | Dermatol. 1996 |
| | | | | | Formation | May; 106(5): 1090-5 |
| 207720_at | 32.353355 | down | LOR | loricrin | Component | J Biol Chem. |
| | | | | | of Cornefied | 1992 Sep. 5; |
| | | | | | envelope | 267(25): 18060-6. |
| 1552319_a_at | 4.8667984 | down | KLK8 | kallikrein- | Break | Journal of |
| | | | | related | down of | Investigative |
| | | | | peptidase 8 | cellular | Dermatology |
| | | | | | adhesion | (2005) 125, |
| | | | | | proteins | 1182-1189 |
| 206033_s_at | 4.676169 | down | DSC3 | desmocollin 3 | Cellular | FASEB J. 1996 |
| | | | | | Adhesion | June; 10(8): 871-81. |
| | | | | | protein | |
| 209873_s_at | 4.6149898 | down | PKP3 | plakophilin 3 | Cellular | FASEB J. 1996 |
| | | | | | Adhesion | June; 10(8): 871-81. |
| | | | | | protein | |
| 209792_s_at | 4.4899607 | down | KLK10 | kallikrein- | | |
| related | Cellular | J Invest | | | | |
| peptidase | Adhesion | Dermatol | | | | |
| 10 | protein | 2003 121: | | | | |
| | | 542-549 | | | | |
| 214370_at | 2.9802194 | up | S100A8 | S100 calcium | Increases | Arch Dermatol |
| | | | | binding | with | Res. 2009 |
| | | | | protein A8 | photoaging- | August; 301(7): |
| | | | | (calgranulim | destroys | 523-9. Epub 2009 |
| | | | | A or cystatin | proteins | May 23. |
| | | | | A) | | |
| 205207_at | 3.1970735 | up | IL6 | Interleukin 6 | Stimulates | Journal of |
| | | | | | MMP's | Investigative |
| | | | | | | Dermatology |
| | | | | | | (2004) 123, |
| | | | | | | 1012-1019 |
| 204990_s_at | 4.5270753 | down | ITGB4 | integrin, beta 4 | Required | J Cell Biol. 1996 |
| | | | | | for cell to | Jul. 2; 134(2): |
| | | | | | cell | 559-572 |
| | | | | | adhesion | |
| 203535_at | 2.590224 | down | S100A9 | S100 calcium | Mediates | Biochem Biophys |
| | | | | binding | fibronectin | Res Commun. |
| | | | | protein A9 | adhesion | 2007 Mar. 2; |
| | | | | (calgranulim | | 354(1): 84-89 |
| | | | | B) | | |
| 1561042_at | 2.1507823 | up | ITGB1 | integrin, beta 1 | Marker for | Br J Dermatol. |
| | | | | | skin | 2003 April; |
| | | | | | structural | 148(4): 770-8 |
| | | | | | damage | |
| | | | | | caused by | |
| | | | | | UV | |
| 211981_at | 2.121121 | up | COL4A1 | Collagen | Skin | J Clin Invest. |
| | | | | Type 4 Alpha 1 | Structural | 1989 March; |
| | | | | | protein | 83(3): 791-795 |

As a gene associated with a skin aging attribute can be a part of a specific biochemical pathway involved in the physical appearance of skin aging that improves the skin attribute in a youthful direction or one that can be a part of a specific biochemical pathway that increases the appearance of skin aging (i.e., changes skin appearance in a non-youthful direction), the intersection processing requires additional logic to include in the gene assembly for a particular skin attribute only those genes that are regulated in a direction reflective of youthful skin appearance. Thus, the genes of Table 3 (see also, FIG. 7 at 756) are processed by a module identifying which genes were reported in the microarray a regulated in a direction (up or down regulated) that is the desired, more youthful direction. This criterion removes from the preliminary gene assembly of Table 3 any gene for which the microarray data shows that it up-regulates a pathway that decreases youthful appearance or that down-regulates a pathway that increases youthful appearance. This directional logic is step 134 of FIG. 1G.

Returning to the simplified, hypothetical example, FIGS. 1E and 1F, rows 215a-d show for each skin attribute the results or the answer to the question, whether or not the gene stays in the group. In the skin structure attribute example 126, gene b has a fold change greater than 2, and the up regulation of that gene from the (hypothetical) gene expression level is consistent with the data from the literature that indicates that up regulation of that gene provides more youthful skin structure. However, gene f has a fold change greater than 2, but the down regulation shown by the (hypothetical) gene expression level for gene f is not consistent with the data from the literature. According to the (hypothetical) literature, down regulation of gene f provides a less youthful skin appearance. Therefore, gene b is kept in the group, while gene f is dropped from the group at this time.

For the skin pigmentation attribute 128 of the simplified example, genes b and c have a fold change greater than 2, and the up regulation of genes b and c from the (hypothetical) gene expression level are consistent with the data from the literature indicating that up regulation of both genes b and c provide more youthful skin structure. Therefore, both genes b and c are kept in the group.

In the skin hydration attribute 130 of the simplified example, genes c and g have a fold change greater than 2, but the up regulation shown by the (hypothetical) gene expression level is not consistent with the data from the literature. According to the (hypothetical) literature, down regulation of genes c and g provide a more youthful skin appearance. Gene e has a fold change greater than 2, and the up regulation of that gene from the (hypothetical) gene expression level is consistent with the data from the (hypothetical) literature indicating that up regulation of that gene provides more youthful skin structure. Therefore, genes c and g are dropped from the group at this time, but gene e is kept in the group.

In the cell turnover attribute 132 of the simplified example, gene e has a fold change greater than 2, and the up regulation of that gene from the (hypothetical) gene expression level is consistent with the data from the (hypothetical) literature indicating that up regulation of that gene provides more youthful skin structure. Gene f has a fold change greater than 2, and the down regulation of that gene from the (hypothetical) gene expression level is consistent with the data from the literature that indicates that down regulation of that gene provides more youthful skin structure. Therefore, both genes e and f are kept in the group.

Table 4 shows the result when the processing logic of the simplified example is applied to the data from actual microarray testing of tissue exposed to salicin and when the skin attribute is “skin structure”, which is the focus of the data set in Table 3. The intersection processing module using the data of Table 3 identifies those genes that not only have an association with skin structure but also have been found in the test data to be up-regulating a pathway that provides more youthful skin structure or genes down-regulating a pathway that provides less youthful skin structure.

Table 4 (see also, FIG. 7 at 758) shows for the Affymetrix testing-derived data a group of genes exhibiting expression levels in a direction reflective of youthful skin appearance for the “skin structure” attribute. The up/down gene regulation shown by Affymetrix testing is thus for some genes consistent with scientific literature as to regulation of the gene in a more “youthful” direction. Table 4 is a subgroup of Table 3 and is a second subset of genes, further defined by applying the method steps sketched in FIGS. 1A-1G of the simplified example (through step 134) for the skin attribute “skin structure”.

Genes in Table 3 not chosen for Table 4 based on the logic requiring the alignment of the Affymetrix testing-derived data with the literature's position on regulation of the gene in a “youthful” direction may nonetheless be considered for additional research on “skin structure” but that must be based on secondary research factors. Table 4 shows only the genes that have the required alignment of “youthful” direction for “skin structure” in both the literature and the Affymetrix testing-derived data.

TABLE 4

| Probe | Fold | | Gene | | | |
|---|---|---|---|---|---|---|
| Set ID | change | Direction | Symbol | Gene Title | Function | Reference |
| 239272_at | 4.074428 | down | MMP28 | matrix | Destroys | Matrix Biol. 2009 |
| | | | | metallopepti- | Collagen | March; 28(2): 74-83. |
| | | | | dase 28 | | Epub 2009 Jan. 20 |
| 208188_at | 3.736902 | down | KRT9 | keratin 9 | structural | Mech Ageing |
| | | | | | protein | Dev. 2008 |
| | | | | | | October; 129(10): |
| | | | | | | 563-71. Epub 2008 |
| | | | | | | Jun. 3. |
| 201820_at | 3.736902 | down | KRT5 | keratin 5 | Structural | Mech Ageing |
| | | | | | protein | Dev. 2008 |
| | | | | | | October; 129(10): |
| | | | | | | 563-71. Epub 2008 |
| | | | | | | Jun. 3. |
| 202643_s_at | 6.915339 | up | TNFAIP3 | tumor | Negative | FEBS Lett. 2003 |
| | | | | necrosis | Feedback | Feb. 11; 536(1-3): |
| | | | | factor, alpha- | of NFKA | 135-40 |
| | | | | induced | (aging | |
| | | | | protein 3 | marker) | |
| 215078_at | 5.712224 | up | SOD2 | superoxide | Protects | Journal of |
| | | | | dismutase 2, | from UV | Investigative |
| | | | | mitochondrial | damage | Dermatology |
| | | | | | | (1994) 102, 476- |
| | | | | | | 480 |
| 215910_s_at | 3.663163 | up | FNDC3A | fibronectin | Collagen | FEBS Lett. 1994 |
| | | | | type III | Assembly | Apr. 18; 343(1): |
| | | | | domain | | 47-50 |
| | | | | containing 3A | | |
| 216005_at | 2.719327 | up | TNC | Tenascin | ECM | Dev Dyn. 2000 |
| | | | | | Remodelling | June; 218(2): 235- |
| | | | | | | 59. |
| 211958_at | 2.711879 | up | IGFBP5 | insulin-like | Collagen | Arthritis Rheum. |
| | | | | growth factor | Deposition | 2006 September; |
| | | | | binding | | 54(9): 3001-10 |
| | | | | protein 5 | | |
| 214702_at | 2.709946 | up | FN1 | fibronectin 1 | Structural | Exp Cell Res. |
| | | | | | protein | 1990 November; |
| | | | | | | 191(1): 8-13 |
| 202724_s_at | 2.098236 | up | FOXO1 | forkhead box | Induces | Am J Physiol Cell |
| | | | | O1 | Collagen | Physiol 292: |
| | | | | | Synthesis | C850-C856, 2007 |
| 201150_s_at | 2.057974 | up | TIMP3 | TIMP | Inhibits | J Invest |
| | | | | metallopepti- | MMP's | Dermatol. 1998 |
| | | | | dase inhibitor 3 | | April; 110(4): 416- |
| | | | | | | 21 |
| 203184_at | 2.028269 | up | FBN2 | fibrillin 2 | Elastin | J Invest Dermatol. |
| | | | | | Fibre | 1996 May; |
| | | | | | Formation | 106(5): 1090-5 |
| 1561042_at | 2.150782 | up | ITGB1 | integrin, beta 1 | Marker for | Br J Dermatol. |
| | | | | | skin | 2003 April; |
| | | | | | structural | 148(4): 770-8 |
| | | | | | damage | |
| | | | | | caused by | |
| | | | | | UV | |
| 211981_at | 2.121121 | up | COL4A1 | Collagen | Skin | J Clin Invest. |
| | | | | Type 4 Alpha 1 | Structural | 1989 March; |
| | | | | | protein | 83(3): 791-795 |

Youthful direction based on published literature.

As can be seen, with a data set like Table 3 derived for other skin attributes, a table like Table 4 can be derived for skin attributes other than “skin structure”.

### Confirmation

### Determination of RNA Quantification for Second Subset of Genes

A gene assembly developed to the status of Table 4 may be further confirmed and refined by a different methodology with a different gene analysis tool, in particular, by performing further skin model testing that takes advantage of the narrowing of focus to a list of genes as in Table 4.

Different methodologies include determining RNA types and levels by RNA quantification metrics including, for example, Northern blot technique, Ribonuclease Protection Assay (RPA) and Real Time Polymerase Chain Reaction (RT-PCR).

Northern blot is a well-known process for detecting and quantifying mRNA levels. The northern blotting technique is often used for comparison of gene expression patterns for different tissue types. In terms of skin genomics, it is less used than the modern techniques but can be used as confirmation step in understanding gene expression in the skin.

Northern blots start with the extraction and isolation of mRNA from the sample. RNA samples are then separated by gel electrophoresis. Once separated, the RNA is then transferred to a positively charged membrane, most often made of nylon. Once transferred to the membrane, RNA is then immobilized to the membrane through covalent linkage with the use of UV light or heat. Hybridization probes (fragment of DNA or RNA used to detect the presence of specific sequences) to be used for the experiments are labeled and placed on the membrane for hybridization. The membrane is then washed to ensure probe binding is strong as well to avoid background signals. The signals are then detected by X-ray film and can be quantified by densitometry. (Alberts, B., et al. Molecular Biology of the Cell, 5th ed. pp. 538-539, New York: Taylor & Francis Group (2008)).

Ribonuclease protection assay is a sensitive technique for detection, quantification and characterization of RNA. Isolated RNA is hybridized to a single stranded cDNA of the gene of interest. After annealing, the sample is subject to enzymatic digestion to remove all single stranded nucleic acids, leaving only double-stranded RNA. The double stranded nucleic acid fragments are then separated on high-resolution polyacylamide gels. Quantification is carried out similar to that of Northern Blot. The assay is much more sensitive than Northern blot, and can be used to quantify mRNAs that are expressed at low levels. (Applied Biosystems, Inc., The Basics: What is a Nuclease Protection Assay?©2010, last accessed May 18, 2010, from http://www.ambion.com/techlib/basics/npa/index.html).

Real Time Polymerase Chain Reaction is a laboratory technique used for DNA quantification, which measures the accumulation of DNA product after each round of PCR amplification. This laboratory technique is also known as quantitative real time polymerase chain reaction (RTQ-PCR/Q-PCR/qPCR) or kinetic polymerase chain reaction, which is used to amplify and simultaneously quantify a targeted DNA molecule. The technique enables both detection and quantification (as absolute number of copies or relative amount when normalized to DNA input or additional normalizing genes) of one or more specific sequences in a DNA sample.

The amplified DNA is detected as the reaction progresses in real time, as compared to standard PCR, where the product of the reaction is detected at its end. Two common methods for detection of products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary DNA target.

Reverse Transcription PCR (RT-PCT) is used for amplifying DNA from RNA. Reverse transcriptase reverse transcribes RNA into cDNA, which is then amplified by PCR. RT-PCR allows for a high sensitivity detection technique, where low copy number or less abundant RNA molecules can be detected. RT-PCR is widely used in expression profiling, to determine the expression of a gene or to identify the sequence of an RNA transcript, including transcription start and termination sites.

Real-time PCR may be combined with reverse transcription to quantify messenger RNA and Non-coding RNA in cells or tissues. Real-time reverse-transcription PCR is also known as qRT-PCR, RRT-PCR, or RT-rt PCR.

In some embodiments, Real Time Reverse Transcriptase Polymerase Chain Reaction (RT-rt-PCR) experiments on the second subset of genes are performed to confirm activity of the skin anti-aging agent acting on the gene.

In some embodiments, determining the levels of expression for the second subset of genes is done by using an RNA quantification metric. Selecting a further set of genes from a previous set of genes in a second sample of human skin tissue is based on measured levels of expression, which meet a criterion of biological relevance.

As seen in FIG. 1G of the simplified example, to perform confirmation of a skin attribute subset, or a preliminary functional youth gene assembly the next step is to expose a second sample of human or human equivalent skin tissue to the agent 136. Preferably this is done with the same skin model as used with the microarray technology for measuring global gene expression. The qualities of the agent (concentration, solvent, etc.) should normally be the same.

In some embodiments, the agent tested is salicin at a concentration of 0.5% salicin, available from Symrise Corporation (Teterborro, N.J.). The salicin is dissolved in water. Affymetrix microarray testing provides results for thousands of genes, whereas RT-rt-PCR testing provides results for a smaller gene group. For RT-rt-PCR testing, about 90 genes are tested at a time for this particular experimental design (other designs may test as many as 390 at a time on the equipment identified below). The experimenter may choose this number based on cost.

To start a process of confirmation using a second gene analysis tool that works with smaller arrays and a different, perhaps more sensitive measurement of regulation by the agent, expose a second sample of human skin tissue to the agent and select a set of candidate genes for confirmation. For example, the set of candidate genes may be the genes of a preliminary functional youth gene assembly of a particular skin attribute, supplemented with a few other genes that are of interest based on secondary research. More that one candidate group may of course be explored by confirmation. For example, a candidate group may be built around the preliminary functional youth gene assembly of each of the skin attributes discussed above: skin structure, skin pigmentation, skin hydration and cell turnover.

The subsets of genes related to a particular skin attribute are conveniently tested on one test card. Referring again to the process schematically shown for a simplified hypothetical gene set in FIGS. 1A-1G, the next step is to determine quantification of RNA and directions of regulation in the second exposed sample for each of the skin attribute subdivisions of the second subset of genes using a determination method different than for the first exposed sample 138, for example, using an RT-rt-PCR method, for each attribute. For the simplified example set of genes of FIG. 1G, we may assume all genes from all four attributes are tested on one chip.

To implement this step, RT-rt-PCR is conducted for specific genes known to be involved in skin aging. In one embodiment Custom TaqMan® Low Density Arrays (TLDA's) were configured using Applied Biosystems validated gene expression assays. The validated gene expression assays contain a TaqMan® fluorescent probe and primers for each target gene. Genes for the TLDA cards are selected based on either, published literature describing the genes functional role in skin cell biology and aging and/or the previous Affymetrix testing results. (See Tables 1-4). Five endogenous control genes may be included on each card. Thus, when a particular gene assembly is tested with RT-rt-PCR, the data resulting may cover more than just the set of genes as in Table 3.

cDNA is synthesized from an aliquot of total RNA using the High Capacity cDNA reverse transcription kit from Applied Biosystems (Foster City, Calif.) according to the manufacturer's suggested protocols. (High-Capacity cDNA Reverse Transcription Kits for 200 and 1000 Reactions Protocol (October, 2006)). cDNA was mixed with TaqMan® Universal Master Mix without UNG and loaded into the wells of the TLDA cards. The cards are run using an Applied Biosystems 7900HT instrument according to the manufacturer's cycling parameters.

As with the microassay, the analysis is done with a skin model exposed to the agent and a reference that is not exposed to the agent. The skin model not exposed to the agent may be used for calibration. The skin model may be human equivalent skin tissue. The target genes get normalized to a stable endogenous control (genes that are invariants in all cell types such as β₃-actin). This normalization is to account for variations that may occur during sample loading. The unexposed information gathered is used for comparison against the tested sample.

The formula for ΔC_{T} (delta cycle threshold) is C_{T} (target) minus C_{T} (endogenous control gene). (ΔC_{T}=C_{T} (target)−C_{T} (endogenous control gene)). The test system, data processing system 710 stores the data then uses process application modules 720 to take the C_{T} values for both the exposed and unexposed and get a ΔΔC_{T} value, which is reported for that specific gene in a log ratio scale. Once this data is collected and stored, such as in database 730 (see FIG. 7), it is analyzed by conducting bioinformatics statistical analysis on data 140. In one embodiment, data analysis is carried out according to the RQ analysis method using RQ Manager and StatMiner (v3.1) software programs.

At least part of the comparing of the data is performed by a computer system, such as the data processing system 710 (see FIG. 7) for performing data access and storage and various computations specified by software (process application modules 720) corresponding to the functions occurring at various described steps of this method. Additional process application modules 722 perform a parametric t-test with a Benjamini and Hochberg false discovery rate correction is performed to identify genes with a statistically significant p value equal to or less than 0.05. The up or down regulation of the gene is also identifiable from the RT-rt-PCR analysis. Results of the change in threshold cycle (ΔC_{T}) values between the exposed and unexposed human skin tissues are calculated by a software program. The selected genes will have a cycle threshold of biological relevance. For these experiments, the selected genes have a cycle threshold of less than about <35, which is a value of biological relevance. In every cycle of PCR (C_{T} value) the amount of DNA is approximately duplicated, thus, the C_{T} is in the logarithmic scale and inversely proportional to the quantity of DNA/RNA. Therefore, high C_{T} values represent low expression while highly expressed genes have low C_{T} values. Comparing the normalized expression of the two conditions it is possible to calculate the fold change of the expression of the gene, ΔΔC_{T} value. The fold change is the expression ratio: if the fold change is positive it means that the gene is up-regulated; if the fold change is negative it means it is down-regulated. This is represented in a log scale.

The C_{T} or cycle threshold is defined as the number of cycles required for the fluorescent signal to cross the threshold. C_{T} levels are inversely proportional to the amount of target DNA in the same. Standard real time reactions undergo 40 cycles of amplification. C_{T}<20 indicate strong positive reactions and an abundance of the targeted DNA. C_{T} values of 30-37 are positive reactions indicative of moderate amounts of target DNA. C_{T} values of 38-40 are weak reactions indicative of minimal amounts of target DNA. The C_{T} values are an criterion of biological relevance. The experimenter optionally chooses a criterion based on biological relevance for gene expression in aging skin.

Table 5 (see also, FIG. 7 at 762) shows a testing-derived example of quantitative measurements of gene expression and the direction of regulation for genes associated with the skin attribute “skin structure”, derived from RT-rt-PCR analysis. As can be seen, the number of genes in Table 5 exceeds that in Table 4. This reflects that, in some cases, it may be useful to add to a test card a gene that did not show a sufficient fold change in the microarray data but is identified with a strong anti-aging effect or is otherwise known to be of possible interest for this skin attribute. It also may be useful to add to a test card a gene that showed a sufficient fold change in microarray data but has not yet been identified with a strong anti-aging effect. Optionally, the experimenter could add to a test card a gene that has a fold change greater than the selected fold change criterion (e.g., 2), but the regulation direction shown by the first gene expression level testing is not consistent with the data from the literature. This can provide a useful second look at a gene.

TABLE 5

| Gene | Gene | | | | |
|---|---|---|---|---|---|
| Symbol | Function | Direction | p-value | Log10RG | Reference |
| COL4A6 | Structural | no data | | | The Journal of Cell |
| | protein in | | | | Biology, Vol 130, |
| | skin | | | | 1219-1229, Copyright © |
| | | | | | 1995 |
| SDC1 | Cell- | down | 0.002811879 | −0.396098529 | Arch Dermatol Res. |
| | extracellular | | | | 2008 August; 300(7): |
| | matrix | | | | 393-395 |
| | interactions | | | | |
| KRT1 | Epidermal | down | 0.001131397 | −1.111598451 | Gene Expr Patterns. |
| | structural | | | | 2005 Aug; 5(6): 801-8 |
| | protein | | | | |
| MMP28 | Destroys | no data | | | Matrix Biol. 2009 |
| | Collagen | | | | Mar; 28(2): 74-83. |
| | | | | | Epub 2009 Jan 20 |
| KRT6B | Epidermal | no data | | | Acta Derm Venereol |
| | structural | | | | 2004; 84: 18-22 |
| | protein | | | | |
| KRT15 | Epidermal | no data | | | J Invest Dermatol. |
| | structural | | | | 1999 Mar; 112(3): 362-9 |
| | protein | | | | |
| COL17A1 | Structural | no data | | | http://ghr.nlm.nih.gov/gene=col17a1 |
| | protein in | | | | |
| | the skin | | | | |
| FILIP1L | Cytoskeleton | no data | | | Nature Genetics 33, |
| | remodelling | | | | 487-491 (2003) |
| | | | | | Published |
| | | | | | online: March 2003;\| |
| | | | | | doi:10.1038/ng1119 |
| DSP | Cellular | down | 0.017260177 | −0.222288576 | FASEB J. 1996 |
| | Adhesion | | | | Jun; 10(8): 871-81. |
| | protein | | | | |
| KRT9 | Structural | no data | | | Mech Ageing Dev. |
| | protein | | | | 2008 |
| | | | | | Oct; 129(10): 563-71. |
| | | | | | Epub 2008 Jun 3. |
| KRT5 | Structural | down | 0.001982117 | −0.444955798 | Mech Ageing Dev. |
| | protein | | | | 2008 |
| | | | | | Oct; 129(10): 563-71. |
| | | | | | Epub 2008 Jun 3. |
| CAT | Protects | down | 0.000701114 | −0.476760771 | Journal of |
| | from UV | | | | Investigative |
| | damage | | | | Dermatology (2006) |
| | | | | | 126, 182-190 |
| TNFAIP3 | Negative | no data | | | FEBS Lett. 2003 Feb |
| | Feedback of | | | | 11; 536(1-3): 135-40 |
| | NFKA | | | | |
| | (aging | | | | |
| | marker) | | | | |
| SOD2 | Protects | up | 0.027437424 | 0.302541956 | Journal of |
| | from UV | | | | Investigative |
| | damage | | | | Dermatology (1994) |
| | | | | | 102, 476-480 |
| FNDC3A | Collagen | no data | | | FEBS Lett. 1994 Apr |
| | Assembly | | | | 18; 343(1): 47-50 |
| FGF2 | Inhibits | no data | | | Arteriosclerosis, |
| | matrix | | | | Thrombosis, and |
| | collagen | | | | Vascular Biology. |
| | synthesis | | | | 1993; 13: 680-686 |
| TNC | ECM | up | 0.001112139 | 0.142852906 | Dev Dyn. 2000 |
| | Remodelling | | | | Jun; 218(2): 235-59. |
| IGFBP5 | Collagen | no data | | | Arthritis Rheum. 2006 |
| | Deposition | | | | Sep; 54(9): 3001-10 |
| FN1 | Structural | up | 0.027437424 | 0.241122656 | Exp Cell Res. 1990 |
| | protein | | | | Nov; 191(1): 8-13 |
| TNFAIP6 | Induces | no data | | | BMC Immunol. 2009 |
| | MMP's | | | | Mar 19; 10: 15 |
| SMAD7 | Negative | up | 0.001982117 | 0.232529868 | J Biol Chem. 2005 |
| | regulator of | | | | Mar 4; 280(9): 8079-85. |
| | collagen | | | | Epub 2004 Dec 3 |
| | synthesis | | | | |
| PRKCA | Stimulates | up | 0.01950064 | 0.274898033 | Free Radic Biol Med. |
| | Collagenase | | | | 1999 Oct; 27(7-8): 729-37 |
| FOXO1 | Induces | up | 0.036848433 | 0.238284357 | Am J Physiol Cell |
| | Collagen | | | | Physiol 292: C850-C856, |
| | Synthesis | | | | 2007 |
| TIMP3 | Inhibits | no data | | | J Invest Dermatol. |
| | MMP's | | | | 1998 Apr; 110(4): 416-21 |
| FBN2 | Elastin Fibre | no data | | | J Invest Dermatol. |
| | Formation | | | | 1996 |
| | | | | | May; 106(5): 1090-5 |
| LOR | Component | down | 0.000701114 | −1.920784953 | J Biol Chem. 1992 |
| | of Cornefied | | | | Sep 5; 267(25): 18060-6. |
| | envelope | | | | |
| KLK8 | Break down | no data | | | Journal of |
| | of cellular | | | | Investigative |
| | adhesion | | | | Dermatology (2005) |
| | proteins | | | | 125, 1182-1189 |
| DSC3 | Cellular | down | 0.005260102 | −0.293838188 | FASEB J. 1996 |
| | Adhesion | | | | Jun; 10(8): 871-81. |
| | protein | | | | |
| PKP3 | Cellular | no data | | | FASEB J. 1996 |
| | Adhesion | | | | Jun; 10(8): 871-81. |
| | protein | | | | |
| KLK10 | Cellular | no data | | | J Invest Dermatol |
| | Adhesion | | | | 2003 121: 542-549 |
| | protein | | | | |
| S100A8 | Increases | down | 0.001131397 | −0.63072926 | Arch Dermatol Res. |
| | with | | | | 2009 Aug; 301(7): 523-9. |
| | photoaging- | | | | Epub 2009 May 23. |
| | destroys | | | | |
| | proteins | | | | |
| IL6 | Stimulates | up | 0.001131397 | 0.932203049 | Journal of |
| | MMP's | | | | Investigative |
| | | | | | Dermatology (2004) |
| | | | | | 123, 1012-1019 |
| ITGB4 | Required for | down | 0.001131397 | −0.828309922 | J Cell Biol. 1996 July |
| | cell to cell | | | | 2; 134(2): 559-572 |
| | adhesion | | | | |
| S100A9 | Mediates | down | 0.001131397 | −0.651297398 | Biochem Biophys Res |
| | fibronectin | | | | Commun. 2007 March |
| | adhesion | | | | 2; 354(1): 84-89 |
| ITGB1 | Marker for | up | 0.01401738 | 9.36E−02 | Br J Dermatol. 2003 |
| | skin | | | | Apr; 148(4): 770-8 |
| | structural | | | | |
| | damage | | | | |
| | caused by | | | | |
| | UV | | | | |
| COL4A1 | Skin | up | 0.016310135 | 0.208638283 | J Clin Invest. 1989 |
| | Structural | | | | March; 83(3): 791-795 |
| | protein | | | | |

Taqman TLDA Analysis
Relative Quantition - T-test with Benjamini and Hochberg correction
HPRT as Endogenous Control Gene
Untreated Control as Calibrator
p value <0.05
Salicin 0.5% vs. Untreated Control

### Selecting Third Subset of Genes

### Functional Youth Gene Assembly

Results from the RT-rt-PCR experiments may or may not confirm that the candidate genes subject to confirmation testing are regulated in the direction reflective of youthful appearing skin based on the published scientific literature.

The RT-rt-PCR data thus provide an additional basis for refining a gene functional youth gene assembly that is derived from steps 102-134 of FIGS. 1A-1G. If a gene as tested for a gene assembly is not confirmed as having the same direction that led to its selection from steps 102-134, it may now be removed as a member of the gene assembly. Because the RT-rt-PCR data provide a new reading on the level of expression, a further expression level threshold can applied as a criterion for membership in an assembly. Genes are selected from those with expression levels determined based on a relative quantification analysis.

Referring now to the simplified example shown in FIG. 1G, the next step is to select from the previously subdivided groups from the second subset of genes (the functional youth gene assemblies), a third subset of genes also subdivided by skin attribute regulated in a direction reflective of youthful skin appearance, with an appropriately selected biological relevance level, e.g., a cycle criterion level (for example, the criterion could be selected at 35 cycles or less). (For PCR data, the lower the cycle number, the stronger the gene expression.)

In FIG. 1G is a table showing a set of hypothetical results for the simplified sample set of genes a-h; in particular, it shows in row 216 ΔC_{T} values for genes b, c, e, f, g and h. (In the simplified example, we have assumed that genes a and d have no values from RT-rt-PCR, as those genes were previously filtered out of the second subset.)

As noted, some genes dropped at earlier stages of the process outlined in FIGS. 1G-1K may be reconsidered by adding them back into the RT-rt-PCR testing. In the simplified example, gene h shows a sufficient fold change in the hypothetical microarray data but has not yet been identified with a strong anti-aging effect. In the simplified example, gene g has a fold change greater than 2, but the regulation direction shown by the first gene expression level testing was not consistent with the data from the literature. Assuming gene g and gene h were reconsidered during the RT-rt-PCR testing, this results in hypothetical data in row 216-218.

Referring to FIGS. 1G-1K describing the simplified example, the confirmation of direction of regulation criterion and the ΔC_{T} criterion are applied to the hypothetical data in row 216-218 to confirm/reject the results shown in the selection of genes in the table at 126 (corresponding to Table 4 derived from Affymetrix microarray testing data) to arrive at the following results for the simplified hypothetical:

Functional youth gene assembly for skin structure 144: Gene b is confirmed because it meets the ΔC_{T} criterion and matches the direction of expression associated with more youthful skin structure per the literature data set on skin structure.

Functional youth gene assembly for skin pigmentation 146: Gene b is confirmed because it meets the ΔC_{T} criterion and matches the direction of expression associated with more youthful skin pigmentation per the literature data set on skin pigmentation. Gene c is not confirmed because it does not meet the ΔC_{T} criterion.

Functional youth gene assembly for skin hydration 148: Gene e is not confirmed because it does not meet the ΔC_{T} criterion. Gene g is added because it meets the ΔC_{T} criterion and matches the direction of expression associated with more youthful skin hydration per the literature data set on skin hydration. This is contrary to the microarray data.

Functional youth gene assembly for cell turnover 150: Gene e is not confirmed because it does not meet the ΔC_{T} criterion; however, if the criterion had been set at 38, it would have met that level. Gene f is confirmed because it meets the ΔC_{T} criterion and matches the direction of expression associated with more youthful skin cell turnover per the literature data set on skin cell turnover.

Genes not chosen for the third subset of genes (gene h) may be considered for additional research based on secondary research factors 152.

Turning to the testing-derived data example (actual Affymetrix data for salicin exposed tissue and data from RT-rt-PCR testing), Table 6 (see also, FIG. 7 at 770) illustrates one embodiment of a functional youth gene assembly selected for the skin attribute “skin structure.” Table 6 thus represents a comparative analysis of the results of Table 5 and Table 4, by confirmation of direction of regulation criterion and the ΔC_{T} criterion applied to the hypothetical data. Thus, a gene appears in Table 6, only if (a) it appears in Table 4 and the data of Table 5 confirm that it have a strong-enough level of expression based on the ΔC_{T} criterion and that the data of Table 5 do not show a regulation direction that contradicts the more youthful direction that was the basis for inclusion in Table 4, or (b) although not in Table 4, it was added to the candidate list for secondary reasons and the result of the RT-rt-PCR testing provides strong evidence that it should be added, including a regulation direction that is consistent the more youthful direction and a strong ΔC_{T} value, exceeding the ΔC_{T} criterion.

TABLE 6

| Gene | Gene | | | | |
|---|---|---|---|---|---|
| Symbol | Description | Direction | p-value | Log10RG | Reference |
| FOXO1 | forkhead | up | 0.036848433 | 0.238284357 | Am J Physiol Cell |
| | box O1 | | | | Physiol 292: |
| | | | | | C850-C856, 2007 |
| ITGB1 | integrin, beta 1 | up | 0.01401738 | 0.093562957 | Br J Dermatol. |
| | | | | | 2003 |
| | | | | | Apr; 148(4): 770-8 |
| FN1 | fibronectin 1 | up | 0.027437424 | 0.241122656 | Exp Cell Res. |
| | | | | | 1990 |
| | | | | | Nov; 191(1): 8-13 |
| SOD2 | superoxide | up | 0.027437424 | 0.302541956 | Journal of |
| | dismutase 2, | | | | Investigative |
| | mitochondrial | | | | Dermatology |
| | | | | | (1994) 102, 476-480 |
| COL4A1 | Collagen Type | up | 0.016310135 | 0.208638283 | J Clin Invest. 1989 |
| | 4 Alpha 1 | | | | March; 83(3): |
| | | | | | 791-795 |
| S100A8 | S100 calcium | down | 0.001131397 | −0.63072926 | Arch Dermatol |
| | binding | | | | Res. 2009 |
| | protein A8 | | | | Aug; 301(7): 523-9. |
| | (calgranulim | | | | Epub 2009 May |
| | A or cystatin | | | | 23. |
| | A) | | | | |

The RT-rt-PCR methodology permits not only a second reading on the activity of genes that have met the criteria for a gene assembly in steps 102-134, it provides an opportunity to test a gene that has not met these criteria, but might meet certain secondary research factors that suggest it may be of interest for a particular gene assembly. Secondary research factors may suggest further testing of genes that have a high fold change without any literature support for their relevance in skin tissue, genes associated with anti-aging mechanisms of action but not thought of as skin-related, or genes that are strongly supported by literature as having an effect on skin aging, but not achieving a significant fold change cutoff in testing as described in steps 102-134 of the simplified example. Genes with significant fold change values that are not identified in the literature as having a favorable impact on a skin attribute may be considered for additional research. In the simplified sample set of genes, gene h not chosen for the third subset of genes is considered for additional research based on secondary research factors 152. See FIGS. 1G-1K. For example, genes such as Klotho (KL) which have published anti-aging benefits in mice may be of interest. Genes such as these may provide insights on identifying and discovering new novel pathways in the skin aging process.

A favorable impact on a skin attribute is a biologically relevant change that establishes a state of an attribute more similar to the non-aged state of the attribute. For example, a favorable impact is recognized when an agent that is applied to the skin results in a more youthful appearance. The present system and method may be used to extend more efficiently the search for agents that cause a favorable impact. For example if the potential of a possible useful agent needs basic exploration, it can be run through the entire method of FIGS. 1A-1K to see how the resulting functional youth gene assembly compares to that of other agents. Using more than one agent to execute the method decreases possible agent bias resulting from using a singular agent to determine a functional youth gene assembly.

For greater efficiency, once researchers have confidence in one or more functional youth gene assemblies, testing of an agent my be done by omitting full genome microarray studies and using only more limited studies for the genes included in one of more of the functional youth gene assemblies.

One outcome of using the entire method described to screen agents that trigger a relevant change in gene expression is to identify genes for further study, even if they are not yet reported in the literature. These may be genes that are not currently associated with any biochemical pathway associated with skin, but may be in the future, as there are advances in technology and further research studies. These genes may optionally be added to an appropriate functional youth gene assembly.

Genes with non-skin related anti-aging mechanisms of action may be subjected to further testing to determine the gene's effect, if any, on skin aging. For example, scientific literature suggests that the β-klotho gene appears to be involved in the aging process. See, U.S. Pat. No. 7,537,903.

Genes that are supported by literature as having an effect on skin aging, but not achieving the biologically relevant fold change cutoff in micro-array testing may be subjected to another round of micro-array testing with different concentrations of the agent or with different anti-aging agent(s).

In some embodiments, the functional youth gene assemblies, the groups of genes identified for a skin attribute in genome-wide microarray tests, are optionally refined based on the results from the RT-rt-PCR experiments. If the literature discloses that a gene with “up” regulation results in better skin structure, and the RT-rt-PCR data shows “down” regulation for this gene, the gene may be set aside for possible further research at a later date. Alternately, if the literature discloses a gene with “up” regulation results in better skin structure, and the RT-rt-PCR data shows “up” regulation for this gene, then the gene may be added to the functional youth gene assembly.

After application of secondary research factors, more genes are optionally added into one or more functional youth gene assembly 154.

A method that utilizes the results of the groups of genes, the functional youth gene assemblies, may be used to guide further research on aging of the skin.

### Data Processing System Implementation

### Data Sets

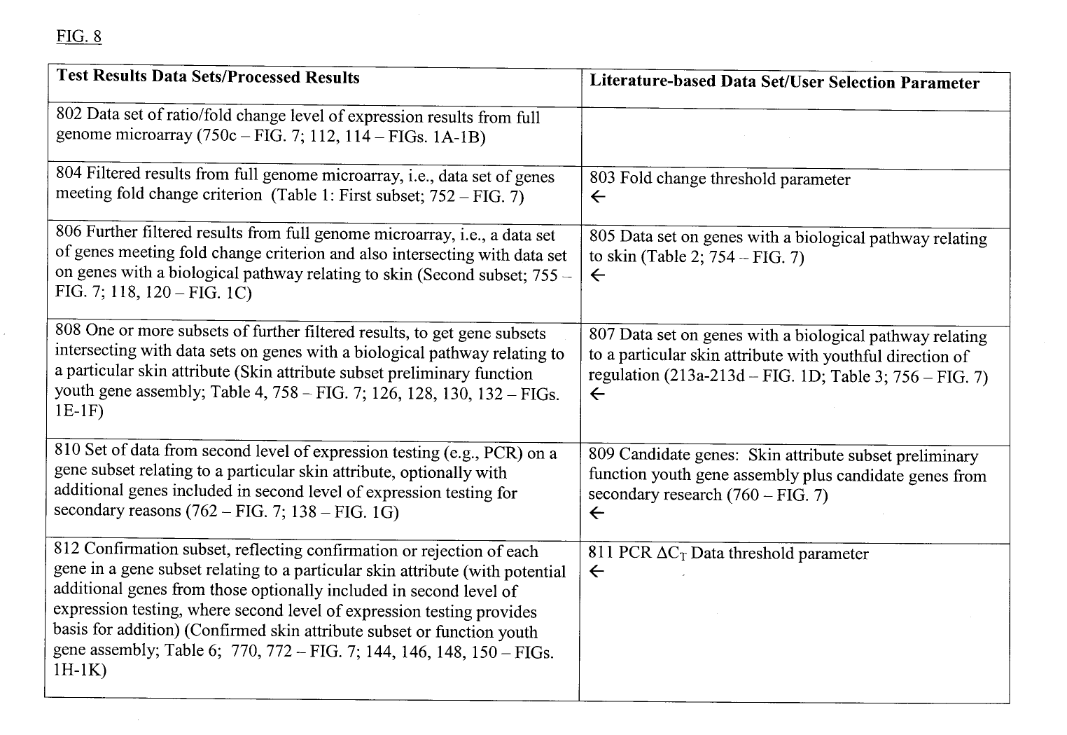

As discussed above, FIG. 7 shows a schematic diagram of a system for carrying out the method disclosed, including data set developed and used as the method proceeds and the test equipment used to develop various sets of data from the tissue samples of the skin models. This system can now be further explained with reference to FIG. 8, that shows the data sets used in the system and method and how they are transformed into the functional youth gene assemblies. The system 700 broadly comprises a data processing system 710 with a CPU and memory, in which there is an operating system 712, and the test equipment that develops data, including a full genome microarray device 780 and a PCR testing device 790. The test equipment is supplied, and the materials to be tested, prepared per the supplier's instructions, include the samples of agent exposed skin model 782 and the non-exposed skin model 784.

The data processing system 710 includes a database 730 that receives and stores the data used in the process described above. The process applications modules 720 execute, including statistics modules 722 and applications using user selected process parameters 724 to perform the flowchart (see FIGS. 1A-1K) processes. The process applications modules 720 access the database 730 using suitable database management protocols 732.

The database stores the various data sets involved including the full genome data sets 750a, 750b developed at the full genome microarray device 780, the calculated ratio data 750c and the fold criterion result data set 752, developed by application of the fold change criterion. The database 730 also stores the pathway criterion data set 754 that identifies the association between a gene and one or more biological pathways and the intersection dataset 755 resulting from the intersection of the fold criterion result data set 752 and the pathway criterion data set 754. The database 730 further stores the skin attribute focus data sets 756 that defines the association between a particular skin attribute that is a under study and genes that are associated with that attribute in the literature. After the intersection analysis of a particular skin attribute focus data set 756 with the skin attribute/regulation direction data and the fold criterion result data 752 (which includes determining alignment of the more youthful regulation direction for the particular biochemical pathways), the developed skin attribute subset 758, representing a preliminary functional youth gene assembly for a particular skin attribute is stored in database 730.

The data processing system's database 730 also receives and holds data relevant to the PCR testing and results of the confirmation analysis for the preliminary functional youth gene assembly. This includes storing the PCR candidate data 760, i.e., the listing of the genes based on the preliminary functional youth gene assembly as supplemented with genes of secondary interest that will be subject to PCR testing under the cycle level criterion or other parameters used in the analysis of the PCR testing data. After the PCR tests have been run, the database 730 receives the PCR cycle data 762 including the associated up/down regulation direction observed from testing. From the PCR cycle data set 762, the processing applications 724 derives the Final Attribute Data 770, 772 for one or more skin attributes.

As noted, stored in memory are the process application modules 720. These are software generally in two categories. A first category is the conventional statistical analysis programs 722, such as GeneSpring GX software (version 10) or other commercial software to perform a parametric t-test with a Benjamini and Hochberg false discovery rate correction. The StatMiner (Version 3) software may be used for analysis of the PCR data. The second category is the flowchart process applications that implement the analysis and steps discussed above and shown in FIGS. 1A-1K. The process application modules 720 that are custom-developed may be written in any suitable language, such as C++, or other languages suitable for the analysis and steps discussed above and shown in FIGS. 1A-1K.

FIG. 8 shows in simplified form the progression of data sets as the system proceeds to execute the method. In particular, FIG. 8 traces the test results data sets 802, 804, 806, 810, 812 and shows the effect of the literature-based data sets and user selected parameters. FIG. 8 shows how the data sets stemming from the full genome microarray data 802, 804, 806, 808 and data set 810, stemming from the PCR instrument, are modified to obtain a confirmed skin attribute subset 812, that is a function youth gene assembly by the data processing steps outlined in the simplified hypothetical example of FIGS. 1A-1K. FIG. 8 also references Tables 1-6 that are based on actual whole genome microarray and PCR testing.

### Screening Method

While a primary use of the present methodology is to develop the functional youth gene assemblies that provide a focus for further gene-level research on skin attributes, the methodology may also be used to screen agents for effectiveness to reduce skin aging. An agent may be chosen for testing to assess the efficacy of the particular agent and to explore the genetic pathway focus of its action. Known anti-aging agents have shown significantly different levels of gene expression in genes associated with a plurality of biochemical pathways of the skin. A screening method of this type could significantly lessen the number of costly and lengthy in vivo testing procedures done on many anti-aging product candidates. For example, many consumer studies on facial anti-aging products run for at least 12 weeks. Provided a reliable functional youth gene assembly is identified, testing the effects of an agent on the biochemical pathways associated with particular genes provides a focused way to develop data on the action of the an anti-aging candidate on a much shorter time frame and provides quantitative data for comparison to other agents.

A screening approach may be used to assess the likelihood of another agent working well in an anti-aging skin care product. A new agent triggering levels of gene expression to a functional youth gene assembly similar to or superior to a known skin anti-aging agent may be considered for further study, while a new agent that does not trigger similar levels of gene expression in those genes in a functional youth gene assembly may not be considered for further research investment.

The screening method may also be used for improving the effective properties of existing anti-aging skin care products, selecting new anti-aging ingredients for products, and selecting blends of anti-aging ingredients for products. From an understanding of which genes and which biochemical pathways have skin anti-aging effects, the properties of an agent as a promoter of a biochemical pathway associated with more youthful appearance or an inhibitor of a biochemical pathway associated with less youthful appearance may be improved. Using the screening method on many possible agent candidates instead of time-consuming clinical testing on fewer agent candidates is both a time-efficient and cost-effective way of performing research and development. The method helps to provide consumers with anti-aging products based on the most recent scientific research.

Other agents and agent blends including, for example, arNOX inhibitory agents derived from plant extracts may be tested. The plant for extract is optionally selected from broccoli, shitake, coleus, rosemary, lotus, artichoke, sea rose tangerine, Oenothera biennis, astaxanthin, red orange, Schisandra chinensis, Lonicera, Fagopyrum, carrot, Narcissus tazetta or olive. The arNOX inhibitory agents optionally include salicylates, for example, salicin, salicylic acid, salicyl hydroxamate, derivatives or combinations thereof.

While one embodiment of the present methodology is to develop the functional youth gene assemblies that provide a focus for further gene-level research on skin attributes, when the methodology is used to screen agents for effectiveness to reduce skin aging, it can assist in the formulation of a composition to reduce skin aging. Once an agent has been identified in testing to have efficacy as a promoter of a biochemical pathway associated with more youthful appearance or an inhibitor of a biochemical pathway associated with less youthful appearance for at least one skin attribute, that agent can be a candidate for an active ingredient in a composition to reduce skin aging. Provided a reliable functional youth gene assembly has been identified and efficacy of an agent on the biochemical pathways associated with particular genes in that assembly has been found, the composition can be targeted specifically to improvement of the skin attribute associated with that functional youth gene assembly. A composition can be formulated that addresses multiple skin attributes, once effective agents for the multiple skin attributes are found by the process and system disclosed herein. A composition can also include a pharmaceutically acceptable carrier. A pharmaceutical acceptable carrier refers to a carrier medium that does not interfere with the effectiveness of the biological activity of the active ingredient, is chemically inert, and is not toxic to the patient to whom it is administered. The type of the carrier may include powders, emollients, lotions, creams, liquids and the like.

Thus, the understanding of which genes and which biochemical pathways have skin anti-aging effects and the properties of an agent as a promoter of a biochemical pathway associated with more youthful appearance or an inhibitor of a biochemical pathway associated with less youthful appearance is improved by the methods discussed herein, this understanding can be translated into compositions that are directed to one or more skin attributes associated with a functional youth gene assembly. It is expected that agents showing an anti-aging efficacy will be derived from broccoli, shitake, coleus, rosemary, lotus, artichoke, sea rose tangerine, Oenothera biennis, astaxanthin, red orange, Schisandra chinensis, Lonicera, Fagopyrum, carrot, Narcissus tazetta or olive. They also may be derived from arNOX inhibitory agents that include salicylates, for example, salicin, salicylic acid, salicyl hydroxamate, derivatives or combinations thereof. These agents and their derivatives may then be deployed in skin anti-aging formulations with a sound basis in research at the genetic level.

### In Vivo Testing

### Evaluation of Composition Including Anti-Aging Agent

For further confirmation of the effects of an agent that is viewed as regulating in a youthful direction the pathways of a functional youth gene assembly for a skin attribute, consumer clinical studies may be conducted with a skin care product including the anti-aging agent tested with in vitro methods. Clinical studies with trained observation and measurement of skin parameters confirm changes in particular skin aging attributes as regulated by a particular functional youth gene assembly.

After a group of genes are selected as a functional youth gene assembly, in vitro studies including the assay methods discussed above, are used to screen agent candidates and limit the amount of in vivo studies used in product development.

A skin care product, ageLOC® Future Serum, including 0.5% salicin is the finished formulation used for evaluation in clinical testing. The ageLOC® Future Serum is commercially available from Nu Skin Enterprises, Inc. (Provo, Utah).

Twenty-nine out of thirty subjects completed the clinical study. Table 7 summarized the demographics of the study participants.

TABLE 7
Study Participant's demographic summary.

| Demographic | | All Subjects |
|---|---|---|
| Summary | | (n = 30) |
| Age (Years) | Mean Age ± Standard | 56.06 ± 7.76 |
| | Deviation | |
| | Minimum Age | 40.44 |
| | Maximum Age | 70.28 |
| Ethnicity | Caucasian | 50% |
| | Asian | 50% |
| Fitzpatrick | I | 5.10% |
| Skin Type | II | 42.40% |
| | III | 47.50% |
| Demographic | | All Subjects |
| Summary | | (n = 30) |

The Fitzpatrick skin classification is based on the skin's unprotected response to the first 30 to 45 minutes of sun exposure after a winter season without sun exposure:

I—Always burns easily; never tans; II—Always burns easily; tans minimally; III—Burns moderately; tans gradually; IV—Burns minimally; always tans well; V—rarely burns; tans profusely; VI—Never burns; deeply pigmented.

Clinical Procedures

At baseline (Visit 1), each prospective subject completed an Eligibility and Health Questionnaire, and read and signed a Confidentially Agreement, a Photography Release Form and an Informed Consent Agreement. Each subject was explained the type of study, the detailed procedures and materials to be tested, along with any known adverse reactions that may result from participation. Subjects arrived at the clinic having refrained from applying any moisturizer to the face at least 3 to 5 days prior to visit and having cleansed the face to remove all makeup at least 30 minutes prior to visit. Subjects were consequently not allowed to use any other topical cosmeceuticals, topical retinoid, or moisturizers during the 12 week duration of the study.

Subjects used the study product on their face twice daily for 12 weeks. Ordinal grading on a 9-point scale (0=none, 1-3=mild, 4-6=moderate, 7-9=severe) of facial fine lines, mottled pigmentation, uneven skin tone, tactile roughness, global firmness appearance, jaw-line contour, radiance and overall appearance was performed by investigator at baseline, week 1, week 4, week 8 and week 12.

Digital high-resolution photography was performed on the front, right and left sides of the face. Each image was taken while the subject's eyes were open. Each subject's baseline photograph was compared to each post-baseline photograph to ensure consistent placement and lighting. Color standards were imaged prior to each study visit.

Corneometry measurements were taken on each subject's left ocular bone (in line with pupil) to measure the moisture content of the stratum corneum.

Ultrasound measurements were taken on the left side of each subject's face to measure density of the facial skin in the crow's feet area. Measurements were taken with the probe oriented perpendicular to the body axis while the subjects were resting supine on a padded patient table.

A single cutometer measurement was taken on the right side of the subject's face, in line with the corner of the eye and the edge of the nostril, to measure the extensibility of the skin.

All clinical and corneometer measurements and evaluations were taken at baseline, week 1 (visit 2), week 4 (visit 3), week 8 (visit 4) and week 12 (visit 5). Ultrasound and cutometer measurements and evaluations were taken at baseline, week 4 (visit 3), week 8 (visit 4) and week 12 (visit 5). Completed patient's diaries were reviewed for compliance at each visit.

Biostatistics

Mean clinical grading and instrumentation scores at each visit were statistically compared to baseline scores using paired t-test. Changes from baseline were considered significant at the p≦0.05 level. Mean percent change from baseline and incidence of positive responders were calculated for all attributes. Comparisons, based on the average from baseline, were made between the test materials using analysis of variance (ANOVA).

Results

Twenty-nine of thirty subjects successfully completed the study with one subject unable to complete due to personal reasons. Compliance assessments indicated that subjects were following test formulation use instructions.

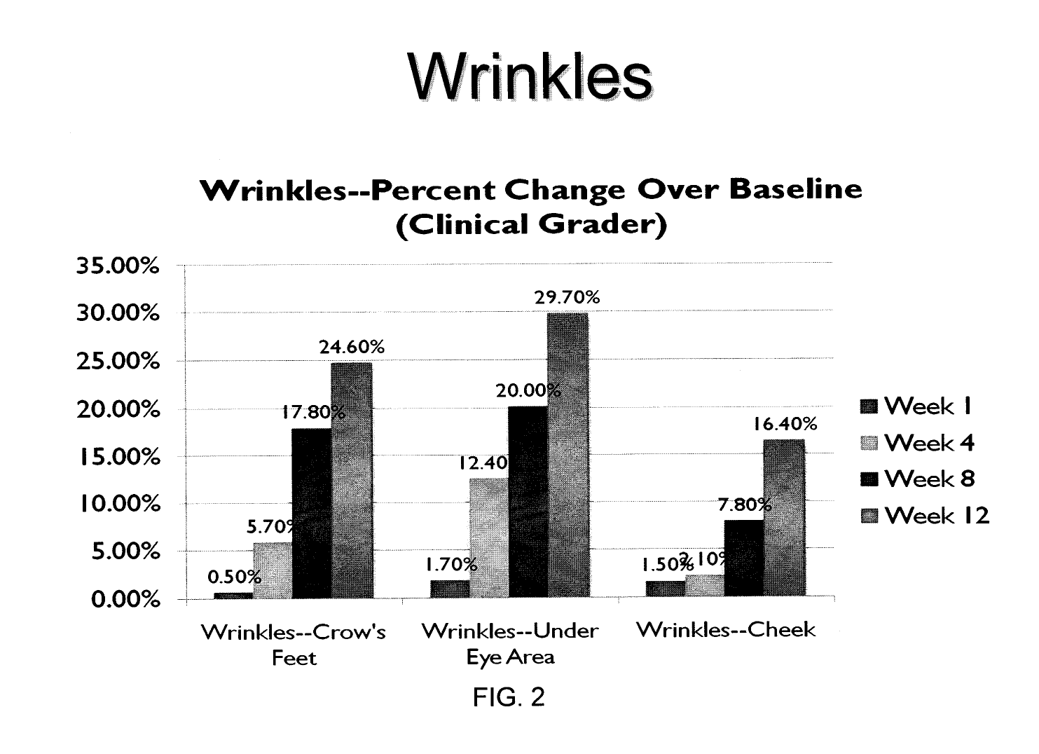

FIG. 2 depicts the result of clinical investigator grading showing a breakdown of the different sub-categories of wrinkles evaluated against baseline at week 1, week 4, week 8 and week 12 time points.

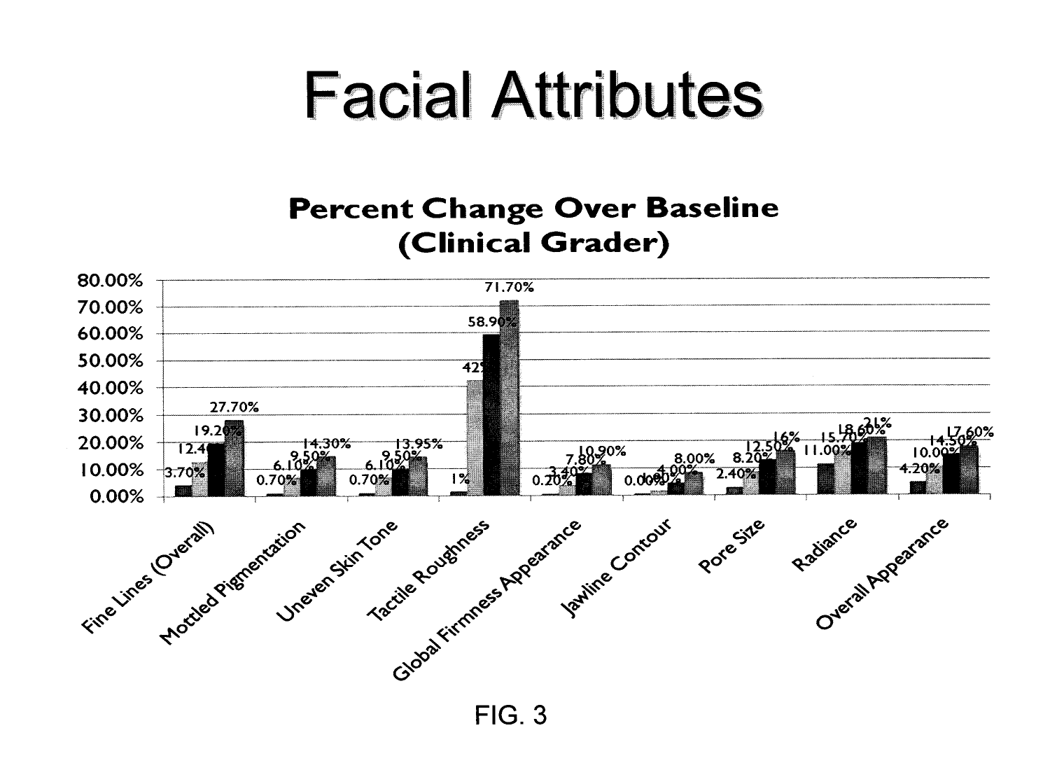

FIG. 3 depicts the result of clinical investigator grading showing change of each investigated parameter (except wrinkles) against baseline at week 1, week 4, week 8 and week 12 time points.

The clinical investigator's facial assessments indicated a statistically significant improvement in facial fine lines, tactile roughness, pore size, radiance and overall appearance at week 1 time point (P≦0.05). All of the benefits continued into weeks 4, 8 and 12. Statistically significant improvement in mottled pigmentation, global firmness, sub-categories of wrinkles and jaw-line contour was recorded at week 4 time point (P≦0.05) and continued through to week 12.

Corneometer measurements indicated statistical improvement in hydration at week 1 time point (P≦0.05). This improvement continued through week 8. Hydration of the stratum corneum was decreased significantly (P≦0.05) at the week 12 time point.

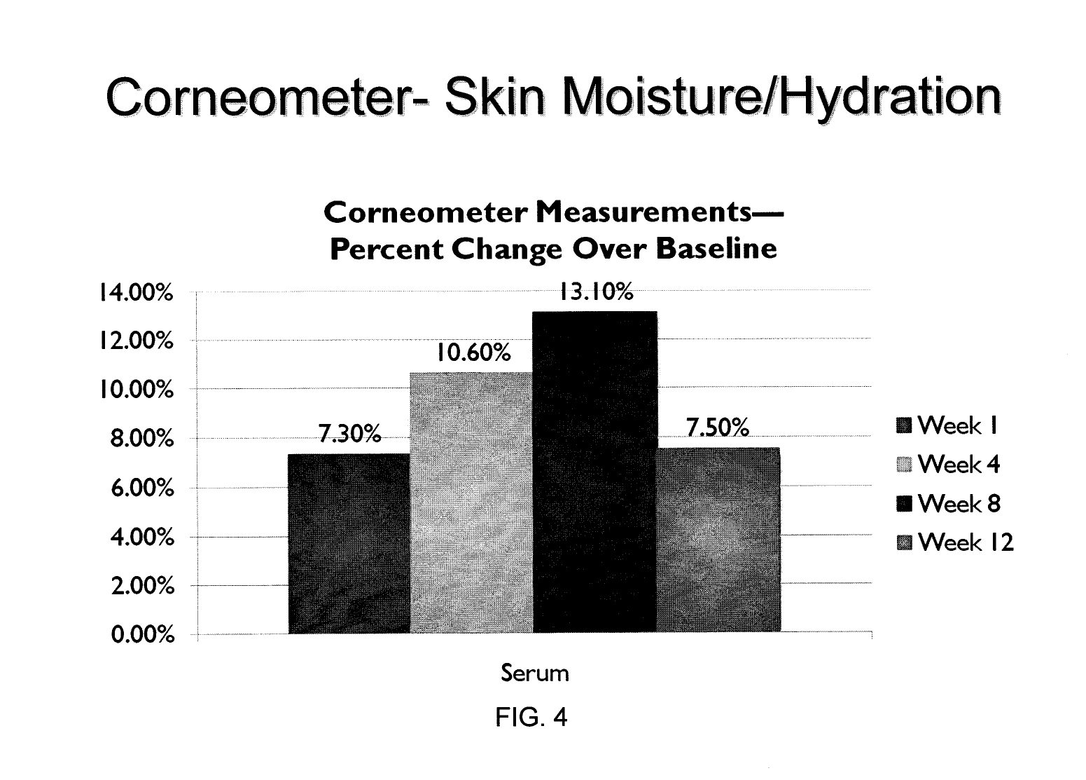

FIG. 4 depicts the result of corneometer grading showing moisture content of the stratum corneum, at week 1, week 4, week 8 and week 12 time points.

Cutometer measurements indicated statistical improvement in extensibility of the skin at week 12 time point (P≦0.05).

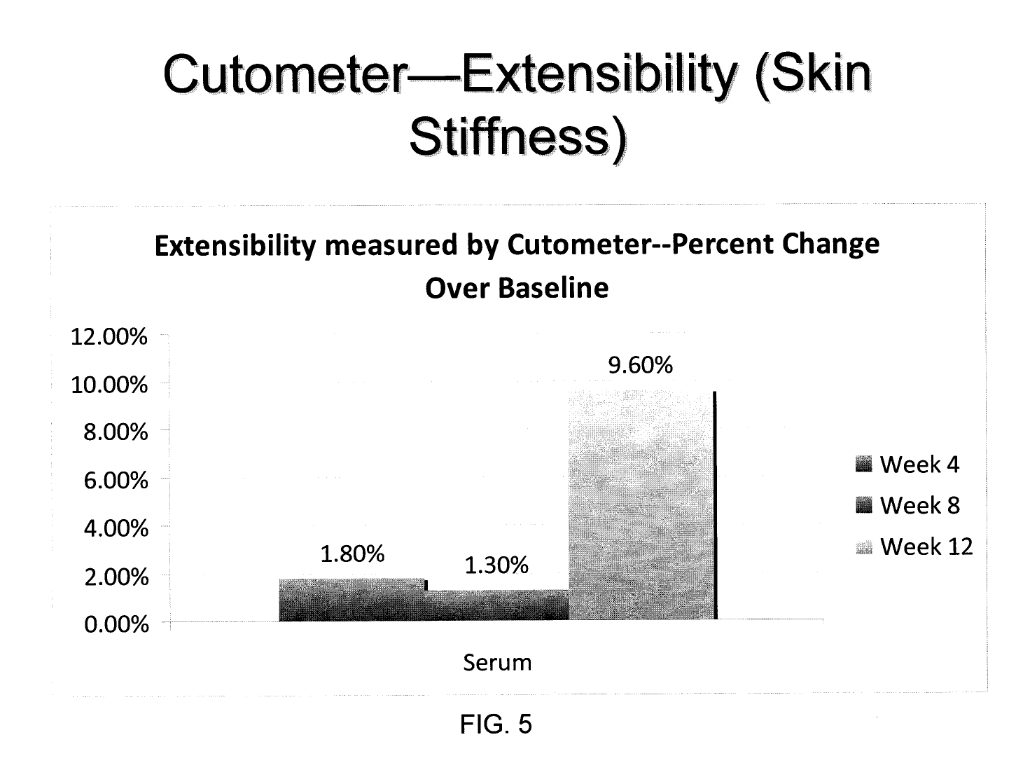

FIG. 5 depicts the results of cutometer readings showing extensibility measurements of the skin, evaluated against baseline at week 4, week 8 and week 12 time points.

Ultrasound measurements indicated statistical improvement in density of the skin at week 4 time point (P≦0.05). This improvement continued through week 12.

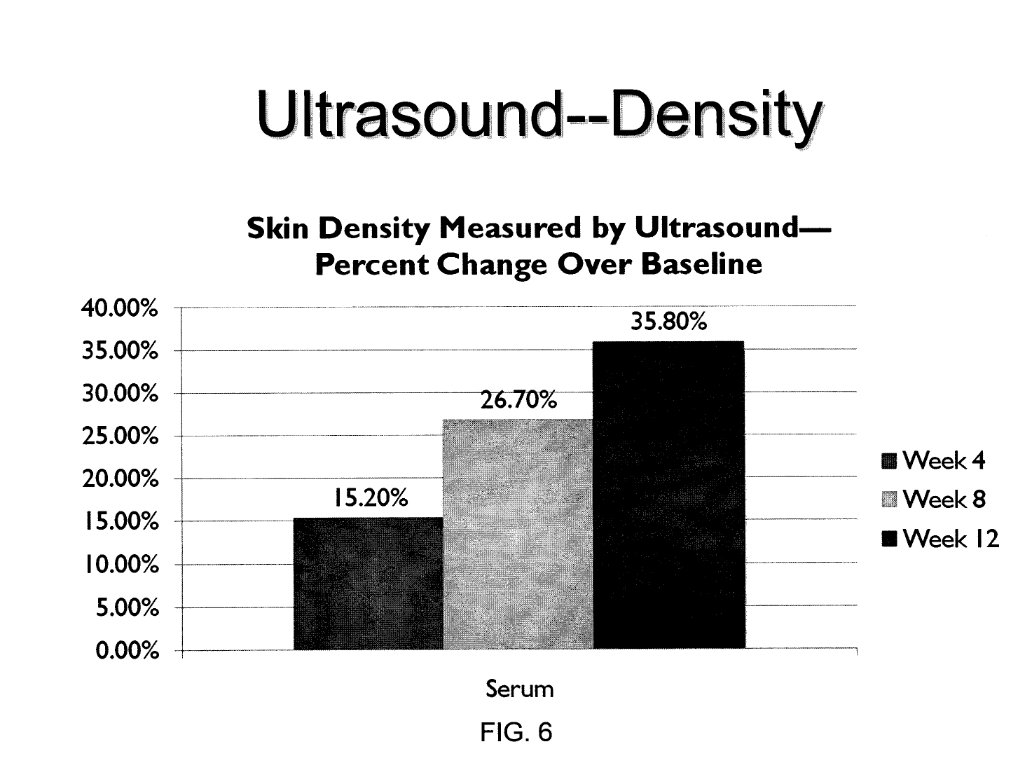

FIG. 6 depicts the result of density evaluation from ultrasound, against baseline at week 4, week 8 and week 12 time points.

No tolerability issues related to erythema, edema and scaling were observed by the investigator. Three subjects reported a slight stinging at week 8 (P≦0.05) and slight itching at week 12 (P≦0.05).

TABLE 8
Mean values of Clinical Grading and Instrumentation

| | | Base- | | | | | | | | | | | | |
|---|---|---|---|---|---|---|---|---|---|---|---|---|---|---|
| | | line | Week 1 | | | Week 4 | | | Week 8 | | | Week 12 | | |
| | | Mean | Mean | | % Change | Mean | | % Change | Mean | | % Change | Mean | | % Change |
| Crows Feet | Wrinkles | 3.29 | 3.21 | | (−0.5%) | 3.00 | [symbol] | (−8.9%) | 2.71 | [symbol] | (−17.8%) | 2.48 | [symbol] | (−24.6%) |
| Under-Eye | Wrinkles | 3.19 | 3.07 | | (−1.7%) | 2.79 | [symbol] | (−12.4%) | 2.55 | [symbol] | (−20.0%) | 2.24 | [symbol] | (−29.7%) |
| Area | | | | | | | | | | | | | | |
| Cheek | Wrinkles | 2.41 | 2.29 | | (−1.5%) | 2.26 | [symbol] | (−6.4%) | 2.12 | [symbol] | (−12.1%) | 2.02 | [symbol] | (−16.4%) |
| Face | Fine Lines | 4.29 | 4.14 | [symbol] | (−3.7%) | 3.76 | [symbol] | (−12.4%) | 3.47 | [symbol] | (−19.2%) | 3.10 | [symbol] | (−27.7%) |
| | (Overall) | | | | | | | | | | | | | |
| | Mottled | 5.05 | 5.05 | | (−0.7%) | 4.74 | [symbol] | (−6.1%) | 4.57 | [symbol] | (−9.5%) | 4.33 | [symbol] | (−14.3%) |
| | Pigmentation | | | | | | | | | | | | | |
| | Uneven Skin | 5.05 | 5.05 | | (−0.7%) | 4.74 | [symbol] | (−6.1%) | 4.57 | [symbol] | (−9.5%) | 4.34 | [symbol] | (−13.9%) |
| | Tone | | | | | | | | | | | | | |
| | Tactile | 3.48 | 2.41 | [symbol] | (−30.7%) | 2.02 | [symbol] | (−42.0%) | 1.43 | [symbol] | (−58.9%) | 0.98 | [symbol] | (−71.7%) |
| | Roughness/ | | | | | | | | | | | | | |
| | Smoothness | | | | | | | | | | | | | |
| | Global | 5.03 | 5.04 | | (0.3%) | 4.86 | [symbol] | (−3.4%) | 4.64 | [symbol] | (−7.8%) | 4.48 | [symbol] | (−10.9%) |
| | Firmness | | | | | | | | | | | | | |
| | Appearance | | | | | | | | | | | | | |
| | Jawline | 5.16 | 5.14 | . | (0.0%) | 5.10 | | (−1.0%) | 4.95 | [symbol] | (−4.0%) | 4.74 | [symbol] | (−8.0%) |
| | Contour | | | | | | | | | | | | | |
| | Pore Size | 4.41 | 4.30 | [symbol] | (−2.4%) | 4.05 | [symbol] | (−8.2%) | 3.86 | [symbol] | (−12.5%) | 3.71 | [symbol] | (−16.0%) |
| | Radiance | 5.81 | 5.20 | [symbol] | (−11.0%) | 4.90 | [symbol] | (−15.7%) | 4.72 | [symbol] | (−18.6%) | 4.59 | [symbol] | (−21.0%) |
| | Overall | 5.47 | 5.21 | [symbol] | (−4.2%) | 4.91 | [symbol] | (−10.0%) | 4.67 | [symbol] | (−14.5%) | 4.50 | [symbol] | (−17.6%) |
| | Appearance | | | | | | | | | | | | | |
| Left Ocular | Corneometer | 56.49 | 60.54 | [symbol] | (7.3%) | 62.49 | [symbol] | (10.6%) | 63.91 | [symbol] | (13.1%) | 52.23 | [symbol] | (−7.5%) |
| Bone | | | | | | | | | | | | | | |

| | | | | | | | | | | | | | |
|---|---|---|---|---|---|---|---|---|---|---|---|---|---|
| Cutometer | Extensibility | 1.15 | N/A | N/A | 1.17 | | (1.8%) | 1.17 | | (1.3%) | 1.26 | [symbol] | (9.6%) |
| Ultrasound | Density | 18.55 | N/A | N/A | 21.38 | [symbol] | (15.2%) | 23.52 | [symbol] | (26.7%) | 25.21 | [symbol] | (35.8%) |

[symbol]

Indicates a statistically significant (p ≦ 0.05) increase compared to Baseline [symbol]
Indicates a statistically significant (p ≦ 0.05) decrease compared to Baseline

All references disclosed herein, whether patent or non-patent, are hereby incorporated by reference as if each was included at its citation, in its entirety.

Although the present disclosure has been described with a certain degree of particularity, it is understood the disclosure has been made by way of example, and changes in detail or structure may be made without departing from the spirit of the disclosure as defined in the appended claims.

Aspects of the disclosure include:

1. A method of testing to identify genes associated with one or more physical attributes of skin aging comprising:
exposing a first sample of human skin tissue to an agent;
determining a first set of expression levels of a plurality of genes in the first sample of human skin;
comparing the first set of expression levels to a second set of expression levels, the second set of expression levels corresponding to expression levels of human skin tissue not exposed to the agent, to identify a first subset of genes having a fold change difference in expression level between the exposed and unexposed samples that meets a first, selected biological relevance level;
selecting from the first subset of genes a second subset of genes, each gene being associated with a biochemical pathway associated with physical appearance of skin aging;
selecting from the second subset of genes, at least one skin attribute subset of genes, each gene in the skin attribute subset being associated with a biochemical pathway relating to the skin attribute that is shown in the comparing step to have been regulated in a more youthful direction for that biochemical pathway and skin attribute;
exposing a second sample of human skin tissue to the agent;
determining the levels of expression for the at least one skin attribute subset of genes in the second sample of human skin tissue using a method for determining expression levels that is different than that used for the first sample of human skin tissue; and
selecting a third subset of genes from the at least one skin attribute subset of genes whose expression levels in the second sample of human skin tissue meet a second, selected biological relevance level and whose direction of regulation conforms to the more youthful direction used in selecting the at least one skin attribute subset of genes.

2. The method of claim 1, wherein the biochemical pathway associated with the physical appearance of skin aging comprises at least one of skin structural protein synthesis, skin structural degradation and maintenance, extracellular matrix assembly, cellular differentiation, skin barrier component synthesis, skin barrier integrity, water regulation, or regulation of melanin production and control.

3. The method of claim 1, wherein the skin attribute for the at least one skin attribute subset of genes is skin structure, skin pigmentation, skin hydration or cell turnover.

4. The method of claim 1, wherein the first, selected biological relevance level is about a two fold difference between the exposed and unexposed samples.

5. The method of claim 1, wherein the human skin tissue comprises skin cells comprising at least one of keratinocytes, fibroblasts, adipocytes, melanocytes or combinations thereof.

6. The method of claim 1, wherein the first set of expression levels of a plurality of genes comprises expression levels for essentially the full human genome.

7. The method of claim 1, wherein the method for determining expression levels that is different than that used for the first sample of human tissue is a method using an RNA quantification metric.

8. The method of claim 1, wherein the step of selecting from the second subset of genes, at least one skin attribute subset of genes, each gene in the skin attribute subset being associated with a biochemical pathway relating to the skin attribute that is shown in the comparing step to have been regulated in a more youthful direction for that biochemical pathway and skin attribute comprising performing this step for a plurality of skin attribute subsets of genes; and
the step selecting a third subset of genes from the at least one skin attribute subset of genes whose expression levels in the second sample of human skin tissue meet a second, selected biological relevance level and whose direction of regulation conforms to the more youthful direction used in selecting the at least one skin attribute subset of genes comprising performing this step for a plurality of skin attribute subsets of genes.

9. The method of claim 8, wherein the plurality of skin attribute subsets of genes are two or more skin attribute subset of genes selected from the group consisting of skin structure, skin pigmentation, skin hydration and cell turnover.

10. The method of claim 1 further comprising determining the levels of expression for additional genes associated with a biochemical pathway associated with skin aging in the second sample of human skin tissue using a method for determining expression levels that is different than that used for the first sample of human tissue; and
selecting for the third subset of genes those genes from the additional genes associated with a biochemical pathway associated with skin aging whose expression levels in the second sample of human skin tissue meet a second, selected biological relevance level and whose direction of regulation conforms to the more youthful direction of regulation of the associated biochemical pathway.

11. A computer based system of testing to identify genes associated with one or more physical attributes of skin aging comprising:
a first instrument for exposing a first sample of human skin tissue to an agent and determining a first set of expression levels of a plurality of genes in the first sample of human skin;
a computer module for comparing the first set of expression levels to a second set of expression levels, the second set of expression levels corresponding to expression levels of human skin tissue not exposed to the agent to identify a first subset of genes having a fold change difference in expression level between the exposed and unexposed samples that meet a first, selected biological relevance level;
a computer module for accessing a stored data set identifying genes, each gene being associated with a biochemical pathway associated with physical appearance of skin aging and for selecting from the first subset a second subset comprising those genes also in the second subset;
a computer module for selecting from the second subset of genes, at least one skin attribute subset of genes, each gene in the skin attribute subset being associated with a biochemical pathway relating to the skin attribute that is shown in the comparing step to have been regulated in a more youthful direction for that biochemical pathway and skin attribute;
a second instrument for exposing a second sample of human skin tissue to the agent and for determining the levels of expression for the at least one skin attribute subset of genes in the second sample of human skin tissue using a method for determining expression levels that is different than that used for the first sample of human tissue; and
a computer module for selecting a third subset of genes from the at least one skin attribute subset of genes whose expression levels in the second sample of human skin tissue meet a second, selected biological relevance level and whose direction of regulation conforms to the more youthful direction used in selecting the at least one skin attribute subset of genes.

12. The system of claim 11, wherein the skin attribute for the at least one skin attribute subset of genes is skin structure, skin pigmentation, skin hydration or cell turnover.

13. The system of claim 11, wherein the first, selected biological relevance level is about a two fold difference between the exposed and unexposed samples.

14. The system of claim 11, wherein the human skin tissue comprises skin cells comprising at least one of keratinocytes, fibroblasts, adipocytes, melanocytes or combinations thereof.

15. The system of claim 11, wherein the first set of expression, levels of a plurality of genes comprises expression levels for essentially the full human genome.

16. The system of claim 11, wherein the second instrument for determining expression levels that is different than that used for the first sample of human tissue is an instrument using an RNA quantification metric.

17. A method of assessing the efficacy of a skin anti-aging agent comprising:
exposing a first sample of human skin tissue to an agent;
determining a first set of expression levels of a plurality of genes in the first sample of human skin;
comparing the first set of expression levels to a second set of expression levels, the second set of expression levels corresponding to expression levels of human skin tissue not exposed to the agent, to identify a first subset of genes having a fold change difference in expression level between the exposed and unexposed samples that meets a first, selected biological relevance level;
selecting from the first subset of genes a second subset of genes, each gene being associated with a biochemical pathway associated with physical appearance of skin aging;
selecting from the second subset of genes, at least one skin attribute subset of genes, each gene in the skin attribute subset being associated with a biochemical pathway relating to the skin attribute that is shown in the comparing step to have been regulated in a more youthful direction for that biochemical pathway and skin attribute;
exposing a second sample of human skin tissue to the agent;
determining the levels of expression for the at least one skin attribute subset of genes in the second sample of human skin tissue using a method for determining expression levels that is different than that used for the first sample of human skin tissue;
selecting a third subset of genes from the at least one skin attribute subset of genes whose expression levels in the second sample of human skin tissue meet a second, selected biological relevance level and whose direction of regulation conforms to the more youthful direction used in selecting the at least one skin attribute subset of genes; and
comparing the third subset of genes to a previously determined third subset of genes for a second agent, thereby showing the efficacy of the skin anti-aging agent.

18. The method of claim 17, wherein the skin attribute for the at least one skin attribute subset of genes is skin structure, skin pigmentation, skin hydration or cell turnover.

19. The method of claim 17, wherein the method for determining expression levels that is different than that used for the first sample of human tissue is a method using an RNA quantification metric.

20. A method of formulating a composition with a plurality of skin anti-aging agents, comprising:
assessing the efficacy of each of two or more skin anti-aging agents by a method comprising:
exposing a first sample of human skin tissue to an agent;
determining a first set of expression levels of a plurality of genes in the first sample of human skin;
comparing the first set of expression levels to a second set of expression levels, the second set of expression levels corresponding to expression levels of human skin tissue not exposed to the agent, to identify a first subset of genes having a fold change difference in expression level between the exposed and unexposed samples that meets a first, selected biological relevance level;
selecting from the first subset of genes a second subset of genes, each gene being associated with a biochemical pathway associated with physical appearance of skin aging;
selecting from the second subset of genes, at least one skin attribute subset of genes, each gene in the skin attribute subset being associated with a biochemical pathway relating to the skin attribute that is shown in the comparing step to have been regulated in a more youthful direction for that biochemical pathway and skin attribute;
exposing a second sample of human skin tissue to the agent;
determining the levels of expression for the at least one skin attribute subset of genes in the second sample of human skin tissue using a method for determining expression levels that is different than that used for the first sample of human skin tissue;
selecting a third subset of genes from the at least one skin attribute subset of genes whose expression levels in the second sample of human skin tissue meet a second, selected biological relevance level and whose direction of regulation conforms to the more youthful direction used in selecting the at least one skin attribute subset of genes; and
comparing the third subset of genes to a previously determined third subset of genes for a second agent, thereby showing the efficacy of the skin anti-aging agent;
selecting from the two or more skin anti-aging agents assessed two or more agents found to have efficacy for at least one skin attribute; and
formulating a composition with such two or more agents found to have efficacy as active ingredients and a pharmaceutically acceptable carrier.

21. The method of claim 20, wherein the skin attribute for the at least one skin attribute subset of genes is skin structure, skin pigmentation, skin hydration or cell turnover and the composition is directed to regulation of the genes in the at least one skin attribute subset of genes in a more youthful direction.

22. The method of claim 20, wherein the method for determining expression levels that is different than that used for the first sample of human tissue is a method using an RNA quantification metric.
